(19) United States
(12) Patent Application Publication
(10) Pub. No.: US 2024/0130962 A1
(43) Pub. Date: Apr. 25, 2024

(54) TRANSDERMAL PENETRANT FORMULATIONS FOR ADMINISTRATION OF MEDICAMENTS

(71) Applicant: Dyve Biosciences, Inc., Camarillo, CA (US)

(72) Inventors: Nathan FITZSIMMONS, Newbury Park, CA (US); Ryan BEAL, Camarillo, CA (US); Charles A. HARBERT, Creede, CO (US); Audrene RICE, Ventura, CA (US); Brandon SAND, Camarillo, CA (US); Parshva KOTHARY, Thousand Oaks, CA (US); Bruce SAND, Westlake Village, CA (US)

(21) Appl. No.: 18/351,192

(22) Filed: Jul. 11, 2023

Related U.S. Application Data
(63) Continuation of application No. PCT/US2022/012330, filed on Jan. 13, 2022.
(60) Provisional application No. 63/137,127, filed on Jan. 13, 2021.
(60) Provisional application No. 63/218,983, filed on Jul. 7, 2021.
(60) Provisional application No. 63/219,280, filed on Jul. 7, 2021.
(60) Provisional application No. 63/227,330, filed on Jul. 29, 2021.
(60) Provisional application No. 63/261,510, filed on Sep. 22, 2021.
(60) Provisional application No. 63/271,014, filed on Oct. 22, 2021.
(60) Provisional application No. 63/287,492, filed on Dec. 8, 2021.

(51) Int. Cl. A61K 9/00; A61K 9/06; A61K 31/196; A61K 31/4015; A61K 31/4035; A61K 31/404; A61K 31/415; A61K 31/4178; A61K 31/4196; A61K 31/436; A61K 31/44; A61K 31/444; A61K 31/4545; A61K 31/4725; A61K 31/506; A61K 31/517; A61K 31/519; A61K 31/522; A61K 31/546; A61K 31/573; A61K 31/575; A61K 31/65; A61K 31/7028; A61K 31/7052; A61K 31/7064; A61K 33/26; A61K 38/07; A61K 38/14; A61K 38/48; A61K 47/10; A61K 47/12; A61K 47/14; A61K 47/24

(52) U.S. Cl. CPC A61K 9/0014; A61K 9/06; A61K 31/196; A61K 31/4015; A61K 31/4035; A61K 31/404; A61K 31/415; A61K 31/4178; A61K 31/4196; A61K 31/436; A61K 31/44; A61K 31/444; A61K 31/4545; A61K 31/4725; A61K 31/506; A61K 31/517; A61K 31/519; A61K 31/522; A61K 31/546; A61K 31/573; A61K 31/575; A61K 31/65; A61K 31/7028; A61K 31/7052; A61K 31/7064; A61K 33/26; A61K 38/07; A61K 38/14; A61K 38/4893; A61K 47/10; A61K 47/12; A61K 47/14; A61K 47/24; C12Y 304/24069

(57) ABSTRACT

The present disclosure relates to a formulations and methods for transdermal delivery of a medicament through the skin of a subject. In aspects, the formulation comprises a therapeutically effective amount of a medicament and a penetrant portion in which the penetrant portion comprises: a phospholipid, a fatty acid ester formed from a low molecular weight alcohol, and a long-chain fatty acids. In some embodiments, the penetrant portion further comprises one or more of a viscosity-improving agent, a penetration enhancer, and an emulsifier.

### CROSS-REFERENCE TO RELATED APPLICATIONS

This application is a continuation of International Application No. PCT/US2022/012330, filed Jan. 13, 2022; and claims the benefit of U.S. Provisional Application No. 63/137,127, filed Jan. 13, 2021; U.S. Provisional Application No. 63/218,983, filed Jul. 7, 2021; U.S. Provisional Application No. 63/219,280, filed Jul. 7, 2021; U.S. Provisional Application No. 63/227,330, filed Jul. 29, 2021; U.S. Provisional Application No. 63/261,510, filed Sep. 22, 2021; U.S. Provisional Application No. 63/271,014, filed Oct. 22, 2021; and U.S. Provisional Application No. 63/287,492, filed Dec. 8, 2021. The entire contents of each of the priority applications are expressly incorporated herein by reference.

### BACKGROUND

Topical administration describes the application of a substance to a surface of the skin. The term is often used to describe the application of a cream, foam, gel, lotion or ointment to the skin or mucous membranes. The high keratinization of skin cells and their dense packing creates, in most cases, a barrier impermeable to penetration. Because of this, most substances are not absorbed through the skin. There is an unmet need for formulations and method for transdermal penetration that work with a variety of medicaments and active agents, which overcome the barrier presented by the stratum corneum as well as the deeper layers of skin, that do so without harsh solvents, and are effective in delivering high molecular weight agents such as peptides, proteins and nucleic acids.

### BRIEF DESCRIPTION OF THE DRAWINGS

FIG. 1 to FIG. 5 are graphs showing plasma pharmacokinetics resulting from topical administration to adult mice of vancomycin-containing formulations.

FIG. 6 to FIG. 11 are graphs showing plasma pharmacokinetics resulting from topical administration to adult mice of neratinib-containing formulations. FIG. 12 is a graph showing plasma pharmacokinetics resulting from oral administration (gavage) to adult mice of a neratinib-containing solution.

FIG. 13 to FIG. 15, FIG. 17, and FIG. 18 are graphs showing plasma pharmacokinetics resulting from topical administration to adult mice of doxycycline-containing formulations. FIG. 16 is a graph showing plasma pharmacokinetics resulting from oral administration (gavage) to adult mice of a doxycycline-containing solution.

FIG. 19 to FIG. 21 are graphs showing plasma pharmacokinetics resulting from topical administration to adult mice of cefepime-containing formulations. FIG. 22 is a graph showing plasma pharmacokinetics resulting from IV administration to adult mice of a cefepime-containing solution.

FIG. 23 to FIG. 25 are graphs showing plasma pharmacokinetics resulting from topical administration to adult mice of eletriptan-containing formulations. FIG. 26 is a graph showing plasma pharmacokinetics resulting from oral administration to adult mice of an eletriptan-containing solution.

### SUMMARY

Aspects of the present disclosure teach certain benefits in construction and use which give rise to the exemplary advantages described below.

The present disclosure relates to systems and methods of transdermal administration of medicaments for a range of benefits.

The present disclosure solves the problems described above by providing transdermal formulations with improved penetration. In at least one embodiment, disclosed herein are transdermal penetrant formulations for transdermal administration of a medicament.

An aspect of the present disclosure is a formulation for transdermal delivery of a medicament through the skin of a subject. The formulation comprises a therapeutically effective amount of a medicament and a penetrant portion. The penetrant portion comprises: a phospholipid, a fatty acid ester formed from a low molecular weight alcohol, and a long-chain fatty acids, and, optionally, one or more of a viscosity-improving agent, a penetration enhancer, and an emulsifier.

In embodiments, the phospholipid is selected from phosphatidylcholine, hydrogenated phosphatidylcholine, phosphatidylserine, phosphatidylethanolamine, phosphatidylinositol, inositol phosphatide, and sphingomyelin. In some cases, the phospholipid is phosphatidylcholine.

In some embodiments, the penetrant portion comprises two or more phospholipids.

In various embodiments, the phospholipid is in an amount from about 3% to about 15% w/w of the formulation.

In certain embodiments, the low molecular weight alcohol is selected from isopropanol, methanol, ethanol, butanol, glycerol, cetyl alcohol.

In embodiments, the low molecular weight alcohol is isopropanol.

In some embodiments, the fatty acid ester is selected from isopropyl palmitate, isopropyl myristate, isopropyl linoleate, isopropyl oleate, ethyl laurate, and ethyl myristate. In some cases, the fatty acid ester is isopropyl palmitate.

In various embodiments, the penetrant portion comprises two or more fatty acid esters.

In certain embodiments, the fatty acid ester is in an amount from about 5% to about 20% w/w of the formulation.

In embodiments, the long-chain fatty acid is selected from a linoleic, oleic, stearic acid, linolenic, palmitic, arachidonic, palmitoleic, myristic, eicosenoic, benehic, euricic, and lignoceric acid. In some cases, the long-chain fatty acid is linoleic acid. In other cases, the long-chain fatty acid is oleic acid. In further cases, the long-chain fatty acid is stearic acid. In some cases, the long-chain fatty acid is obtained from safflower oil or almond oil.

In some embodiments, the long-chain fatty acid is in an amount from about 0.1% to about 10% w/w of the formulation.

In various embodiments, the penetrant portion comprises two or more long-chain fatty acids.

In certain embodiments, the penetrant portion comprises a viscosity-improving agent.

In embodiments, the viscosity-improving agent is a poloxamer. In some cases, the poloxamer is selected from poloxamer 407, poloxamer 188, poloxamer 184, and poloxamer 124.

In some embodiments, the viscosity-improving agent is a surfactant. In some cases, the surfactant is selected from sodium lauryl sulfate (sodium dodecyl sulfate); polyoxyethylated castor oil derivatives such as HCO-60 surfactant; nonoxynol; octoxynol; phenylsulfonate; poloxamers such as Pluronic® F68, Pluronic® F127, and Pluronic® L62; polyoleates; Rewopal® HVIO, sodium laurate, sodium oleate; sorbitan dilaurate; sorbitan dioleate; sorbitan monolaurate such as Span® 20; sorbitan monooleates; sorbitan trilaurate; sorbitan trioleate; sorbitan monopalmitate such as Span® 40; sorbitan stearate such as Span® 85; polyethylene glycol nonylphenyl ether such as Synperonic® NP; p-(1,1,3,3-tetramethylbutyl)-phenyl ether such as Triton™ X-100; and polysorbates such as polyoxyethylene (20) sorbitan monolaurate such as Tween® 20, polysorbate 40 (polyoxyethylene (20) sorbitan monopalmitate) such as Tween® 40, polysorbate 60 (polyoxyethylene (20) sorbitan monostearate) such as Tween® 60, polysorbate 80 (polyoxyethylene (20) sorbitan monooleate) such as Tween® 80, and polyoxyethylenesorbitan trioleate such as Tween® 85. In some cases, the surfactant is sodium lauryl sulfate.

In embodiments, the penetrant portion comprises two or more viscosity-improving agents.

In certain embodiments, the viscosity-improving agent is in an amount from about 5% to about 20% w/w of the formulation.

In embodiments, the penetrant portion comprises a penetration enhancer. In some embodiments, the penetration enhancer is an alcohol or a terpene. In some cases, the penetration enhancer as an alcohol is selected from benzyl alcohol, ethanol, propylene glycol, and polyethylene glycol. In various cases, the penetration enhancer is benzyl alcohol. In some cases, the penetration enhancer as a terpene is selected from limonene, menthol, borneol, and camphor. In various embodiments, the penetration enhancer further acts as a preservative.

In some embodiments, the penetrant portion comprises two or more penetration enhancers.

In various embodiments, the penetration enhancer is in an amount from about 0.5% to about 5% w/w of the formulation.

In certain embodiments, the penetrant portion comprises at least one penetration enhancer and at least one viscosity-improving agent.

In embodiments, the penetrant portion comprises an emulsifier.

In some embodiments, the emulsifier is selected from polyglyceryl-4-laurate, polyglyceryl-4-oleate, span 60, cetyl alcohol, and polyglyceryl-3-oleate.

In various embodiments, the penetrant portion comprises two or more penetration enhancers.

In certain embodiments, the emulsifier is in an amount from about 0.5 to about 10% w/w of the formulation.

In embodiments, the penetrant portion comprises at least one emulsifier and at least one viscosity-improving agent.

In some embodiments, the penetrant portion comprises at least one emulsifier and at least one penetration enhancer.

In various embodiments, the penetrant portion comprises at least one emulsifier, at least one viscosity-improving agent, and at least one penetration enhancer.

Another aspect of the present disclosure is a formulation for transdermal delivery of a medicament through the skin of a subject. The formulation comprises a therapeutically effective amount of a medicament and a penetrant portion in which the penetrant portion comprises: a phospholipid, a fatty acid ester formed from a low molecular weight alcohol, and a long-chain fatty acids. In this aspect, phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil.

A further aspect of the present disclosure is a formulation for transdermal delivery of a medicament through the skin of a subject. The formulation comprises a therapeutically effective amount of a medicament and a penetrant portion in which the penetrant portion comprises: a phospholipid, a fatty acid ester formed from a low molecular weight alcohol, and a long-chain fatty acids, and one or more of a viscosity-improving agent, a penetration enhancer, and an emulsifier. In this aspect, phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil; polyglyceryl-4-laurate, polyglyceryl-4-oleate, span 60, cetyl alcohol, or polyglyceryl-3-oleate is the penetration enhancer; a poloxamer (e.g., poloxamer 407, poloxamer 188, poloxamer 184, and poloxamer 124) or sodium lauryl sulfate is the viscosity-improving agent; benzyl alcohol, ethanol, propylene glycol, polyethylene glycol, limonene, menthol, borneol, or camphor is the penetration enhancer.

In certain embodiments, the penetrant portion is in an amount from about 70% to about 98% w/w of the formulation.

In embodiments, the penetrant portion comprises water.

In some embodiments, the penetrant portion comprises water in an amount from about 50% to about 80% w/w of the formulation.

In various embodiments, the formulation comprises a phospholipid, an emollient/moisturizer, a fatty acid, an alcohol, an oil, a surfactant, water, and a medicament.

An additional aspect of the present disclosure is a formulation for transdermal delivery of a medicament through the skin of a subject. The formulation comprises a phospholipid in an amount from about 5% to about 15% w/w of the formulation; an emollient/moisturizer in an amount from about 10% to about 20% w/w of the formulation; a fatty acid in an amount from about 0.5% to about 2% w/w of the formulation; an alcohol in an amount from about 0.5% to about 2% w/w of the formulation; an oil in an amount from about 1% to about 5% w/w of the formulation; a surfactant in an amount from about 0.5% to about 2% w/w of the formulation; water in an amount from about 30% to about 80% w/w of the formulation; and a therapeutically effective amount of a medicament in an amount from about 0.001% to about 30% w/w of the formulation. In some cases, the medicament is in an amount from about 0.001% to about 0.01% w/w of the formulation. In other cases, the medicament is in an amount from about 0.011% to about 0.1% w/w of the formulation. In various cases, the medicament is in an amount from about 0.11% to about 1.0% w/w of the formulation. In further cases, the medicament is in an amount from about 1% to about 10% w/w of the formulation. In additional cases, the medicament is in an amount from about 11% to about 20% w/w of the formulation. In alternate cases, the medicament is in an amount from about 21% to about 30% w/w of the formulation.

In an aspect, the present disclosure provides a formulation for transdermal delivery of a medicament through the skin of a subject. The formulation comprises phosphatidylcholine in an amount of about 7.64% w/w of the formulation; isopropyl palmitate in an amount of about 13.30% w/w of the formulation; stearic acid in an amount of about 0.62% w/w of the formulation; benzyl alcohol in an amount of about 1.39% w/w of the formulation; safflower oil in an amount of about 2.93% w/w of the formulation; oleic acid in an amount of about 0.97% w/w of the formulation; polyglyceryl-4 laurate in an amount of about 1.06% w/w of the formulation; deionized water in an amount of about 60.84% w/w of the formulation; poloxamer 407 in an amount of about 9.25% w/w of the formulation; and a therapeutically effective amount of a medicament in an amount of about 2% w/w of the formulation. In some cases, rather than the medicament being in an amount of about 2% w/w of the formulation, the amount of the medicament is less than about 2% and the amount of water is increased proportionally. In other cases, rather than the medicament being in an amount of about 2% w/w of the formulation, the amount of the medicament is greater than about 2% and the amount of water is decreased proportionally. In various cases, the amount of the medicament is less than about 30%.

In another aspect, the present disclosure provides a formulation for transdermal delivery of a medicament through the skin of a subject. The formulation comprises phosphatidylcholine in an amount of about 7.66% w/w of the formulation; isopropyl palmitate in an amount of about 13.34% w/w of the formulation; benzyl alcohol in an amount of about 1.39% w/w of the formulation; stearic acid in an amount of about 0.68% w/w of the formulation; carthamus tinctorius (safflower) oil in an amount of about 2.79% w/w of the formulation; polyglyceryl-4 laurate in an amount of about 1.07% w/w of the formulation; oleic acid in an amount of about 1.06% w/w of the formulation; deionized water in an amount of about 61.73% w/w of the formulation; poloxamer 407 in an amount of about 9.28% w/w of the formulation; and a therapeutically effective amount of a medicament in an amount of about 1.00% w/w of the formulation. In some case, rather than the medicament being in an amount of about 1% w/w of the formulation, the amount of the medicament is less than about 1% and the amount of water is increased proportionally. In other cases, rather than the medicament being in an amount of about 1% w/w of the formulation, the amount of the medicament is greater than about 1% and the amount of water is decreased proportionally. In various cases, the amount of the medicament is less than about 30%.

In a further aspect, the present disclosure provides a formulation for transdermal delivery of a medicament through the skin of a subject. The formulation comprises a therapeutically effective amount of a medicament and a penetrant portion in which the penetrant portion comprises: phosphatidylcholine in an amount from about 3% to about 15% w/w of the formulation; isopropyl palmitate in an amount from about 5% to about 20% w/w of the formulation; stearic acid in an amount from about 0.1% to about 10% w/w of the formulation; benzyl alcohol in an amount from about 0.5% to about 5% w/w of the formulation; polyglyceryl-4 laurate in an amount from about 0.5% to about 10% w/w of the formulation; and poloxamer 407 in an amount from about 5% to about 20% w/w of the formulation.

In certain embodiments, the formulation has a pH from about 7 to about 10.5.

In embodiments, the formulation has a pH from about 9 to about 11.

In embodiments, the transdermal formulations of the present disclosure deliver medicaments of various sizes. As examples, the medicaments having a molecular weight of less than about 500 Da, e.g., Nicotine (162.2 Da), Diphenhydramine Hydrochloride (291.8 Da), and Hydrocortisone (362.5 Da); medicaments having a molecular weight from about 500 Da to about 1000 Da, e.g., Sildenafil Citrate (666.7 Da), Neratinib (557 Da), and Doxycycline hyclate (512.9 Da); and medicaments having a molecular weight greater than about 1000 Da, e.g., Cyclosporine A (1202.6 Da), Vancomycin Hydrochloride (1485.70 Da), and RBD protein (10,000+Da).

In various embodiments, the medicament is at least one of (3S,4S)-8-(6-amino-5-((2-amino-3-chloropyridin-4-yl)thio)pyrazin-2-yl)-3-methyl-2-oxa-8-azaspiro[4.5]decan-4-amine; (S)-3-amino-6-methoxy-N-(3,3,3-trifluoro-2-hydroxy-2-methylpropyl)-5-(trifluoromethyl)picolinamide; 2-fluoro-N-methyl-4-[7-quinolin-6-yl-methyl)-imidazo[1,2-B][1,2,4]triazin-2yl]benzamide; 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide; 4-(7-Hydroxy-2-isopropyl-4-oxo-4H-quinazolin-3-yl)-benzonitrile; Abacavir; Abiraterone Acetate; abiratone acetate; abobotulinum toxin A; Acalabrutinib; Acarbose; Acetaminophen; Acetazolamide; Acetylsalicylic Acid; acitretin; acyclovir; adalimumab; adapalene; adapalene; Afoxolaner; agalsidase beta; Albendazole; Alectinib; Alectinib; alendronate sodium; alglucosidase alfa; alitretinoin; Alogliptin; alpelisib; Aluminum Hydroxide; Ambrisentan; Amiodarone; Amitriptyline; Amlodipine; Amlodipine; Amlodipine besylate; amlodopine besylate; amorolfine hydrochloride; Amoxicillin; Amphotericin B; Amphotericin B liposome; ampicillin; Amprenavir; antihemophilic factor, Fc fusion protein; apalutamide; Apatinib; apixaban; apremilast; Aprepitant; Aripiprazole; Artemether; aspirin; Atazanavir; atenolol; Atomoxetine; Atorvastatin; Atorvastatin; Avapritinib; Axitinib (Inlyta); Azacitidine; Azathioprine; azelaic acid; Azilsartan medoxomil; azithromycin; baclofen; baricitinib; basiliximab; Batimastat (BB-94); becaplermin; Belumosudil; Bendamustine; benzoyl peroxide; benzoyl peroxide; betamethasone dipropionate; betamethasone valerate; bexarotene; Bictegravir; Bimatoprost; binimetinib (Mektovi); Bortezomib; Bortezomib (Velcade); Bosentan; Bosutinib (Bosulif); botulinum toxin A; branched chain amino acids; Brexpiprazole; Brimonidine; brimonidine tartrate; brodalumab; buproprion; Cabazitaxel; Cabozantinib; Cabozantinib (Cometriz); Calcifediol; calcipotriene; Calcipotriene; Calcipotriol; calcitonin; calcitriol; calcium carbonate; Canagliflozin; canakinumab; Capecitabine; Capmatinib; capsaicin; Carbamazepine; Carbidopa; Carfilzomib; Carfilzomib (Kyprolis); Cariprazine; carvedilol; CD-12681; CEE-321; cefazolin; cefepime; Cefoperazone; cefotaxime sodium; celecoxib; celecoxib; cemiplimab; Ceritinib; Chlorothiazide; Chlorpromazine; Chlorthalidone; cinacalcet; ciprofloxacin; Cisapride; citalopram; citric acid; clarithromycin; Clavulanate; Clindamycin phosphate; Clobazam; clobetasol propionate; Clofazimine; clomiphene; clonidine; Clopidogrel; clopidogrel bisulfate; cobicistat; Cobicistat; Colchocine; Colistin; Corticotropin; crisaborole; crizanlizumab; Crizotinib (Xalkori); cyclobenzaprine; cyclosporine; Dabigatran Etexilate; Dabrafenib; Dabrafenib; dabrafenib; dabrafenib; dacomitinib (Vizimpro); dalbavancin; dalfopristin; Danazol; Dapagliflozin; Dapsone; daptomycin; Darunavir; Dasatinib; decitabine; defatted Peanut (Arachis hypogaea) Flour; deferasirox; delgocitinib; deoxycholic acid; Desipramine; desonide; Dexlansoprazole; Dexmethylphenidate; dextroamphetamine/amphetamine salts; Diclofenac; diclofenac sodium; diflucortolone; Diflucortolone; Difunisal; Digoxin; Diloxanide; Dimethyl Fumarate; diphenhydramine hydrochloride; diphtheria vaccine; docetaxel; Dolutegravir; Dolutegravir; Donepezil; Doxercalciferol; doxycycline; doxycycline ER; Doxycycline Hyclate (B); Doxycycline Monohydrate (A); Dronabinol; Dronedarone; Drospirenone; Dukoral/ShanChol cholera vaccine; Duloxetine; dupilumab; durvalumab; Dutasteride; Duvelisib; ecallantide; Edoxaban; Efavirenz; Efavirenz; Elafibranor; elagolix sodium; Elbasvir; Eletriptan; Eltrombopag; Elvitegravir; Empagliflozin; Emtricitabine; emtricitabine; Emtricitabine; Emtricitabine; Emtricitabine; Emtricitabine; Emtricitabine; Emtricitabine; encorafenib (Braftovi); enfuviritide; enoxaparin sodium; Entecavir; entrectinib; Enzalutamide; Epinephrine; erdafitinib; erenumab; Eribulin; Erlotinib; Erlotinib HCl (Tarceva); Ertapenem; Erythromycin; escitalopram; esketamine (ketamine); Esomeprazole; esopremazole; estradiol; Estrogens—conjugated; etanercept; Ethinyl Estradiol; Ethinyl Estradiol; Ethinyl Estradiol; Etonogestrel; Etonogestrel; Etravirine; etretinate; Everolimus; Everolimus (Afinitor); Everolimus (Votubia, Zortress/Certican); Ezetimibe; Ezetimibe; Ezetimibe; Famotidine; fasinumab; Febuxostat; Fedratinib; Fenofibrate; Ferric Carboxymaltose; fexofenadine hydrochloride; filgotinib; filgrastim; fingolimod; fluocinolone acetonide; fluocinolone acetonide; fluoxetine; Flurbiprofen; Folic acid; Forskolin; Fostamatinib; Fulvestrant; furosemide; fusidic acid; fusidic acid; gabapentin; Ganetespib; Gefitinib (Iressa); Gilteritinib; glasdegib (Daurismo) or glasdegib maleate; Glecaprevir; Glibenclamide; Glimepiride; glipizide; glucagon; glucosamine; glutaric anhydride; glycopyrrolate; Goserelin; goserelin LA; granisetron; Grazoprevir; Griseofulvin; halobetasol propionate; Haloperidol; heparin sodium; heplisav-B vaccine; hyaluronic acid; Hydrochlorothiazide; Hydrochlorothiazide; Hydrochlorothiazide; Hydrochlorothiazide; hydrocortisone; hydrocortisone acetate; hydroquinone; hydroxychloroquine; Hydroxyprogesterone; Ibrutinib; Ibrutinib (Imbruvica); ibuprofen; icatibant acetate; Idelalisib; Imatinib; Imatinib (Gleevec); imatinib mesylate; imiglucerase; inclisiran; Indinavir; Indomethacin; infliximab; ingenol mebutate; inotersen; insulin A; insuline glargine; interferon beta-Ib; Iopanoic acid; Irbesartan; isoconazole; isoconazole; Isotretinoin; Itraconazole; Ivacaftor; Ivacaftor; ivermectin; Ixazomib; ketoconazole; ketones; ketoprofen; Keyruvia; Lacosamide; lamictal; Lamivudine; Lansoprazole; Lapatinib; Lapatinib (Tykerb); larotrectinib; Ledipasvir; leflunomide; Lenalidomide; Lenvatinib; LEO-138559; LEO-152020; Leuprolide; Levetiracetam; levodopa; Levodopa/Benserazide; Levonorgestrel; Levothyroxine; Linagliptin; linezolid; lisinopril; L-lysine free base; lofexidine; Lopinavir; Loratadine; lorlatinib (Lorviqua); Losartan; Lovastatin; Lubiprostone; Lumacaftor/Ivacaftor; Lumefantrine; Lurasidone; Luspatercept; lymecycline; Macitentan; magnesium; magnesium lactate; Marimastat (BB-2516); Marizomib (NPI-0052); Mebendazole; Mefloquine; melatonin; meloxicam; memantine hydrochloride; meningococcal[serotype b] vaccine; Mesalazine; Metformin; Metformin; metformin; Metformin; methotrexate; methyl aminolevulinate hydrochloride; Methylphenidate; methylprednisolone; Metoprolol succinate; metronidazole; Midostaurin; minoxidil; Mirabegron; mizolastine MR; Montelukast; Mycophenolate Mofetil; N-(3-(2-(2-hydroxyethoxy)-6-morpholinopyridin-4-yl)-4-methylphenyl)-2-(trifluoromethyl)-isonicotinamide; N-(3-(6-Amino-5-(2-(N-methylacrylamido)ethoxy)pyrimidin-4-yl)-5-fluoro-2-methylphenyl)-4-cyclopropyl-2-fluorobenzamide; N-[4-(Chlorodifluoromethoxy)phenyl]-6-[(3R)-3-hydroxpyrrolidin-1-yl]-5-(1H-pyrazol-5-yl)-pyridine-3-carboxamide hydrochloride; Nalidixic acid (Quinolone antibiotic); naloxone; Naproxen; Navitoclax (ABT-263); Nebivolol; Nelfinavir; nemolizumab; neomycin; Neovastat (AE-941); Neratinib—Free Base; Neratinib (Nerlynx); Neratinib Maleate; Nevirapine; Niclosamide; Nifedipine; nilotinib; Nilotinib; Nilotinib (Tasigna); Nintedanib; niratimib; Nitric Oxide; Nitrofurantoin; Norethindrone Acetate; nusinersen; NVP-AUY922; Nystatin; Obatoclax (GX15-070); octreotide acetate; ofatumumab; Ofloxacin; Olanzapine; olaparib; Olmesartan Medoxomil; olumacostat; omalizumab; Oseltamivir; Osimertinib; osimertinib mesylate; Oxaprozin; Ozanimod; Paclitaxel; Paclitaxel Protein Bound; Palbociclib (Ibrance); Paliperidone Palmitate; Pantoprazole; parathyroid hormone; Paricalcitol; patisiran; pazopanib; Pazopanib (Votrient); pazopanib hydrochloride; Peficitinib; pegfilgrastim; Pembrolizumab (Keytruda); Pemetrexed; Pemigatinib; Perifosine; Pexidartinib; Phenazopyridine; Phenytoin; Pibrentasvir; Piperacillin; Pirfenidone; Piroxicam; Pomalidomide; Ponatinib; Ponatinib (lclusig); Posaconazole; Pralsetinib; pravastatin; prednicarbate; prednisone; pregabalin; pregabalin; Prinomastat (AG-3340); Progesterone; propanolol; protamine sulfate; psilocybin; Pyrantel; Pyrimethamine; Quetiapine; Quinupristin; Raloxifene; Raltegravir; ranibizumab; ranitidine; Ranolazine; Rebimastat (BMS-275291); Regorafenib; Regorafenib (Stivarga); relugolix (Orgovyx); remibrutinib; resiquimod; retinal; Retinol; ribociclib; ribociclib succinate; Rifampin; Rilpivirine; Rilpivirine; Rilpivirine; Rimegepant; Riociguat; Risdiplam; Risperidone; Ritonavir; Rivaroxaban; Rosuvastatin; rosuvastatin calcium; Rotigotine; roxadustat; rucaparib camsylate (Rubraca); ruxolitinib; Ruxolitinib (Jafaki); Sacubitril, valsartan; salicylic acid; Sapropterin; Saquinavir; sarilumab; Saxagliptin, Metformin; secukinumab; Selexipag; selinexor; Selpercatinib; selpercatinib (LOXO-292); Selumetinib; semaglutide; sertraline; Sevoflurane; sildenafil; Sildenafil Citrate; Simvastatin; siponimod; Siremadlin; Sirolimus; Sitagliptin; Sitagliptin phosphate monohydrate; Sodium bicarbonate or sodium carbonate; sodium deoxycholate; sodium nitrate; Sofosbuvir; Sofosbuvir; Sofosbuvir; Solifenacin; somatropin; sonidegib phosphate; Sorafenib; Sorafenib (Nexavar); spartalizumab; Spironolactone; sufentanil citrate; Sugammadex; sulbactam; Sulbactam; Sulfadiazine; Sulfamethoxazole; sulfasalazine; sumatriptan; Sunitinib; Sunitinib; Sunitinib (sutent); sunitinib malate (Sutent); Tacrolimus; TAF; TAF; TAF; TAF; talazoparib—Talzenna); Talinolol; Tamoxifen; Tamsulosin; tazarotene; tazobactam; TDF; TDF; Temsirolimus (CCl-779, Torisel); Teneligliptin; tenofovir alafenamide; Tenofovir Disoproxil Fumarate; Tepotinib; terbinafine; Terfenadine; Teriflunomide; Testosterone; Tezacaftor; THC; ticagrelor; tigecycline; Timolol; timolol maleate; Tipranavir; tisagenlecleucel; Tisagenlecleucel (Kymriah); Tivozanib; tofacitinib citrate; Tolvaptan; tralokinumab; tramadol; Trametinib; Trametinib; trametinib; Trametinib; Trametinib (Mekinist); tranexamic acid; Travoprost; trazodone; tretinoin; tretinoin; triancinolone; Triclabendazole; trifarotene; Trimethoprim; Triptorelin; tris; Tucatinib; ubrogepant; Umbralisib; upadacitinib; valproic acid; Valsartan; valsartan; Valsartan; valsartan; vancomycin; Vancomycin Hydrochloride (A); Vancomycin Hydrochloride (B); Vandetanib; Vandetenib (Caprelsa); Varenicline; Vasopressin; Velpatasvir; Velpatasvir; Vemurafenib; Venetoclax; Verapamil hydrochloride; Vilazodone; Vildagliptin; vismodegib; vitamin B6; vitamin D; Vonoprazan; Voriconazole; Vortioxetine; Voxilaprevir; Warfarin Sodium; Zanubrutinib; Zinc; zoledronic acid; and Zolpidem.

In certain embodiments, a formulation further comprises an at least second medicament. In some cases, the first and the at least second medicament are selected from the group consisting of Abacavir and Dolutegravir and Lamivudine; adapalene and benzoyl peroxide; Amlodipine and Valsartan; amlodopine besylate and valsartan; Amoxicillin and Clavulanate; ampicillin and sulbactam; Artemether and Lumefantrine; betamethasone valerate and fusidic acid; Bictegravir and Emtricitabine and TAF; Bimatoprost and timolol maleate; Brimonidine and Timolol; Calcipotriene and betamethasone dipropionate; Canagliflozin and Metformin; Carbidopa and levodopa; Cefoperazone and Sulbactam; Dabrafenib and Trametinib; Darunavir and cobicistat and emtricitabine and tenofovir alafenamide; Diflucortolone and isoconazole; Drospirenone and Ethinyl Estradiol; Efavirenz and Emtricitabine and TDF; Elbasvir and Grazoprevir; Elvitegravir and Cobicistat and Emtricitabine and TAF; Emtricitabine and Rilpivirine and TAF; Emtricitabine and TAF; Emtricitabine and Tenofovir Disoproxil Fumarate; Ethinyl Estradiol and Etonogestrel; Ezetimibe and Atorvastatin; Ezetimibe and Simvastatin; fluocinolone acetonide and hydroquinone and tretinoin; fusidic acid and hydrocortisone acetate; Glecaprevir and Pibrentasvir; Hydrochlorothiazide and Valsartan; Irbesartan and Hydrochlorothiazide; Ledipasvir and Sofosbuvir; Linagliptin and Metformin; Losartan and Hydrochlorothiazide; Metformin and Sitagliptin; Norethindrone Acetate and Ethinyl Estradiol; Piperacillin and tazobactam; pregabalin and celecoxib; Quinupristin and dalfopristin; Rilpivirine and Emtricitabine and TDF; Sofosbuvir and Velpatasvir; Sofosbuvir and Velpatasvir and Voxilaprevir; Tezacaftor and Ivacaftor; trametinib and dabrafenib; Trametinib and dabrafenib; and Zinc and magnesium and vitamin B6.

In embodiments, transdermal delivery provides systemic administration of the medicament.

In yet another aspect, the present disclosure provides a method for transdermally delivering at least one medicament. The method comprises a step of applying to the skin of a subject an effective amount of any herein disclosed formulation.

An aspect of the present disclosure is a method for treating a disease or disorder or reducing a symptom thereof. The method comprises a steps of administering to a subject in need thereof any herein disclosed transdermal formulation and administering to the subject in need thereof a composition comprising an one or more medicaments selected from Table 1. In some case, the transdermal formulation is administered before, contemporary with, or after the composition is administered. In various cases, the amount of the one or more medicaments is the effective dose of the medicament as described in Table 1. In some cases, the composition is administered by the standard route for the medicament. In some cases, the standard route is oral, topical, enteral, parenteral, by intravenous injection or infusion, by intraperitoneal injection, by intramuscular injection, or by subcutaneous injection. In various cases, the composition is a liquid, a suspension, a gel, a geltab, a semisolid, a tablet, a sachet, a lozenge, a pill, or a capsule.

Another aspect of the present disclosure is a use of any herein disclosed transdermal formulation in a method for treating a disease or disorder or reducing a symptom thereof.

A further aspect of the present disclosure is a method for manufacturing a medicament for treating a disease or disorder or reducing a symptom thereof comprising combining a penetrant portion as recited in any herein disclosed formulation with one or more medicaments recited in Table 1.

Any aspect or embodiment described herein can be combined with any other aspect or embodiment as disclosed herein.

### DETAILED DESCRIPTION

### Introduction

The term transdermal administration refers to applying a substance onto the skin so that it is absorbed into the body for local or systemic distribution. A transdermal solution (e.g., cream, ointment, or lotion) or transdermal patch is typically placed on one's skin. The solution or patch includes a medicament that is released into the skin. As the layers of skin absorb the solution, the medicament is absorbed via the blood vessels into the bloodstream. From there, the substance can be circulated through the body.

There are obvious advantages to transdermal administration of medicaments. The consumer does not have to schedule and remember to consume doses of pills. Further, transdermal administration is not affected by stomach or digestive issues. Administration across the skin enables drugs to avoid degradation in the gastrointestinal tract or liver. Transdermal delivery is therefore of particular interest for molecules with limited systemic bioavailabilities and short half-lives. Drugs that are absorbed slowly can be more effective. With a transdermal patch or cream, a medicament can be released in small quantities over a long period of time.

Transdermal administration can be effective in administering hydrophobic chemicals such as steroid hormones. For example, transdermal patches are a common means of administering steroidal drugs for birth control, hormone replacement therapy and prevention of motion sickness. Common medicaments that can be administered by transdermal patches include pain relievers, nicotine, hormones, and drugs to treat angina and motion sickness.

Medicaments that are not hydrophobic chemicals are typically unsuited for topical administration. To be effective, the active drug or agent in a topical composition must penetrate the skin, which is structurally complex and relatively thick. Molecules moving through the skin must first penetrate the stratum corneum and any material on its surface. The molecules must then penetrate the epidermis, the papillary dermis, and the capillary walls into the vascular system or lymphatic system. To be absorbed, the molecules must overcome a different resistance to penetration in each layer.

Strategies have been devised to improve transdermal administration of medicaments. These strategies can be categorized as either physical, chemical, mechanical or biochemical. Combinations of these strategies can also increase efficacy or extend the time for transdermal delivery. Physical techniques include abrasion and tape stripping, which physically break open the skin. Another physical method is prolonged occlusion, which alters the barrier properties of the stratum corneum. After 24 to 28 hours of occlusion with resultant hydration, corneocytes swell, intercellular spaces become distended, and the lacunar network becomes dilated. Distention of the lacunae eventually leads to connections with an otherwise discontinuous system. This creates pores in the stratum corneum interstices through which polar and non-polar substances can penetrate more easily.

Lecithin organogel (LO) is a common component of transdermal penetrants. LOs can help deliver different agents through the skin because they possess both the properties of oil and aqueous based formulations. LOs are typically gels that have an organic medium in liquid phase. They can have jelly-like structure that consists of three-dimensional networks of entangled reverse cylindrical micelles, which immobilize the continuous phase and thus convert from liquid to viscous gel.

Other approaches include the use of Chemical Permeation Enhancers. Chemical Permeation Enhancers (CPEs) are molecules that interact with the constituents of skin's outermost layer, the stratum corneum (SC), and increase its permeability. However, despite efforts at improving them, CPEs are minimally effective in increasing the rate at which drugs permeate the skin. CPEs can also cause skin damage, irritation and sensitization. Further, they are generally ineffective with high molecular weight drugs such as peptides, proteins and nucleic acids.

Although a variety of methods can be used to enhance transdermal drug delivery, these methods have limitations. Most efforts to enhance transdermal penetration have focused on the outermost layer of the skin, the stratum corneum. They typically rely on harsh solvents (e.g., alcohols, DMSO) or patch-based systems. This approach limits the molecular size, lipophilicity, and potency of drugs that can be used. In essence, current approaches are largely limited to small, lipophilic, and highly potent drugs.

### Illustrative Aspects Of the Present Disclosure

The present disclosure provides improved formulations and method for transdermal penetration that work with a variety of medicaments and active agents. These formulations and methods overcome the barrier presented by the stratum corneum as well as the deeper layers of skin. Further, it does so without harsh solvents and are effective in delivering high molecular weight agents such as peptides, proteins and nucleic acids.

An aspect of the present disclosure is a formulation for transdermal delivery of a medicament through the skin of a subject. The formulation comprises a therapeutically effective amount of a medicament and a penetrant portion. The penetrant portion comprises: a phospholipid, a fatty acid ester formed from a low molecular weight alcohol, and a long-chain fatty acids, and, optionally, one or more of a viscosity-improving agent, a penetration enhancer, and an emulsifier.

Another aspect of the present disclosure is a formulation for transdermal delivery of a medicament through the skin of a subject. The formulation comprises a therapeutically effective amount of a medicament and a penetrant portion in which the penetrant portion comprises: a phospholipid, a fatty acid ester formed from a low molecular weight alcohol, and a long-chain fatty acids. In this aspect, phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil.

A further aspect of the present disclosure is a formulation for transdermal delivery of a medicament through the skin of a subject. The formulation comprises a therapeutically effective amount of a medicament and a penetrant portion in which the penetrant portion comprises: a phospholipid, a fatty acid ester formed from a low molecular weight alcohol, and a long-chain fatty acids, and one or more of a viscosity-improving agent, a penetration enhancer, and an emulsifier. In this aspect, phosphatidylcholine, hydrogenated phosphatidylcholine, phosphatidylserine, phosphatidylethanolamine, phosphatidylinositol, inositol phosphatide, or sphingomyelin is the phospholipid; isopropyl palmitate, isopropyl myristate, isopropyl linoleate, isopropyl oleate, ethyl laurate, or ethyl myristate is the fatty acid ester; and a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil; polyglyceryl-4-laurate, polyglyceryl-4-oleate, span 60, cetyl alcohol, or polyglyceryl-3-oleate is the penetration enhancer; a poloxamer (e.g., poloxamer 407, poloxamer 188, poloxamer 184, and poloxamer 124) or sodium lauryl sulfate is the viscosity-improving agent; benzyl alcohol, ethanol, propylene glycol, polyethylene glycol, limonene, menthol, borneol, or camphor is the penetration enhancer.

An additional aspect of the present disclosure is a formulation for transdermal delivery of a medicament through the skin of a subject. The formulation comprises a phospholipid in an amount from about 5% to about 15% w/w of the formulation; an emollient/moisturizer in an amount from about 10% to about 20% w/w of the formulation; a fatty acid in an amount from about 0.5% to about 2% w/w of the formulation; an alcohol in an amount from about 0.5% to about 2% w/w of the formulation; an oil in an amount from about 1% to about 5% w/w of the formulation; a surfactant in an amount from about 0.5% to about 2% w/w of the formulation; water in an amount from about 30% to about 80% w/w of the formulation; and a therapeutically effective amount of a medicament in an amount from about 0.001% to about 30% w/w of the formulation. In some cases, the medicament is in an amount from about 0.001% to about 0.01% w/w of the formulation. In other cases, the medicament is in an amount from about 0.011% to about 0.1% w/w of the formulation. In various cases, the medicament is in an amount from about 0.11% to about 1.0% w/w of the formulation. In further cases, the medicament is in an amount from about 1% to about 10% w/w of the formulation. In additional cases, the medicament is in an amount from about 11% to about 20% w/w of the formulation. In alternate cases, the medicament is in an amount from about 21% to about 30% w/w of the formulation.

In an aspect, the present disclosure provides a formulation for transdermal delivery of a medicament through the skin of a subject. The formulation comprises phosphatidylcholine in an amount of about 7.64% w/w of the formulation; isopropyl palmitate in an amount of about 13.30% w/w of the formulation; stearic acid in an amount of about 0.62% w/w of the formulation; benzyl alcohol in an amount of about 1.39% w/w of the formulation; safflower oil in an amount of about 2.93% w/w of the formulation; oleic acid in an amount of about 0.97% w/w of the formulation; polyglyceryl-4 laurate in an amount of about 1.06% w/w of the formulation; deionized water in an amount of about 60.84% w/w of the formulation; poloxamer 407 in an amount of about 9.25% w/w of the formulation; and a therapeutically effective amount of a medicament in an amount of about 2% w/w of the formulation. In some cases, rather than the medicament being in an amount of about 2% w/w of the formulation, the amount of the medicament is less than about 2% and the amount of water is increased proportionally. In other cases, rather than the medicament being in an amount of about 2% w/w of the formulation, the amount of the medicament is greater than about 2% and the amount of water is decreased proportionally. In various cases, the amount of the medicament is less than about 30%.

In another aspect, the present disclosure provides a formulation for transdermal delivery of a medicament through the skin of a subject. The formulation comprises phosphatidylcholine in an amount of about 7.66% w/w of the formulation; isopropyl palmitate in an amount of about 13.34% w/w of the formulation; benzyl alcohol in an amount of about 1.39% w/w of the formulation; stearic acid in an amount of about 0.68% w/w of the formulation; carthamus tinctorius (safflower) oil in an amount of about 2.79% w/w of the formulation; polyglyceryl-4 laurate in an amount of about 1.07% w/w of the formulation; oleic acid in an amount of about 1.06% w/w of the formulation; deionized water in an amount of about 61.73% w/w of the formulation; poloxamer 407 in an amount of about 9.28% w/w of the formulation; and a therapeutically effective amount of a medicament in an amount of about 1.00% w/w of the formulation. In some case, rather than the medicament being in an amount of about 1% w/w of the formulation, the amount of the medicament is less than about 1% and the amount of water is increased proportionally. In other cases, rather than the medicament being in an amount of about 1% w/w of the formulation, the amount of the medicament is greater than about 1% and the amount of water is decreased proportionally. In various cases, the amount of the medicament is less than about 30%.

In a further aspect, the present disclosure provides a formulation for transdermal delivery of a medicament through the skin of a subject. The formulation comprises a therapeutically effective amount of a medicament and a penetrant portion in which the penetrant portion comprises: phosphatidylcholine in an amount from about 3% to about 15% w/w of the formulation; isopropyl palmitate in an amount from about 5% to about 20% w/w of the formulation; stearic acid in an amount from about 0.1% to about 10% w/w of the formulation; benzyl alcohol in an amount from about 0.5% to about 5% w/w of the formulation; polyglyceryl-4 laurate in an amount from about 0.5% to about 10% w/w of the formulation; and poloxamer 407 in an amount from about 5% to about 20% w/w of the formulation.

In yet another aspect, the present disclosure provides a method for transdermally delivering at least one medicament. The method comprises a step of applying to the skin of a subject an effective amount of any herein disclosed formulation.

An aspect of the present disclosure is a method for treating a disease or disorder or reducing a symptom thereof. The method comprises a steps of administering to a subject in need thereof any herein disclosed transdermal formulation and administering to the subject in need thereof a composition comprising an one or more medicaments selected from Table 1. In some case, the transdermal formulation is administered before, contemporary with, or after the composition is administered. In various cases, the amount of the one or more medicaments is the effective dose of the medicament as described in Table 1. In some cases, the composition is administered by the standard route for the medicament. In some cases, the standard route is oral, topical, enteral, parenteral, by intravenous injection or infusion, by intraperitoneal injection, by intramuscular injection, or by subcutaneous injection. In various cases, the composition is a liquid, a suspension, a gel, a geltab, a semisolid, a tablet, a sachet, a lozenge, a pill, or a capsule.

Another aspect of the present disclosure is a use of any herein disclosed transdermal formulation in a method for treating a disease or disorder or reducing a symptom thereof.

A further aspect of the present disclosure is a method for manufacturing a medicament for treating a disease or disorder or reducing a symptom thereof comprising combining a penetrant portion as recited in any herein disclosed formulation with one or more medicaments recited in Table 1.

Any aspect or embodiment described herein can be combined with any other aspect or embodiment as disclosed herein.

### Transdermal Delivery Formulation Components

Embodiments include a transdermal lotion or cream for administration of medicaments to a subject. It is placed on the skin to deliver a specific dose of an agent through the skin. The agent can be delivered across the skin into a localized subdermal location. For example, a lotion can alleviate inflammation from an autoimmune response. The lotion or cream can be applied directly to the affected area. Alternatively, the agent can enter the circulation for systemic distribution.

In an alternative embodiment, an agent can be administered using a transdermal or medicated adhesive patch. To release an agent, a patch can utilize a porous membrane covering a reservoir of the agent. Alternatively, the agent can be embedded in layers of the adhesive that release the agent as they dissolve or melt.

An advantage of a transdermal drug delivery route over other types of delivery is that the formulation can provide a controlled release of the agent. Conventional transdermal delivery systems are generally ineffective for use with agents and medications that are large molecules and/or hydrophilic molecules.

There are other advantages to transdermal administration of medicaments. Proteins and peptides used, for example, in aging treatments can be degraded by the gastric acid and enzymes. Transdermal administration is not affected by stomach or digestive issues. Further, people can benefit from drugs that are absorbed slowly and regularly. With a transdermal formulation, a medicament can be released in small quantities over a long period of time.

Other advantages are related to dosing. Large doses of agents can cause dose-dependent toxicity in many cases. For example, oral administration of vitamin A can result in hypervitaminosis A. The main problems associated with the vitamin A are its half-life, fast absorption (due to lipophilicity) and its toxicity (due to high loading and frequent dosing). Also, some drugs undergo first-pass metabolism, which prevents their delivery to the desired site of action. Furthermore, many hydrophilic or lipophilic drugs show either poor dissolution or poor absorption on oral administration. With a transdermal formulation, the effective concentration of an agent can be applied at the desired site without painful delivery.

Additionally, the transdermal formulations of the present disclosure are able to deliver an active agent into an animal's bloodstream and thereby provide systemic administration of the agent (e.g., a medicament disclosed in Table 1). In particular, the transdermal formulations of the present disclosure can provide a higher concentration of a molecule (especially one that is insoluble) and can provide systemic administration for a molecule that would be poorly absorbed by the gut epithelium. Together, molecules that are not suitable for enteral delivery or are suitable but at a low dosage can be systemically administered via transdermal formulations of the present disclosure.

In one embodiment, medicaments are supplied via transdermal administration. There are many occasions in which the formulations of the invention are useful. For athletes, a transdermal delivery formulation can deliver to tired muscles sufficient amounts of a neutralizing agent for lactic acid, such as a ketone component, to relieve the burning sensation felt by the athlete due to the buildup of lactic acid. This permits the athlete to continue to perform at optimum level for longer periods of time. In addition, athletes or others “working out” are expending high amounts of energy and are in need of energy generation, especially in those areas of their musculature that are involved in performing workouts and, therefore, need to consume large numbers of calories. These nutrients can be supplied directly rather than requiring oral ingestion which is counterproductive and relatively slow.

The transdermal formulations of the present disclosure may deliver medicaments of various sizes. As examples, the medicaments having a molecular weight of less than 500 Da, e.g., Nicotine (162.2 Da), Diphenhydramine Hydrochloride (291.8 Da), and Hydrocortisone (362.5 Da); medicaments having a molecular weight from 500 Da to 1000 Da, e.g., Sildenafil Citrate (666.7 Da), Neratinib (557 Da), and Doxycycline hyclate (512.9 Da); and medicaments having a molecular weight greater than 1000 Da, e.g., Cyclosporine A (1202.6 Da), Vancomycin Hydrochloride (1485.70 Da), and RBD protein (10,000+Da).

The medicament can be (3S,4S)-8-(6-amino-5-((2-amino-3-chloropyridin-4-yl)thio)pyrazin-2-yl)-3-methyl-2-oxa-8-azaspiro[4.5]decan-4-amine; (S)-3-amino-6-methoxy-N-(3,3,3-trifluoro-2-hydroxy-2-methylpropyl)-5-(trifluoromethyl)picolinamide; 2-fluoro-N-methyl-4-[7-quinolin-6-yl-methyl)-imidazo[1,2-B][1,2,4]triazin-2yl]benzamide; 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide; 4-(7-Hydroxy-2-isopropyl-4-oxo-4H-quinazolin-3-yl)-benzonitrile; Abacavir; Abiraterone Acetate; abiratone acetate; abobotulinum toxin A; Acalabrutinib; Acarbose; Acetaminophen; Acetazolamide; Acetylsalicylic Acid; acitretin; acyclovir; adalimumab; adapalene; adapalene; Afoxolaner; agalsidase beta; Albendazole; Alectinib; Alectinib; alendronate sodium; alglucosidase alfa; alitretinoin; Alogliptin; alpelisib; Aluminum Hydroxide; Ambrisentan; Amiodarone; Amitriptyline; Amlodipine; Amlodipine; Amlodipine besylate; amlodopine besylate; amorolfine hydrochloride; Amoxicillin; Amphotericin B; Amphotericin B liposome; ampicillin; Amprenavir; antihemophilic factor, Fc fusion protein; apalutamide; Apatinib; apixaban; apremilast; Aprepitant; Aripiprazole; Artemether; aspirin; Atazanavir; atenolol; Atomoxetine; Atorvastatin; Atorvastatin; Avapritinib; Axitinib (Inlyta); Azacitidine; Azathioprine; azelaic acid; Azilsartan medoxomil; azithromycin; baclofen; baricitinib; basiliximab; Batimastat (BB-94); becaplermin; Belumosudil; Bendamustine; benzoyl peroxide; benzoyl peroxide; betamethasone dipropionate; betamethasone valerate; bexarotene; Bictegravir; Bimatoprost; binimetinib (Mektovi); Bortezomib; Bortezomib (Velcade); Bosentan; Bosutinib (Bosulif); botulinum toxin A; branched chain amino acids; Brexpiprazole; Brimonidine; brimonidine tartrate; brodalumab; buproprion; Cabazitaxel; Cabozantinib; Cabozantinib (Cometriz); Calcifediol; calcipotriene; Calcipotriene; Calcipotriol; calcitonin; calcitriol; calcium carbonate; Canagliflozin; canakinumab; Capecitabine; Capmatinib; capsaicin; Carbamazepine; Carbidopa; Carfilzomib; Carfilzomib (Kyprolis); Cariprazine; carvedilol; CD-12681; CEE-321; cefazolin; cefepime; Cefoperazone; cefotaxime sodium; celecoxib; celecoxib; cemiplimab; Ceritinib; Chlorothiazide; Chlorpromazine; Chlorthalidone; cinacalcet; ciprofloxacin; Cisapride; citalopram; citric acid; clarithromycin; Clavulanate; Clindamycin phosphate; Clobazam; clobetasol propionate; Clofazimine; clomiphene; clonidine; Clopidogrel; clopidogrel bisulfate; cobicistat; Cobicistat; Colchocine; Colistin; Corticotropin; crisaborole; crizanlizumab; Crizotinib (Xalkori); cyclobenzaprine; cyclosporine; Dabigatran Etexilate; Dabrafenib; Dabrafenib; dabrafenib; dabrafenib; dacomitinib (Vizimpro); dalbavancin; dalfopristin; Danazol; Dapagliflozin; Dapsone; daptomycin; Darunavir; Dasatinib; decitabine; defatted Peanut (Arachis hypogaea) Flour; deferasirox; delgocitinib; deoxycholic acid; Desipramine; desonide; Dexlansoprazole; Dexmethylphenidate; dextroamphetamine/amphetamine salts; Diclofenac; diclofenac sodium; diflucortolone; Diflucortolone; Difunisal; Digoxin; Diloxanide; Dimethyl Fumarate; diphenhydramine hydrochloride; diphtheria vaccine; docetaxel; Dolutegravir; Dolutegravir; Donepezil; Doxercalciferol; doxycycline; doxycycline ER; Doxycycline Hyclate (B); Doxycycline Monohydrate (A); Dronabinol; Dronedarone; Drospirenone; Dukoral/ShanChol cholera vaccine; Duloxetine; dupilumab; durvalumab; Dutasteride; Duvelisib; ecallantide; Edoxaban; Efavirenz; Efavirenz; Elafibranor; elagolix sodium; Elbasvir; Eletriptan; Eltrombopag; Elvitegravir; Empagliflozin; Emtricitabine; emtricitabine; Emtricitabine; Emtricitabine; Emtricitabine; Emtricitabine; Emtricitabine; Emtricitabine; encorafenib (Braftovi); enfuviritide; enoxaparin sodium; Entecavir; entrectinib; Enzalutamide; Epinephrine; erdafitinib; erenumab; Eribulin; Erlotinib; Erlotinib HCl (Tarceva); Ertapenem; Erythromycin; escitalopram; esketamine (ketamine); Esomeprazole; esopremazole; estradiol; Estrogens—conjugated; etanercept; Ethinyl Estradiol; Ethinyl Estradiol; Ethinyl Estradiol; Etonogestrel; Etonogestrel; Etravirine; etretinate; Everolimus; Everolimus (Afinitor); Everolimus (Votubia, Zortress/Certican); Ezetimibe; Ezetimibe; Ezetimibe; Famotidine; fasinumab; Febuxostat; Fedratinib; Fenofibrate; Ferric Carboxymaltose; fexofenadine hydrochloride; filgotinib; filgrastim; fingolimod; fluocinolone acetonide; fluocinolone acetonide; fluoxetine; Flurbiprofen; Folic acid; Forskolin; Fostamatinib; Fulvestrant; furosemide; fusidic acid; fusidic acid; gabapentin; Ganetespib; Gefitinib (Iressa); Gilteritinib; glasdegib (Daurismo) or glasdegib maleate; Glecaprevir; Glibenclamide; Glimepiride; glipizide; glucagon; glucosamine; glutaric anhydride; glycopyrrolate; Goserelin; goserelin LA; granisetron; Grazoprevir; Griseofulvin; halobetasol propionate; Haloperidol; heparin sodium; heplisav-B vaccine; hyaluronic acid; Hydrochlorothiazide; Hydrochlorothiazide; Hydrochlorothiazide; Hydrochlorothiazide; hydrocortisone; hydrocortisone acetate; hydroquinone; hydroxychloroquine; Hydroxyprogesterone; Ibrutinib; Ibrutinib (Imbruvica); ibuprofen; icatibant acetate; Idelalisib; Imatinib; Imatinib (Gleevec); imatinib mesylate; imiglucerase; inclisiran; Indinavir; Indomethacin; infliximab; ingenol mebutate; inotersen; insulin A; insuline glargine; interferon beta-Ib; Iopanoic acid; Irbesartan; isoconazole; isoconazole; Isotretinoin; Itraconazole; Ivacaftor; Ivacaftor; ivermectin; Ixazomib; ketoconazole; ketones; ketoprofen; Keyruvia; Lacosamide; lamictal; Lamivudine; Lansoprazole; Lapatinib; Lapatinib (Tykerb); larotrectinib; Ledipasvir; leflunomide; Lenalidomide; Lenvatinib; LEO-138559; LEO-152020; Leuprolide; Levetiracetam; levodopa; Levodopa/Benserazide; Levonorgestrel; Levothyroxine; Linagliptin; linezolid; lisinopril; L-lysine free base; lofexidine; Lopinavir; Loratadine; lorlatinib (Lorviqua); Losartan; Lovastatin; Lubiprostone; Lumacaftor/Ivacaftor; Lumefantrine; Lurasidone; Luspatercept; lymecycline; Macitentan; magnesium; magnesium lactate; Marimastat (BB-2516); Marizomib (NPI-0052); Mebendazole; Mefloquine; melatonin; meloxicam; memantine hydrochloride; meningococcal[serotype b] vaccine; Mesalazine; Metformin; Metformin; metformin; Metformin; methotrexate; methyl aminolevulinate hydrochloride; Methylphenidate; methylprednisolone; Metoprolol succinate; metronidazole; Midostaurin; minoxidil; Mirabegron; mizolastine MR; Montelukast; Mycophenolate Mofetil; N-(3-(2-(2-hydroxyethoxy)-6-morpholinopyridin-4-yl)-4-methylphenyl)-2-(trifluoromethyl)-isonicotinamide; N-(3-(6-Amino-5-(2-(N-methylacrylamido)ethoxy)pyrimidin-4-yl)-5-fluoro-2-methylphenyl)-4-cyclopropyl-2-fluorobenzamide; N-[4-(Chlorodifluoromethoxy)phenyl]-6-[(3R)-3-hydroxpyrrolidin-1-yl]-5-(1H-pyrazol-5-yl)-pyridine-3-carboxamide hydrochloride; Nalidixic acid (Quinolone antibiotic); naloxone; Naproxen; Navitoclax (ABT-263); Nebivolol; Nelfinavir; nemolizumab; neomycin; Neovastat (AE-941); Neratinib—Free Base; Neratinib (Nerlynx); Neratinib Maleate; Nevirapine; Niclosamide; Nifedipine; nilotinib; Nilotinib; Nilotinib (Tasigna); Nintedanib; niratimib; Nitric Oxide; Nitrofurantoin; Norethindrone Acetate; nusinersen; NVP-AUY922; Nystatin; Obatoclax (GX15-070); octreotide acetate; ofatumumab; Ofloxacin; Olanzapine; olaparib; Olmesartan Medoxomil; olumacostat; omalizumab; Oseltamivir; Osimertinib; osimertinib mesylate; Oxaprozin; Ozanimod; Paclitaxel; Paclitaxel Protein Bound; Palbociclib (Ibrance); Paliperidone Palmitate; Pantoprazole; parathyroid hormone; Paricalcitol; patisiran; pazopanib; Pazopanib (Votrient); pazopanib hydrochloride; Peficitinib; pegfilgrastim; Pembrolizumab (Keytruda); Pemetrexed; Pemigatinib; Perifosine; Pexidartinib; Phenazopyridine; Phenytoin; Pibrentasvir; Piperacillin; Pirfenidone; Piroxicam; Pomalidomide; Ponatinib; Ponatinib (lclusig); Posaconazole; Pralsetinib; pravastatin; prednicarbate; prednisone; pregabalin; pregabalin; Prinomastat (AG-3340); Progesterone; propanolol; protamine sulfate; psilocybin; Pyrantel; Pyrimethamine; Quetiapine; Quinupristin; Raloxifene; Raltegravir; ranibizumab; ranitidine; Ranolazine; Rebimastat (BMS-275291); Regorafenib; Regorafenib (Stivarga); relugolix (Orgovyx); remibrutinib; resiquimod; retinal; Retinol; ribociclib; ribociclib succinate; Rifampin; Rilpivirine; Rilpivirine; Rilpivirine; Rimegepant; Riociguat; Risdiplam; Risperidone; Ritonavir; Rivaroxaban; Rosuvastatin; rosuvastatin calcium; Rotigotine; roxadustat; rucaparib camsylate (Rubraca); ruxolitinib; Ruxolitinib (Jafaki); Sacubitril, valsartan; salicylic acid; Sapropterin; Saquinavir; sarilumab; Saxagliptin, Metformin; secukinumab; Selexipag; selinexor; Selpercatinib; selpercatinib (LOXO-292); Selumetinib; semaglutide; sertraline; Sevoflurane; sildenafil; Sildenafil Citrate; Simvastatin; siponimod; Siremadlin; Sirolimus; Sitagliptin; Sitagliptin phosphate monohydrate; Sodium bicarbonate or sodium carbonate; sodium deoxycholate; sodium nitrate; Sofosbuvir; Sofosbuvir; Sofosbuvir; Solifenacin; somatropin; sonidegib phosphate; Sorafenib; Sorafenib (Nexavar); spartalizumab; Spironolactone; sufentanil citrate; Sugammadex; sulbactam; Sulbactam; Sulfadiazine; Sulfamethoxazole; sulfasalazine; sumatriptan; Sunitinib; Sunitinib; Sunitinib (sutent); sunitinib malate (Sutent); Tacrolimus; TAF; TAF; TAF; TAF; talazoparib—Talzenna); Talinolol; Tamoxifen; Tamsulosin; tazarotene; tazobactam; TDF; TDF; Temsirolimus (CCl-779, Torisel); Teneligliptin; tenofovir alafenamide; Tenofovir Disoproxil Fumarate; Tepotinib; terbinafine; Terfenadine; Teriflunomide; Testosterone; Tezacaftor; THC; ticagrelor; tigecycline; Timolol; timolol maleate; Tipranavir; tisagenlecleucel; Tisagenlecleucel (Kymriah); Tivozanib; tofacitinib citrate; Tolvaptan; tralokinumab; tramadol; Trametinib; Trametinib; trametinib; Trametinib; Trametinib (Mekinist); tranexamic acid; Travoprost; trazodone; tretinoin; tretinoin; triancinolone; Triclabendazole; trifarotene; Trimethoprim; Triptorelin; tris; Tucatinib; ubrogepant; Umbralisib; upadacitinib; valproic acid; Valsartan; valsartan; Valsartan; valsartan; vancomycin; Vancomycin Hydrochloride (A); Vancomycin Hydrochloride (B); Vandetanib; Vandetenib (Caprelsa); Varenicline; Vasopressin; Velpatasvir; Velpatasvir; Vemurafenib; Venetoclax; Verapamil hydrochloride; Vilazodone; Vildagliptin; vismodegib; vitamin B6; vitamin D; Vonoprazan; Voriconazole; Vortioxetine; Voxilaprevir; Warfarin Sodium; Zanubrutinib; Zinc; zoledronic acid; and Zolpidem.

In some cases, a therapeutic comprises two or more medicaments. In these cases, the first and the at least second medicament are selected from the group consisting of Abacavir and Dolutegravir and Lamivudine; adapalene and benzoyl peroxide; Amlodipine and Valsartan; amlodopine besylate and valsartan; Amoxicillin and Clavulanate; ampicillin and sulbactam; Artemether and Lumefantrine; betamethasone valerate and fusidic acid; Bictegravir and Emtricitabine and TAF; Bimatoprost and timolol maleate; Brimonidine and Timolol; Calcipotriene and betamethasone dipropionate; Canagliflozin and Metformin; Carbidopa and levodopa; Cefoperazone and Sulbactam; Dabrafenib and Trametinib; Darunavir and cobicistat and emtricitabine and tenofovir alafenamide; Diflucortolone and isoconazole; Drospirenone and Ethinyl Estradiol; Efavirenz and Emtricitabine and TDF; Elbasvir and Grazoprevir; Elvitegravir and Cobicistat and Emtricitabine and TAF; Emtricitabine and Rilpivirine and TAF; Emtricitabine and TAF; Emtricitabine and Tenofovir Disoproxil Fumarate; Ethinyl Estradiol and Etonogestrel; Ezetimibe and Atorvastatin; Ezetimibe and Simvastatin; fluocinolone acetonide and hydroquinone and tretinoin; fusidic acid and hydrocortisone acetate; Glecaprevir and Pibrentasvir; Hydrochlorothiazide and Valsartan; Irbesartan and Hydrochlorothiazide; Ledipasvir and Sofosbuvir; Linagliptin and Metformin; Losartan and Hydrochlorothiazide; Metformin and Sitagliptin; Norethindrone Acetate and Ethinyl Estradiol; Piperacillin and tazobactam; pregabalin and celecoxib; Quinupristin and dalfopristin; Rilpivirine and Emtricitabine and TDF; Sofosbuvir and Velpatasvir; Sofosbuvir and Velpatasvir and Voxilaprevir; Tezacaftor and Ivacaftor; trametinib and dabrafenib; Trametinib and dabrafenib; and Zinc and magnesium and vitamin B6.

Throughout this disclosure, a medicament, agent, or other ingredient is described as being provided at specific ranges of doses, amounts, concentrations, percentages, units, volumes, and the like. For example, a medicament may be provided at range of doses of about 1 mg to about 10 mg. As used herein a range from about 1 mg to about 10 mg includes all dosages therebetween and any subranges therebetween. More specifically, the dosages may be about 1 mg, about 1.01 mg, about 1.02 mg, about 1.03 mg, about 1.04 mg, about 1.05 mg, about 1.06 mg, about 1.07 mg, about 1.08 mg, about 1.09 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, about 2.1 mg, about 2.2 mg, about 2.3 mg, about 2.4 mg, about 2.5 mg, about 2.6 mg, about 2.7 mg, about 2.8 mg, about 2.9 mg, about 3 mg, about 3.1 mg, about 3.2 mg, about 3.3 mg, about 3.4 mg, about 3.5 mg, about 3.6 mg, about 3.7 mg, about 3.8 mg, about 3.9 mg, about 4 mg, about 4.1 mg, about 4.2 mg, about 4.3 mg, about 4.4 mg, about 4.5 mg, about 4.6 mg, about 4.7 mg, about 4.8 mg, about 4.9 mg, about 5 mg, about 5.1 mg, about 5.2 mg, about 5.3 mg, about 5.4 mg, about 5.5 mg, about 5.6 mg, about 5.7 mg, about 5.8 mg, about 5.9 mg, about 6 mg, about 6.1 mg, about 6.2 mg, about 6.3 mg, about 6.4 mg, about 6.5 mg, about 6.6 mg, about 6.7 mg, about 6.8 mg, about 6.9 mg, about 7 mg, about 7.1 mg, about 7.2 mg, about 7.3 mg, about 7.4 mg, about 7.5 mg, about 7.6 mg, about 7.7 mg, about 7.8 mg, about 7.9 mg, about 8 mg, about 8.1 mg, about 8.2 mg, about 8.3 mg, about 8.4 mg, about 8.5 mg, about 8.6 mg, about 8.7 mg, about 8.8 mg, about 8.9 mg, about 9 mg, about 9.1 mg, about 9.2 mg, about 9.3 mg, about 9.4 mg, about 9.5 mg, about 9.6 mg, about 9.7 mg, about 9.8 mg, about 9.9 mg, about 10 mg, and any dosage therebetween. Moreover, the ranges may be from about 1 mg to about 2 mg, from about 1 mg to about 3 mg, from about 1 mg to about 5 mg, from about 1 mg to about 7 mg, from about 1 mg to about 8 mg, from about 2 mg to about 3 mg, from about 2 mg to about 4 mg, from about 2 mg to about 6 mg, from about 2 mg to about 8 mg, from about 2 mg to about 9 mg, from about 2 mg to about 5 mg, from about 2 mg to about 7 mg, from about 3 mg to about 4 mg, from about 3 mg to about 5 mg, from about 3 mg to about 7 mg, from about 3 mg to about 9 mg, from about 3 mg to about 10 mg, from about 3 mg to about 6 mg, from about 3 mg to about 8 mg, from about 4 mg to about 5 mg, from about 4 mg to about 6 mg, from about 4 mg to about 8 mg, from about 4 mg to about 10 mg, from about 4 mg to about 7 mg, from about 4 mg to about 9 mg, from about 5 mg to about 6 mg, from about 5 mg to about 7 mg, from about 5 mg to about 9 mg, from about 5 mg to about 8 mg, from about 5 mg to about 10 mg, from about 6 mg to about 7 mg, from about 6 mg to about 8 mg, from about 6 mg to about 10 mg, from about 6 mg to about 9 mg, from about 7 mg to about 8 mg, from about 7 mg to about 9 mg, from about 7 mg to about 10 mg, from about 8 mg to about 9 mg, from about 8 mg to about 10 mg, or from about 9 mg to about 10 mg, and any subrange therebetween.

The amount of a medicament in a transdermal delivery formulation disclosed herein may comprise a percentage of a formulation or solution from about 0.001% to about 30% (w/w) or (w/v), e.g., about 0.0005%, 0.0006%, 0.0007%, 0.0008%, 0.0009%, 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, or 30%, or any (w/w) or (w/v) therebetween. The medicaments and their various dose ranges listed in Table 1 herein could be used in an above-mentioned dose or any dose from about 0.001% to about 30%. As examples, the various doses listed above would be applicable to fulvestrant, eltrombopag, selpercatinib (LOXO-292), ertapenem, erlotinib, sugammadex, nebivolol, azilsartan medoxomil, nifedipine, triptorelin, elagolix sodium, olmesartan medoxomil, oseltamivir, selinexor, levetiracetam, leuprolide, goserelin, lenvatinib, ethinyl estradiol (etonogestrel), paclitaxel protein bound, bendamustine, acyclovir, adalimumab, alpelisib, ampicillin (sulbactam), azithromycin, branched chain amino acids, calcium carbonate, capsaicin, cemiplimab, citric acid, clonidine, crisaborole, docetaxel, ecallantide, erenumab, etanercept, fasinumab, filgrastim, forskalin, fulvestrant, glutaric anhydride, glycopyrrolate, goserelin LA, heparin sodium, infliximab, ketoprofen, linezolid, L-lysine free base, magnesium lactate, metronidazole, olaparib, olumacostat, omalizumab, parathyroid homone, piperacillin (tazobactam) quinupristin (dalfopristin), salicylic acid, sarilumab, secukinumab, sodium nitrate, sulfasalazine, tigecycline, tisagenlecleucel, tranexamic acid, triamcinolone, vitamin D, zinc, magnesium, vitamin B6, aspirin, becaplermin, buproprion, cefazolin, clarithromycin, cyclobenzaprine, furosemide, gabapentin, halobetasol propionate, inotersen, levodopa (carbidopa), melatonin, meloxicam, neomycin, neratinib, patisiran, prednisone, ribociclib, sonidegib phosphate, sufentanil citrate, tramadol, trametinib+dabrafenib, zoledronic acid or niratimib, glecaprevir, pibrentasvir, sofosbuvir, velpatasvir, emtricitabine, tenofovir disoproxil fumarate, lenalidomide, abacavir, dolutegravir, lamivudine, paliperidone palmitate, sitagliptin phosphate monohydrate, empagliflozin, dolutegravir, linagliptin, metformin, elvitegravir, cobicistat, emtricitabine, TAF, metformin, sitagliptin, esomeprazole, acarbose, afoxolaner, agalsidase beta, alglucosidase alfa, alogliptin, antihemophilic factor, Fc fusion protein, aprepitant, aripiprazole, atazanavir, atomoxetine, azacitidine, basiliximab, bictegravir, emtricitabine, TAF, bimatoprost, timolol maleate, bosentan, brexpiprazole, brimonidine, timolol, calcipotriol, calcitonin, canagliflozin, metformin, canakinumab, cariprazine, clobazam, clomiphene, clopidogrel bisulfate, crizanlizumab, dapagliflozin, darunavir, cobicistat, emtricitabine, tenofovir alafenamide, dexlansoprazole, dexmethylphenidate, diphtheria vaccine, donepezil, dronedarone, dukarol/shanchol cholera vaccine, duloxetine, dupilumab, durvalumab, efavirenz, emtricitabine, TAF, elafibranor, elbasvir, grazoprevir, eltrombopag, emtricitabine, rilpivirine, TAF, emtricitabine, TAF, entecavir, erenumab, esketamine (ketamine), ezetimibe, ezetimibe, atorvastatin, ezetimibe, simvastatin, febuxostat, glimepiride, glucosamine, granisetron, heplisav-b vaccine, imiglucerase, inclisiran, insuline glargine, interferon beta-lb, ketones, lamictal, ledipasvir, sofosbuvir, lofexidine, losartan, hydrochlorothiazide, lubiprostone, lurasidone, luspatercept, memantine hydrochloride, mesalazine, methylphenidate, mirabegron, montelukast, mycophenolate mofetil, naloxone, nilotinib, nitric oxide, octreotide acetate, ofatumumab, olanzapine, osimertinib mesylate, pantoprazole, pegfilgrastim, psilocybin, quetiapine, raltegravir, ranibizumab, ranolazine, rilpivirine, rilpivirine, emtricitabine, TDF, risperidone, rosuvastatin calcium, roxadustat, sapropterin, saxagliptin, metformin, semaglutide, sevoflurane, sofosbuvir, velpatasvir, voxilaprevir, somatropin, spartalizumab, teneligliptin, tezacaftor, ivacaftor, THC, ticagrelor, tolvaptan, travoprost, vasopressin, vildagliptin, vilazodone, vonoprazan, vortioxetine, zolpidem, atenolol, carvedilol, citalopram, dextroamphetamine/amphetamine salts, enfuviritide, escitalopram, esopremazole, fluoxetine, givosiran, glipizide, glucagon, lisinopril, nifedipine, pravastatin, propanolol, ranitidine, sertraline, trazodone, valsartan, calcipotriene, diflucortolone, methylprednisolone, delgocitinib, isoconazole, azelaic acid, calcipotriene (with or without betamethasone dipropionate), diflucortolone (with or without isoconazole), betamethasone valerate (with or without fusidic acid), fusidic acid (with or without hydrocortisone acetate), hydrocortisone acetate (with or without fusidic acid), fusidic acid (with or without betamethasone valerate), betamethasone dipropionate (with or without calcipotriene), isoconazole (with or without diflucortolone), baclofen, ingenol mebutate, octreotide acetate, Epinephrine, fingolimod, Trametinib with dabrafenib, Trametinib, dabrafenib (with or without trametinib), Amlodipine besylate, apixaban, amlodopine besylate (with or without valsartan), ruxolitinib, Everolimus (Afinitor/Votubia with Zortress/Certican), Siremadlin, valsartan (with or without amlodopine besylate), Eletriptan, valsartan, Venetoclax, eltrombopag, sildenafil, remibrutinib, decitabine, canakinumab, Sacubitril (with or without valsartan), vildagliptin, Midostaurin, Dabrafenib, Roxadustat, protamine sulfate, heparin sodium, omalizumab, imatinib mesylate, calcitonin, diclofenac sodium, sumatriptan, Progesterone, deferasirox, azacitidine, doxycycline, secukinumab, cyclosporine, pazopanib hydrochloride, celecoxib, brodalumab, Ofatumumab, pregabalin, tralokinumab, crizanlizumab, daptomycin, nilotinib, vancomycin, cefepime, metformin, ribociclib succinate.

The effective dose of a medicament and/or active agent may be an amount from about 0.0001 mg to about 10 mg, e.g., about 0.0001 mg, 0.0002 mg, 0.0003 mg, 0.0004 mg, 0.0005 mg, 0.0006 mg, 0.0007 mg, 0.0008 mg, 0.0009 mg, 0.001 mg, 0.002 mg, 0.003 mg, 0.004 mg, 0.005 mg, 0.006 mg, 0.007 mg, 0.008 mg, 0.009 mg, 0.01 mg, 0.015 mg, 0.02 mg, 0.025 mg, 0.03 mg, 0.035 mg, 0.04 mg, 0.045 mg, 0.05 mg, 0.055 mg, 0.06 mg, 0.065 mg, 0.07 mg, 0.075 mg, 0.08 mg, 0.085 mg, 0.09 mg, 0.095 mg, 0.1 mg, 0.15 mg, 0.2 mg, 0.25 mg, 0.3 mg, 0.35 mg, 0.4 mg, 0.45 mg, 0.5 mg, 0.55 mg, 0.6 mg, 0.65 mg, 0.7 mg, 0.75 mg, 0.8 mg, 0.85 mg, 0.9 mg, 0.95 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, or 10 mg, or any dose therebetween. Various drugs and their dose ranges listed in Table 1 could be used in an above-mentioned dose. As examples, the various doses listed above would be applicable to Lenalidomide, Empagliflozin, Linagliptin, Metformin, Elvitegravir, Cobicistat, Emtricitabine, TAF, Aripiprazole, Brexpiprazole, Cariprazine, Clobazam, Dapagliflozin, Dexmethylphenidate, Donepezil, Entecavir, Ezetimibe, Ezetimibe, Atorvastatin, Ezetimibe, Simvastatin, Glimepiride, granisetron, interferon beta-lb, lofexidine, Lubiprostone, memantine hydrochloride, Montelukast, naloxone, octreotide acetate, Olanzapine, pegfilgrastim, ranibizumab, Risperidone, rosuvastatin calcium, Saxagliptin, Metformin, semaglutide, somatropin, THC, Tolvaptan, Vortioxetine, Zolpidem, carvedilol, dextroamphetamine/amphetamine salts, escitalopram, esopremazole, fluoxetine, glipizide, glucagon, lisinopril, nifedipine, pravastatin, Nebivolol, Nifedipine, Triptorelin, Leuprolide, Goserelin, Lenvatinib, Ethinyl Estradiol, Etonogestrel, capsaicin, clonidine, fasinumab, filgrastim, glycopyrrolate, goserelin LA, olumacostat, parathyroid hormone, triamcinolone, vitamin D, zinc, magnesium, vitamin B6, becaplermin, cyclobenzaprine, halobetasol propionate, Carbidopa, levodopa, melatonin, meloxicam, prednisone, sufentanil citrate, trametinib+dabrafenib, zoledronic acid, calcitonin, everolimus (Afinitor/Votubia with Zortress/Certican), trametinib (alone or with dabrafenib), Amlodipine besylate (with valsartan), apixaban, amlodopine besylate, ruxolitinib, Siremadlin (alone or with valstartan), Eletriptan, valsartan (alone or with amlodopine besylate), and venetoclax.

The effective dose of a medicament and/or active agent may be an amount from about 10 mg to about 1000 mg, e.g., about 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 520 mg, 540 mg, 560 mg, 580 mg, 600 mg, 620 mg, 640 mg, 660 mg, 680 mg, 700 mg, 720 mg, 740 mg, 760 mg, 780 mg, 800 mg, 820 mg, 840 mg, 860 mg, 880 mg, 900 mg, 920 mg, 940 mg, 960 mg, 980 mg, or 1000 mg, or any dose therebetween. Various drugs and their dose ranges listed in Table 1 could be used in an above-mentioned dose. As examples, the various doses listed above would be applicable to Glecaprevir, Pibrentasvir, Sofosbuvir, Velpatasvir, Emtricitabine, Tenofovir Disoproxil Fumarate, Lenalidomide, Abacavir, Dolutegravir, Lamivudine, Paliperidone Palmitate, Sitagliptin phosphate monohydrate, Empagliflozin, Dolutegravir, Linagliptin, Metformin, Elvitegravir, Cobicistat, Emtricitabine, TAF, Metformin, Sitagliptin, Esomeprazole, Acarbose, Afoxolaner, agalsidase beta, alglucosidase alfa, Alogliptin, Aprepitant, Aripiprazole, Atazanavir, Atomoxetine, Azacitidine, basiliximab, Bictegravir, Emtricitabine, TAF, Bosentan, Canagliflozin, Metformin, Canakinumab, Clobazam, clomiphene, clopidogrel bisulfate, crizanlizumab, Dapagliflozin, darunavir, cobicistat, emtricitabine, tenofovir alafenamide, Dexlansoprazole, Dexmethylphenidate, Donepezil, Dronedarone, Duloxetine, dupilumab, durvalumab, Efavirenz, Emtricitabine, TDF, Elbasvir, Grazoprevir, eltrombopag, Emtricitabine, Rilpivirine, TAF, Emtricitabine, TAF, erenumab, esketamine (ketamine), Ezetimibe, Ezetimibe, Atorvastatin, Ezetimibe, Simvastatin, Febuxostat, Glucosamine, Inclisiran, lamictal, Ledipasvir, Sofosbuvir, Losartan, Hydrochlorothiazide, Lurasidone, Luspatercept, memantine hydrochloride, Methylphenidate, Mirabegron, Montelukast, Mycophenolate Mofetil, nilotinib, ofatumumab, Olanzapine, osimertinib mesylate, Pantoprazole, Quetiapine, Raltegravir, Ranolazine, Rilpivirine, Rilpivirine, Emtricitabine, TDF, Risperidone, rosuvastatin calcium, roxadustat, Sapropterin, Saxagliptin, Metformin, Sofosbuvir, Velpatasvir, Voxilaprevir, spartalizumab, Teneligliptin, Tezacaftor, Ivacaftor, ticagrelor, Tolvaptan, Vildagliptin, Vilazodone, Vonoprazan, Vortioxetine, Zolpidem, atenolol, carvedilol, citalopram, dextroamphetamine/amphetamine salts, enfuviritide, escitalopram, esopremazole, fluoxetine, givosiran, glipizide, glucagon, lisinopril, nifedipine, pravastatin, propanolol, ranitidine, sertraline, trazodone, valsartan, Fulvestrant, Eltrombopag, selpercatinib (LOXO-292), Ertapenem, Erlotinib, Sugammadex, Nebivolol, Azilsartan medoxomil, Nifedipine, Triptorelin, elagolix sodium, Olmesartan Medoxomil, Oseltamivir, selinexor, Levetiracetam, Leuprolide, Lenvatinib, Paclitaxel Protein Bound, Bendamustine, acyclovir, adalimumab, alpelisib, ampicillin, sulbactam, azithromycin, branched chain amino acids, calcium carbonate, capsaicin, cemiplimab, citric acid, crisaborole, docetaxel, ecallantide, erenumab, etanercept, fasinumab, Forskolin, fulvestrant, glutaric anhydride, glycopyrrolate, goserelin LA, heparin sodium, infliximab, ketoprofen, linezolid, L-lysine free base, magnesium lactate, metronidazole, olaparib, olumacostat, omalizumab, piperacillin, tazobactam, quinupristin, dalfopristin, salicylic acid, sarilumab, secukinumab, sodium nitrate, sulfasalazine, tigecycline, tranexamic acid, triamcinolone, zinc, magnesium, vitamin B6, aspirin, buproprion, cefazolin, clarithromycin, cyclobenzaprine, furosemide, gabapentin, inotersen, Carbidopa, levodopa, meloxicam, neomycin, neratinib, patisiran, prednisone, ribociclib, sonidegib phosphate, tramadol, trametinib+dabrafenib, niratimib, dabrafenib (with trametinib), Amlodipine besylate, apixaban, amlodopine besylate (with or without valsartan), ruxolitinib, Everolimus (Afinitor/Votubia, Zortress/Certican), Siremadlin, Eletriptan, valsartan, Venetoclax, eltrombopag, sildenafil, remibrutinib, decitabine, canakinumab, Sacubitril (with or without valsartan), vildagliptin, Midostaurin, Dabrafenib, Roxadustat, protamine sulfate, heparin sodium, omalizumab, imatinib mesylate, diclofenac sodium, sumatriptan, Progesterone, deferasirox, azacitidine, doxycycline, secukinumab, cyclosporine, pazopanib hydrochloride, celecoxib, brodalumab, Ofatumumab, pregabalin, tralokinumab, crizanlizumab, daptomycin, nilotinib, vancomycin, cefepime, metformin, ribociclib succinate.

The effective dose of a medicament and/or active agent may be an amount from about 1 g to about 30 g, e.g., about 1 g, 1.1 g, 1.2 g, 1.3 g, 1.4 g, 1.5 g, 1.6 g, 1.7 g, 1.8 g, 1.9 g, 2 g, 2.1 g, 2.2 g, 2.3 g, 2.4 g, 2.5 g, 2.6 g, 2.7 g, 2.8 g, 2.9 g, 3 g, 3.1 g, 3.2 g, 3.3 g, 3.4 g, 3.5 g, 3.6 g, 3.7 g, 3.8 g, 3.9 g, 4 g, 4.1 g, 4.2 g, 4.3 g, 4.4 g, 4.5 g, 4.6 g, 4.7 g, 4.8 g, 4.9 g, 5 g, 5.2 g, 5.4 g, 5.6 g, 5.8 g, 6 g, 6.2 g, 6.4 g, 6.6 g, 6.8 g, 7 g, 7.2 g, 7.4 g, 7.6 g, 7.8 g, 8 g, 8.2 g, 8.4 g, 8.6 g, 8.8 g, 9 g, 9.2 g, 9.4 g, 9.6 g, 9.8 g, 10 g, 11 g, 12 g, 13 g, 14 g, 15 g, 16 g, 17 g, 18 g, 19 g, 20 g, 21 g, 22 g, 23 g, 24 g, 25 g, 26 g, 27 g, 28 g, 29 g, or 30 g, or any dose therebetween. Various drugs and their dose ranges listed in Table 1 could be used in an above-mentioned dose. As examples, the various doses listed above would be applicable to Linagliptin, Metformin, alglucosidase alfa, Canagliflozin, Metformin, darunavir, cobicistat, emtricitabine, tenofovir alafenamide, Mesalazine, Ofatumumab, Quetiapine, Raltegravir, Ranolazine, Sapropterin, Ertapenem, Acyclovir, ampicillin, sulbactam, branched chain amino acids, calcium carbonate, citric acid, metronidazole, tranexamic acid, cefazolin, neomycin, imatinib mesylate, cyclosporine, pazopanib hydrochloride, Ofatumumab, vancomycin, cefepime, and metformin.

The effective dose of a medicament and/or active agent may be an amount from about 5 IU/kg to about 100 IU/kg, e.g., about 5 IU/kg, 10 IU/kg, 15 IU/kg, 20 IU/kg, 25 IU/kg, 30 IU/kg, 35 IU/kg, 40 IU/kg, 45 IU/kg, 50 IU/kg, 55 IU/kg, 60 IU/kg, 65 IU/kg, 70 IU/kg, 75 IU/kg, 80 IU/kg, 85 IU/kg, 90 IU/kg, 95 IU/kg, or 100 IU/kg, or any dose therebetween. Various drugs and their dose ranges listed in Table 1 could be used in an above-mentioned dose. As examples, the various doses listed above would be applicable to antihemophilic factor, Fc fusion protein, calcitonin, and heparin sodium.

The effective dose of a medicament and/or agent may be an amount from about 0.1 mg/ml to about 10 mg/ml, e.g., about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, 2.4 mg/ml, 2.5 mg/ml, 2.6 mg/ml, 2.7 mg/ml, 2.8 mg/ml, 2.9 mg/ml, 3 mg/ml, 3.1 mg/ml, 3.2 mg/ml, 3.3 mg/ml, 3.4 mg/ml, 3.5 mg/ml, 3.6 mg/ml, 3.7 mg/ml, 3.8 mg/ml, 3.9 mg/ml, 4 mg/ml, 4.1 mg/ml, 4.2 mg/ml, 4.3 mg/ml, 4.4 mg/ml, 4.5 mg/ml, 4.6 mg/ml, 4.7 mg/ml, 4.8 mg/ml, 4.9 mg/ml, 5 mg/ml, 5.1 mg/ml, 5.2 mg/ml, 5.3 mg/ml, 5.4 mg/ml, 5.5 mg/ml, 5.6 mg/ml, 5.7 mg/ml, 5.8 mg/ml, 5.9 mg/ml, 6 mg/ml, 6.1 mg/ml, 6.2 mg/ml, 6.3 mg/ml, 6.4 mg/ml, 6.5 mg/ml, 6.6 mg/ml, 6.7 mg/ml, 6.8 mg/ml, 6.9 mg/ml, 7 mg/ml, 7.1 mg/ml, 7.2 mg/ml, 7.3 mg/ml, 7.4 mg/ml, 7.5 mg/ml, 7.6 mg/ml, 7.7 mg/ml, 7.8 mg/ml, 7.9 mg/ml, 8 mg/ml, 8.1 mg/ml, 8.2 mg/ml, 8.3 mg/ml, 8.4 mg/ml, 8.5 mg/ml, 8.6 mg/ml, 8.7 mg/ml, 8.8 mg/ml, 8.9 mg/ml, 9 mg/ml, 9.1 mg/ml, 9.2 mg/ml, 9.3 mg/ml, 9.4 mg/ml, 9.5 mg/ml, 9.6 mg/ml, 9.7 mg/ml, 9.8 mg/ml, 9.9 mg/ml, or 10 mg/ml, or any dose therebetween. Various drugs and their dose ranges listed in Table 1 could be used in an above-mentioned dose. As examples, the various doses listed above would be applicable to bimatoprost, timolol maleate, Brimonidine, and Timolol.

The effective amount of a medicament and/or active agent in a formulation may be an amount from about 0.001% to about 10%, e.g., about 0.001%, 0.002%, 0.003%, 0.004%, 0.005%, 0.006%, 0.007%, 0.008%, 0.009%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, or 10%, or any dose therebetween. Various drugs and their dose ranges listed in Table 1 could be used in an above-mentioned dose. As examples, the various doses listed above would be applicable to Calcipotriol, Sevoflurane, Travoprost, calcipotriene, diflucortolone, methylprednisolone, delgocitinib, isoconazole, azelaic acid, calcipotriene (with or without betamethasone dipropionate), diflucortolone (with or without isoconazole), betamethasone valerate (with or without fusidic acid), fusidic acid (with or without hydrocortisone acetate), hydrocortisone acetate (with or without fusidic acid), fusidic acid (with or without betamethasone valerate), betamethasone dipropionate (with or without calcipotriene), and isoconazole (with or without diflucortolone).

The effective dose of calcitonin may be an amount from about 50 IU to about 150, e.g., about 50 IU, 55 IU, 60 IU, 65 IU, 70 IU, 75 IU, 80 IU, 85 IU, 90 IU, 95 IU, 100 IU, 105 IU, 110 IU, 115 IU, 120 IU, 125 IU, 130 IU, 135 IU, 140 IU, 145 IU, or 150 IU, or any dose therebetween.

The effective dose of a medicament and/or active agent may be an amount from about 0.1 ml to about 5 ml, e.g., about 0.1 ml, 0.2 ml, 0.3 ml, 0.4 ml, 0.5 ml, 0.6 ml, 0.7 ml, 0.8 ml, 0.9 ml, 1 ml, 1.1 ml, 1.2 ml, 1.3 ml, 1.4 ml, 1.5 ml, 1.6 ml, 1.7 ml, 1.8 ml, 1.9 ml, 2 ml, 2.1 ml, 2.2 ml, 2.3 ml, 2.4 ml, 2.5 ml, 2.6 ml, 2.7 ml, 2.8 ml, 2.9 ml, 3 ml, 3.1 ml, 3.2 ml, 3.3 ml, 3.4 ml, 3.5 ml, 3.6 ml, 3.7 ml, 3.8 ml, 3.9 ml, 4 ml, 4.1 ml, 4.2 ml, 4.3 ml, 4.4 ml, 4.5 ml, 4.6 ml, 4.7 ml, 4.8 ml, 4.9 ml, or 5 ml, or any dose therebetween. Various drugs and their dose ranges listed in Table 1 could be used in an above-mentioned dose. As examples, the various doses listed above would be applicable to diphtheria vaccine, Dukarol/ShanChol cholera vaccine, and heplisav-B vaccine.

The effective dose of a medicament and/or active agent may be an amount from about 50 U to about 6000 U, e.g., about 50 U, 100 U, 150 U, 200 U, 250 U, 300 U, 350 U, 400 U, 450 U, 500 U, 550 U, 600 U, 650 U, 700 U, 750 U, 800 U, 850 U, 900 U, 950 U, 1000 U, 1100 U, 1200 U, 1300 U, 1400 U, 1500 U, 1600 U, 1700 U, 1800 U, 1900 U, 2000 U, 2100 U, 2200 U, 2300 U, 2400 U, 2500 U, 2600 U, 2700 U, 2800 U, 2900 U, 3000 U, 3100 U, 3200 U, 3300 U, 3400 U, 3500 U, 3600 U, 3700 U, 3800 U, 3900 U, 4000 U, 4100 U, 4200 U, 4300 U, 4400 U, 4500 U, 4600 U, 4700 U, 4800 U, 4900 U, 5000 U, 5100 U, 5200 U, 5300 U, 5400 U, 5500 U, 5600 U, 5700 U, 5800 U, 5900 U, or 6000 U, or any dose therebetween. Various drugs and their dose ranges listed in Table 1 could be used in an above-mentioned dose. As examples, the various doses listed above would be applicable to imiglucerase and insuline glargine.

The effective dose of nitric oxide may be about 20 ppm maintained for about 14 days.

The effective dose of vasopressin may be an amount from about 0.005 unit/minute to about 0.15 unit/minute, e.g., about 0.005 unit/minute, 0.006 unit/minute, 0.007 units/minute, 0.008 unit/minute, 0.009 unit/minute, 0.01 unit/minute, 0.015 unit/minute, 0.02 unit/minute, 0.025 unit/minute, 0.03 unit/minute, 0.035 unit/minute, 0.04 unit/minute, 0.045 unit/minute, 0.05 unit/minute, 0.055 unit/minute, 0.06 unit/minute, 0.065 unit/minute, 0.07 unit/minute, 0.075 unit/minute, 0.08 unit/minute, 0.085 unit/minute, 0.09 unit/minute, 0.095 unit/minute, 0.105 unit/minute, 0.110 unit/minute, 0.115 unit/minute, 0.12 unit/minute, 0125 unit/minute, 0.13 unit/minute, 0.135 unit/minute, 0.14 unit/minute, 0.145 unit/minute, or 0.15 unit/minute, or any dose therebetween.

### Listing of Transdermally Administered Medicaments

Table 1 list agents that can be administered transdermally according to some embodiments. The “% solution” indicates the relative dosing which is the amount in the transdermal solution. Notably, when a row in Table 1's medicament column recites two or more agents that are separated by a plus sign, as in “Glecaprevir+Pibrentasvir” and “Abacavir+Dolutegravir+Lamivudine”, these series of agents may be used alone or in combination. Thus, Glecaprevir may be used alone or may be used in combination with Pibrentasvir. Moreover, when two or more effective doses are separated by a comma or commas (in a row of the “Effective Dose” column), the first effective dose refers to the first listed agent, the second effective dose refers to the second listed agent, as so forth; the first listed effective dose can be for the first listed agent when alone or when in the stated combination and/or the second listed effective dose can be for the second listed agent when alone or when in the stated combination. And, for values in a row in the “% Solution (relative dosing)” column, when two or more % solutions are separated by a comma or commas, the first % solution refers to the first listed agent, the second effective % solution refers to the second listed agent, as so forth; the first listed “% Solution can be for the first listed agent when alone or when in the stated combination and/or the second listed “% Solution can be for the second listed agent when alone or when in the stated combination. Thus, the row in Table 1 describing “Glecaprevir+Pibrentasvir”, the effective dose for Glecaprevir is 150-450 mg and its % solution is 15% to 30% and the effective dose for Pibrentasvir is 60-180 mg and its % solution is 6% to 18%. As mentioned above, these effective dosages and % solutions may be for each agent alone or when the two (or more agents) are combined.

TABLE 1

| | Medicament | Effective Dose | | % Solution (Relative Dosing) | |
|---|---|---|---|---|---|
| 1 | (3S,4S)-8-(6-amino-5-((2-amino-3- | | | | |
| | chloropyridin-4-yl)thio)pyrazin-2- | | | | |
| | yl)-3-methyl-2-oxa-8- | | | | |
| | azaspiro[4.5]decan-4-amine | | | | |
| 2 | (S)-3-amino-6-methoxy-N-(3,3,3- | | | | |
| | trifluoro-2-hydroxy-2- | | | | |
| | methylpropyl)-5- | | | | |
| | (trifluoromethyl)picolinamide | | | | |
| 3 | 2-fluoro-N-methyl-4-[7-quinolin-6- | | | | |
| | yl-methyl)-imidazo [1,2- | | | | |
| | B][1,2,4]triazin-2yl]benzamide | | | | |
| 4 | 4-(3-amino-6-((1S,3S,4S)-3-fluoro- | | | | |
| | 4-hydroxycyclohexyl)pyrazin-2- | | | | |
| | yl)-N-((S)-1-(3-bromo-5- | | | | |
| | fluorophenyl)-2- | | | | |
| | (methylamino)ethyl)-2- | | | | |
| | fluorobenzamide | | | | |
| 5 | 4-(7-Hydroxy-2-isopropyl-4-oxo- | | | | |
| | 4H-quinazolin-3-yl)-benzonitrile | | | | |
| 6 | Abacavir + Dolutegravir + | 300-900 mg, 25-75 mg, | | 30%, 2.5-7.5%, 15-30% | |
| | Lamivudine | 150-450 mg | | | |
| 7 | Abiraterone Acetate | 250-1500 | mg | 25-30% | |
| 8 | abiratone acetate | 500-1500 | mg | 30% | |
| 9 | abobotulinum toxin A | 0.001-0.390 | ug | <0.00004% | |
| 10 | Acalabrutinib | | | | |
| 11 | Acarbose | 50-150 | mg | 5-15% | |
| 12 | Acetaminophen | 500-1500 | mg | 30% | |
| 13 | Acetazolamide | | | | |
| 14 | Acetylsalicylic Acid | 325-975 | mg | 30% | |
| 15 | acitretin | | | | |
| 16 | acyclovir | 100-1200 | mg | 10-30% | |
| 17 | adalimumab | 20-240 | mg | 2-24% | |
| 18 | adapalene | 0.1-0.3% | | 0.05-0.45% | |
| 19 | adapalene + benzoyl peroxide | 0.1-0.3%, | 2.5% | 0.05-0.45%, | 1.25-3.75% |
| 20 | Afoxolaner | 130-390 | mg | 13-30% | |
| 21 | agalsidase beta | 30-90 | mg | 3-9% | |
| 22 | Albendazole | | | | |
| 23 | Alectinib | 300-900 | mg | 30% | |
| 24 | Alectinib | | | | |
| 25 | alendronate sodium | 5-15 | mg | 0.5-1.5% | |
| 26 | alglucosidase alfa | 600-1800 | mg | 30% | |
| 27 | alitretinoin | | | | |
| 28 | Alogliptin | 12.5-37.5 | mg | 1.25-3.75% | |
| 29 | alpelisib | 150-450 | mg | 15-30% | |
| 30 | Aluminum Hydroxide | | | | |
| 31 | Ambrisentan | 2.5-7.5 | mg | 0.25-0.75% | |
| 32 | Amiodarone | | | | |
| 33 | Amitriptyline | | | | |
| 34 | Amlodipine | 2.5-15 | mg | 0.25-1.5% | |
| 35 | Amlodipine besylate | 1.25-15 | mg | 0.125-1.5% | |
| 36 | Amlodipine + Valsartan | 2.5-15 mg, | 80-480 mg | 0.25-1.5%, | 8-30% |
| 37 | amlodopine besylate + valsartan | 2.5-15 mg, | 80-480 mg | 0.25-1.5%, | 8-30% |
| 38 | amorolfine hydrochloride | 5% | | 2.5-7.5% | |
| 39 | Amoxicillin + Clavulanate | 400-1200 | mg | 30% | |
| 40 | Amphotericin B | | | | |
| 41 | Amphotericin B liposome | 187.5-562.5 | mg | 18.75-30% | |
| 42 | ampicillin + sulbactam | 0.5-3 g, | 0.25-1.5 g | 0.05-0.3%, | 0.03-0.15% |
| 43 | Amprenavir | | | | |
| 44 | antihemophilic factor, Fc fusion | 25-65 | IU/kg | | |
| | protein | | | | |
| 45 | apalutamide | 120-360 | mg | 12-30% | |
| 46 | Apatinib | | | | |
| 47 | apixaban | 1.25-7.5 | mg | 0.125-0.75% | |
| 48 | apremilast | 15-45 | mg | 1.5-4.5% | |
| 49 | Aprepitant | 40-187.5 | mg | 4-18.75% | |
| 50 | Aripiprazole | 1-45 | mg | 0.1-4.5% | |
| 51 | Artemether + Lumefantrine | | | | |
| 52 | aspirin | 250-750 | mg | 25-30% | |
| 53 | Atazanavir | 150-600 | mg | 15-30% | |
| 54 | atenolol | 25-150 | mg | 2.5-15% | |
| 55 | Atomoxetine | 20-150 | mg | 2-15% | |
| 56 | Atorvastatin | 10-120 | mg | 1-12% | |
| 57 | Avapritinib | | | | |
| 58 | Axitinib (Inlyta) | 2.5-7.5 | mg | 0.25-0.75% | |
| 59 | Azacitidine | 62.5-255 | mg | 6.25-25.5% | |
| 60 | Azathioprine | | | | |
| 61 | azelaic acid | 15% | | 7.5-22.5% | |
| 62 | Azilsartan medoxomil | 40-120 | mg | 4-12% | |
| 63 | azithromycin | 125-750 | mg | 12.5-30% | |
| 64 | baclofen | 0.0125-0.150 | mg | 0.00125-0.015% | |
| 65 | baricitinib | 1-3 | mg | 0.1-0.3% | |
| 66 | basiliximab | 10-30 | mg | 1-3% | |
| 67 | Batimastat (BB-94) | 250-750 | mg | 25-30% | |
| 68 | becaplermin | 0.0005-0.0015 | | 0.005-0.015% | |
| 69 | Belumosudil | | | | |
| 70 | Bendamustine | 95-285 | mg | 9.5-28.5% | |
| 71 | benzoyl peroxide | 2.5-10% | | 1.25-15% | |
| 72 | betamethasone valerate + fusidic | 0.1%, | 2% | 0.05-0.15%, | 1-3% |
| | acid | | | | |
| 73 | bexarotene | | | | |
| 74 | Bictegravir + Emtricitabine + TAF | 25-75 mg, 100-300 mg, | | 2.5-7.5%, 10-30%, 1.25-3.75% | |
| | | 12.5-37.5 mg | | | |
| 75 | Bimatoprost + timolol maleate | One drop of 0.3 mg/mL | | | |
| | | bimatoprost and 5 mg/mL | | | |
| | | timolol solution | | | |
| 76 | binimetinib (Mektovi) | | | | |
| 77 | Bortezomib | 1.24-3.72 | mg | 0.124-0.372% | |
| 78 | Bortezomib (Velcade) | 1.75-5.25 | mg | 0.18-0.53% | |
| 79 | Bosentan | 31.25-187.5 | mg | 3.125-18.75% | |
| 80 | Bosutinib (Bosulif) | 200-900 | mg | 20-30% | |
| 81 | botulinum toxin A | 0.73-2.19 | ng | 0.000000073-0.000000219% | |
| 82 | branched chain amino acids | 7-21 | g | 30% | |
| 83 | Brexpiprazole | 0.25-6 | mg | 0.025-0.6% | |
| 84 | brimonidine tartrate | 0.33% | | 0.15-0.48% | |
| 85 | Brimonidine + Timolol | One drop of 2 mg/mL | | | |
| | | brimonidine tartrate and 5 | | | |
| | | mg/mL timolol solution | | | |
| 86 | brodalumab | 105-315 | mg | 10.5-30% | |
| 87 | buproprion | 75-300 | mg | 7.5-30% | |
| 88 | Cabazitaxel | 20-60 | mg | 2-6% | |
| 89 | Cabozantinib | | | | |
| 90 | Cabozantinib (Cometriz) | 70-210 | mg | 7-21% | |
| 91 | Calcifediol | | | | |
| 92 | calcipotriene | 0.01% | | 0.0025-0.0075% | |
| 93 | Calcipotriene + betamethasone | 0.005%, | 0.064% | 0.0025-0.0075%, | 0.032-0.096% |
| | dipropionate | | | | |
| 94 | Calcipotriol | Thin layer of 0.005% | | 0.0025-0.0075% | |
| 95 | calcitonin | cream 50-150 IU or | | 0.0008%-0.0025% | |
| | | 0.008-0.025 mg | | | |
| 96 | calcitriol | 0.0003% | | 0.00015-0.00045% | |
| 97 | calcium carbonate | 0.5-2 | g | 30% | |
| 98 | Canagliflozin + Metformin | 25-75 mg, | 1000-3000 mg | 2.5-7.5%, | 30% |
| 99 | canakinumab | 75-225 | mg | 7.5-22.5% | |
| 100 | Capecitabine | 1200-3600 | mg | 30% | |
| 101 | Capmatinib | | | | |
| 102 | capsaicin | 0.1-1.5 | mg | 0.01-0.15% | |
| 103 | Carbamazepine | | | | |
| 104 | Carbidopa + levodopa | 5-37.5 mg, | 50-150 mg | 0.5-3.75%, | 5-15% |
| 105 | Carfilzomib | 19-57 | mg | 1.9-5.7% | |
| 106 | Carfilzomib (Kyprolis) | 10-90 | mg | 1-9% | |
| 107 | Cariprazine | 0.75-9 | mg | 0.075-0.9% | |
| 108 | carvedilol | 1.5-75 | mg | 0.15-7.5% | |
| 109 | CD-12681 | | | <30% | |
| 110 | CEE-321 | | | | |
| 111 | cefazolin | 125-1500 | mg | 12.5-30% | |
| 112 | cefepime | 250-2000 | mg | 25-30% | |
| 113 | Cefoperazone + Sulbactam | 1-3 mg, | 2-6 mg | 0.1-0.3%, | 0.2-0.6% |
| 114 | cefotaxime sodium | 250-2000 | | 25-30% | |
| 115 | celecoxib | 100-300 | mg | 10-30% | |
| 116 | cemiplimab | 175-525 | mg | 17.5-30% | |
| 117 | Ceritinib | | | | |
| 118 | Chlorothiazide | | | | |
| 119 | Chlorpromazine | | | | |
| 120 | Chlorthalidone | | | | |
| 121 | cinacalcet | 110-270 | mg | 11-27% | |
| 122 | ciprofloxacin | 250-1250 | mg | 25-30% | |
| 123 | Cisapride | | | | |
| 124 | citalopram | 10-90 | mg | 1-9% | |
| 125 | citric acid | 500-1500 | mg | 30% | |
| 126 | clarithromycin | 250-1000 | mg | 25-30% | |
| 127 | Clindamycin phosphate | 5-15 | mg | 0.5-1.5% | |
| 128 | Clobazam | 5-60 | mg | 0.5-6% | |
| 129 | clobetasol propionate | 0.05% | | 0.025-0.075% | |
| 130 | Clofazimine | | | | |
| 131 | clomiphene | 25-150 | mg | 2.5-15% | |
| 132 | clonidine | 0.05-3.0 | mg | 0.005-0.3% | |
| 133 | Clopidogrel | 75-450 | mg | 7.5-30% | |
| 134 | clopidogrel bisulfate | 37.5-112.5 | mg | 3.75-11.25% | |
| 135 | Colchocine | | | | |
| 136 | Colistin | | | | |
| 137 | Corticotropin | 0.12-6.24 | mg | 0.012-0.624% | |
| 138 | crisaborole | 10-30 | mg | 1-3% | |
| 139 | crizanlizumab | 155-465 | mg | 15.5-30% | |
| 140 | Crizotinib (Xalkori) | 125-375 | mg | 12.5-30% | |
| 141 | cyclobenzaprine | 5-90 | mg | 0.5-9% | |
| 142 | cyclosporine | 90-1650 | mg | 9-30% | |
| 143 | Dabigatran Etexilate | 37.5-225 | mg | 3.75-22.5% | |
| 144 | Dabrafenib | 25-225 | mg | 7.5-22.5% | |
| 145 | Dabrafenib + Trametinib | 75-225 mg, | 1-3 mg | 7.5-22.5%, | 0.1-0.3% |
| 146 | dacomitinib (Vizimpro) | | | | |
| 147 | dalbavancin | 300-1200 | mg | ~30% | |
| 148 | Danazol | | | | |
| 149 | Dapagliflozin | 2.5-15 | mg | 0.25-1.5% | |
| 150 | Dapsone | | | | |
| 151 | daptomycin | 125-562.5 | mg | 12.5-30% | |
| 152 | Darunavir + cobicistat + | 400-1200 mg, 75-225 mg, | | 30%, 7.5-22.5%, | |
| | emtricitabine + tenofovir | 100-300 mg, 5-15 mg | | 10-30%, 0.5-1.5% | |
| | alafenamide | | | | |
| 153 | Dasatinib | 70-210 | mg | 7-21% | |
| 154 | decitabine | 12.5-51 | mg | 1.25-5.1% | |
| 155 | defatted Peanut (Arachis hypogaea) | 3-300 mg q.d. | | 1-5% | |
| | Flour | | | | |
| 156 | deferasirox | 62.5-750 | mg | 6.25-30% | |
| 157 | delgocitinib | 0.50% | | 0.25-0.75% | |
| 158 | deoxycholic acid | 50-150 | mg | 5-15% | |
| 159 | Desipramine | | | | |
| 160 | desonide | 0.05% | | 0.025-0.075% | |
| 161 | Dexlansoprazole | 15-90 | mg | 1.5-9% | |
| 162 | Dexmethylphenidate | 1.25-15 | mg | 0.125-1.5% | |
| 163 | dextroamphetamine/amphetamine | 1.25-90 | mg | 0.125-9% | |
| | salts | | | | |
| 164 | Diclofenac | | | | |
| 165 | diclofenac sodium | 50-150 | mg | 5-15% | |
| 166 | diflucortolone | 0.10% | | 0.05-0.15% | |
| 167 | Diflucortolone + isoconazole | 0.1%, | 1% | 0.05-0.15%, | 0.5-1.5% |
| 168 | Difunisal | | | | |
| 169 | Digoxin | | | | |
| 170 | Diloxanide | | | | |
| 171 | Dimethyl Fumarate | 120-360 | mg | 12-30% | |
| 172 | diphenhydramine hydrochloride | 10-30 | mg | 1-3% | |
| 173 | diphtheria vaccine | 0.5 mL injection | | | |
| 174 | docetaxel | 70-210 | mg | 7-21% | |
| 175 | Dolutegravir | 25-75 | mg | 2.5-7.5% | |
| 176 | Donepezil | 2.5-34.5 | mg | 0.25-3.45% | |
| 177 | Doxercalciferol | | | | |
| 178 | doxycycline | 75-300 | mg | 7.5-30% | |
| 179 | doxycycline ER | 20-60 | mg | 2-6% | |
| 180 | Doxycycline Hyclate (B) | 8.66 | | 15% | |
| 181 | Doxycycline Monohydrate (A) | 15.61 | | <1% | |
| 182 | Dronabinol | | | | |
| 183 | Dronedarone | 200-600 | mg | 20-30% | |
| 184 | Drospirenone + Ethinyl Estradiol | 1.5-4.5 mg, | 0.01-0.03 mg | 0.15-0.45%, | 0.001-0.003% |
| 185 | Dukoral/ShanChol cholera vaccine | 1.5 mL orally | | | |
| 186 | Duloxetine | 15-180 | mg | 1.5-18% | |
| 187 | dupilumab | 150-900 | mg | 15-30% | |
| 188 | durvalumab | 310-930 | mg | 30% | |
| 189 | Dutasteride | 0.25-0.75 | mg | 0.025-0.075% | |
| 190 | Duvelisib | | | | |
| 191 | ecallantide | 15-45 | mg | 1.5-4.5% | |
| 192 | Edoxaban | 30-90 | mg | 3-9% | |
| 193 | Efavirenz | | | | |
| 194 | Efavirenz + Emtricitabine + TDF | 300-900 mg, 100-300 mg, | | 30%, 10-30%, 15-30% | |
| | | 150-450 mg | | | |
| 195 | Elafibranor | | | | |
| 196 | elagolix sodium | 75-300 | mg | 7.5-30% | |
| 197 | Elbasvir + Grazoprevir | 25-75 mg, | 50-150 mg | 2.5-7.5%, | 5-15% |
| 198 | Eletriptan | 10-120 | mg | 1-12% | |
| 199 | Eltrombopag | 25-75 | mg | 2.5-7.5% | |
| 200 | Elvitegravir + Cobicistat + | 75-225 mg, 75-225 mg, | | 7.5-22.5%, 7.5-22.5%, 10-30%, | |
| | Emtricitabine + TAF | 100-300 mg, 5-15 mg | | 0.5-1.5% | |
| 201 | Empagliflozin | 5-37.5 | mg | 0.5-3.75% | |
| 202 | Emtricitabine + Rilpivirine + TAF | 100-300 mg, 12.5-37.5 mg, | | 10-30%, 1.25-3.75%, 1.25-3.75% | |
| | | 12.5-37.5 mg | | | |
| 203 | Emtricitabine + TAF | 100-300 mg, | 12.5-37.5 mg | 10-30%, | 1.25-3.75% |
| 204 | Emtricitabine + Tenofovir | 100-300 mg, | 150-450 mg | 10-30%, | 15-30% |
| | Disoproxil Fumarate | | | | |
| 205 | encorafenib (Braftovi) | | | | |
| 206 | enfuviritide | 45-135 | mg | 4.5-13.5% | |
| 207 | enoxaparin sodium | 20-60 | mg | 2-6% | |
| 208 | Entecavir | 0.5-1.5 | mg | 0.05-0.15% | |
| 209 | entrectinib | 300-900 | mg | 30% | |
| 210 | Enzalutamide | 80-240 | mg | 8-24% | |
| 211 | Epinephrine | 0.15-0.45 | mg | 0.015-0.045% | |
| 212 | erdafitinib | 4-12 | mg | 0.4-1.2% | |
| 213 | erenumab | 35-210 | mg | 7.5-21% | |
| 214 | Eribulin | 1.3-3.9 | mg | 0.13-0.39% | |
| 215 | Erlotinib | 50-225 | mg | 5-22.5% | |
| 216 | Erlotinib HCl (Tarceva) | 75-225 | mg | 7.5-22.5% | |
| 217 | Ertapenem | 0.5-1.5 | g | 30% | |
| 218 | Erythromycin | | | | |
| 219 | escitalopram | 5-30 | mg | 0.5-3% | |
| 220 | esketamine (ketamine) | 14-42 | mg | 1.4-4.2% | |
| 221 | Esomeprazole | 10-60 | mg | 1-6% | |
| 222 | esopremazole | 5-60 | mg | 0.5-6% | |
| 223 | estradiol | 1-3 | mg | 0.1-0.3% | |
| 224 | Estrogens − conjugated | 0.15-0.625 | mg | 0.015-0.0625% | |
| 225 | etanercept | 25-75 | mg | 2.5-7.5% | |
| 226 | Ethinyl Estradiol + Etonogestrel | 0.06-0.18 mg, | 0.008-0.023 mg | 0.006-0.018%, | 0.0008-0.0023% |
| 227 | Etonogestrel | 35-70 | mcg | 0.0035-0.0070% | |
| 228 | Etravirine | | | | |
| 229 | etretinate | | | | |
| 230 | Everolimus | 2.5-30 | mg | 0.25-3% | |
| 231 | Everolimus (Votubia, Zortress/ | 0.375-1.5 mg (Zortress) | | 0.0375-0.15% | |
| | Certican) | | | | |
| 232 | Everolimus (Afinitor) | 5-15 mg or | 2.5-30 mg | 0.25-3% or 0.5-1.5% | |
| 233 | Ezetimibe | 5-15 | mg | 0.5-1.5% | |
| 234 | Ezetimibe + Atorvastatin | 5-15 mg, | 5-120 mg | 0.5-1.5%, | 0.5-12% |
| 235 | Ezetimibe + Simvastatin | 5-15 mg, | 5-120 mg | 0.5-1.5%, | 0.5-12% |
| 236 | Famotidine | | | | |
| 237 | fasinumab | 3-12 | mg | 0.3-1.2% | |
| 238 | Febuxostat | 20-120 | mg | 2-12% | |
| 239 | Fedratinib | | | | |
| 240 | Fenofibrate | | | | |
| 241 | Ferric Carboxymaltose | 375-1125 | mg | 30% | |
| 242 | fexofenadine hydrochloride | 30-270 | mg | 3-27% | |
| 243 | filgotinib | 100-300 | mg | 10-30% | |
| 244 | filgrastim | 0.3-0.9 | mg | 0.03-0.09% | |
| 245 | fingolimod | 0.25-0.75 | mg | 0.025-0.075% | |
| 246 | fluocinolone acetonide | 0.01% | | 0.005-0.015% | |
| 247 | fluocinolone acetonide + | 0.01%, 4%, 0.05% | | 0.005-0.015%, 2-6%, 0.025-0.075% | |
| | hydroquinone + tretinoin | | | | |
| 248 | fluoxetine | 2.5-90 | mg | 0.25-9% | |
| 249 | Flurbiprofen | | | | |
| 250 | Folic acid | | | | |
| 251 | Forskolin | 50-375 | mg | 5-30% | |
| 252 | Fostamatinib | | | | |
| 253 | Fulvestrant | 125-750 | mg | 12.5-30% | |
| 254 | furosemide | 10-120 | mg | 1-12% | |
| 255 | fusidic acid + hydrocortisone | 2%, | 1% | 1-3%, | 0.5-1.5% |
| | acetate | | | | |
| 256 | gabapentin | 150-900 | mg | 15-30% | |
| 257 | Ganetespib | 125-375 | mg | 12.5-30% | |
| 258 | Gefitinib (Iressa) | 125-375 | mg | 12.5-30% | |
| 259 | Gilteritinib | | | | |
| 260 | glasdegib (Daurismo) or glasdegib | 50-150 | mg | 5-15% | |
| | maleate | | | | |
| 261 | Glecaprevir + Pibrentasvir | 150-450 mg, | 60-180 mg | 15-30%, | 6-18% |
| 262 | Glibenclamide | | | | |
| 263 | Glimepiride | 0.5-6 | mg | 0.05-0.6% | |
| 264 | glipizide | 2.5-30 | mg | 0.25-3% | |
| 265 | glucagon | 0.25-1.5 | mg | 0.025-0.15% | |
| 266 | glucosamine | 250-750 | mg | 25-30% | |
| 267 | glutaric anhydride | 10-300 | mg | 1-30% | |
| 268 | glycopyrrolate | 0.1-0.3 | mg | 0.01-0.03% | |
| 269 | Goserelin | 1.8-5.4 | mg | 0.18-0.54% | |
| 270 | goserelin LA | 1.8-5.4 | mg | 0.18-0.54% | |
| 271 | granisetron | 0.5-1.5 | mg | 0.05-0.15% | |
| 272 | Griseofulvin | | | | |
| 273 | halobetasol propionate | 0.025-0.075 | mg | 0.003-0.008% | |
| 274 | Haloperidol | | | | |
| 275 | heparin sodium | 10-40 | mg | 1-4% | |
| 276 | heplisav-B vaccine | 0.5 mL injection | | | |
| 277 | hyaluronic acid | | | | |
| 278 | Hydrochlorothiazide | | | | |
| 279 | Hydrochlorothiazide + Valsartan | 160-480 mg, | 12.5-37 mg | 16-30%, | 1.25-3.75% |
| 280 | hydrocortisone | 12.5-37.5 | mg | 1.25-3.75% | |
| 281 | hydroxychloroquine | 200-800 | mg | 20-30% | |
| 282 | Hydroxyprogesterone | 50-300 | mg | 5-30% | |
| 283 | Ibrutinib | 280-840 | mg | 28-30% | |
| 284 | Ibrutinib (Imbruvica) | 280-840 | mg | 28-30% | |
| 285 | ibuprofen | 50-150 | mg | 5-15% | |
| 286 | icatibant acetate | 15-45 | mg | 1.5-4.5% | |
| 287 | Idelalisib | | | | |
| 288 | Imatinib | 50-1200 | mg | 5-30% | |
| 289 | Imatinib (Gleevec) | 200-600 | mg | 20-30% | |
| 290 | imatinib mesylate | 50-1200 | mg | 5-30% | |
| 291 | imiglucerase | 465-5,580 | U | | |
| 292 | inclisiran | 142-426 | mg | 14.2-30% | |
| 293 | Indinavir | | | | |
| 294 | Indomethacin | | | | |
| 295 | infliximab | 155-465 | mg | 15.5-30% | |
| 296 | ingenol mebutate | 0.015-0.05% | | 0.0075-0.075% | |
| 297 | inotersen | 142-426 | mg | 14.2-30% | |
| 298 | insulin A | 0.38-1.15 | mg | 0.038-0.115% | |
| 299 | insuline glargine | 150-450 | U | | |
| 300 | interferon beta-1b | 0.03125-0.375 | mg | 0.003-0.038% | |
| 301 | Iopanoic acid | | | | |
| 302 | Irbesartan + Hydrochlorothiazide | 75-450 mg, | 6.25-37.5 mg | 7.5-30%, | 0.625-3.75% |
| 303 | isoconazole | 1% | | 0.5-1.5% | |
| 304 | Isotretinoin | | | | |
| 305 | Itraconazole | | | | |
| 306 | Ivacaftor | 75-225 | mg | 7.5-22.5% | |
| 307 | ivermectin | 1% | | 0.5-1.5% | |
| 308 | Ixazomib | 2-6 | mg | 0.2-0.6% | |
| 309 | ketoconazole | 10-30 | mg | 1-3% | |
| 310 | ketones | | | | |
| 311 | ketoprofen | 12.5-75 | mg | 1.25-7.5% | |
| 312 | Keyruvia | | | | |
| 313 | Lacosamide | 25-600 | mg | 2.5-30% | |
| 314 | lamictal | 4.5-112.5 | mg | 0.45-11.25% | |
| 315 | Lansoprazole | | | | |
| 316 | Lapatinib | | | | |
| 317 | Lapatinib (Tykerb) | 750-2250 | mg | 30% | |
| 318 | larotrectinib | 50-150 | mg | 5-15% | |
| 319 | Ledipasvir + Sofosbuvir | 45-135 mg, | 200-600 mg | 4.5-13.5%, | 20-30% |
| 320 | leflunomide | 10-30 | mg | 1-3% | |
| 321 | Lenalidomide | 5-37.5 | mg | 0.5-3.75% | |
| 322 | Lenvatinib | 9-36 | mg | 0.9-3.6% | |
| 323 | LEO-138559 | | | | |
| 324 | LEO-152020 | | | | |
| 325 | Leuprolide | 3.75-11.25 | | 0.38-1.13% | |
| 326 | Levetiracetam | 250-750 | mg | 25-30% | |
| 327 | Levodopa/Benserazide | 50-1000 mg, | 25-250 mg | 5-30%, | 2.5-25% |
| 328 | Levonorgestrel | 0.375-1.025 | mg | 0.0375-0.1025% | |
| 329 | Levothyroxine | 12.5-450 | mcg | 0.00125-0.045% | |
| 330 | Linagliptin + Metformin | 1.25-3.75 mg, | 500-1500 mg | 0.125-0.375%, | 30% |
| 331 | linezolid | 250-750 | mg | 25-30% | |
| 332 | lisinopril | 5-120 | mg | 0.5-12% | |
| 333 | L-lysine free base | 1.5-4.5 | g | 30% | |
| 334 | lofexidine | 0.27-0.81 | mg | 0.027-0.081% | |
| 335 | Lopinavir | | | | |
| 336 | Loratadine | | | | |
| 337 | lorlatinib (Lorviqua) | | | | |
| 338 | Losartan + Hydrochlorothiazide | 12.5-75 mg, | 6.25-75 mg | 1.25-7.5%, | 0.625-7.5% |
| 339 | Lovastatin | | | | |
| 340 | Lubiprostone | 0.012-0.036 | mg | 0.001-0.004% | |
| 341 | Lumacaftor/Ivacaftor | 100-300 mg, | 62.5-187.5 mg | 10-30%, | 6.25-18.75% |
| 342 | Lurasidone | 20-240 | mg | 2-24% | |
| 343 | Luspatercept | 12.5-112.5 | mg | 1.25-11.25% | |
| 344 | lymecycline | 150-450 | mg | 15-30% | |
| 345 | Macitentan | 5-15 | mg | 0.5-1.5% | |
| 346 | magnesium lactate | 40-120 | mg | 4-12% | |
| 347 | Marimastat (BB-2516) | 5-37.5 | mg | 0.5-3.75% | |
| 348 | Marizomib (NPI-0052) | 0.5-1.5 | mg | 0.05-0.15% | |
| 349 | Mebendazole | | | | |
| 350 | Mefloquine | | | | |
| 351 | melatonin | 1-10 | mg | 0.1-1% | |
| 352 | meloxicam | 3.75-22.5 | mg | 0.38-2.25% | |
| 353 | memantine hydrochloride | 2.5-15 | mg | 0.25-1.5% | |
| 354 | meningococcal [serotype b] vaccine | | | | |
| 355 | Mesalazine | 1.2-7.2 | g | 30% | |
| 356 | metformin | 250-4500 | mg | 25-30% | |
| 357 | Metformin + Sitagliptin | 250-750 mg, | 25-75 mg | 25-30%, | 2.5-7.5% |
| 358 | methotrexate | 1.25-3.75 | mg | 0.125-0.375% | |
| 359 | methyl aminolevulinate | 16% | | 8-24% | |
| | hydrochloride | | | | |
| 360 | Methylphenidate | 10-90 | mg | 1-9% | |
| 361 | methylprednisolone | 0.10% | | 0.05-0.015% | |
| 362 | Metoprolol succinate | 25-150 | mg | 2.5-15% | |
| 363 | metronidazole | 125-2000 | mg | 12.5-30% | |
| 364 | Midostaurin | 25-150 | mg | 2.5-15% | |
| 365 | minoxidil | 25-75 | mg | 2.5-7.5% | |
| 366 | Mirabegron | 12.5-75 | mg | 1.25-7.5% | |
| 367 | mizolastine MR | 5-15 | mg | 0.5-1.5% | |
| 368 | Montelukast | 2-15 | mg | 0.2-1.5% | |
| 369 | Mycophenolate Mofetil | 500-1500 | mg | 30% | |
| 370 | N-(3-(2-(2-hydroxyethoxy)-6- | | | | |
| | morpholinopyridin-4-yl)-4- | | | | |
| | methylphenyl)-2-(trifluoromethyl)- | | | | |
| | isonicotinamide | | | | |
| 371 | N-(3-(6-Amino-5-(2-(N- | | | | |
| | methylacrylamido)ethoxy)pyrimidin- | | | | |
| | 4-yl)-5-fluoro-2-methylphenyl)- | | | | |
| | 4-cyclopropyl-2-fluorobenzamide | | | | |
| 372 | N-[4- | | | | |
| | (Chlorodifluoromethoxy)phenyl]-6- | | | | |
| | [(3R)-3-hydroxpyrrolidin-1-yl]-5- | | | | |
| | (1H-pyrazol-5-yl)-pyridine-3- | | | | |
| | carboxamide hydrochloride | | | | |
| 373 | Nalidixic acid (Quinolone | | | | |
| | antibiotic) | | | | |
| 374 | naloxone | 2-6 | mg | 0.2-0.6% | |
| 375 | Naproxen | | | | |
| 376 | Navitoclax (ABT-263) | 125-375 | mg | 12.5-30% | |
| 377 | Nebivolol | 2.5-30 | mg | 0.25-3% | |
| 378 | Nelfinavir | | | | |
| 379 | nemolizumab | | | <30% | |
| 380 | neomycin | 500-4500 | mg | 30% | |
| 381 | Neovastat (AE-941) | 60-360 | mg | 6-30% | |
| 382 | Neratinib (Nerlynx) | 40 mg-360 mg, | 240 mg | 12-30% | |
| 383 | Neratinib − Free Base | 3.5 | | <1% | |
| 384 | Neratinib Maleate | 4.23 | | <1% | |
| 385 | Nevirapine | | | | |
| 386 | Niclosamide | | | | |
| 387 | Nifedipine | 5-45 | mg | 0.5-4.5% | |
| 388 | nilotinib | 150-600 | mg | 15-30% | |
| 389 | Nilotinib | | | | |
| 390 | Nilotinib (Tasigna) | 200-600 | mg | 20-30% | |
| 391 | Nintedanib | | | | |
| 392 | niratimib | 50-225 | mg | 5-22.5% | |
| 393 | Nitric Oxide | 20 ppm maintained | | | |
| | | for 14 days | | | |
| 394 | Nitrofurantoin | | | | |
| 395 | Norethindrone Acetate + Ethinyl | 0.5-1.5 mg, | 10-30 mcg | 0.05-0.155%, | 0.001-0.003% |
| | Estradiol | | | | |
| 396 | nusinersen | 6-18 | mg | 0.6-1.8% | |
| 397 | NVP-AUY922 | 60-180 | mg | 6-18% | |
| 398 | Nystatin | | | | |
| 399 | Obatoclax (GX15-070) | 1-3 | g | 0.1-0.3% | |
| 400 | octreotide acetate | 0.025-0.900 | mg | 0.003-0.09% | |
| 401 | ofatumumab | 150-3000 | mg | 15-30% | |
| 402 | Ofloxacin | | | | |
| 403 | Olanzapine | 1.25-22.5 | mg | 0.125-2.25% | |
| 404 | olaparib | 150-450 | mg | 15-30% | |
| 405 | Olmesartan Medoxomil | 10-60 | mg | 1-6% | |
| 406 | olumacostat | 3.75-11.25 | mg | 0.38-1.13% | |
| 407 | omalizumab | 37.5-900 | mg | 3.75-30% | |
| 408 | Oseltamivir | 37.5-112.5 | mg | 3.75-11.25% | |
| 409 | Osimertinib | 40-120 | mg | 4-12% | |
| 410 | osimertinib mesylate | 40-120 | mg | 4-12% | |
| 411 | Oxaprozin | | | | |
| 412 | Ozanimod | 0.46-1.38 | mg | 0.046-0.138% | |
| 413 | Paclitaxel | | | | |
| 414 | Paclitaxel Protein Bound | 120-360 | mg | 12-30% | |
| 415 | Palbociclib (Ibrance) | 62.5-187.5 | mg | 6.25-18.75% | |
| 416 | Paliperidone Palmitate | 78-351 | mg | 7.8-30% | |
| 417 | Pantoprazole | 20-60 | mg | 2-6% | |
| 418 | parathyroid hormone | 0.025-0.075 | mg | 0.003-0.008% | |
| 419 | Paricalcitol | | | | |
| 420 | patisiran | 15-45 | mg | 1.5-4.5% | |
| 421 | pazopanib | 200-1200 | mg | 20-30% | |
| 422 | Pazopanib (Votrient) | 400-1200 | mg | 30% | |
| 423 | pazopanib hydrochloride | 100-1200 | mg | 10-30% | |
| 424 | Peficitinib | | | | |
| 425 | pegfilgrastim | 3-9 | mg | 0.3-0.9% | |
| 426 | Pembrolizumab (Keytruda) | | | | |
| 427 | Pemetrexed | 475-1425 | mg | 30% | |
| 428 | Pemigatinib | | | | |
| 429 | Perifosine | 75-225 | mg | 7.5-22.5% | |
| 430 | Pexidartinib | | | | |
| 431 | Phenazopyridine | | | | |
| 432 | Phenytoin | | | | |
| 433 | Piperacillin + tazobactam | 1.5-4.5 g, | 188-563 mg | 30%, | 18.8-30% |
| 434 | Pirfenidone | 133.5-400.5 | mg | 13.35-30% | |
| 435 | Piroxicam | | | | |
| 436 | Pomalidomide | 2-6 | mg | 0.2-0.6% | |
| 437 | Ponatinib | | | | |
| 438 | Ponatinib (lclusig) | 22.5-67.5 | mg | 2.25-6.75% | |
| 439 | Posaconazole | 50-450 | mg | 5-30% | |
| 440 | Pralsetinib | | | | |
| 441 | pravastatin | 5-120 | mg | 0.5-12% | |
| 442 | prednicarbate | 0.05-0.15 | mg | 0.005-0.015% | |
| 443 | prednisone | 5-90 | mg | 0.5-9% | |
| 444 | pregabalin | 150-450 | mg | 15-30% | |
| 445 | pregabalin + celecoxib | 150-450 mg, | 100-300 mg | 15-30%, | 10-30% |
| 446 | Prinomastat (AG-3340) | 7.5-22.5 | mg | 0.75-2.25% | |
| 447 | Progesterone | 50-300 | mg | 5-30% | |
| 448 | propanolol | 20-960 | mg | 2-30% | |
| 449 | protamine sulfate | 25-375 | mg | 2.5-30% | |
| 450 | psilocybin | | | | |
| 451 | Pyrantel | | | | |
| 452 | Pyrimethamine | | | | |
| 453 | Quetiapine | 12.5-1200 | mg | 1.25-30% | |
| 454 | Quinupristin + dalfopristin | 90-270 mg, | 210-630 mg | 9-27%, | 21-30% |
| 455 | Raloxifene | | | | |
| 456 | Raltegravir | 200-1200 | mg | 20-30% | |
| 457 | ranibizumab | 0.15-0.75 | mg | 0.015-0.075% | |
| 458 | ranitidine | 50-450 | mg | 5-30% | |
| 459 | Ranolazine | 250-1500 | mg | 25-30% | |
| 460 | Rebimastat (BMS-275291) | 600-1, 800 | mg | 30% | |
| 461 | Regorafenib | 80-240 | mg | 8-24% | |
| 462 | Regorafenib (Stivarga) | 80-240 | mg | 8-24% | |
| 463 | relugolix (Orgovyx) | | | | |
| 464 | remibrutinib | 12.5-37.5 | mg | 1.25-3.75% | |
| 465 | resiquimod | | | <30% | |
| 466 | retinal | | | | |
| 467 | Retinol | | | | |
| 468 | ribociclib | 300-900 | mg | 30% | |
| 469 | ribociclib succinate | 300-900 | mg | 30% | |
| 470 | Rifampin | | | | |
| 471 | Rilpivirine | 12.5-37.5 | mg | 1.25-3.75% | |
| 472 | Rilpivirine + Emtricitabine + TDF | 12.5-37.5 mg, 100-300 mg, | | 1.25-3.75%, 10-30%, 15-30% | |
| | | 150-450 mg | | | |
| 473 | Rimegepant | 37.5-102.5 | mg | 3.75-10.25% | |
| 474 | Riociguat | 0.5-2.5 | mg | 0.05-0.25% | |
| 475 | Risdiplam | 2.5-7.5 | mg | 0.25-0.75% | |
| 476 | Risperidone | 0.125-24 | mg | 0.013-2.4% | |
| 477 | Ritonavir | | | | |
| 478 | Rivaroxaban | 10-30 | mg | 1-3% | |
| 479 | Rosuvastatin | 5-60 | mg | 0.5-6% | |
| 480 | rosuvastatin calcium | 2.5-60 | mg | 0.25-6% | |
| 481 | Rotigotine | 2.25-19 | mg | 0.225-1.9% | |
| 482 | roxadustat | 25-270 | mg | 2.5-27% | |
| 483 | rucaparib camsylate (Rubraca) | | | | |
| 484 | ruxolitinib | 2.5-30 | mg | 0.25-3% | |
| 485 | Ruxolitinib (Jafaki) | 5-30 | mg | 0.5-3% | |
| 486 | Sacubitril, valsartan | 24.5-145.5 mg, | 25.5-154.5 | 2.45-14.55%, | 2.55-15.45% |
| 487 | salicylic acid | 10-300 | mg | 1-30% | |
| 488 | Sapropterin | 150-1800 | mg | 15-30% | |
| 489 | Saquinavir | | | | |
| 490 | sarilumab | 100-300 | mg | 10-30% | |
| 491 | Saxagliptin, Metformin | 1.25-7.5 mg, | 250-1500 mg | 0.125-0.75%, | 25-30% |
| 492 | secukinumab | 150-450 | mg | 15-30% | |
| 493 | Selexipag | 0.1-2.4 | mg | 0.01-0.24% | |
| 494 | selinexor | 40-120 | mg | 4-12% | |
| 495 | Selpercatinib | | | | |
| 496 | selpercatinib (LOXO-292) | 60-240 | mg | 6-24% | |
| 497 | Selumetinib | | | | |
| 498 | semaglutide | 0.125-1.5 | mg | 0.013-0.15% | |
| 499 | sertraline | 12.5-300 | mg | 1.25-30% | |
| 500 | Sevoflurane | 0.7-4.9% vaporized | | | |
| | | in O2 gas | | | |
| 501 | sildenafil | 12.5-150 | mg | 1.25-15% | |
| 502 | Sildenafil Citrate | 3.6 | | <1% | |
| 503 | siponimod | 1-3 | mg | 0.1-0.3% | |
| 504 | Siremadlin | 5-60 | mg | 0.5-6% | |
| 505 | Sirolimus | | | | |
| 506 | Sitagliptin phosphate monohydrate | 12.5-150 | mg | 1.25-15% | |
| 507 | Sodium bicarbonate or sodium | | | 1% to 50% | |
| | carbonate | | | | |
| 508 | sodium deoxycholate | 50 | mg | 5-15% | |
| 509 | sodium nitrate | 298-893 | mg | 30% | |
| 510 | Sofosbuvir + Velpatasvir | 200-600 mg, | 50-150 mg | 20-30%, | 5-15% |
| 511 | Sofosbuvir + Velpatasvir + | 200-600 mg, 50-150 mg, | | 20-30%, 5-15%, 5-15% | |
| | Voxilaprevir | 50-150 mg | | | |
| 512 | Solifenacin | 2.5-15 | mg | 0.25-1.5% | |
| 513 | somatropin | 0.12-6.24 | mg | 0.012-0.624% | |
| 514 | sonidegib phosphate | 100-300 | mg | 10-30% | |
| 515 | Sorafenib | 200-600 | mg | 20-30% | |
| 516 | Sorafenib (Nexavar) | 200-600 | mg | 20-30% | |
| 517 | spartalizumab | 150-450 | mg | 15-30% | |
| 518 | Spironolactone | | | | |
| 519 | sufentanil citrate | 0.03-0.18 | mg | 0.003-0.018% | |
| 520 | Sugammadex | 125-375 | mg | 12.5-30% | |
| 521 | Sulfadiazine | | | | |
| 522 | Sulfamethoxazole | | | | |
| 523 | sulfasalazine | 250-1000 | mg | 25-30% | |
| 524 | sumatriptan | 50-150 | mg | 5-15% | |
| 525 | Sunitinib | 25-75 | mg | 2.5-7.5% | |
| 526 | Sunitinib | | | | |
| 527 | Sunitinib (sutent) | 25-75 | mg | 2.5-7.5% | |
| 528 | sunitinib malate (Sutent) | | | | |
| 529 | Tacrolimus | 6-18 | mg | 0.6-1.8% | |
| 530 | talazoparib − Talzenna) | | | | |
| 531 | Talinolol | | | | |
| 532 | Tamoxifen | | | | |
| 533 | Tamsulosin | 0.2-1.2 | mg | 0.02-0.12% | |
| 534 | tazarotene | | | | |
| 535 | Temsirolimus (CCI-779, Torisel) | 12.5-37.5 | mg | 1.25-3.75% | |
| 536 | Teneligliptin | 50-150 | mg | 5-15% | |
| 537 | Tepotinib | | | | |
| 538 | terbinafine | 5-15 | mg | 0.5-1.5% | |
| 539 | Terfenadine | | | | |
| 540 | Teriflunomide | 3.5-21 | mg | 0.35-2.1% | |
| 541 | Testosterone | 25-600 | mg | 2.5-30% | |
| 542 | Tezacaftor + Ivacaftor | 50-150 mg, | 75-225 mg | 5-15%, | 7.5-22.5% |
| 543 | THC | 1.25-3.75 | mg | 0.125-0.375% | |
| 544 | ticagrelor | 30-270 | mg | 3-27% | |
| 545 | tigecycline | 25-150 | mg | 2.5-15% | |
| 546 | Tipranavir | | | | |
| 547 | tisagenlecleucel | | | | |
| 548 | Tisagenlecleucel (Kymriah) | | | | |
| 549 | Tivozanib | | | | |
| 550 | tofacitinib citrate | 5-15 | mg | 0.5-1.5% | |
| 551 | Tolvaptan | 7.5-90 | mg | 0.75-9% | |
| 552 | tralokinumab | 150-900 | mg | 15-30% | |
| 553 | tramadol | 25-150 | mg | 2.5-15% | |
| 554 | Trametinib | 1-3 | mg | 0.1-0.3% | |
| 555 | Trametinib (Mekinist) | 1-3 | mg | 0.1-0.3% | |
| 556 | trametinib + dabrafenib | 1-3 mg, | 75-225 mg | 0.1-0.3%, | 7.5-22.5% |
| 557 | Trametinib + dabrafenib | 1-3 mg, | 75-225 mg | 0.1-0.3%, | 7.5-22.5% |
| 558 | tranexamic acid | 50-1500 | mg | 5-30% | |
| 559 | Travoprost | One drop of | | | |
| | | 0.004% solution | | | |
| 560 | trazodone | 75-600 | mg | 7.5-30% | |
| 561 | tretinoin | 5-15 | mg | 0.5-1.5% | |
| 562 | triancinolone | 2.5-100 | mg | 0.25-10% | |
| 563 | Triclabendazole | | | | |
| 564 | trifarotene | 0.005% | | 0.0025-0.0075% | |
| 565 | Trimethoprim | | | | |
| 566 | Triptorelin | 3.75-33.75 | mg | 0.38-3.38% | |
| 567 | tris | 7-21 | g | ~30% | |
| 568 | Tucatinib | | | | |
| 569 | ubrogepant | 25-150 | mg | 2.5-10% | |
| 570 | Umbralisib | | | | |
| 571 | upadacitinib | 7.5-22.5 | mg | 0.75-2.25% | |
| 572 | valproic acid | 450-1350 | mg | 30% | |
| 573 | valsartan | 10-480 | mg | 1-30% | |
| 574 | vancomycin | 250-2000 | mg | 25-30% | |
| 575 | Vancomycin Hydrochloride (A) | 8 | | 71% | |
| 576 | Vancomycin Hydrochloride (B) | 15 | | 35% | |
| 577 | Vandetanib | | | | |
| 578 | Vandetenib (Caprelsa) | 150-450 | mg | 15-30% | |
| 579 | Varenicline | 0.25-1.5 | mg | 0.025-0.15% | |
| 580 | Vasopressin | 0.005-0.15 | units | | |
| 581 | Vemurafenib | | | | |
| 582 | Venetoclax | 10-600 | mg | 1-30% | |
| 583 | Verapamil hydrochloride | | | | |
| 584 | Vilazodone | 10-60 | mg | 1-6% | |
| 585 | Vildagliptin | 25-75 | mg | 2.5-7.5% | |
| 586 | vismodegib | 75-225 | mg | 7.5-22.5% | |
| 587 | vitamin D | 0.0075-0.03 | mg | 0.0008-0.003% | |
| 588 | Vonoprazan | 10-30 | mg | 1-3% | |
| 589 | Voriconazole | 100-375 | mg | 10-30% | |
| 590 | Vortioxetine | 2.5-30 | mg | 0.25-3% | |
| 591 | Warfarin Sodium | | | | |
| 592 | Zanubrutinib | | | | |

| 593 | Zinc + magnesium + vitamin B6 | 10-30 mg, 55-165 mg, | | 1-3%, 5.5-16.5%, 0.15-3% |
|---|---|---|---|---|
| | | 1.5-30 mg | | |
| 594 | zoledronic acid | 2-6 | mg | 0.2-0.6% |
| | Zolpidem | 2.5-15 | mg | 0.25-1.5% |

### Transdermal Delivery of Aqueous Insoluble Molecules

Various transdermal formulations of the present disclosure are able to successfully deliver (at relatively high bioavailability) a range of actives that are either entirely insoluble, at least partially, or majority insoluble. This ability to deliver insoluble actives is contrary to the commonly-held belief that actives generally need to be solubilized for transdermal drug delivery.

There are a number of pharmacologically active medicaments which are poorly soluble in in aqueous media and/or acidic solutions which have proved challenging to deliver to a subject either transdermally or orally, due to the inability of the drug to be absorbed by the small intestine, or inability of the medicament to be solubilized in a transdermal formulation and penetrate the skin. In general, it has previously been thought that pharmacologically active medicaments must be wholly or partially dissolved in an aqueous solution in order to be absorbed by a subject and achieve a reasonably high bioavailability in a subject. In the case of orally administered medicaments, it is thought a drug must be wholly or partially soluble in an aqueous medium in order dissolved in the stomach and absorbed by the small intestine. In the case of transdermally administered drugs, it is thought that a medicament must be wholly or mostly soluble in the formulation in order to penetrate the skin and achieve a high or relatively high bioavailability in a subject.

Such medicaments which are poorly soluble in aqueous medium are drugs which fall into Class 2 and Class 4 of the Biopharmaceutical Classification System (BCS). The BCS is a scientific framework which predicts in vivo drug performance via in vitro measurements of solubility and permeation. Solubility is the extent a drug can dissolve in gastrointestinal (GI) fluid and permeation is the extent solubilized drug can pass across membranes of cells lining the GI tract. Medicaments have a high solubility according to BCS if less than 250 mL of an aqueous media (pH between 1-7.5) dissolves the highest API prescription dose. Class 2 drugs and Class 4 drugs have low solubilities as they do not meet this solubility criteria. Class 2 drugs have high permeability whereas class 4 drugs have low permeability.

Various transdermal formulations of the present disclosure are effective in delivering class 2 and class 4 insoluble molecules at a high or relatively high bioavailability in a subject. Example 32 illustrates essentially insoluble class 2 or class 4 molecules which are delivered at a high or relatively high bioavailability in a subject by the transdermal formulations of the present disclosure and some partially soluble molecules that are delivered at an improved bioavailability in a subject. In Example 32, the following molecules were included in a transdermal formulation comprising one of Hydrocortisone, Sildenafil Citrate, Cyclosporine, Eletriptan, Neratinib Maleate, or Free Base Neratinib. Notably, the following molecules normally do not solubilize in aqueous medium at >2% (hydrocortisone), >1% (sildenafil citrate, cyclosporine, or apixaban), >0.1% (neratinib maleate or neratinib free base), or >0.002% (eletriptan).

In addition to the above insoluble Class 2 and Class 4 molecules illustrated in Example 32, the pharmacologically active medicaments listed in the below table also achieve high or reasonably high bioavailability when administered with the transdermal formulations of present disclosure.

| | | BCS |
|---|---|---|
| Type of API | API Examples | Class |
| NSAID | Ibuprofen | 2 |
| | Diclofenac | 2 |
| | Difunisal | 2 |
| | Flurbiprofen | 2 |
| | Indomethacin | 2 |
| | Naproxen | 2 |
| | Oxaprozin | 2 |
| | Piroxicam | 2 |
| HIV Antiviral/Protease | Indinavir | 4 |
| Inhibitors | Nelfinavir | 4 |
| | Ritonavir | 4 |
| | Saquinavir | 4 |
| | Nevirapine | 2 |
| | Efavirenz | 2/4 |
| | Lopinavir | 2/4 |
| | Etravirine | 4 |
| | Amprenavir | 2 |
| | Tipranavir | 4 |
| Antacid/GI agent/ | Aluminum Hydroxide | 4 |
| Antihistamine | Famotidine | 4 |
| | Terfenadine | 2 |
| | Loratadine | 2 |
| Diuretic | Furosemide | 4 |
| | Acetazolamide | 4 |
| | Spironolactone | 2/4 |
| | Hydrochlorothiazide | 4 |
| | Chlorothiazide | 4 |
| Vitamins/Retinoids | Retinol | 2/4 |
| | Folic acid | 2/4 |
| | Calcitriol | 2/4 |
| | Isotretinoin | 2 |
| | Doxercalciferol | 2/4 |
| | Paricalcitol | 4 |
| | Calcifediol | 2/4 |
| Antihypertensive/Ca- | Nifedipine | 2 |
| Channel blocker | Verapamil hydrochloride | 1/2 |
| Anticonvulsant/Anti- | Carbamazepine | 2 |
| epileptic | Phenytoin | 2 |
| | Valproic acid | 2 |
| Antibiotic | Sulfamethoxazole | 2 |
| | Trimethoprim | 2 |
| | Ciprofloxacin | 2/4 |
| | Azithromycin | 2 |
| | Erythromycin | 2 |
| | Neomycin | 4 |
| | Ofloxacin | 2 |
| | Nitrofurantoin | 2 |
| | Sulfadiazine | 2/4 |
| | Colistin | 4 |
| | Nalidixic acid | 2 |
| | (Quinolone antibiotic) | |
| | Dapsone | 2 |
| Antimalarial | Artemether + Lumefantrine | 2/4 |
| | Mefloquine | 2/4 |
| Antifungal | Griseofulvin | 2 |
| | Nystatin | 3/4 |
| | Itraconazole | 2 |
| | Ketoconazole | 2 |
| | Amphotericin B | 4 |
| Antidiabetic | Glibenclamide | 2 |
| | Glipizide | 2 |
| Antitubercular antibiotics | Clofazimine | 2/4 |
| | Rifampin | 2 |
| Immunosuppressive | Azathioprine | 4 |
| | Cyclosporine | 4 |
| | Sirolimus | 2 |
| | Tacrolimus | 2 |
| Antiprotozoal | Diloxanide | 2/4 |
| Antiparasitic/anthelmintic | Ivermectin | 2/4 |
| | Niclosamide | 2/3 |
| | Pyrantel | 2/4 |
| | Triclabendazole | 2/4 |
| | Albendazole | 2/4 |
| | Mebendazole | 2/4 |
| Antiparasitic toxoplasmosis | Pyrimethamine | 2/4 |
| Radiocontrast agent | Iopanoic acid | 2 |
| Antidepressant | Amitriptyline | 1/2 |
| | Chlorpromazine | 2/4 |
| Antipsychotic (schizophrenia) | Haloperidol | 2/4 |
| DMARD/anti-rheumatic | Sulfasalazine | 2/4 |
| Anticoagulant | Warfarin Sodium | 1/2 |
| Anti-hypertensive + Diuretic | Chlorthalidone | 4 |
| Chemotherapy | Paclitaxel | 4 |
| | Docetaxel | 4 |
| | Methotrexate | 3/4 |
| Antiemetic | Aprepitant | 4 |
| Antiarrhythmic | Amiodarone | 2 |
| | Digoxin | 2 |
| Statin | Atorvastatin | 2 |
| | Lovastatin | 2 |
| Beta Blocker | Carvedilol | 2 |
| | Talinolol | 2 |
| Gastroprokinetic agent | Cisapride | 2 |
| Hormone | Danazol | 2 |
| | Progesterone | 2 |
| Proton-pump inhibitor | Lansoprazole | 2 |
| Analgesic | Phenazopyridine | 2 |
| Selective Estrogen | Raloxifene | 2 |
| Receptor Modulator (SERM) | Tamoxifen | 2 |
| Cannabinoid | Dronabinol | 2/4 |
| 5-alpha reductase inhibitor | Dutasteride | 2/4 |
| Laxative | Lubiprostone | 2/4 |
| Cholesterol Medication/ | Fenofibrate | 2 |
| antilipemic agent | | |
| Androgen Receptor Inhibitor | Enzalutamide | 2 |

In addition to the above insoluble Class 2 and Class 4 medicaments, kinase inhibitors are especially known for being insoluble medicaments which are difficult to deliver to a subject. Similar to the above, the below kinase inhibitors also achieve high or reasonably high bioavailability when administered with the transdermal formulations of present embodiments. The kinase inhibitors are of the same class of drug, and/or the same class of the Biopharmaceutical Classification System as one or more of the medicaments listed above in the below Table.

| | Kinase Inhibitor | BCS |
|---|---|---|
| Target Receptors | Examples | Class |
| ABL | Bosutinib | 4 |
| | Ponatinib | 2 |
| ABL, PDGFR, KIT | Nilotinib | 4 |
| ABL, PDGFR, KIT, SRC | Dasatinib | 2 |
| ALK | Ceritinib | 4 |
| | Alectinib | 4 |
| | Lorlatinib | 4 |
| ALK, MET | Crizotinib | 4 |
| BRAF | Vemurafenib | 4 |
| | Dabrafenib | 2 |
| BTK | Ibrutinib | 2 |
| | Acalabrutinib | 2 |
| | Zanubrutinib | 2 |
| CDK4/6 | Palbociclib | 4 |
| | Ribociclib | 4 |
| CSFIR, c-KIT, FLT3 | Pexidartinib | 2 |
| EGFR | Neratinib | 4 |
| | Gefitinib | 2 |
| | Erlotinib | 2 |
| EGFR, ERBB2 | Lapatinib | 4 |
| ERBB2 | Tucatinib | 2 |
| FGFR | Pemigatinib | 2 |
| FLT3, RTKs | Gilteritinib | 4 |
| JAK | Peficitinib | 4 |
| JAK3, FLT3, BRD4 | Fedratinib | 2/4 |
| MEK | Trametinib | 2 |
| MEK1/2 | Selumetinib | 4 |
| MET ex 14 alterations | Tepotinib | 4 |
| mTOR | Sirolimus | 2 |
| mutant BRAF | Encorafenib | 2 |
| mutant EGFR | Dacomitinib | 2 |
| mutant MET | Capmatinib | 2/4 |
| mutants KIT, PDGFR | Avapritinib | 2 |
| NTRK1/2/3, ROS1, ALK | Entrectinib | 2 |
| PI3Kδ | Idelalisib | 2 |
| | Umbralisib | 2 |
| PI3Kδ/γ | Duvelisib | 4 |
| PI3Kα | Alpelisib | 2 |
| PKC, FLT3 | Midostaurin | 2 |
| RET | Pralsetinib | 2 |
| RET fusions | Selpercatinib | 2 |
| RET, VEGFR FGFR, EGFR | Vandetanib | 2 |
| ROCK2/mTor inhibitor | Belumosudil | 4 |
| SYK | Fostamatinib | 4 |
| VEGFR | Tivozanib | 2/4 |
| VEGFR, PDGFR, FGFR | Nintedanib | 2/4 |
| VEGFR, KIT, PDGFR, RET, | Sunitinib | 4 |
| CSF1R, FLT3 | Axitinib | 2 |
| VEGFR2, PDGFR, KIT FLT3 | Cabozantinib | 2 |
| VEGFR2 Tie2 | Regorafenib | 2 |
| VEGFR2, PDGFR, c-KIT | Pazopanib | 2 |
| VEGFR2, KIT, Src | Apatinib | 2 |
| VEGFR2, PDGFR, KIT, FLT3, BRAF | Sorafenib | 2/4 |
| VEGFRs | Lenvatinib | 2/4 |

Without wishing to be bound by theory, the mechanism of action in which the insoluble class 2 or class 4 medicaments are delivered at a high or reasonably high bioavailability transdermally may include one or more ingredients in the transdermal formulations of present embodiments partially breaking down one or more layers of dermal tissue (e.g., stratum coreum, stratum lucidum, stratum granulosum, stratum spinosum, stratum basale) permitting the medicament to penetrate the dermis, and enter the targeted tissue at a high local concentration, providing a high or reasonably high bioavailability in the targeted tissue. In other embodiments, the mechanism of action may include one or more ingredients in the transdermal formulations of present embodiments partially breaking down one or more layers of dermal tissue (e.g., stratum coreum, stratum lucidum, stratum granulosum, stratum spinosum, stratum basale) in order to permitting the medicament to penetrate the dermis, and enter the blood stream as to delivery an insoluble class 2 or class 4 medicaments systemically while bypassing the GI track.

Any herein-disclosed transdermal formulation can provide systemic administration of a medicament (e.g., a medicament disclosed in Table 1) via transdermal delivery of the medicament.

### Wound Healing

Various transdermal formulations of the present disclosure are effective in promoting wound healing. Without wishing to be bound by theory, the mechanism of action may be one or both reducing infection, reducing inflammation, and helping the body heal.

For example, without being bound to a particular theory, one possible mechanism in which the transdermal formulations of the present disclosure may aid wound healing is by modulating the microenvironment of the wounded tissue to a physiological normal pH, and/or reducing the body's inflammatory response using a buffering agent and/or by delivering one or more of the medicaments listed in Table 1 which may have a wound healing effect. Various medicaments listed in Table 1 may directly promote wound healing. In some cases, the medicament is an antibiotic or other anti-infective agent. In some cases, the medicament is a steroid, retinoid, or other dermatological enhancing agent. In some cases, the medicament is a buffering agent (e.g., NaHCO₃). Nonlimiting examples of antibiotics and other anti-infective agents include vancomycin, doxycycline, ampicillin, sulbactam, amoxicillin, azithromycin, clavulanate, piperacillin, tazobactam, Amphotericin B, clarithromycin, daptomycin, doxycycline ER, ivermectin, lymecycline, neomycin, and tigecycline. These medicaments may be used alone or in any combination.

Inflammation is a natural response to a physical trauma experienced by a subject and is an important aspect of the body's healing response. Aspects of inflammation which may be associated with wound healing include acidification of the wounded tissue microenvironment in and around the wounded tissue, overproduction of white blood cells, activation of macrophages, production of cytokines, activation of neutrophils, eosinophils, lymphocytes, plasma cells, myeloid granulocytes, and histocytes, increased blood flow, swelling, stiffness, fatigue, fever, and pain, among other symptoms, which may vary depending on the type and severity of the wound, including whether or not the wound is infected. Injured tissue tends to provoke the body's inflammatory response, and the ordinary course of wound healing advances from an inflammation stage following injury, to tissue formation stage, and then to a tissue remodeling stage.

In some cases, a subject may partially or entirely fail to advance from the inflammation stage to tissue formation stage, which may halt or slow the wound healing process; or may advance to the tissue formation or tissue remodeling stages without exiting the inflammation stage. The inflammation of the injured tissue may then become chronic and begin to damage the body's healthy cell tissues, including newly produced cell tissues, and newly remodeled cell tissues, possibly leading to DNA damage, tissue death, and formation of scar tissue. In some embodiments, the wounded tissue may partially or entirely fail to advance from the inflammation stage, and may continue to have an acidic tissue microenvironment in and around the wounded tissue. In some embodiments, the acidic tissue microenvironment may be the result of ischemia-driven tissue acidification. The creation and maintenance of an acidic tissue microenvironment may trigger inflammatory processes in the wounded tissue on the ongoing bases, stimulation of the production of inflammatory cytokines by the stroma or endothelium in wounded tissue. In a healthy regeneration and remodeling response, neoangiogenesis will occur, and the wounded tissue will return to a physiological pH (e.g., neutral or slightly basic, pH 7.4).

However, when the acidic tissue microenvironment, or the ischemia-driven tissue acidification, fails to resolve as the wound attempts to heal, the wounded tissue remains partially inflamed as it advances to the tissue formation and tissue remodeling stages, and the inflammatory processes that initially benefitted the wound healing process may then begin to damage the new tissue. For example, the maintenance of the acidic tissue microenvironment may continue to stimulate inflammatory processes such as the production of inflammatory cytokines, overproduction of white blood cells, activation of macrophages, activation of neutrophils, swelling, stiffness, and pain, among other possible inflammatory processes. The various inflammatory processes may damage newly formed tissue, or slow the tissue formation and tissue remodeling processes. For example, overproduction of white blood cells, and activation of macrophages, activation of neutrophils may target and phagocytose newly formed cells or newly remodeled tissue; or ongoing swelling and stiffness of a wound may reduce blood flow and the influx of fresh oxygen and nutrients required by the wounded tissue to heal; or ongoing stiffness and pain may prevent the subject from moving or exercising the tissue as it heals as may be necessary to stimulate blood flow and the influx of fresh oxygen and nutrients, or to otherwise stimulate the formation of healthy tissue which requires a biomechanically normal levels of stress and strain upon the tissue in order to facilitate proper gene transcription and proper tissue formation.

In some embodiments, the transdermal formulations of the present disclosure are effective in promoting wound healing by modulating the acidic tissue microenvironment with a buffering agent to a neutral pH, a slightly basic pH, a basic pH, or a physiological pH (e.g., about 7.4). In some embodiments, the modulating the acidic tissue microenvironment with a buffering agent to a higher pH may reduce or eliminate one or more inflammatory processes, which may aid in wound healing. Possible mechanisms of buffering agents aiding wound healing by reducing inflammation may include deactivating macrophages and/or neutrophils, reducing the production of white blood cells, decreasing swelling, stiffness, and pain, and increasing blood flow to the wounded tissue.

In some embodiments, the transdermal formulations of the present disclosure are effective in promoting wound healing by delivering an anti-infective agent to prevent the wound from getting infected. In some embodiments, the anti-infective agent is an antibiotic, as described above. Possible mechanisms of anti-infective agent aiding wound healing includes preventing the uncontrolled growth of bacteria in wounded tissue, reducing inflammation associated with infection, and diverting the body's resource from fighting infection to cell division, tissue production, and tissue remodeling.

In some embodiments, the transdermal formulations of the present disclosure directed to wound healing comprise both an anti-infective agent, another medicament, and/or a buffering agent. In some embodiments, the transdermal formulations of the present disclosure directed to wound healing comprise both an antibiotic and a buffering agent. In some embodiments, the transdermal formulations of the present disclosure comprise one of the medicaments listed in Table 1 and a buffering agent, e.g., NaHCO₃. In some embodiments, the transdermal formulations of the present disclosure directed to wound healing comprise both an antibiotic, another medicament of Table 1, and a buffering agent. Possible mechanisms of a combination of buffering agents, antibiotics, and other medicaments of Table 1 aiding wound healing include those set forth above, as well as synergistic effects resulting from the combination of the antibiotic and buffering agent. In some embodiments, the combination of a buffering agent, an antibiotic, and/or another medicament of Table 1 in the transdermal formulations of the present disclosure provide a greater wound healing effect (e.g., reduce the time to wound closure, and restoration of healthy, functional tissue), by an amount that is 10, 20, 30, 40, 50, 60, or 75% greater than either the use of an antibiotic or buffering agent separately, or when compared to healing an untreated wound.

In some embodiments, the transdermal formulations of the present disclosure are effective in promoting wound healing by delivery of a dermatological restoring agent to the wounded tissue, for example, delivery of steroid or retinoid to dermal tissue. In some embodiments, the steroid or retinoid may aid the wound healing process by stimulating the production of collagen in the newly formed dermal tissue without resulting in the overproduction of scar tissue.

In some embodiments, the wound which is aided by the transdermal formulations of the present disclosure is a diabetic foot ulcer. A diabetic foot ulcer is an open sore or wound that occurs in approximately 15 percent of patients with diabetes, and is commonly located on the bottom of the foot. Of those who develop a foot ulcer, six percent will be hospitalized due to infection or other ulcer-related complication. In some embodiments, the use of a transdermal formulation described herein comprising a buffering agent, an antibiotic, both a buffering agent and an antibiotic, or other combinations of the medicaments listed in Table 1, may aid in the healing of the diabetic foot ulcer, may reduce the time to close an open diabetic foot ulcer, or may reduce the time to the formation of healthy or physiologically functional tissue. In some embodiments, the use of a transdermal formulation described herein comprising a buffering agent, an antibiotic, and/or another medicament listed in Table 1, treats a diabetic foot ulcer. In some embodiments, the use of a transdermal formulation described herein comprising a buffering agent, an antibiotic, and/or another medicament listed in Table 1, prevents a diabetic foot ulcer. In some embodiments, the use of a transdermal formulation described herein comprising a buffering agent, an antibiotic, and/or another medicament listed in Table 1, reduces the likelihood of a diabetic foot ulcer by 25, 50, 75, 85, 90, or 95%. In some embodiments, the use of a transdermal formulation described herein comprising a buffering agent, an antibiotic, and/or another medicament listed in Table 1, reduces the likelihood of infection from a diabetic foot ulcer by 25, 50, 75, 85, 90, or 95%. In some embodiments, the use of a transdermal formulation described herein comprising a buffering agent, an antibiotic, and/or another medicament listed in Table 1, reduces the likelihood of hospitalization due to the infection of a diabetic foot ulcer by 25, 50, 75, 85, 90, or 95%.

### Cancers and Tumors

Cancer is generally defined as a group of diseases involving abnormal cell growth with the potential to invade or spread to other parts of the body. Conventional cancer treatments are directed at removing cancerous tissue and preventing it from spreading. Such treatment options include surgery, chemotherapy, radiation therapy, hormonal therapy, targeted therapy and palliative care. Treatments are usually pursued based on the type, location and grade of the cancer as well as the patient's health and preferences.

A promising area for the development of treatments includes targeted therapies using small molecules. Many embodiments provided herein are directed to various methods of treating cancer and/or tumors. An exemplary embodiment of a method of treating cancer in a patient according to the invention comprises administering topically and/or transdermally an effective amount of a transdermal delivery formulation comprising one or small molecules to a patient in need thereof, wherein said administration is effective to inhibit or prevent the growth of a tumor or tumor cells.

Another embodiment is directed to a method of preventing metastasis of tumors comprising administering topically and/or transdermally an effective amount of a transdermal delivery formulation comprising one or more small molecules to a patient in need thereof, where the administration is effective to inhibit or prevents the metastasis of tumors or cancer cells.

Another embodiment is directed to a method of preventing the intravasation of tumor cells comprising administering topically and/or transdermally an effective amount of a transdermal delivery formulation comprising one or more small molecules to a patient in need thereof, where the administration is effective to inhibit or prevent the intravasation of tumor cells.

Another embodiment is directed to a method of treatment of cancer, the method comprising i) selecting a therapeutic agent (e.g. a small molecule kinase inhibitor) described herein and formulating the therapeutic agent in a transdermal delivery formulation comprising one or more small molecules, and iii) administering the formulation topically and/or transdermally in an amount effective to inhibit or prevent the growth of a tumor or tumor cells.

Another embodiment is directed to a method of improving, extending the duration of remission, or maintaining remission of a cancer or tumor comprising administering topically and/or transdermally an effective amount of a transdermal delivery formulation comprising one or more small molecules to a patient in need thereof, where administration is effective to improve, extend the duration of remission, or maintain remission of a cancer or tumor.

In other embodiments, a method of treating cancer in a patient comprises administering topically and/or transdermally an effective amount of a transdermal delivery formulation comprising one or more small molecules to a patient in need thereof, where the administration is effective to alter the activity of one or more cell signaling proteins in the patient. The altered protein activity inhibits the growth of a solid tumor or cancer cells.

In other embodiments, a method of targeting a serine/threonine/tyrosine kinase in a patient is provided. These embodiments generally comprise administering topically and/or transdermally an effective amount of a transdermal delivery formulation comprising one or more small molecules to a patient in need thereof, wherein the administration is effective inhibit critical cancer targets, such as serine/threonine/tyrosine kinases in the patient. In other embodiments, a method of inhibiting or preventing the metastasis of tumors in a patient is provided.

In other embodiments, a method of inhibiting or preventing the intravasation of tumor cells in a patient is provided. These embodiments generally comprise administering topically and/or transdermally an effective amount of a transdermal delivery formulation comprising one or more small molecules to a patient in need thereof, wherein the administration is effective to inhibit or prevent the intravasation of tumor cells.

Formulations provided herein are used in methods of treating many cancers, including but not limited to breast cancer, prostate cancer, pancreatic cancer, lung cancer, bladder cancer, skin cancer, colorectal cancer, kidney cancer, liver cancer, and thyroid cancer.

Formulations provided herein are also used in methods of treating a cancer or tumor, including adrenocortical carcinoma, basal cell carcinoma, bladder cancer, bone cancer, brain tumor, breast cancer, cervical cancer, colon cancer, colorectal cancer, esophageal cancer, retinoblastoma, gastric (stomach) cancer, gastrointestinal tumors, glioma, head and neck cancer, hepatocellular (liver) cancer, islet cell tumors (endocrine pancreas), kidney (renal cell) cancer, laryngeal cancer, non-small cell lung cancer, small cell lung cancer, medulloblastoma, melanoma, pancreatic cancer, prostate cancer, renal cancer, rectal cancer and thyroid cancer.

Another example of a biological therapeutic agent that is used for the treatment of certain cancers in certain embodiments are angiogenesis inhibitors. Accordingly, formulations of the invention can also be combined with angiogenesis inhibitors to increase anti-tumor effects. Angiogenesis is the growth of new blood vessels. This process allows tumors to grow and metastasize. Inhibiting angiogenesis can help prevent metastasis, and stop the spread of tumors cells. Angiogenesis inhibitors include, but are not limited to, angiostatin, endostatin, thrombospondin, platelet factor 4, Cartilage-derived inhibitor (CDI), retinoids, Interleukin-12, tissue inhibitor of metalloproteinase 1, 2 and 3 (TIMP-1, TIMP-2, and TIMP-3) and proteins that block the angiogenesis signaling cascade, such as anti-VEGF (Vascular Endothelial Growth Factor) and IFN-alpha. Angiogenesis inhibitors can be administered or co-administered with tumor specific constructs, including antigen-binding constructs capable of mediating, for example, ADCC and/or complement fixation or chemotherapy-conjugated antigen-binding of the invention to combat various types of cancers, for example, solid tumor cancers such as lung and breast cancer. Other examples of biological therapeutic agents include inhibitors of E-cadherin and of epidermal growth factor receptor (EGFR). Known inhibitors include erlotinib, an anti-integrin drug (Cilengitide), Cariporide, Eniporide and Amiloride.

In another aspect, formulations of the invention can be administered or co-administered with disease modifying anti-rheumatic agents (DMAR agents) for the treatment of rheumatoid arthritis, psoriasis, ulcerative colitus, systemic lupus erythematosus (SLE), Crohn's disease, ankylosing spondylitis, and various inflammatory disease processes. In such treatment, the constructs, for example, antigen-binding constructs, of the invention are commonly administered in conjunction with compounds such as azathioprine, cyclosporin, gold, hydroxychloroquine, methotrexate, penicallamine, sulphasalazine, and the like.

In another aspect, formulations provided herein can be used with palliative (non-radical) operations to surgically remove tumors. In this aspect, one or more formulations of the invention can be administered before and after surgical extractions of tumors in order to reduce the likelihood of metastasis and reoccurrence by killing any cancer cells that were not removed during the surgery. Other diseases, conditions, and disorders described herein can be treated with formulations and methods provided herein.

Another example of a biological therapeutic agent that is used for the treatment of certain cancers in certain embodiments are angiogenesis inhibitors. Accordingly, formulations of the invention can also be combined with angiogenesis inhibitors to increase anti-tumor effects. Angiogenesis is the growth of new blood vessels. This process allows tumors to grow and metastasize. Inhibiting angiogenesis can help prevent metastasis, and stop the spread of tumors cells. Angiogenesis inhibitors include, but are not limited to, angiostatin, endostatin, thrombospondin, platelet factor 4, Cartilage-derived inhibitor (CDI), retinoids, Interleukin-12, tissue inhibitor of metalloproteinase 1, 2 and 3 (TIMP-1, TIMP-2, and TIMP-3) and proteins that block the angiogenesis signaling cascade, such as anti-VEGF (Vascular Endothelial Growth Factor) and IFN-alpha. Angiogenesis inhibitors can be administered or co-administered with tumor specific constructs, including antigen-binding constructs capable of mediating, for example, ADCC and/or complement fixation or chemotherapy-conjugated antigen-binding of the invention to combat various types of cancers, for example, solid tumor cancers such as lung and breast cancer. Other examples of biological therapeutic agents include inhibitors of E-cadherin and of epidermal growth factor receptor (EGFR). Known inhibitors include erlotinib, an anti-integrin drug (Cilengitide), Cariporide, Eniporide and Amiloride.

In another aspect, formulations of the invention can be administered or co-administered with disease modifying anti-rheumatic agents (DMAR agents) for the treatment of rheumatoid arthritis, psoriasis, ulcerative colitis, systemic lupus erythematosus (SLE), Crohn's disease, ankylosing spondylitis, and various inflammatory disease processes. In such treatment, the constructs, for example, antigen-binding constructs, of the invention are commonly administered in conjunction with compounds such as azathioprine, cyclosporin, gold, hydroxychloroquine, methotrexate, penicallamine, sulphasalazine, and the like.

In another aspect, formulations provided herein can be used with palliative (non-radical) operations to surgically remove tumors. In this aspect, one or more formulations of the invention can be administered before and after surgical extractions of tumors in order to reduce the likelihood of metastasis and reoccurrence by killing any cancer cells that were not removed during the surgery. Other diseases, conditions, and disorders described herein can be treated with formulations and methods provided herein.

Formulations provided herein are also used in methods of treating a cancer or tumor, including but not limited to Adrenocortical Carcinoma, Basal Cell Carcinoma, Bladder Cancer, Bone Cancer, Brain Tumor, Breast Cancer, Cervical Cancer, Colon Cancer, Colorectal Cancer, Esophageal Cancer, Retinoblastoma, Gastric (Stomach) Cancer, Gastrointestinal Tumors, Glioma, Head and Neck Cancer, Hepatocellular (Liver) Cancer, Islet Cell Tumors (Endocrine Pancreas), Kidney (Renal Cell) Cancer, Laryngeal Cancer, Non-small Cell Lung Cancer, Small Cell Lung Cancer, Medulloblastoma, Melanoma, Pancreatic Cancer, Prostate Cancer, Renal Cancer, Rectal cancer, and Thyroid Cancer.

While preferred embodiments of the methods provided herein are typically directed to a particular cancer, solid tumor or grouping thereof, a more complete but still non-limiting listing of suitable cancers and tumors that may be tested for effectiveness according to embodiments provided herein includes the following: lymphoblastic leukemia (ALL), acute myeloid leukemia (AML), adrenocortical carcinoma, aids-related cancers, Kaposi sarcoma (soft tissue sarcoma), aids-related lymphoma (lymphoma), primary CNS lymphoma (lymphoma), anal cancer, astrocytomas, atypical teratoid/rhabdoid tumor, childhood, central nervous system (brain cancer), basal cell carcinoma, bile duct cancer, bladder cancer. childhood bladder cancer, bone cancer (includes Ewing sarcoma and osteosarcoma and malignant fibrous histiocytoma), brain tumors, breast cancer, childhood breast cancer, bronchial tumors, Burkitt lymphoma (non-Hodgkin lymphoma, carcinoid tumor (gastrointestinal), childhood carcinoid tumors, cardiac (heart) tumors, central nervous system tumors. atypical teratoid/rhabdoid tumor, childhood (brain cancer), embryonal tumors, childhood (brain cancer), germ cell tumor (childhood brain cancer), primary CNS lymphoma, cervical cancer, childhood cervical cancer, cholangiocarcinoma, chordoma (childhood), chronic lymphocytic leukemia (CLL), chronic myelogenous leukemia (cml), chronic myeloproliferative neoplasms, colorectal cancer, childhood colorectal cancer, craniopharyngioma (childhood brain cancer), cutaneous t-cell lymphoma, ductal carcinoma in situ (DCIS), embryonal tumors, (childhood brain CNS cancers), endometrial cancer (uterine cancer), ependymoma, esophageal cancer, childhood esophageal cancer, esthesioneuroblastoma (head and neck cancer), Ewing sarcoma (bone cancer), extracranial germ cell tumors, extragonadal germ cell tumors, eye cancer, childhood intraocular melanoma, intraocular melanoma, retinoblastoma, fallopian tube cancer, fibrous histiocytoma of bone (malignant, and osteosarcoma), gallbladder cancer, gastric (stomach) cancer, childhood gastric (stomach) cancer, gastrointestinal carcinoid tumor, gastrointestinal stromal tumors (gist) (soft tissue sarcoma), childhood gastrointestinal stromal tumors, germ cell tumors, childhood central nervous system germ cell tumors, childhood extracranial germ cell tumors, extragonadal germ cell tumors, ovarian germ cell tumors, testicular cancer, gestational trophoblastic disease, hairy cell leukemia, head and neck cancer, heart tumors, hepatocellular (liver) cancer, histiocytosis (Langerhans cell cancer), Hodgkin lymphoma, hypopharyngeal cancer (head and neck cancer), intraocular melanoma, childhood intraocular melanoma, islet cell tumors, (pancreatic neuroendocrine tumors), Kaposi sarcoma (soft tissue sarcoma), kidney (renal cell) cancer, Langerhans cell histiocytosis, laryngeal cancer (head and neck cancer), leukemia, lip and oral cavity cancer (head and neck cancer), liver cancer, lung cancer (non-small cell and small cell), childhood lung cancer, lymphoma, male breast cancer, malignant fibrous histiocytoma of bone and osteosarcoma, melanoma, childhood melanoma, melanoma (intraocular eye), childhood intraocular melanoma, Merkel cell carcinoma (skin cancer), mesothelioma, childhood mesothelioma, metastatic cancer, metastatic squamous neck cancer with occult primary (head and neck cancer), midline tract carcinoma with nut gene changes, mouth cancer (head and neck cancer), multiple endocrine neoplasia syndromes—see unusual cancers of childhood, multiple myeloma/plasma cell neoplasms, mycosis fungoides (lymphoma), myelodysplastic syndromes, myelodysplastic/myeloproliferative neoplasms, myelogenous leukemia, chronic (CML), myeloid leukemia, (acute AML), myeloproliferative neoplasms, nasal cavity and paranasal sinus cancer (head and neck cancer), nasopharyngeal cancer (head and neck cancer), neuroblastoma, non-Hodgkin lymphoma, non-small cell lung cancer, oral cancer (lip and oral cavity cancer and oropharyngeal cancer), osteosarcoma and malignant fibrous histiocytoma of bone, ovarian cancer, childhood ovarian cancer, pancreatic cancer, childhood pancreatic cancer, pancreatic neuroendocrine tumors (islet cell tumors), papillomatosis, paraganglioma, childhood paraganglioma, paranasal sinus and nasal cavity cancer, parathyroid cancer, penile cancer, pharyngeal cancer, pheochromocytoma, childhood pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, recurrent cancer, renal cell (kidney) cancer, retinoblastoma, rhabdomyosarcoma, salivary gland cancer, sarcoma, childhood rhabdomyosarcoma (soft tissue sarcoma), childhood vascular tumors (soft tissue sarcoma), Ewing sarcoma (bone cancer), Kaposi sarcoma (soft tissue sarcoma), osteosarcoma (bone cancer), soft tissue sarcoma, uterine sarcoma, Sézary syndrome (lymphoma), skin cancer, childhood skin cancer, small cell lung cancer, small intestine cancer, soft tissue sarcoma, squamous cell carcinoma of the skin, squamous neck cancer with occult primary, stomach (gastric) cancer, childhood stomach, t-cell lymphoma, testicular cancer, childhood testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter kidney (renal cell cancer), ureter and renal pelvis (transitional cell cancer kidney renal cell cancer), urethral cancer, uterine cancer (endometrial), uterine sarcoma, vaginal cancer, childhood vaginal cancer, vascular tumors (soft tissue sarcoma), vulvar cancer, and Wilms tumor (and other childhood kidney tumors).

Bortezomib and Carfilzomib target proteosomes; Batimastat, Neovastat, Prinomastat, Rebimastat, Ganetespib and NVP-AUY922 target MMPs and HSPs; Obatoclax and Navitoclax target apoptosis. The molecular target of the remaining agents is Tyrosine & Serine/Threonine kinases.

While preferred embodiments of the methods provided herein are typically directed to a particular cancer, solid tumor or grouping thereof, a more complete but still non-limiting listing of suitable cancers and tumors that may be tested for effectiveness according to embodiments provided herein includes the following: lymphoblastic leukemia (ALL), acute myeloid leukemia (AML), adrenocortical carcinoma, aids-related cancers, Kaposi sarcoma (soft tissue sarcoma), aids-related lymphoma (lymphoma), primary CNS lymphoma (lymphoma), anal cancer, astrocytomas, atypical teratoid/rhabdoid tumor, childhood, central nervous system (brain cancer), basal cell carcinoma, bile duct cancer, bladder cancer. childhood bladder cancer, bone cancer (includes Ewing sarcoma and osteosarcoma and malignant fibrous histiocytoma), brain tumors, breast cancer, childhood breast cancer, bronchial tumors, Burkitt lymphoma (non-Hodgkin lymphoma, carcinoid tumor (gastrointestinal), childhood carcinoid tumors, cardiac (heart) tumors, central nervous system tumors. atypical teratoid/rhabdoid tumor, childhood (brain cancer), embryonal tumors, childhood (brain cancer), germ cell tumor (childhood brain cancer), primary CNS lymphoma, cervical cancer, childhood cervical cancer, cholangiocarcinoma, chordoma (childhood), chronic lymphocytic leukemia (CLL), chronic myelogenous leukemia (cml), chronic myeloproliferative neoplasms, colorectal cancer, childhood colorectal cancer, craniopharyngioma (childhood brain cancer), cutaneous t-cell lymphoma, ductal carcinoma in situ (DCIS), embryonal tumors, (childhood brain CNS cancers), endometrial cancer (uterine cancer), ependymoma, esophageal cancer, childhood esophageal cancer, esthesioneuroblastoma (head and neck cancer), Ewing sarcoma (bone cancer), extracranial germ cell tumors, extragonadal germ cell tumors, eye cancer, childhood intraocular melanoma, intraocular melanoma, retinoblastoma, fallopian tube cancer, fibrous histiocytoma of bone (malignant, and osteosarcoma), gallbladder cancer, gastric (stomach) cancer, childhood gastric (stomach) cancer, gastrointestinal carcinoid tumor, gastrointestinal stromal tumors (gist) (soft tissue sarcoma), childhood gastrointestinal stromal tumors, germ cell tumors, childhood central nervous system germ cell tumors, childhood extracranial germ cell tumors, extragonadal germ cell tumors, ovarian germ cell tumors, testicular cancer, gestational trophoblastic disease, hairy cell leukemia, head and neck cancer, heart tumors, hepatocellular (liver) cancer, histiocytosis (Langerhans cell cancer), Hodgkin lymphoma, hypopharyngeal cancer (head and neck cancer), intraocular melanoma, childhood intraocular melanoma, islet cell tumors, (pancreatic neuroendocrine tumors), Kaposi sarcoma (soft tissue sarcoma), kidney (renal cell) cancer, Langerhans cell histiocytosis, laryngeal cancer (head and neck cancer), leukemia, lip and oral cavity cancer (head and neck cancer), liver cancer, lung cancer (non-small cell and small cell), childhood lung cancer, lymphoma, male breast cancer, malignant fibrous histiocytoma of bone and osteosarcoma, melanoma, childhood melanoma, melanoma (intraocular eye), childhood intraocular melanoma, Merkel cell carcinoma (skin cancer), mesothelioma, childhood mesothelioma, metastatic cancer, metastatic squamous neck cancer with occult primary (head and neck cancer), midline tract carcinoma with nut gene changes, mouth cancer (head and neck cancer), multiple endocrine neoplasia syndromes—see unusual cancers of childhood, multiple myeloma/plasma cell neoplasms, mycosis fungoides (lymphoma), myelodysplastic syndromes, myelodysplastic/myeloproliferative neoplasms, myelogenous leukemia, chronic (CML), myeloid leukemia, (acute AML), myeloproliferative neoplasms, nasal cavity and paranasal sinus cancer (head and neck cancer), nasopharyngeal cancer (head and neck cancer), neuroblastoma, non-Hodgkin lymphoma, non-small cell lung cancer, oral cancer (lip and oral cavity cancer and oropharyngeal cancer), osteosarcoma and malignant fibrous histiocytoma of bone, ovarian cancer, childhood ovarian cancer, pancreatic cancer, childhood pancreatic cancer, pancreatic neuroendocrine tumors (islet cell tumors), papillomatosis, paraganglioma, childhood paraganglioma, paranasal sinus and nasal cavity cancer, parathyroid cancer, penile cancer, pharyngeal cancer, pheochromocytoma, childhood pheochromocytoma, pituitary tumor, plasma cell neoplasm/multiple myeloma, pleuropulmonary blastoma, pregnancy and breast cancer, primary central nervous system (CNS) lymphoma, primary peritoneal cancer, prostate cancer, rectal cancer, recurrent cancer, renal cell (kidney) cancer, retinoblastoma, rhabdomyosarcoma, salivary gland cancer, sarcoma, childhood rhabdomyosarcoma (soft tissue sarcoma), childhood vascular tumors (soft tissue sarcoma), Ewing sarcoma (bone cancer), Kaposi sarcoma (soft tissue sarcoma), osteosarcoma (bone cancer), soft tissue sarcoma, uterine sarcoma, Sézary syndrome (lymphoma), skin cancer, childhood skin cancer, small cell lung cancer, small intestine cancer, soft tissue sarcoma, squamous cell carcinoma of the skin, squamous neck cancer with occult primary, stomach (gastric) cancer, childhood stomach, t-cell lymphoma, testicular cancer, childhood testicular cancer, throat cancer, nasopharyngeal cancer, oropharyngeal cancer, hypopharyngeal cancer, thymoma and thymic carcinoma, thyroid cancer, transitional cell cancer of the renal pelvis and ureter kidney (renal cell cancer), ureter and renal pelvis (transitional cell cancer kidney renal cell cancer), urethral cancer, uterine cancer (endometrial), uterine sarcoma, vaginal cancer, childhood vaginal cancer, vascular tumors (soft tissue sarcoma), vulvar cancer, and Wilms tumor (and other childhood kidney tumors).

In another, formulations and/or compounds provided herein are co-administered or administered to an animal, subject or patient in conjunction with one or more chemotherapeutic compounds such as alkylating agents, antibodies and related agents with anti-tumor properties, anthracyclines, antimetabolites, antitumor antibiotics, aromatase inhibitors, cytoskeletal disruptors (e.g. taxanes), epothilones, histone deacetylace inhibitors, kinase inhibitors, nucleoside analogues, topoisomerase inhibitors, retinoids, vinca alkaloids and derivatives, and the like. The administration or co-administration of one or more formulation or composition of the invention and one or more chemotherapeutic agents can be used for the treatment of tumors or cancer in an animal, subject or patient.

As an example, alkylating agents can be administered or co-administered with or as part of a formulation provided herein. Examples of an alkylating agents that can be co-administered include mechlorethamine, chlorambucil, ifosfamide, melphalan, busulfan, carmustine, lomustine, procarbazine, dacardazine, cisplatin, carboplatin, mitomycin C, cyclophosphamide, ifosfamide, thiotepa, and dacarbazine, and analogues thereof. See for example U.S. Pat. No. 3,046,301 describing the synthesis of chlorambucil, U.S. Pat. No. 3,732,340 describing the synthesis of ifosfamide, U.S. Pat. No. 3,018,302 for the synthesis of cyclophosphamide, U.S. Pat. No. 3,032,584 describing the synthesis of melphalan, and Braunwald et al., “Harrison's Principles of Internal Medicine,” 15th Ed., McGraw-Hill, New York, N.Y., pp. 536-544 (2001) for clinical aspects of cyclophosphamide, chlorambucil, melphalan, ifosfamide, procarbazine, hexamethylmelamine, cisplatin, and carboplatin. Examples of nucleoside analogs include, but are not limited to, fludarabine pentostatin, methotrexate, fluorouracil, fluorodeoxyuridine, CB3717, azacitidine, cytarabine, floxuridine, mercaptopurine, 6-thioguanine, cladribine, and analogues thereof.

In another aspect, formulations provided herein are administered with chemosensitising agents such as those described for example in U.S. Pat. No. 3,923,785 describing the synthesis of pentostatin, U.S. Pat. No. 4,080,325 describing the synthesis of methotrexate, U.S. Pat. No. 2,802,005 describing the synthesis of fluorouracil, and Braunwald et al., “Harrison's Principles of Internal Medicine,” 15th Ed., McGraw-Hill, New York, N.Y., pp. 536-544 (2001) for clinical aspects of methotrexate, 5-fluorouracil, cytosine arabinoside, 6-mercaptopurine, 6-thioguanine, and fludarabine phosphate. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992), incorporated by reference herein.

In another aspect, formulations provided herein can be administered or co-administered with diterpene compounds, including but not limited to paclitaxel, docetaxel, cabazitaxel, and the like.

In another aspect, formulations provided herein can be administered or co-administered with compounds that inhibit topoisomerase II or compounds that otherwise interact with nucleic acids in cells. Such compounds include, for example, doxorubicin, epirubicin, etoposide, teniposide, mitoxantrone, and analogues thereof. In one example, this combination is used in treatment to reduce tumor cell contamination of peripheral blood progenitor cells (PBSC) in conjunction with high-dose chemotherapy and autologous stem cell support (HDC-ASCT). See U.S. Pat. No. 6,586,428 to Geroni et al.

In another aspect, formulations provided herein can be administered or co-administered with immunotherapeutic agents. Immunotherapy has become a promising approach to treat cancer. Kruger C., et al., Immune based therapies in cancer, Histol. Histopathol, 2007, v22, 687-696. The types of immunotherapies used to treat cancer and can be categorized as active, passive or hybrid (active and passive). Active immunotherapy directs the immune system to attack tumor cells by targeting TAAs. Passive immunotherapies enhance existing anti-tumor responses and include the use of cytokines, checkpoint inhibitors, monoclonal antibodies, lymphocytes and cytokines. A suitable immunotherapeutic agent or immunotherapy may be a biologic or biologically active agent such as an antibody or modified antibody or cell based therapy such as chimeric antigen receptor therapy (CAR-T). It is recognized that there may be overlap in categorizing and classifying such agent as biological agents, immunotherapeutic agents, cell-based therapeutics, biological therapeutic agents and the like. Examples of approved antibody immunotherapeutics include, alemtuzumab, atezolizumab, avelumab, ipilimumab, durvalumab, nivolumab, ofatumumab, and rituximab, and trastuzumab. These and others are suitable for use in certain embodiments provided herein.

In another aspect, formulations can be administered or co-administered with biological therapeutic agents and other therapeutic drugs. For example, virulizin (Lorus Therapeutics), which is believed to stimulate the release of tumor necrosis factor, TNF-α, by tumor cells in vitro and stimulate activation of macrophage cells. This can be used in combination with one or more formulation of the invention to increase cancer cell apoptosis and treat various types of cancers including pancreatic cancer, malignant melanoma, Kaposi's sarcoma (KS), lung cancer, breast cancer, uterine, ovarian and cervical cancer. Another example is CpG 7909 (Coley Pharmaceutical Group), which is believed to activate NK cells and monocytes and enhance ADCC. Cytokines such as interferons and interleukins (e.g. EPO, thrombopoietin) are biological agents useful certain embodiments in combination with one or more formulation of the invention. Other types of suitable biological therapeutic agents include RNA and protein bases-agents such as enzymes. These therapeutic agents and others can also be used in combination with formulations provided herein.

Table 2A lists several small molecule inhibitors for cancer therapy along with their specific target and type of cancer. According to some embodiments, the agents are administered transdermally. Table 1 lists other anti-cancer molecules and, in some cases, the effective doses and percent of a transdermal formulation.

TABLE 2A

| Small Molecule Drug | Specific Target | Cancer Targeted |
|---|---|---|
| Imatinib (Gleevec) | Bcr-Abl | Philadelphia chromosome-positive chronic |
| | | myelogenous leukemia |
| Gefitinib (Iressa) | EGFR | Non-small cell lung cancer |
| Erlotinib (Tarceva) | EGFR | Non-small cell lung cancer, pancreatic cancer |
| Sunitinib (sutent) | VEGFR2, RET PDGFR, | Renal cell carcinoma |
| | FLT-1, KIT, CSF-1 | |
| Lapatinib (Tykerb) | EGFR, HER2/neu | Breast cancer |
| Nilotinib (Tasigna) | Bcr-Abl, KIT, LCK | Chronic myelogenous leukemia |
| Sorafenib (Nexavar) | B-Raf, VEGFR2, EGFR, | Hepatocellular carcinoma |
| | PDGFR | |
| Temsirolimus (CCl-779) | mTOR | Renal cell carcinoma |
| Everolimus (afinitor) | mTOR | Renal cell carcinoma |
| Pazopanib (Votrient) | c-KIT, FGFR, PDGFR and | Renal cell carcinoma, soft tissue sarcoma |
| | VEGFR | |
| Crizotinib (Xalkori) | HGFR | Non-small cell lung cancer |
| Ruxolitinib (jafaki) | Jak1, Jak2 | Primary Myelofibrosis |
| Vandetenib (Caprelsa) | VEGFR, EGFR, RET | Medullary thyroid cancer |
| Axitinib (Inlyta) | VEGFR1-3, cKIT, PDGFR | Renal cell carcinoma |
| Bosutinib (Bosulif) | Src, Bcr-Abl | Philadelphia chromosome-positive chronic |
| | | myelogenous leukemia |
| Cabozantinib (Cometriz) | c.Met, VEGFR2 | Medullary thyroid cancer |
| Ponatinib (lclusig) | Bcr-Abl | Chronic myeloid leukemia, acute lymphoblastic |
| | | leukemia |
| Regorafenib (Stivagra) | VEGFR1-3, c-KIT, TIE-2, | Metastatic colorectal cancer |
| | PDGFR-β, FGFR-1, RET, | |
| | Raf-1, B-RAF | |
| Ibrutinib (Imbruvica) | Bruton's tyrosine | Mantle cell lymphoma |
| | kinase | |
| Trametinib (Mekinist) | MEK | B-RAF 600E/K-mutant metastatic melanoma |
| Perifosine | Akt | Colorectal cancer, Multiple myeloma, Colon cancer |
| Bortezomib (Velcade) | 26S proteosome | Multiple myeloma |
| Carfilzomib (Kyprolis) | 20S proteosome | Multiple myeloma |
| Marizomib (NPI-0052) | 20S proteosome | Multiple myeloma |
| Batimastat (BB-94) | Broad spectrum | Various tumors |
| | MMPs | |
| Neovastat (AE-941) | MMPs 1, 9, 12; VEGFR-2 | Renal cell carcinoma, non- small cell lymphoma |
| Prinomastat (AG-3340) | MMPs 2, 3, 9, 13 and 14 | Renal cell carcinoma |
| Rebimastat (BMS- | MMPs 1, 2, 8, 9 and 14 | Advanced non-small cell lung cancer |
| 275291) | | |
| Ganetespib | HSP 90 | Multiple cancers |
| NVP-AUY922 | HSP 90 | Various tumors |
| Marimastat (BB-2516) | Broad spectrum | |
| | MMPs | |
| Obatoclax (GX15-070) | Bcl-2 family of | Leukemia, lymphoma, myelofibrosis |
| | proteins | |
| Navitoclax (ABT-263) | Bcl-sL, Bcl-2 and Bcl- w | Lymphoma and chronic lymphocytic leukemia |

Table 2B below lists possible indications for some of the drugs listed in Table 1.

| Drug Name | Indications |
|---|---|
| doxycycline ER | rosacea |
| mizolastine MR | chronic urticaria or hives |
| lymecycline | acne vulgaris, urinary tract infections, |
| | gastroenteritis, adnexitis, chlamydial |
| | conjunctivitis, skin and soft tissue |
| | infections, otitis externa, otitis media |
| abobotulinum toxin A | glabellar lines, cervical dystonia, limb |
| | spasticity |
| adapalene + benzoyl peroxide | acne vulgaris |
| fluocinolone acetonide + | melasma |
| hydroquinone + tretinoin | |
| benzoyl peroxide | acne vulgaris |
| adapalene | acne vulgaris |
| trifarotene | acne vulgaris, congenital ichthyosis |
| metronidazole | rosacea |
| ivermectin | rosacea |
| fluocinolone acetonide | seborrhea |
| brimonidine tartrate | rosacea |
| calcitriol | plaque psoriasis |
| clobetasol propionate | plaque psoriasis |
| desonide | psoriasis, atopic dermatitis |
| amorolfine hydrochloride | onychomycosis, dermatophytosis, tinea |
| | pedis, candidiasis |
| methyl aminolevulinate | actinic keratosis, squamous cell carcinoma |
| hydrochloride | |

Another aspect of the present disclosure is a method for treating a disease or disorder or reducing a symptom thereof. The method comprising steps of administering to a subject in need thereof any herein-disclosed transdermal formulation and administering to the subject in need thereof a composition comprising an one or more medicaments selected from Table 1.

In embodiments of the method, the transdermal formulation is administered before, contemporary with, or after the composition is administered.

In some embodiments of the method, the amount of the one or more medicament is the effective dose of the medicament as described in Table 1.

In various embodiments of the method, the composition is administered by the standard route for the medicament, e.g., the standard route is oral, topical, enteral, parenteral, by intravenous injection or infusion, by intraperitoneal injection, by intramuscular injection, or by subcutaneous injection. The composition may be a liquid, a suspension, a gel, a geltab, a semisolid, a tablet, a sachet, a lozenge, a pill, or a capsule.

In embodiments of the method, the formulation comprises a medicament selected from (3S,4S)-8-(6-amino-5-((2-amino-3-chloropyridin-4-yl)thio)pyrazin-2-yl)-3-methyl-2-oxa-8-azaspiro[4.5]decan-4-amine; (S)-3-amino-6-methoxy-N-(3,3,3-trifluoro-2-hydroxy-2-methylpropyl)-5-(trifluoromethyl)picolinamide; 2-fluoro-N-methyl-4-[7-quinolin-6-yl-methyl)-imidazo[1,2-B][1,2,4]triazin-2yl]benzamide; 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide; 4-(7-Hydroxy-2-isopropyl-4-oxo-4H-quinazolin-3-yl)-benzonitrile; Abacavir; Abiraterone Acetate; abiratone acetate; abobotulinum toxin A; Acalabrutinib; Acarbose; Acetaminophen; Acetazolamide; Acetylsalicylic Acid; acitretin; acyclovir; adalimumab; adapalene; adapalene; Afoxolaner; agalsidase beta; Albendazole; Alectinib; Alectinib; alendronate sodium; alglucosidase alfa; alitretinoin; Alogliptin; alpelisib; Aluminum Hydroxide; Ambrisentan; Amiodarone; Amitriptyline; Amlodipine; Amlodipine; Amlodipine besylate; amlodopine besylate; amorolfine hydrochloride; Amoxicillin; Amphotericin B; Amphotericin B liposome; ampicillin; Amprenavir; antihemophilic factor, Fc fusion protein; apalutamide; Apatinib; apixaban; apremilast; Aprepitant; Aripiprazole; Artemether; aspirin; Atazanavir; atenolol; Atomoxetine; Atorvastatin; Atorvastatin; Avapritinib; Axitinib (Inlyta); Azacitidine; Azathioprine; azelaic acid; Azilsartan medoxomil; azithromycin; baclofen; baricitinib; basiliximab; Batimastat (BB-94); becaplermin; Belumosudil; Bendamustine; benzoyl peroxide; benzoyl peroxide; betamethasone dipropionate; betamethasone valerate; bexarotene; Bictegravir; Bimatoprost; binimetinib (Mektovi); Bortezomib; Bortezomib (Velcade); Bosentan; Bosutinib (Bosulif); botulinum toxin A; branched chain amino acids; Brexpiprazole; Brimonidine; brimonidine tartrate; brodalumab; buproprion; Cabazitaxel; Cabozantinib; Cabozantinib (Cometriz); Calcifediol; calcipotriene; Calcipotriene; Calcipotriol; calcitonin; calcitriol; calcium carbonate; Canagliflozin; canakinumab; Capecitabine; Capmatinib; capsaicin; Carbamazepine; Carbidopa; Carfilzomib; Carfilzomib (Kyprolis); Cariprazine; carvedilol; CD-12681; CEE-321; cefazolin; cefepime; Cefoperazone; cefotaxime sodium; celecoxib; celecoxib; cemiplimab; Ceritinib; Chlorothiazide; Chlorpromazine; Chlorthalidone; cinacalcet; ciprofloxacin; Cisapride; citalopram; citric acid; clarithromycin; Clavulanate; Clindamycin phosphate; Clobazam; clobetasol propionate; Clofazimine; clomiphene; clonidine; Clopidogrel; clopidogrel bisulfate; cobicistat; Cobicistat; Colchocine; Colistin; Corticotropin; crisaborole; crizanlizumab; Crizotinib (Xalkori); cyclobenzaprine; cyclosporine; Dabigatran Etexilate; Dabrafenib; Dabrafenib; dabrafenib; dabrafenib; dacomitinib (Vizimpro); dalbavancin; dalfopristin; Danazol; Dapagliflozin; Dapsone; daptomycin; Darunavir; Dasatinib; decitabine; defatted Peanut (Arachis hypogaea) Flour; deferasirox; delgocitinib; deoxycholic acid; Desipramine; desonide; Dexlansoprazole; Dexmethylphenidate; dextroamphetamine/amphetamine salts; Diclofenac; diclofenac sodium; diflucortolone; Diflucortolone; Difunisal; Digoxin; Diloxanide; Dimethyl Fumarate; diphenhydramine hydrochloride; diphtheria vaccine; docetaxel; Dolutegravir; Dolutegravir; Donepezil; Doxercalciferol; doxycycline; doxycycline ER; Doxycycline Hyclate (B); Doxycycline Monohydrate (A); Dronabinol; Dronedarone; Drospirenone; Dukoral/ShanChol cholera vaccine; Duloxetine; dupilumab; durvalumab; Dutasteride; Duvelisib; ecallantide; Edoxaban; Efavirenz; Efavirenz; Elafibranor; elagolix sodium; Elbasvir; Eletriptan; Eltrombopag; Elvitegravir; Empagliflozin; Emtricitabine; emtricitabine; Emtricitabine; Emtricitabine; Emtricitabine; Emtricitabine; Emtricitabine; Emtricitabine; encorafenib (Braftovi); enfuviritide; enoxaparin sodium; Entecavir; entrectinib; Enzalutamide; Epinephrine; erdafitinib; erenumab; Eribulin; Erlotinib; Erlotinib HCl (Tarceva); Ertapenem; Erythromycin; escitalopram; esketamine (ketamine); Esomeprazole; esopremazole; estradiol; Estrogens—conjugated; etanercept; Ethinyl Estradiol; Ethinyl Estradiol; Ethinyl Estradiol; Etonogestrel; Etonogestrel; Etravirine; etretinate; Everolimus; Everolimus (Afinitor); Everolimus (Votubia, Zortress/Certican); Ezetimibe; Ezetimibe; Ezetimibe; Famotidine; fasinumab; Febuxostat; Fedratinib; Fenofibrate; Ferric Carboxymaltose; fexofenadine hydrochloride; filgotinib; filgrastim; fingolimod; fluocinolone acetonide; fluocinolone acetonide; fluoxetine; Flurbiprofen; Folic acid; Forskolin; Fostamatinib; Fulvestrant; furosemide; fusidic acid; fusidic acid; gabapentin; Ganetespib; Gefitinib (Iressa); Gilteritinib; glasdegib (Daurismo) or glasdegib maleate; Glecaprevir; Glibenclamide; Glimepiride; glipizide; glucagon; glucosamine; glutaric anhydride; glycopyrrolate; Goserelin; goserelin LA; granisetron; Grazoprevir; Griseofulvin; halobetasol propionate; Haloperidol; heparin sodium; heplisav-B vaccine; hyaluronic acid; Hydrochlorothiazide; Hydrochlorothiazide; Hydrochlorothiazide; Hydrochlorothiazide; hydrocortisone; hydrocortisone acetate; hydroquinone; hydroxychloroquine; Hydroxyprogesterone; Ibrutinib; Ibrutinib (Imbruvica); ibuprofen; icatibant acetate; Idelalisib; Imatinib; Imatinib (Gleevec); imatinib mesylate; imiglucerase; inclisiran; Indinavir; Indomethacin; infliximab; ingenol mebutate; inotersen; insulin A; insuline glargine; interferon beta-lb; Iopanoic acid; Irbesartan; isoconazole; isoconazole; Isotretinoin; Itraconazole; Ivacaftor; Ivacaftor; ivermectin; Ixazomib; ketoconazole; ketones; ketoprofen; Keyruvia; Lacosamide; lamictal; Lamivudine; Lansoprazole; Lapatinib; Lapatinib (Tykerb); larotrectinib; Ledipasvir; leflunomide; Lenalidomide; Lenvatinib; LEO-138559; LEO-152020; Leuprolide; Levetiracetam; levodopa; Levodopa/Benserazide; Levonorgestrel; Levothyroxine; Linagliptin; linezolid; lisinopril; L-lysine free base; lofexidine; Lopinavir; Loratadine; lorlatinib (Lorviqua); Losartan; Lovastatin; Lubiprostone; Lumacaftor/Ivacaftor; Lumefantrine; Lurasidone; Luspatercept; lymecycline; Macitentan; magnesium; magnesium lactate; Marimastat (BB-2516); Marizomib (NPI-0052); Mebendazole; Mefloquine; melatonin; meloxicam; memantine hydrochloride; meningococcal[serotype b] vaccine; Mesalazine; Metformin; Metformin; metformin; Metformin; methotrexate; methyl aminolevulinate hydrochloride; Methylphenidate; methylprednisolone; Metoprolol succinate; metronidazole; Midostaurin; minoxidil; Mirabegron; mizolastine MR; Montelukast; Mycophenolate Mofetil; N-(3-(2-(2-hydroxyethoxy)-6-morpholinopyridin-4-yl)-4-methylphenyl)-2-(trifluoromethyl)-isonicotinamide; N-(3-(6-Amino-5-(2-(N-methylacrylamido)ethoxy)pyrimidin-4-yl)-5-fluoro-2-methylphenyl)-4-cyclopropyl-2-fluorobenzamide; N-[4-(Chlorodifluoromethoxy)phenyl]-6-[(3R)-3-hydroxpyrrolidin-1-yl]-5-(1H-pyrazol-5-yl)-pyridine-3-carboxamide hydrochloride; Nalidixic acid (Quinolone antibiotic); naloxone; Naproxen; Navitoclax (ABT-263); Nebivolol; Nelfinavir; nemolizumab; neomycin; Neovastat (AE-941); Neratinib—Free Base; Neratinib (Nerlynx); Neratinib Maleate; Nevirapine; Niclosamide; Nifedipine; nilotinib; Nilotinib; Nilotinib (Tasigna); Nintedanib; niratimib; Nitric Oxide; Nitrofurantoin; Norethindrone Acetate; nusinersen; NVP-AUY922; Nystatin; Obatoclax (GX15-070); octreotide acetate; ofatumumab; Ofloxacin; Olanzapine; olaparib; Olmesartan Medoxomil; olumacostat; omalizumab; Oseltamivir; Osimertinib; osimertinib mesylate; Oxaprozin; Ozanimod; Paclitaxel; Paclitaxel Protein Bound; Palbociclib (Ibrance); Paliperidone Palmitate; Pantoprazole; parathyroid hormone; Paricalcitol; patisiran; pazopanib; Pazopanib (Votrient); pazopanib hydrochloride; Peficitinib; pegfilgrastim; Pembrolizumab (Keytruda); Pemetrexed; Pemigatinib; Perifosine; Pexidartinib; Phenazopyridine; Phenytoin; Pibrentasvir; Piperacillin; Pirfenidone; Piroxicam; Pomalidomide; Ponatinib; Ponatinib (lclusig); Posaconazole; Pralsetinib; pravastatin; prednicarbate; prednisone; pregabalin; pregabalin; Prinomastat (AG-3340); Progesterone; propanolol; protamine sulfate; psilocybin; Pyrantel; Pyrimethamine; Quetiapine; Quinupristin; Raloxifene; Raltegravir; ranibizumab; ranitidine; Ranolazine; Rebimastat (BMS-275291); Regorafenib; Regorafenib (Stivarga); relugolix (Orgovyx); remibrutinib; resiquimod; retinal; Retinol; ribociclib; ribociclib succinate; Rifampin; Rilpivirine; Rilpivirine; Rilpivirine; Rimegepant; Riociguat; Risdiplam; Risperidone; Ritonavir; Rivaroxaban; Rosuvastatin; rosuvastatin calcium; Rotigotine; roxadustat; rucaparib camsylate (Rubraca); ruxolitinib; Ruxolitinib (Jafaki); Sacubitril, valsartan; salicylic acid; Sapropterin; Saquinavir; sarilumab; Saxagliptin, Metformin; secukinumab; Selexipag; selinexor; Selpercatinib; selpercatinib (LOXO-292); Selumetinib; semaglutide; sertraline; Sevoflurane; sildenafil; Sildenafil Citrate; Simvastatin; siponimod; Siremadlin; Sirolimus; Sitagliptin; Sitagliptin phosphate monohydrate; Sodium bicarbonate or sodium carbonate; sodium deoxycholate; sodium nitrate; Sofosbuvir; Sofosbuvir; Sofosbuvir; Solifenacin; somatropin; sonidegib phosphate; Sorafenib; Sorafenib (Nexavar); spartalizumab; Spironolactone; sufentanil citrate; Sugammadex; sulbactam; Sulbactam; Sulfadiazine; Sulfamethoxazole; sulfasalazine; sumatriptan; Sunitinib; Sunitinib; Sunitinib (sutent); sunitinib malate (Sutent); Tacrolimus; TAF; TAF; TAF; TAF; talazoparib—Talzenna); Talinolol; Tamoxifen; Tamsulosin; tazarotene; tazobactam; TDF; TDF; Temsirolimus (CCl-779, Torisel); Teneligliptin; tenofovir alafenamide; Tenofovir Disoproxil Fumarate; Tepotinib; terbinafine; Terfenadine; Teriflunomide; Testosterone; Tezacaftor; THC; ticagrelor; tigecycline; Timolol; timolol maleate; Tipranavir; tisagenlecleucel; Tisagenlecleucel (Kymriah); Tivozanib; tofacitinib citrate; Tolvaptan; tralokinumab; tramadol; Trametinib; Trametinib; trametinib; Trametinib; Trametinib (Mekinist); tranexamic acid; Travoprost; trazodone; tretinoin; tretinoin; triancinolone; Triclabendazole; trifarotene; Trimethoprim; Triptorelin; tris; Tucatinib; ubrogepant; Umbralisib; upadacitinib; valproic acid; Valsartan; valsartan; Valsartan; valsartan; vancomycin; Vancomycin Hydrochloride (A); Vancomycin Hydrochloride (B); Vandetanib; Vandetenib (Caprelsa); Varenicline; Vasopressin; Velpatasvir; Velpatasvir; Vemurafenib; Venetoclax; Verapamil hydrochloride; Vilazodone; Vildagliptin; vismodegib; vitamin B6; vitamin D; Vonoprazan; Voriconazole; Vortioxetine; Voxilaprevir; Warfarin Sodium; Zanubrutinib; Zinc; zoledronic acid; and Zolpidem and the medicament of the composition is selected from (3S,4S)-8-(6-amino-5-((2-amino-3-chloropyridin-4-yl)thio)pyrazin-2-yl)-3-methyl-2-oxa-8-azaspiro[4.5]decan-4-amine; (S)-3-amino-6-methoxy-N-(3,3,3-trifluoro-2-hydroxy-2-methylpropyl)-5-(trifluoromethyl)picolinamide; 2-fluoro-N-methyl-4-[7-quinolin-6-yl-methyl)-imidazo[1,2-B][1,2,4]triazin-2yl]benzamide; 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide; 4-(7-Hydroxy-2-isopropyl-4-oxo-4H-quinazolin-3-yl)-benzonitrile; Abacavir; Abiraterone Acetate; abiratone acetate; abobotulinum toxin A; Acalabrutinib; Acarbose; Acetaminophen; Acetazolamide; Acetylsalicylic Acid; acitretin; acyclovir; adalimumab; adapalene; adapalene; Afoxolaner; agalsidase beta; Albendazole; Alectinib; Alectinib; alendronate sodium; alglucosidase alfa; alitretinoin; Alogliptin; alpelisib; Aluminum Hydroxide; Ambrisentan; Amiodarone; Amitriptyline; Amlodipine; Amlodipine; Amlodipine besylate; amlodopine besylate; amorolfine hydrochloride; Amoxicillin; Amphotericin B; Amphotericin B liposome; ampicillin; Amprenavir; antihemophilic factor, Fc fusion protein; apalutamide; Apatinib; apixaban; apremilast; Aprepitant; Aripiprazole; Artemether; aspirin; Atazanavir; atenolol; Atomoxetine; Atorvastatin; Atorvastatin; Avapritinib; Axitinib (Inlyta); Azacitidine; Azathioprine; azelaic acid; Azilsartan medoxomil; azithromycin; baclofen; baricitinib; basiliximab; Batimastat (BB-94); becaplermin; Belumosudil; Bendamustine; benzoyl peroxide; benzoyl peroxide; betamethasone dipropionate; betamethasone valerate; bexarotene; Bictegravir; Bimatoprost; binimetinib (Mektovi); Bortezomib; Bortezomib (Velcade); Bosentan; Bosutinib (Bosulif); botulinum toxin A; branched chain amino acids; Brexpiprazole; Brimonidine; brimonidine tartrate; brodalumab; buproprion; Cabazitaxel; Cabozantinib; Cabozantinib (Cometriz); Calcifediol; calcipotriene; Calcipotriene; Calcipotriol; calcitonin; calcitriol; calcium carbonate; Canagliflozin; canakinumab; Capecitabine; Capmatinib; capsaicin; Carbamazepine; Carbidopa; Carfilzomib; Carfilzomib (Kyprolis); Cariprazine; carvedilol; CD-12681; CEE-321; cefazolin; cefepime; Cefoperazone; cefotaxime sodium; celecoxib; celecoxib; cemiplimab; Ceritinib; Chlorothiazide; Chlorpromazine; Chlorthalidone; cinacalcet; ciprofloxacin; Cisapride; citalopram; citric acid; clarithromycin; Clavulanate; Clindamycin phosphate; Clobazam; clobetasol propionate; Clofazimine; clomiphene; clonidine; Clopidogrel; clopidogrel bisulfate; cobicistat; Cobicistat; Colchocine; Colistin; Corticotropin; crisaborole; crizanlizumab; Crizotinib (Xalkori); cyclobenzaprine; cyclosporine; Dabigatran Etexilate; Dabrafenib; Dabrafenib; dabrafenib; dabrafenib; dacomitinib (Vizimpro); dalbavancin; dalfopristin; Danazol; Dapagliflozin; Dapsone; daptomycin; Darunavir; Dasatinib; decitabine; defatted Peanut (Arachis hypogaea) Flour; deferasirox; delgocitinib; deoxycholic acid; Desipramine; desonide; Dexlansoprazole; Dexmethylphenidate; dextroamphetamine/amphetamine salts; Diclofenac; diclofenac sodium; diflucortolone; Diflucortolone; Difunisal; Digoxin; Diloxanide; Dimethyl Fumarate; diphenhydramine hydrochloride; diphtheria vaccine; docetaxel; Dolutegravir; Dolutegravir; Donepezil; Doxercalciferol; doxycycline; doxycycline ER; Doxycycline Hyclate (B); Doxycycline Monohydrate (A); Dronabinol; Dronedarone; Drospirenone; Dukoral/ShanChol cholera vaccine; Duloxetine; dupilumab; durvalumab; Dutasteride; Duvelisib; ecallantide; Edoxaban; Efavirenz; Efavirenz; Elafibranor; elagolix sodium; Elbasvir; Eletriptan; Eltrombopag; Elvitegravir; Empagliflozin; Emtricitabine; emtricitabine; Emtricitabine; Emtricitabine; Emtricitabine; Emtricitabine; Emtricitabine; Emtricitabine; encorafenib (Braftovi); enfuviritide; enoxaparin sodium; Entecavir; entrectinib; Enzalutamide; Epinephrine; erdafitinib; erenumab; Eribulin; Erlotinib; Erlotinib HCl (Tarceva); Ertapenem; Erythromycin; escitalopram; esketamine (ketamine); Esomeprazole; esopremazole; estradiol; Estrogens—conjugated; etanercept; Ethinyl Estradiol; Ethinyl Estradiol; Ethinyl Estradiol; Etonogestrel; Etonogestrel; Etravirine; etretinate; Everolimus; Everolimus (Afinitor); Everolimus (Votubia, Zortress/Certican); Ezetimibe; Ezetimibe; Ezetimibe; Famotidine; fasinumab; Febuxostat; Fedratinib; Fenofibrate; Ferric Carboxymaltose; fexofenadine hydrochloride; filgotinib; filgrastim; fingolimod; fluocinolone acetonide; fluocinolone acetonide; fluoxetine; Flurbiprofen; Folic acid; Forskolin; Fostamatinib; Fulvestrant; furosemide; fusidic acid; fusidic acid; gabapentin; Ganetespib; Gefitinib (Iressa); Gilteritinib; glasdegib (Daurismo) or glasdegib maleate; Glecaprevir; Glibenclamide; Glimepiride; glipizide; glucagon; glucosamine; glutaric anhydride; glycopyrrolate; Goserelin; goserelin LA; granisetron; Grazoprevir; Griseofulvin; halobetasol propionate; Haloperidol; heparin sodium; heplisav-B vaccine; hyaluronic acid; Hydrochlorothiazide; Hydrochlorothiazide; Hydrochlorothiazide; Hydrochlorothiazide; hydrocortisone; hydrocortisone acetate; hydroquinone; hydroxychloroquine; Hydroxyprogesterone; Ibrutinib; Ibrutinib (Imbruvica); ibuprofen; icatibant acetate; Idelalisib; Imatinib; Imatinib (Gleevec); imatinib mesylate; imiglucerase; inclisiran; Indinavir; Indomethacin; infliximab; ingenol mebutate; inotersen; insulin A; insuline glargine; interferon beta-Ib; Iopanoic acid; Irbesartan; isoconazole; isoconazole; Isotretinoin; Itraconazole; Ivacaftor; Ivacaftor; ivermectin; Ixazomib; ketoconazole; ketones; ketoprofen; Keyruvia; Lacosamide; lamictal; Lamivudine; Lansoprazole; Lapatinib; Lapatinib (Tykerb); larotrectinib; Ledipasvir; leflunomide; Lenalidomide; Lenvatinib; LEO-138559; LEO-152020; Leuprolide; Levetiracetam; levodopa; Levodopa/Benserazide; Levonorgestrel; Levothyroxine; Linagliptin; linezolid; lisinopril; L-lysine free base; lofexidine; Lopinavir; Loratadine; lorlatinib (Lorviqua); Losartan; Lovastatin; Lubiprostone; Lumacaftor/Ivacaftor; Lumefantrine; Lurasidone; Luspatercept; lymecycline; Macitentan; magnesium; magnesium lactate; Marimastat (BB-2516); Marizomib (NPI-0052); Mebendazole; Mefloquine; melatonin; meloxicam; memantine hydrochloride; meningococcal[serotype b] vaccine; Mesalazine; Metformin; Metformin; metformin; Metformin; methotrexate; methyl aminolevulinate hydrochloride; Methylphenidate; methylprednisolone; Metoprolol succinate; metronidazole; Midostaurin; minoxidil; Mirabegron; mizolastine MR; Montelukast; Mycophenolate Mofetil; N-(3-(2-(2-hydroxyethoxy)-6-morpholinopyridin-4-yl)-4-methylphenyl)-2-(trifluoromethyl)-isonicotinamide; N-(3-(6-Amino-5-(2-(N-methylacrylamido)ethoxy)pyrimidin-4-yl)-5-fluoro-2-methylphenyl)-4-cyclopropyl-2-fluorobenzamide; N-[4-(Chlorodifluoromethoxy)phenyl]-6-[(3R)-3-hydroxpyrrolidin-1-yl]-5-(1H-pyrazol-5-yl)-pyridine-3-carboxamide hydrochloride; Nalidixic acid (Quinolone antibiotic); naloxone; Naproxen; Navitoclax (ABT-263); Nebivolol; Nelfinavir; nemolizumab; neomycin; Neovastat (AE-941); Neratinib—Free Base; Neratinib (Nerlynx); Neratinib Maleate; Nevirapine; Niclosamide; Nifedipine; nilotinib; Nilotinib; Nilotinib (Tasigna); Nintedanib; niratimib; Nitric Oxide; Nitrofurantoin; Norethindrone Acetate; nusinersen; NVP-AUY922; Nystatin; Obatoclax (GX15-070); octreotide acetate; ofatumumab; Ofloxacin; Olanzapine; olaparib; Olmesartan Medoxomil; olumacostat; omalizumab; Oseltamivir; Osimertinib; osimertinib mesylate; Oxaprozin; Ozanimod; Paclitaxel; Paclitaxel Protein Bound; Palbociclib (Ibrance); Paliperidone Palmitate; Pantoprazole; parathyroid hormone; Paricalcitol; patisiran; pazopanib; Pazopanib (Votrient); pazopanib hydrochloride; Peficitinib; pegfilgrastim; Pembrolizumab (Keytruda); Pemetrexed; Pemigatinib; Perifosine; Pexidartinib; Phenazopyridine; Phenytoin; Pibrentasvir; Piperacillin; Pirfenidone; Piroxicam; Pomalidomide; Ponatinib; Ponatinib (lclusig); Posaconazole; Pralsetinib; pravastatin; prednicarbate; prednisone; pregabalin; pregabalin; Prinomastat (AG-3340); Progesterone; propanolol; protamine sulfate; psilocybin; Pyrantel; Pyrimethamine; Quetiapine; Quinupristin; Raloxifene; Raltegravir; ranibizumab; ranitidine; Ranolazine; Rebimastat (BMS-275291); Regorafenib; Regorafenib (Stivarga); relugolix (Orgovyx); remibrutinib; resiquimod; retinal; Retinol; ribociclib; ribociclib succinate; Rifampin; Rilpivirine; Rilpivirine; Rilpivirine; Rimegepant; Riociguat; Risdiplam; Risperidone; Ritonavir; Rivaroxaban; Rosuvastatin; rosuvastatin calcium; Rotigotine; roxadustat; rucaparib camsylate (Rubraca); ruxolitinib; Ruxolitinib (Jafaki); Sacubitril, valsartan; salicylic acid; Sapropterin; Saquinavir; sarilumab; Saxagliptin, Metformin; secukinumab; Selexipag; selinexor; Selpercatinib; selpercatinib (LOXO-292); Selumetinib; semaglutide; sertraline; Sevoflurane; sildenafil; Sildenafil Citrate; Simvastatin; siponimod; Siremadlin; Sirolimus; Sitagliptin; Sitagliptin phosphate monohydrate; Sodium bicarbonate or sodium carbonate; sodium deoxycholate; sodium nitrate; Sofosbuvir; Sofosbuvir; Sofosbuvir; Solifenacin; somatropin; sonidegib phosphate; Sorafenib; Sorafenib (Nexavar); spartalizumab; Spironolactone; sufentanil citrate; Sugammadex; sulbactam; Sulbactam; Sulfadiazine; Sulfamethoxazole; sulfasalazine; sumatriptan; Sunitinib; Sunitinib; Sunitinib (sutent); sunitinib malate (Sutent); Tacrolimus; TAF; TAF; TAF; TAF; talazoparib—Talzenna); Talinolol; Tamoxifen; Tamsulosin; tazarotene; tazobactam; TDF; TDF; Temsirolimus (CCl-779, Torisel); Teneligliptin; tenofovir alafenamide; Tenofovir Disoproxil Fumarate; Tepotinib; terbinafine; Terfenadine; Teriflunomide; Testosterone; Tezacaftor; THC; ticagrelor; tigecycline; Timolol; timolol maleate; Tipranavir; tisagenlecleucel; Tisagenlecleucel (Kymriah); Tivozanib; tofacitinib citrate; Tolvaptan; tralokinumab; tramadol; Trametinib; Trametinib; trametinib; Trametinib; Trametinib (Mekinist); tranexamic acid; Travoprost; trazodone; tretinoin; tretinoin; triancinolone; Triclabendazole; trifarotene; Trimethoprim; Triptorelin; tris; Tucatinib; ubrogepant; Umbralisib; upadacitinib; valproic acid; Valsartan; valsartan; Valsartan; valsartan; vancomycin; Vancomycin Hydrochloride (A); Vancomycin Hydrochloride (B); Vandetanib; Vandetenib (Caprelsa); Varenicline; Vasopressin; Velpatasvir; Velpatasvir; Vemurafenib; Venetoclax; Verapamil hydrochloride; Vilazodone; Vildagliptin; vismodegib; vitamin B6; vitamin D; Vonoprazan; Voriconazole; Vortioxetine; Voxilaprevir; Warfarin Sodium; Zanubrutinib; Zinc; zoledronic acid; and Zolpidem. Any medicament from Table 1 can be used in a transdermal formulation as a co-therapy with a composition comprising any medicament from Table 1. In some cases, the medicament in the formulation is the same as the medicament in the composition. In other cases, the medicament in the formulation is different from the medicament in the composition. In yet other cases, the medicament in the formulation is also the medicament in the composition and the composition further comprises another medicament from Table 1. In further cases, the medicament in the composition is also the medicament in the formulation and the formulation further comprises another medicament from Table 1.

Transdermal formulations of the present disclosure (and comprising a medicament of Table 1) may be administered in a co-therapy with pembrolizumab (Keytruda). Generally, pembrolizumab is administered by intravenous infusion. Therefore, in embodiments, a transdermal formulation comprising a medicament of Table 1 is co-administered with an intravenous infusion composition comprising pembrolizumab.

A transdermal delivery formulation containing iron can be formulated at acidic pH to minimize the spontaneous oxidation Fe(II) into Fe(III). Suitable nonlimiting exemplary iron chelators include deferoxamine, ethylenediaminetetraacetic acid (EDTA), 1,2-diethyl-3-hydroxypyridin-4-one (CP94), Desferal, Deferiprone and Deferasirox, succimer, trientine, Desferrithiocin, Clioquinol, O-trensox, Tachpyr, Dexrazoxane, Triapine, Pyridoxal isonicotinoyl hydrazone, Di-2-pyridylketone thiosemicarbazone series, Flavan-3-ol, Curcumin, Apocynin, Kolaviron, Floranol, Baicalein, Baicalin, ligustrazine, Quercetin, Epigallocatechin gallate, Theaflavin, Phytic acid, and Genistein. Suitable nonlimiting exemplary antioxidants include glutathione, vitamin C, vitamin E, superoxide dismutase, catalase, pNaKtide, Butylated hydroxytoluene, Butylated hydroxyanisole, tert-Butylhydroquinone, HP beta CD, resveratrol, retinol, coenzyme q10, niacinamide, polyphenols, flavonoids, beta-carotene, lutein, and lycopene.

In any of the anesthetic compositions of a transdermal delivery formulation, it may be desirable to administer the epinephrine in tandem with a transdermal anesthetic. Alternatively, treatment of the epinephrine with a chelator, such as the iron chelator Desferal® may stabilize the epinephrine sufficiently to include it in the transdermal delivery formulation.

A suitable dose of iron or an iron containing transdermal delivery formulation administered topically as a transdermal delivery formulation for a subject (e.g. a human patient) is at least about 500 mg, at least about 750 mg, at least about 1000 mg, at least about 1.5 g, at least about 2.0 g, at least about 2.5 g, at least about 3.0 g, at least about 3.5 g, at least about 4.0 g, at least about 4.5 g, at least about 5.0 g, at least about 6.0 g, at least about 7.0 g, at least about 8.0 g, at least about 9.0 g, at least about 10.0 g, at least about 11 g, at least about 12 g, at least about 13 g, at least about 14 g, at least about 15 g, at least about 20 g, at least about 25 g, at least about 30 g, at least about 35 g, at least about 40 g, at least about 45 g, at least about 50 g, or more. This dose is typically administered daily, twice a day, or three times a day, but it may also be administered four times a day, five times a day, or more than five times a day.

Alternatively, a suitable daily dose of iron or an iron containing transdermal delivery formulation administered topically as a transdermal delivery formulation for a subject is at least about 10 mg/kg, at least about 25 mg/kg, at least about 30 mg/kg, at least about 35 mg/kg, at least about 40 mg/kg, at least about 45 mg/kg, at least about 50 mg/kg, at least about 55 mg/kg, at least about 60 mg/kg, at least about 65 mg/kg, at least about 70 mg/kg, at least about 75 mg/kg, at least about 80 mg/kg, at least about 90 mg/kg, at least about 100 mg/kg, at least about 125 mg/kg, at least about 150 mg/kg, at least about 160 mg/kg, at least about 170 mg/kg, at least about 175 mg/kg, at least about 180 mg/kg, at least about 190 mg/kg, at least about 200 mg/kg, at least about 225 mg/kg, at least about 250 mg/kg, at least about 275 mg/kg, at least about 300 mg/kg, at least about 325 mg/kg, at least about 350 mg/kg, at least about 375 mg/kg, at least about 400 mg/kg, at least about 425 mg/kg, at least about 450 mg/kg, at least about 475 mg/kg, up to at least about 500 mg/kg or more.

In another aspect, a suitable daily dose of iron or an iron containing transdermal delivery formulation administered topically as a transdermal delivery formulation for a subject is about 10 mg/kg to about 1.0 g/kg, and more typically the daily dose is about 10 mg/kg to about 500 mg/kg, about 25 mg/kg to about 500 mg/kg, about 50 mg/kg to about 300 mg/kg, about 75 mg/kg to about 300 mg/kg, about 75 mg/kg to about 250 mg/kg, about 100 mg/kg to about 300 mg/kg, about 75 mg/kg to about 200 mg/kg, about 100 mg/kg to about 200 mg/kg, or alternative ranges.

A transdermal delivery formulation comprise mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize one or more distinct permeation enhancers.

Emergency medical treatment of individuals requiring, for example, blood balancing agents including electrolytes and readily-metabolized nutrients, such as glucose, that would otherwise be administered intravenously can instead be non-invasively treated by massaging the formulation through the skin and thus permitting systemic delivery so that levels in the bloodstream are altered.

In some embodiments, the components for athletic performance include beta-alanine, L-carnitine, adenosine triphosphate, dextrose, creatine monohydrate, beta hydroxy-betamethylbutyrate (HMB), branched chain amino acids (leucine, isoleucine, valine), glutathione, sodium phosphate, and caffeine. Components for medical nutrition include amino acids, dextrose, lipids, Na⁺, K⁺, Ca²⁺, Mg²⁺, acetate, Cl⁻, P, multivitamin, and trace elements. While components for weight loss include conjugated linoleic acids, ephedra, caffeine, and salicin.

Embodiments include a transdermal lotion or cream for administration of medicaments to a subject. It is placed on the skin to deliver a specific dose of an agent through the skin. The agent can be delivered across the skin into a localized subdermal location. For example, a lotion can alleviate inflammation from an autoimmune response. The lotion or cream can be applied directly to the affected area. Alternatively, the agent can enter the circulation for systemic distribution.

In an alternative embodiment, an agent can be administered using a transdermal or medicated adhesive patch. To release an agent, a patch can utilize a porous membrane covering a reservoir of the agent. Alternatively, the agent can be embedded in layers of the adhesive that release the agent as they dissolve or melt.

An advantage of a transdermal drug delivery route over other types of delivery is that the formulation can provide a controlled release of the agent. Conventional transdermal delivery systems are generally ineffective for use with agents and medications that are large molecules and/or hydrophilic molecules.

There are other advantages to transdermal administration of medicaments. Proteins and peptides used, for example, in aging treatments can be degraded by the gastric acid and enzymes. Transdermal administration is not affected by stomach or digestive issues. Further, people can benefit from drugs that are absorbed slowly and regularly. With a transdermal formulation, a medicament can be released in small quantities over a long period of time.

Other advantages are related to dosing. Large doses of agents can cause dose-dependent toxicity in many cases. For example, oral administration of vitamin A can result in hypervitaminosis A. The main problems associated with the vitamin A are its half-life, fast absorption (due to lipophilicity) and its toxicity (due to high loading and frequent dosing). Also, some drugs undergo first-pass metabolism, which prevents their delivery to the desired site of action. Furthermore, many hydrophilic or lipophilic drugs show either poor dissolution or poor absorption on oral administration. With a transdermal formulation, the effective concentration of an agent can be applied at the desired site without painful delivery.

In one embodiment, medicaments are supplied via transdermal administration. There are many occasions in which the formulations of the invention are useful. For athletes, a transdermal delivery formulation can deliver to tired muscles sufficient amounts of a neutralizing agent for lactic acid, such as a ketone component, to relieve the burning sensation felt by the athlete due to the buildup of lactic acid. This permits the athlete to continue to perform at optimum level for longer periods of time. In addition, athletes or others “working out” are expending high amounts of energy and are in need of energy generation, especially in those areas of their musculature that are involved in performing workouts and, therefore, need to consume large numbers of calories. These nutrients can be supplied directly rather than requiring oral ingestion which is counterproductive and relatively slow.

The relationship between abnormal protein phosphorylation and disease cause or prognosis is well known. Protein kinases are a large and diverse family of enzymes that catalyze protein phosphorylation and play an important role in cell signaling.

Protein kinases therefore represent an important group of drug targets. The widespread occurrence of overactive protein kinases in cancer cells suggests that molecules that inhibit these enzymes might act as antitumor agents. Various protein kinase inhibitors have been used clinically in the treatment of diseases such as cancer and chronic inflammatory diseases including diabetes and stroke.

Protein kinases can exert positive or negative regulatory effects depending on the target protein. Protein kinases are involved in specific signal transduction pathways that regulate cell functions, which include metabolism, cell cycle progression, cell adhesion, vascular function, apoptosis and angiogenesis. Cellular signaling dysfunction is associated with many diseases, including cancer and diabetes. Regulation of signal transduction by cytokines and the relationship between signal molecules and proto-oncogenes and tumor suppressor genes have also been demonstrated. The relationship between diabetes and related conditions and deregulation levels of protein kinases has also been demonstrated. See, for example, Sridhar et al., Pharmaceutical Research, 17 (1): 1345-1353 (2000). Viral infections and associated conditions are also implicated in the regulation of protein kinases. Park et al., Cell, 101 (7): 777-787 (2000).

Protein kinases can be divided into broad groups based on the identity of the amino acids that they target (serine/threonine, tyrosine, lysine and histidine). For example, tyrosine kinases include receptor tyrosine kinases (RTK) such as growth factors and non-receptor tyrosine kinases such as the src kinase family. There are also bispecific protein kinases that target both tyrosine and serine/threonine, such as cyclin dependent kinases (CDK) and mitogen activated protein kinases (MAPK). Certain cells contain many protein kinases, some of which phosphorylate other protein kinases. Some protein kinases phosphorylate a wide variety of proteins, others phosphorylate only one protein. As expected, there are many classes of protein kinases. Upon receiving a signal, some proteins may undergo autophosphorylation.

Protein tyrosine kinases (PTKs) constitute a large family of kinases that regulate cells to cellular signals involved in proliferation, differentiation, adhesion, motility and death. Tyrosine kinase members include, Yes, BMX, Syk, EphA1, FGFR3, RKY, MUSK, JAK1 and FGFR. Tyrosine kinases are divided into two classes: receptor tyrosine kinases and non-receptor tyrosine kinases. The tyrosine kinase family is large, consisting of at least 90 characterized kinases of at least 58 receptor kinases and at least 32 non-receptor kinases, totaling at least 30 subfamilies. Tyrosine kinases are involved in diabetes and cancer and have been linked to various congenital syndromes.

Non-receptor tyrosine kinases are a group of intracellular enzymes that do not contain extracellular and transmembrane sequences. Currently, over 32 non-receptor tyrosine kinase families have been identified, for example, Src, Btk, Csk, ZAP70, Kak family. The Src family of non-receptor tyrosine kinase families is the largest and includes Src, Yes, Fyn, Lyn, Lek, Blk, Hck, Fgr and Yrk protein tyrosine kinases. The Src family of kinases has been linked to carcinogenesis, cell proliferation and tumor progression. Many protein tyrosine kinases have been shown to be involved in cell signaling pathways involved in various pathological conditions including cancer, hyperproliferative diseases and immune diseases.

Other proteins that play an important role in cell signaling include matrix metalloproteinases (MMPs), heat shock proteins (HSPs) and proteosome proteins. Matrix metalloproteinases (MMPs), also known as matrix metallopeptidases or matrixins, are metalloproteinases that are calcium-dependent zinc-containing endopeptidases; other family members are adamalysins, serralysins, and astacins. The MMPs belong to a larger family of proteases known as the metzincin superfamily. Collectively, these enzymes can degrade extracellular matrix proteins and process a number of bioactive molecules. They are known to be involved in the cleavage of cell surface receptors, the release of apoptotic ligands (such as the FAS ligand), and chemokine/cytokine inactivation. MMPs are also thought to play a major role in cell behaviors such as cell proliferation, migration (adhesion/dispersion), differentiation, angiogenesis, apoptosis, and host defense.

Heat shock proteins (HSPs) are a family of proteins that are produced by cells in response to exposure to stressful conditions. They were first described in relation to heat shock but are now known to also be expressed during other stresses including exposure to cold, UV light and during wound healing or tissue remodeling. Many members of this group perform chaperone functions by stabilizing new proteins to ensure correct folding or by helping to refold proteins that were damaged by the cell stress. Proteasomes are protein complexes which degrade unneeded or damaged proteins by proteolysis, a chemical reaction that breaks peptide bonds. Enzymes that help such reactions are called proteases. Proteasomes are part of a major mechanism by which cells regulate the concentration of particular proteins and degrade misfolded proteins.

Embodiments include transdermal administration of a medicament to modulate one or more of (a) a serine/threonine/tyrosine kinase, (b) a matrix metalloproteinase (MMP), (c) a heat shock protein (HSP), or (d) a proteosome. The medicament can be a small molecule cancer drug that is administered using a formulation as described herein.

### Transdermal Delivery Formulation Components

Embodiments include a transdermal lotion or cream for administration of a medicament to a subject. It is placed on the skin to deliver a specific dose of an agent through the skin. The agent can be delivered across the skin into a localized subdermal location.

In an alternative embodiment, an agent can be administered using a transdermal or medicated adhesive patch. To release an agent, a patch can utilize a porous membrane covering a reservoir of the agent. Alternatively, the agent can be embedded in layers of the adhesive that release the agent as they dissolve or melt.

An advantage of a transdermal drug delivery route over other types of delivery is that the formulation can provide a controlled release of the agent. Conventional transdermal delivery systems are generally ineffective for use with agents and medications that are large molecules and/or hydrophilic molecules.

There are other advantages to transdermal administration of medicaments. Small molecules can be inactivated or degraded by the stomach or liver. Transdermal administration is not affected by stomach or digestive issues. Further, people can benefit from drugs that are absorbed slowly and regularly. With a transdermal formulation, a medicament can be released in small quantities over a long period of time.

Other advantages are related to dosing. Large doses of agents can cause dose-dependent toxicity in many cases. For example, oral administration of vitamin A can result in hypervitaminosis A. The main problems associated with the vitamin A are its half-life, fast absorption (due to lipophilicity) and its toxicity (due to high loading and frequent dosing). Also, some drugs undergo first-pass metabolism, which prevents their delivery to the desired site of action. Furthermore, many hydrophilic or lipophilic drugs show either poor dissolution or poor absorption on oral administration. With a transdermal formulation, the effective concentration of an agent can be applied at the desired site without painful delivery.

In one embodiment, the present disclosure demonstrates transdermal delivery of an agent such as a medicament without many of the negative effects on color, smell, grittiness and stability driven by the use of lecithin organogel. Moreover, the methods describe improved transdermal penetration.

In an embodiment, a transdermal delivery formulation contains a phosphatide in a concentration of at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

Phosphatides—Soy lecithin contains about 57.5% w/w phosphatides. The primary phosphatides found in Soy Lecithin are inositol phosphatides (20.5% w/w of Soy lecithin), phosphatidylcholine (20%), and phosphatidylethanolamine (11% w/w of Soy lecithin). In some embodiments, phosphatidylcholine is used for the full amount (57.5% w/w of Soy lecithin) as it is known to aide in skin penetration. Other phosphatides include phosphatidic acid, phosphatidylserine and phosphatidylinositol.

In various embodiments, a formulation lacks a natural (e.g., plant or animal derived) lecithin.

In an embodiment, a transdermal delivery formulation contains a sterol or benzyl alcohol in a concentration of at least 1%, at least 2%, at least 3%, at least 4%, at least 5%, at least 6%, at least 7%, at least 8%, at least 9%, at least 10%, at least 15%, at least 20%, at least 25%, at least 30% or more w/w of the transdermal delivery formulation.

Sterols—Soy lecithin contains about 2.5% w/w sterols. In some embodiments, benzyl alcohol is used in substitution of the sterol in a transdermal delivery formulation to act as a penetration enhancer. In another embodiment, a sterol is cholesterol, ergosterol, hopanoids, hydroxysteroid, phytosterol and/or other steroids.

In an embodiment, a transdermal delivery formulation contains a carbohydrate in a concentration of at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

Carbohydrates—Soy lecithin contains about 5% w/w free carbohydrates. In some embodiments, glucose is used in substitution of a free carbohydrate to maintain the ratio of sugars in the transdermal delivery formulation disclosed herein. In another embodiment, a carbohydrate is a monosaccharide, a disaccharide, a polyol, a malto-oligosaccharide, an oligosaccharide, a starch, a polysaccharide. In a further embodiment, a carbohydrate is glucose, galactose, fructose, xylose, sucrose, lactose, maltose, trehalose, sorbitol, mannitol, maltodextrins, raffinose, stachyose, fructo-oligosaccharide, amylose, amylopectin, modified starches, glycogen, cellulose, hemicellulose, pectin and/or hydrocolloid.

In various embodiments, a transdermal delivery formulation contains no glucose.

Moisture—In some embodiments, the transdermal delivery formulation maintains the about 1% w/w of water contained in Soy lecithin.

In an embodiment, a transdermal delivery formulation contains water in a concentration of at least 0.1%, at least 0.2%, at least 0.3%, at least 0.4%, at least 0.5%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70% or more w/w of the transdermal delivery formulation.

Fatty acids—Soy lecithin contains about 34% w/w fatty acids, including 18-19% w/w linoleic acid, 1-2% w/w alpha-linoleic acid, 8-9% w/w oleic acid, about 5% w/w Palmitic acid, and 1-2% w/w stearic acids. In some embodiments, the fatty acids are similar to the fatty acids contained in soy lecithin. In an embodiment, alpha-linoleic is removed from the transdermal delivery formulation as it is known to oxidize and can become rancid. In some embodiments, the amount of stearic acid has been increased (i.e., enhancing stability of the formulation) or linoleic acid (i.e., enhances skin penetration). In some embodiments, a seed oil such as purified safflower oil is used in a transdermal delivery formulation due to its similarity to the fatty acids found in Soy lecithin, its relative availability and its low cost. In some embodiments, the fatty acid content of a transdermal formulation can be adjusted with a different seed oil through the addition of smaller amounts of the fatty acids disclosed herein.

In various embodiments, a formulation lacks a natural (e.g., plant or animal derived) lecithin.

In a further embodiment, a fatty acid is a saturated or an unsaturated fatty acid. In another embodiment, an unsaturated fatty acid is myristoleic acid, palmitoleic acid, sapienic acid, oleic acid, elaidic acid, vaccenic acid, linoleic acid, linoelaidic acid, α-Linolenic acid, arachidonic acid, eicosapentaenoic acid, erucic acid and/or docosahexaenoic acid. In an embodiment, a saturated fatty acid is caprylic acid, capric acid, lauric acid, myristic acid, palmitic acid, stearic acid, arachidic acid, behenic acid, lignoceric acid and/or cerotic acid. In another embodiment, the fatty acid is a dietary fat and include duct fat, lard, tallow, butter, coconut oil, cocoa butter, palm kernel oil, palm oil, cottonseed oil, wheat germ oil, soybean oil, olive oil, corn oil, sunflower oil, safflower oil, hemp oil and/or canola/rapeseed oil.

In some embodiments, carotenoids are excluded from the formulations disclosed.

In an embodiment, a transdermal delivery formulation comprises the components of Table 3A:

TABLE 3A
General Formulation

| Ingredient | Weight (%) |
|---|---|
| Phospholipid | 5-15% |
| Emollient/moisturizer | 10-20% |
| Fatty acid | 0.5-2% |
| Alcohol | 0.5-2% |
| Oil | 1-5% |
| Surfactant | 0.5-2% |
| Deionized Water | 50-80% |
| Medicament | 0.001%-30%. |
| Total | 100.00% |

In Table 3A, where an ingredient has weight percent that ranges from 5 to 15%, as an example, that ingredient may be present in the formulation at any percentage (w/w or w/v) from about 5% to about 15%. The weight percentage may be about 5% to about 15%. The weight percentage may be about 5% to about 6%, about 5% to about 7%, about 5% to about 8%, about 5% to about 9%, about 5% to about 10%, about 5% to about 11%, about 5% to about 12%, about 5% to about 13%, about 5% to about 14%, about 5% to about 15%, about 6% to about 7%, about 6% to about 8%, about 6% to about 9%, about 6% to about 10%, about 6% to about 11%, about 6% to about 12%, about 6% to about 13%, about 6% to about 14%, about 6% to about 15%, about 7% to about 8%, about 7% to about 9%, about 7% to about 10%, about 7% to about 11%, about 7% to about 12%, about 7% to about 13%, about 7% to about 14%, about 7% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 11% to about 12%, about 11% to about 13%, about 11% to about 14%, about 11% to about 15%, about 12% to about 13%, about 12% to about 14%, about 12% to about 15%, about 13% to about 14%, about 13% to about 15%, or about 14% to about 15%. The weight percentage may be about 5%, about 6%, about 7%, about 8%, about 9%, about 10%, about 11%, about 12%, about 13%, about 14%, or about 15%. The weight percentage may be at least about 5%, about 6%, about 7%, about 8%, about 9%, about 10%, about 11%, about 12%, about 13%, or about 14%. The weight percentage may be at most about 6%, about 7%, about 8%, about 9%, about 10%, about 11%, about 12%, about 13%, about 14%, or about 15%. Moreover, the weight percentage may be about 5% to about 6%. The weight percentage may be about 5% to about 5.1%, about 5% to about 5.2%, about 5% to about 5.3%, about 5% to about 5.4%, about 5% to about 5.5%, about 5% to about 5.6%, about 5% to about 5.7%, about 5% to about 5.8%, about 5% to about 5.9%, about 5% to about 6%, about 5.1% to about 5.2%, about 5.1% to about 5.3%, about 5.1% to about 5.4%, about 5.1% to about 5.5%, about 5.1% to about 5.6%, about 5.1% to about 5.7%, about 5.1% to about 5.8%, about 5.1% to about 5.9%, about 5.1% to about 6%, about 5.2% to about 5.3%, about 5.2% to about 5.4%, about 5.2% to about 5.5%, about 5.2% to about 5.6%, about 5.2% to about 5.7%, about 5.2% to about 5.8%, about 5.2% to about 5.9%, about 5.2% to about 6%, about 5.3% to about 5.4%, about 5.3% to about 5.5%, about 5.3% to about 5.6%, about 5.3% to about 5.7%, about 5.3% to about 5.8%, about 5.3% to about 5.9%, about 5.3% to about 6%, about 5.4% to about 5.5%, about 5.4% to about 5.6%, about 5.4% to about 5.7%, about 5.4% to about 5.8%, about 5.4% to about 5.9%, about 5.4% to about 6%, about 5.5% to about 5.6%, about 5.5% to about 5.7%, about 5.5% to about 5.8%, about 5.5% to about 5.9%, about 5.5% to about 6%, about 5.6% to about 5.7%, about 5.6% to about 5.8%, about 5.6% to about 5.9%, about 5.6% to about 6%, about 5.7% to about 5.8%, about 5.7% to about 5.9%, about 5.7% to about 6%, about 5.8% to about 5.9%, about 5.8% to about 6%, or about 5.9% to about 6%. The weight percentage may be about 5%, about 5.1%, about 5.2%, about 5.3%, about 5.4%, about 5.5%, about 5.6%, about 5.7%, about 5.8%, about 5.9%, or about 6%. The weight percentage may be at least about 5%, about 5.1%, about 5.2%, about 5.3%, about 5.4%, about 5.5%, about 5.6%, about 5.7%, about 5.8%, or about 5.9%. The weight percentage may be at most about 5.1%, about 5.2%, about 5.3%, about 5.4%, about 5.5%, about 5.6%, about 5.7%, about 5.8%, about 5.9%, or about 6%. Further, the weight percentage may be about 5% to about 5.1%. The weight percentage may be about 5% to about 5.01%, about 5% to about 5.02%, about 5% to about 5.03%, about 5% to about 5.04%, about 5% to about 5.05%, about 5% to about 5.06%, about 5% to about 5.07%, about 5% to about 5.08%, about 5% to about 5.09%, about 5% to about 5.1%, about 5.01% to about 5.02%, about 5.01% to about 5.03%, about 5.01% to about 5.04%, about 5.01% to about 5.05%, about 5.01% to about 5.06%, about 5.01% to about 5.07%, about 5.01% to about 5.08%, about 5.01% to about 5.09%, about 5.01% to about 5.1%, about 5.02% to about 5.03%, about 5.02% to about 5.04%, about 5.02% to about 5.05%, about 5.02% to about 5.06%, about 5.02% to about 5.07%, about 5.02% to about 5.08%, about 5.02% to about 5.09%, about 5.02% to about 5.1%, about 5.03% to about 5.04%, about 5.03% to about 5.05%, about 5.03% to about 5.06%, about 5.03% to about 5.07%, about 5.03% to about 5.08%, about 5.03% to about 5.09%, about 5.03% to about 5.1%, about 5.04% to about 5.05%, about 5.04% to about 5.06%, about 5.04% to about 5.07%, about 5.04% to about 5.08%, about 5.04% to about 5.09%, about 5.04% to about 5.1%, about 5.05% to about 5.06%, about 5.05% to about 5.07%, about 5.05% to about 5.08%, about 5.05% to about 5.09%, about 5.05% to about 5.1%, about 5.06% to about 5.07%, about 5.06% to about 5.08%, about 5.06% to about 5.09%, about 5.06% to about 5.1%, about 5.07% to about 5.08%, about 5.07% to about 5.09%, about 5.07% to about 5.1%, about 5.08% to about 5.09%, about 5.08% to about 5.1%, or about 5.09% to about 5.1%. The weight percentage may be about 5%, about 5.01%, about 5.02%, about 5.03%, about 5.04%, about 5.05%, about 5.06%, about 5.07%, about 5.08%, about 5.09%, or about 5.1%. The weight percentage may be at least about 5%, about 5.01%, about 5.02%, about 5.03%, about 5.04%, about 5.05%, about 5.06%, about 5.07%, about 5.08%, or about 5.09%. The weight percentage may be at most about 5.01%, about 5.02%, about 5.03%, about 5.04%, about 5.05%, about 5.06%, about 5.07%, about 5.08%, about 5.09%, or about 5.1%.

The other ranges recited in Table 3A include similar ranges and subranges and values within ranges.

In some cases, the medicament is in an amount from about 0.001% to about 0.01% w/w of the formulation, in an amount from about 0.011% to about 0.1% w/w of the formulation, in an amount from about 0.11% to about 1.0% w/w of the formulation, in an amount from about 1% to about 10% w/w of the formulation, in an amount from about 11% to about 20% w/w of the formulation, or in an amount from about 21% to about 30% w/w of the formulation.

In various embodiments, a transdermal delivery formulation comprises the components of Table 3B:

TABLE 3B
General Formulation

| Ingredient | Weight (%) |
|---|---|
| Phospholipid | 3-15% |
| Fatty acid ester | 5-20% |
| Long-chain fatty acids | 0.1-10% |
| Water | 30-90% |
| Medicament | 0.001%-30%. |
| Optionally: | |
| viscosity-improving agent | 5-20% |
| penetration enhancer | 0.5-5% |
| emulsifier | 0.5-10% |
| Total | 100.00% |

In Table 3B, where an ingredient has weight percent that ranges from 5 to 20% (e.g., for the Fatty acid ester and viscosity-improving agent), as an example, that ingredient may be present in the formulation at any percentage (w/w or w/v) from about 5% to about 20%. The weight percentage may be about 5% to about 20%. The weight percentage may be about 5% to about 6%, about 5% to about 7%, about 5% to about 8%, about 5% to about 9%, about 5% to about 10%, about 5% to about 11%, about 5% to about 12%, about 5% to about 13%, about 5% to about 14%, about 5% to about 15%, about 6% to about 7%, about 6% to about 8%, about 6% to about 9%, about 6% to about 10%, about 6% to about 11%, about 6% to about 12%, about 6% to about 13%, about 6% to about 14%, about 6% to about 15%, about 7% to about 8%, about 7% to about 9%, about 7% to about 10%, about 7% to about 11%, about 7% to about 12%, about 7% to about 13%, about 7% to about 14%, about 7% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 10% to about 16%, about 10% to about 17%, about 10% to about 18%, about 10% to about 19%, about 10% to about 20%, about 11% to about 12%, about 11% to about 13%, about 11% to about 14%, about 11% to about 15%, about 11% to about 16%, about 11% to about 17%, about 11% to about 18%, about 11% to about 19%, about 11% to about 20%, about 12% to about 13%, about 12% to about 14%, about 12% to about 15%, about 12% to about 16%, about 12% to about 17%, about 12% to about 18%, about 12% to about 19%, about 12% to about 20%, about 13% to about 14%, about 13% to about 15%, about 13% to about 16%, about 13% to about 17%, about 13% to about 18%, about 13% to about 19%, about 13% to about 20%, about 14% to about 15%, about 14% to about 16%, about 14% to about 17%, about 14% to about 18%, about 14% to about 19%, about 14% to about 20%, about 15% to about 16%, about 15% to about 17%, about 15% to about 18%, about 15% to about 19%, about 15% to about 20%, about 16% to about 17%, about 16% to about 18%, about 16% to about 19%, about 16% to about 20%, about 17% to about 18%, about 17% to about 19%, about 17% to about 20%, about 18% to about 19%, about 18% to about 20%, or about 19% to about 20%, and any range therebetween. The weight percentage may be about 5%, about 6%, about 7%, about 8%, about 9%, about 10%, about 11%, about 12%, about 13%, about 14%, about 15%, about 16%, about 17%, about 18%, about 19%, or about 20%. The weight percentage may be at least about 5%, about 6%, about 7%, about 8%, about 9%, about 10%, about 11%, about 12%, about 13%, or about 14%. The weight percentage may be at most about 6%, about 7%, about 8%, about 9%, about 10%, about 11%, about 12%, about 13%, about 14%, about 15%, about 16%, about 17%, about 18%, about 19%, or about 20%. Moreover, the weight percentage may be about 5% to about 6%. The weight percentage may be about 5% to about 5.1%, about 5% to about 5.2%, about 5% to about 5.3%, about 5% to about 5.4%, about 5% to about 5.5%, about 5% to about 5.6%, about 5% to about 5.7%, about 5% to about 5.8%, about 5% to about 5.9%, about 5% to about 6%, about 5.1% to about 5.2%, about 5.1% to about 5.3%, about 5.1% to about 5.4%, about 5.1% to about 5.5%, about 5.1% to about 5.6%, about 5.1% to about 5.7%, about 5.1% to about 5.8%, about 5.1% to about 5.9%, about 5.1% to about 6%, about 5.2% to about 5.3%, about 5.2% to about 5.4%, about 5.2% to about 5.5%, about 5.2% to about 5.6%, about 5.2% to about 5.7%, about 5.2% to about 5.8%, about 5.2% to about 5.9%, about 5.2% to about 6%, about 5.3% to about 5.4%, about 5.3% to about 5.5%, about 5.3% to about 5.6%, about 5.3% to about 5.7%, about 5.3% to about 5.8%, about 5.3% to about 5.9%, about 5.3% to about 6%, about 5.4% to about 5.5%, about 5.4% to about 5.6%, about 5.4% to about 5.7%, about 5.4% to about 5.8%, about 5.4% to about 5.9%, about 5.4% to about 6%, about 5.5% to about 5.6%, about 5.5% to about 5.7%, about 5.5% to about 5.8%, about 5.5% to about 5.9%, about 5.5% to about 6%, about 5.6% to about 5.7%, about 5.6% to about 5.8%, about 5.6% to about 5.9%, about 5.6% to about 6%, about 5.7% to about 5.8%, about 5.7% to about 5.9%, about 5.7% to about 6%, about 5.8% to about 5.9%, about 5.8% to about 6%, or about 5.9% to about 6%. The weight percentage may be about 5%, about 5.1%, about 5.2%, about 5.3%, about 5.4%, about 5.5%, about 5.6%, about 5.7%, about 5.8%, about 5.9%, or about 6%. The weight percentage may be at least about 5%, about 5.1%, about 5.2%, about 5.3%, about 5.4%, about 5.5%, about 5.6%, about 5.7%, about 5.8%, or about 5.9%. The weight percentage may be at most about 5.1%, about 5.2%, about 5.3%, about 5.4%, about 5.5%, about 5.6%, about 5.7%, about 5.8%, about 5.9%, or about 6%. Further, the weight percentage may be about 5% to about 5.1%. The weight percentage may be about 5% to about 5.01%, about 5% to about 5.02%, about 5% to about 5.03%, about 5% to about 5.04%, about 5% to about 5.05%, about 5% to about 5.06%, about 5% to about 5.07%, about 5% to about 5.08%, about 5% to about 5.09%, about 5% to about 5.1%, about 5.01% to about 5.02%, about 5.01% to about 5.03%, about 5.01% to about 5.04%, about 5.01% to about 5.05%, about 5.01% to about 5.06%, about 5.01% to about 5.07%, about 5.01% to about 5.08%, about 5.01% to about 5.09%, about 5.01% to about 5.1%, about 5.02% to about 5.03%, about 5.02% to about 5.04%, about 5.02% to about 5.05%, about 5.02% to about 5.06%, about 5.02% to about 5.07%, about 5.02% to about 5.08%, about 5.02% to about 5.09%, about 5.02% to about 5.1%, about 5.03% to about 5.04%, about 5.03% to about 5.05%, about 5.03% to about 5.06%, about 5.03% to about 5.07%, about 5.03% to about 5.08%, about 5.03% to about 5.09%, about 5.03% to about 5.1%, about 5.04% to about 5.05%, about 5.04% to about 5.06%, about 5.04% to about 5.07%, about 5.04% to about 5.08%, about 5.04% to about 5.09%, about 5.04% to about 5.1%, about 5.05% to about 5.06%, about 5.05% to about 5.07%, about 5.05% to about 5.08%, about 5.05% to about 5.09%, about 5.05% to about 5.1%, about 5.06% to about 5.07%, about 5.06% to about 5.08%, about 5.06% to about 5.09%, about 5.06% to about 5.1%, about 5.07% to about 5.08%, about 5.07% to about 5.09%, about 5.07% to about 5.1%, about 5.08% to about 5.09%, about 5.08% to about 5.1%, or about 5.09% to about 5.1%. The weight percentage may be about 5%, about 5.01%, about 5.02%, about 5.03%, about 5.04%, about 5.05%, about 5.06%, about 5.07%, about 5.08%, about 5.09%, or about 5.1%. The weight percentage may be at least about 5%, about 5.01%, about 5.02%, about 5.03%, about 5.04%, about 5.05%, about 5.06%, about 5.07%, about 5.08%, or about 5.09%. The weight percentage may be at most about 5.01%, about 5.02%, about 5.03%, about 5.04%, about 5.05%, about 5.06%, about 5.07%, about 5.08%, about 5.09%, or about 5.1%.

The other ranges recited in Table 3B (e.g., 3-15% for the phospholipids; 0.1-10% for the Long-chain fatty acids; 30-90% for the water; 0.05%-5% for the PDE5 inhibitor; 0.5-5% for the penetration enhancer; and 0.5-10% for the emulsifier) recited in the above table include similar ranges and subranges and values within ranges. The present disclosure contemplates all similar ranges and subranges and values within ranges for each ingredient included in a formulation.

In some cases, the medicament is in an amount from about 0.001% to about 0.01% w/w of the formulation, in an amount from about 0.011% to about 0.1% w/w of the formulation, in an amount from about 0.11% to about 1.0% w/w of the formulation, in an amount from about 1% to about 10% w/w of the formulation, in an amount from about 11% to about 20% w/w of the formulation, or in an amount from about 21% to about 30% w/w of the formulation. The present disclosure contemplates all similar ranges and subranges and values within ranges for the PDE5 inhibitor or inhibitors included in a formulation.

In another embodiment, a transdermal delivery formulation comprises the components of Table 4:

TABLE 4
Medicament “A” Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius (safflower) oil | 2.93% |
| Oleic Acid | 0.97% |
| Polyglyceryl-4 Laurate | 1.06% |
| Deionized Water | 60.84% |
| Active Agent | 2.00% |
| Poloxamer 407 | 9.25% |
| Total | 100.00% |

In another embodiment, a transdermal delivery formulation comprises the components of Table 4:

TABLE 5
Medicament “B” Formulation

| Ingredient | Weight (%) |
|---|---|
| Phosphatidylcholine | 7.66% |
| Isopropyl Palmitate | 13.34% |
| Benzyl Alcohol | 1.39% |
| Stearic Acid | 0.68% |
| Carthamus Tinctorius (safflower) oil | 2.79% |
| Polyglyceryl-4 Laurate | 1.07% |
| Oleic Acid | 1.06% |
| Deionized Water | 61.73% |
| Active Agent | 1.00% |
| Poloxamer 407 | 9.28% |
| Total | 100.00% |

In some cases for the Medicament “A” Formulation or Medicament “B” Formulation, rather than the medicament being in an amount or about 1% or about 2% w/w of the formulation, the amount of the medicament is less than about 1% and the amount of water is increased proportionally. Alternately, rather than the medicament being in an amount or about 1% or about 2% w/w of the formulation, the amount of the medicament is greater than about 2% and the amount of water is decreased proportionally.

In some embodiments, carotenoids are excluded from the formulations disclosed. In an embodiment, a transdermal delivery formulation comprises the components of Table 6.

TABLE 6
Ketoconazole Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius (safflower) oil | 2.93% |
| Oleic Acid | 0.97% |
| Polyglyceryl-4 Laurate | 1.06% |
| Deionized Water | 60.84% |
| Ketoconazole | 2.00% |
| Poloxamer 407 | 9.25% |
| Total | 100.00% |

In some embodiments, carotenoids are excluded from the formulations disclosed. In an embodiment, a transdermal delivery formulation comprises the components of Table 7.

TABLE 7
Hydrocortisone Formulation

| Ingredient | Weight (%) |
|---|---|
| Phosphatidylcholine | 7.66% |
| Isopropyl Palmitate | 13.34% |
| Benzyl Alcohol | 1.39% |
| Stearic Acid | 0.68% |
| Carthamus Tinctorius (safflower) oil | 2.79% |
| Polyglyceryl-4 Laurate | 1.07% |
| Oleic Acid | 1.06% |
| Deionized Water | 61.73% |
| Hydrocortisone | 1.00% |
| Poloxamer 407 | 9.28% |
| Total | 100.00% |

In an aspect, the concentration of phosphatidylcholine in a transdermal delivery formulation is at least 10%, at least 15%, at least 20%, at least 25%, at least 28.75%, at least 30%, at least 35%, at least 40% or more. In an aspect, the concentration of phosphatidylcholine in a transdermal delivery formulation is not more than 10%, not more than 15%, not more than 20%, not more than 25%, not more than 28.75%, not more than 30%, not more than 35%, not more than 40% or more. In an aspect, the concentration of phosphatidylcholine in a transdermal delivery formulation is about 10%, about 15%, about 20%, about 25%, at least 28.75%, about 30%, about 35%, about 40% or more. In an aspect, the concentration of Phosphatidylcholine in a transdermal delivery formulation is from 10% to 40%, is from 15% to 35%, is from 20% to 30%, is from 25% to 30%, is from 28% to 29%.

In a further embodiment, the concentration of benzyl alcohol in a transdermal formulation is at least 0.25%, at least 0.5%, at least 0.75%, at least 1%, at least 2%, at least 2.5%, at least 3%, at least 4%, at least 5% or more. In an embodiment, the concentration of Benzyl Alcohol in a transdermal formulation is about 0.25%, about 0.5%, about 0.75%, about 1%, about 2%, about 2.5%, about 3%, about 4%, about 5% or more. In another embodiment, the concentration of Benzyl Alcohol in a transdermal formulation is at from 0.25% to 5%; from 0.5% to 4%, from 0.75% to 3%, from 1% to 2.5% or from 0.5% to 2%. In a further embodiment, the concentration of Benzyl Alcohol in a transdermal formulation is no more than 0.25%, no more than 0.5%, no more than 0.75%, no more than 1%, no more than 2%, no more than 2.5%, no more than 3%, no more than 4%, no more than 5%.

In an embodiment, the concentration of deionized water in a transdermal formulation is at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5% or more. In an embodiment, the concentration of Deionized Water in a transdermal formulation is about 0.1%, about 0.2%, about 0.3%, about 0.4%, about 0.5%, about 0.6%, about 0.7%, about 0.8%, about 0.9%, about 1%, about 2%, about 3%, about 4%, about 5% or more. In an embodiment, the concentration of deionized water in a transdermal formulation is from 0.1% to 5%, from 0.2% to 4%, from 0.3% to 3%, 0.4%-2%, 0.5% to 1%, from 0.6% t 0.9%, from 0.7% to 0.8%, from 0.4% to 1.5%, from 0.3% to 0.7% or from 0.4% to 0.6%. In an embodiment, the concentration of deionized water in a transdermal formulation is no more than 0.1%, no more than 0.2%, no more than 0.3%, no more than 0.4%, no more than 0.5%, no more than 0.6%, no more than 0.7%, no more than 0.8%, no more than 0.9%, no more than 1%, no more than 2%, no more than 3%, no more than 4%, no more than 5% or more.

In an aspect, the concentration of safflower oil in a transdermal delivery formulation is at least 1%, at least 5%, at least 7.5%, at least 10%, at least 11%, at least 11.06%, at least 12%, at least 13%, at least 14%, at least 15%, at least 16%, at least 17%, at least 18%, at least 19%, at least 20% or more. In an aspect, the concentration of Safflower oil in a transdermal delivery formulation is about 1%, about 5%, about 7.5%, about 10%, about 11%, about 11.06%, about 12%, about 13%, about 14%, about 15%, about 16%, about 17%, about 18%, about 19%, about 20% or more. In an aspect, the concentration of Safflower oil in a transdermal delivery formulation is from 1% to 20%, from 5% to 19%, from 7.5% to 18%, from 10% to 17%, from 11% to 16%, from 11.06%, 12% from 11% to 12%, from 12% to 14%, from 13% to 14%, from 10% to 12%, from 10.5% to 12.5% or from 11% to 11.25%. In an aspect, the concentration of safflower oil in a transdermal delivery formulation is no more than 1%, no more than 5%, no more than 7.5%, no more than 10%, no more than 11%, no more than 11.06%, no more than 12%, no more than 13%, no more than 14%, no more than 15%, no more than 16%, no more than 17%, no more than 18%, no more than 19%, no more than 20%.

In a further aspect, the concentration of oleic acid in a transdermal delivery formulation is at least 1%, at least 2%, at least 3%, at least 3.65%, at least 4%, at least 5%, at least 6%, at least 7%, at least 8%, at least 9%, at least 10% or more. In a further aspect, the concentration of oleic acid in a transdermal delivery formulation is about 1%, about 2%, about 3%, about 3.65%, about 4%, about 5%, about 6%, about 7%, about 8%, about 9%, about 10% or more. In a further aspect, the concentration of Oleic Acid in a transdermal delivery formulation is no more than 1%, no more than 2%, no more than 3%, no more than 3.65%, no more than 4%, no more than 5%, no more than 6%, no more than 7%, no more than 8%, no more than 9%, no more than 10% or more. In another aspect, the concentration of Oleic Acid in a transdermal formulation is from 1% to 10%, from 2% to 9%, from 2% to 3%, from 3% to 4%, from 3% to 8%, from 4% to 7%, from 5% to 6%, from 2 to 2.5% or from 2.5% to 4%.

In another aspect, the concentration of stearic acid in a transdermal formulation is at least 1%, at least 2%, at least 2.34%, at least 3%, at least 4%, at least 5%, at least 6%, at least 7%, at least 8%, at least 9%, at least 10% or more. In another aspect, the concentration of stearic acid in a transdermal formulation is no more than 1%, no more than 2%, no more than 2.34%, no more than 3%, no more than 4%, no more than 5%, no more than 6%, no more than 7%, no more than 8%, no more than 9%, no more than 10% or more. In another aspect, the concentration of stearic acid in a transdermal formulation is about 1%, about 2%, about 2.34%, about 3%, about 4%, about 5%, about 6%, about 7%, about 8%, about 9%, about 10% or more. In another aspect, the concentration of stearic acid in a transdermal formulation is from 1% to 10%, from 2% to 9%, from 2% to 3%, from 2.34% to 2.5%, from 3% to 8%, from 4% to 7%, from 5% to 6% or from 1.5% to 2.5%.

In an aspect, the concentration of isopropyl palmitate in a transdermal formulation is at least 10%, at least 20%, at least 25%, at least 30%, at least 40%, at least 45%, at least 50%, at least 55%, at least 60%, at least 65%, at least 70%, at least 75% or more. In an aspect, the concentration of isopropyl palmitate in a transdermal formulation is about 10%, about 20%, about 25%, about 30%, about 40%, about 45%, about 50%, about 55%, about 60%, about 65%, about 70%, about 75% or more. In an aspect, the concentration of isopropyl palmitate in a transdermal formulation is no more than 10%, no more than 20%, no more than 25%, no more than 30%, no more than 40%, no more than 45%, no more than 50%, no more than 55%, no more than 60%, no more than 65%, no more than 70%, no more than 75% or more. In an aspect, the concentration of isopropyl palmitate in a transdermal formulation is from 10% to 75%, from 20% to 70%, from 25% to 65%, from 30% to 60%, from 40% to 55%, from 45% to 50%, from 40% to 60%, from 45% to 55% or from 47% to 53%.

In an aspect, the concentration of poloxamer 407 in a transdermal delivery formulation is at least 10%, at least 15%, at least 20%, at least 25%, at least 28.75%, at least 30%, at least 35%, at least 40% or more. In an aspect, the concentration of poloxamer 407 in a transdermal delivery formulation is not more than 10%, not more than 15%, not more than 20%, not more than 25%, not more than 28.75%, not more than 30%, not more than 35%, not more than 40% or more. In an aspect, the concentration of poloxamer 407 in a transdermal delivery formulation is about 10%, about 15%, about 20%, about 25%, at least 28.75%, about 30%, about 35%, about 40% or more. In an aspect, the concentration of poloxamer 407 in a transdermal delivery formulation is from 10% to 40%, is from 15% to 35%, is from 20% to 30%, is from 25% to 30%, is from 28% to 29%.

In another aspect, the formulation includes glucose. The concentration of glucose in a transdermal delivery formulation can be, for example, at least 1%, at least 2%, at least 2.5%, at least 3%, at least 4%, at least 5%, at least 6%, at least 7%, at least 8%, at least 9% or more. In another aspect, the concentration of glucose in a transdermal delivery formulation is about 1%, about 2%, about 2.5%, about 3%, about 4%, about 5%, about 6%, about 7%, about 8%, about 9% or more. In another aspect, the concentration of glucose in a transdermal delivery formulation is no more than 1%, no more than 2%, no more than 2.5%, no more than 3%, no more than 4%, no more than 5%, no more than 6%, no more than 7%, no more than 8%, no more than 9% or more. In another aspect, the concentration of glucose in a transdermal delivery formulation is from 1% to 10%, is from 2% to 9%, is from 2.5% to 5%, is from 2% to 3%, is from 3% to 8%, if from 4% to 7%, if from 5% to 6%, is from 2% to 4%, is from 1.5% to 3.5%. In various embodiments, a transdermal delivery formulation contains no glucose.

Certain components or ingredients of a transdermal delivery formulation provided herein may be supplemented with components described in greater detail in the inventor's related applications mentioned above, including U.S. application Ser. No. 16/132,358 filed Sep. 14, 2018, entitled ‘Methods and Formulations For Transdermal Administration Of Buffering Agents’, PCT/US18/51250 filed Sep. 14, 2018, entitled ‘Methods of Administration and Treatment’, and PCT/US18/28017 by Bruce Sand filed Apr. 17, 2018, entitled ‘Parental non-systemic administration of buffering agents for inhibiting metastasis of solid tumors, hyperpigmentation and gout’, each incorporated by reference in their entirety.

A transdermal delivery formulation can include mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

In some embodiments, a transdermal delivery formulation comprises phosphatidylcholine in amount less than 12% w/w or 18% w/w of the formulation. In some embodiments, the transdermal delivery formulation comprises a phospholipid in amount less than 12% w/w or 18% w/w of the formulation. In some embodiments, the transdermal delivery formulation comprises a mixture of tridecane and undecane in amount less than 2% w/w, 5% w/w, or 8% w/w of the formulation. In some embodiments, the formulation comprises Cetiol Ultimate® in an amount less than about 2% w/w, 5% w/w, or 10% w/w, or an equivalent mixture of tridecane and undecane. In some embodiments, the transdermal delivery formulation comprises cetyl alcohol in amount less than 2% w/w, 5% w/w, or 8% w/w of the formulation. In some embodiments, the transdermal delivery formulation comprises benzyl alcohol in an amount less than about 2% w/w, 5% w/w, or 8% w/w. In some embodiments, the transdermal delivery formulation comprises stearic acid in an amount less than 2% w/w, 5% w/w, or 8% w/w of the formulation.

For topical administration, and in particular transdermal administration, a transdermal delivery formulation will comprise penetrants including either or both chemical penetrants (CPEs) and peptide-based cellular penetrating agents (CPPs) that encourage transmission across the dermis and/or across membranes including cell membranes, as would be the case in particular for administration by suppository or intranasal administration, but for transdermal administration as well. In some embodiments, suitable penetrants include those that are described in the above-referenced US2009/0053290, WO2014/209910, and WO2017/127834. In addition to transdermal delivery formulations with penetrants, transdermal delivery can be affected by mechanically disrupting the surface of the skin to encourage penetration, or simply by supplying the formulation applied to the skin under an occlusive patch.

Alternatively, the transdermal delivery formulation comprises a completion component as well as one or more electrolytes sufficient to impart viscosity and viscoelasticity, one or more surfactants and an alcohol. The completion component can be a polar liquid, a non-polar liquid or an amphiphilic substance. The penetrant may further comprise a keratinolytic agent effective to reduce thiol linkages, disrupt hydrogen bonding and/or effect keratin lysis and/or a cell penetrating peptide (sometimes referred to as a skin-penetrating peptide) and/or a permeation enhancer.

Suitable gelling components also include isopropyl palmitate, ethyl laurate, ethyl myristate and isopropyl myristate. In some embodiments, a transdermal delivery formulation comprises a gelling agent in an amount less than 5% w/w of a transdermal delivery formulation. Certain hydrocarbons, such as cyclopentane, cyclooctane, trans-decalin, trans-pinane, n-pentane, n-hexane, n-hexadecane may also be used. In some embodiments, the transdermal delivery formulation comprises a mixture of xanthan gum, sclerotium gum, pullulan, or a combination thereof in an amount less than 2% w/w, 5% w/w, or 10% w/w of the formulation. In some embodiments, a transdermal delivery formulation comprises Siligel™ in an amount from about 1 to about 5% w/w or from about 5 to about 15% w/w, or an equivalent mixture of xanthan gum, sclerotium gum, and pullulan. In some embodiments, a transdermal delivery formulation comprises a mixture of caprylic triglycerides and capric triglycerides in amount less than 2% w/w, 8% w/w, or 10% w/w of the formulation. In some embodiments, a transdermal delivery formulation comprises Myritol® 312 in an amount from about 0.5 to about 10% w/w, or an equivalent mixture of caprylic triglycerides and capric triglycerides.

In some embodiments, a transdermal delivery formulation is in an amount from about 10 to about 90% w/w or from about 10 to about 50% w/w of the formulation or at least 10% w/w, at least 20% w/w, at least 30% w/w, at least 40% w/w, at least 50% w/w, at least 60% w/w, at least 70% w/w, at least 80% w/w, at least 90% w/w or at least 95% w/w. In some embodiments, a transdermal delivery formulation comprises phosphatidyl choline in amount less than 7% w/w, less than 8% w/w, less than 9% w/w, less than 10% w/w, less than 11% w/w, less than 12% w/w, less than 13% w/w, less than 14% w/w, less than 15% w/w, less than 16% w/w, less than 17% w/w or less than 18% w/w of the formulation. In some embodiments, a transdermal delivery formulation comprises a phospholipid in amount less than 20% w/w, less than 30% w/w, less than 40% w/w, less than or 50% w/w of the formulation. In some embodiments, a transdermal delivery formulation comprises a mixture of tridecane and undecane in amount less than 2% w/w, 3% w/w, 4% w/w, 5% w/w, 6% w/w, 7% w/w, or 8% w/w of the formulation. In some embodiments, the formulation comprises Cetiol Ultimate® in an amount less than about 2% w/w, 3% w/w, 4% w/w, 5% w/w, 6% w/w, 7% w/w, 8% w/w, 9% w/w, or 10% w/w, or an equivalent mixture of tridecane and undecane. In some embodiments, a transdermal delivery formulation comprises cetyl alcohol in amount less than 2% w/w, 3% w/w, 4% w/w, 5% w/w, 6% w/w, 7% w/w, 8% w/w, 9% w/w, or 10% w/w of the formulation. In some embodiments, the formulation comprises benzyl alcohol in an amount less than about 2% w/w, 3% w/w, 4% w/w, 5% w/w, 6% w/w, 7% w/w, 8% w/w, 9% w/w, or 10% w/w. In some embodiments, a transdermal delivery formulation comprises stearic acid in an amount less than 2% w/w, 3% w/w, 4% w/w, 5% w/w, 6% w/w, 7% w/w, 8% w/w, 9% w/w, or 10% w/w of the formulation. In some embodiments, the transdermal delivery formulation comprises phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 30% w/w or in amount less than 12% w/w of the formulation.

An additional component in a transdermal delivery formulation of the disclosure is an alcohol. Benzyl alcohol and ethanol are illustrated in the Examples. In particular, derivatives of benzyl alcohol which contain substituents on the benzene ring, such as halo, alkyl and the like. The weight percentage of benzyl or other related alcohol in the final composition is 0.5-20% w/w, and again, intervening percentages such as 1% w/w, 2% w/w, 3% w/w, 4% w/w, 5% w/w, 6% w/w, 7% w/w, 8% w/w, 9% w/w, or 10% w/w, and other intermediate weight percentages are included. Due to the aromatic group present in a transdermal delivery formulation such as benzyl alcohol, the molecule has a polar end (the alcohol end) and a non-polar end (the benzene end). This enables the agent to dissolve a wider variety of transdermal delivery formulation components.

In some embodiments, as noted above, the performance of a transdermal delivery formulation is further improved by including a nonionic detergent and polar gelling agent or including a powdered surfactant. In both aqueous and anhydrous forms of the composition, detergents, typically nonionic detergents are added. In general, the nonionic detergent should be present in an amount from about 1% w/w to about 30% w/w of a transdermal delivery formulation. Typically, in the compositions wherein a transdermal delivery formulation is topped off with a polar or aqueous solution containing detergent, the amount of detergent is relatively low—e.g., 2-25% w/w, or 5-15% w/w or 7-12% w/w of a transdermal delivery formulation. However, in compositions that are essentially anhydrous and are topped-off by powdered detergent, relatively higher percentages are usually used—e.g., 20-60% w/w.

In some embodiments, a transdermal delivery formulation further comprises a detergent portion in an amount from about 1 to about 70% w/w or from about 1 to about 60% w/w of a transdermal delivery formulation. In some embodiments, the nonionic detergent provides suitable handling properties whereby the formulations are gel-like or creams at room temperature. To exert this effect, the detergent, typically a poloxamer, is present in an amount from about 2 to about 12% w/w of a transdermal delivery formulation, preferably from about 5 to about 25% w/w in polar formulations. In the anhydrous forms of the compositions, the detergent is added in powdered or micronized form to bring the composition to 100% and higher amounts are used. In compositions with polar constituents, rather than bile salts, the nonionic detergent is added as a solution to bring the composition to 100%. If smaller amounts of detergent solutions are needed due to high levels of the remaining components, more concentrated solutions of the nonionic detergent are employed. Thus, for example, the percent detergent in the solution may be 10% to 40% or 20% or 30% and intermediate values depending on the percentages of the other components.

Suitable nonionic detergents include poloxamers such as the non-ionic surfactant Pluronic® and any other surfactant characterized by a combination of hydrophilic and hydrophobic moieties. Poloxamers are triblock copolymers of a central hydrophobic chain of polyoxypropylene flanked by two hydrophilic chains of polyethyleneoxide. Other nonionic surfactants include long chain alcohols and copolymers of hydrophilic and hydrophobic monomers where blocks of hydrophilic and hydrophobic portions are used.

In some embodiments, a transdermal delivery formulation also contains surfactant, typically, nonionic surfactant at 2-25% w/w of a transdermal delivery formulation along with a polar solvent wherein the polar solvent is present in an amount at least in molar excess of the nonionic surfactant. In these embodiments, typically, the composition comprises the above-referenced amounts of a transdermal delivery formulation and benzyl alcohol along with a sufficient amount of a polar solution, typically an aqueous solution or polyethylene glycol solution that itself contains 10%-40% of surfactant, typically nonionic surfactant to bring the composition to 100%.

Other examples of surfactants include polyoxyethylated castor oil derivatives such as HCO-60 surfactant sold by the HallStar Company; nonoxynol; octoxynol; phenylsulfonate; poloxamers such as those sold by BASF as Pluronic® F68, Pluronic® F127, and Pluronic® L62; polyoleates; Rewopal® HVIO, sodium laurate, sodium lauryl sulfate (sodium dodecyl sulfate); sodium oleate; sorbitan dilaurate; sorbitan dioleate; sorbitan monolaurate such as Span® 20 sold by Sigma-Aldrich; sorbitan monooleates; sorbitan trilaurate; sorbitan trioleate; sorbitan monopalmitate such as Span® 40 sold by Sigma-Aldrich; sorbitan stearate such as Span® 85 sold by Sigma-Aldrich; polyethylene glycol nonylphenyl ether such as Synperonic® NP sold by Sigma-Aldrich; p-(1,1,3,3-tetramethylbutyl)-phenyl ether sold as Triton™ X-100 sold by Sigma-Aldrich; and polysorbates such as polyoxyethylene (20) sorbitan monolaurate sold as Tween® 20, polysorbate 40 (polyoxyethylene (20) sorbitan monopalmitate) sold as Tween® 40, polysorbate 60 (polyoxyethylene (20) sorbitan monostearate) sold as Tween® 60, polysorbate 80 (polyoxyethylene (20) sorbitan monooleate) sold as Tween® 80, and polyoxyethylenesorbitan trioleate sold as Tween® 85 by Sigma-Aldrich. The weight percentage range of nonionic surfactant is in the range of 3% w/w-15% w/w, and again includes intermediate percentages such as 5% w/w, 7% w/w, 10% w/w, 12% w/w, and the like. In some embodiments, the detergent portion comprises a nonionic surfactant in an amount from about 1 to about 30% w/w of the formulation, and a polar solvent in an amount less than 5% w/w of the formulation. In some embodiments, the nonionic surfactant is a poloxamer and the polar solvent is water, an alcohol, or a combination thereof. In some embodiments, the detergent portion comprises poloxamer, propylene glycol, glycerin, ethanol, 50% w/v sodium hydroxide solution, or a combination thereof. In some embodiments, the detergent portion comprises glycerin in an amount less than 3% w/w of the formulation.

In the presence of a polar gelling agent, such as water, glycerol, ethylene glycol or formamide, a micellular structure is also often achieved. Typically, the polar agent is in molar excess of the nonionic detergent. The inclusion of the nonionic detergent/polar gelling agent combination results in a more viscous and cream-like or gel-like formulation which is suitable for application directly to the skin. This is typical of the aqueous forms of the composition.

In some embodiments other additives are included such as a gelling agent, a dispersing agent and a preservative. An example of a suitable gelling agent is hydroxypropylcellulose, which is generally available in grades from viscosities of from about 5 cps to about 25,000 cps such as about 1500 cps. All viscosity measurements are assumed to be made at room temperature unless otherwise stated. The concentration of hydroxypropylcellulose may range from about 1% w/w to about 2% w/w of the composition. Other gelling agents are known in the art and can be used in place of, or in addition to hydroxypropylcellulose. An example of a suitable dispersing agent is glycerin. Glycerin is typically included at a concentration from about 5% w/w to about 25% w/w of the composition. A preservative may be included at a concentration effective to inhibit microbial growth, ultraviolet light and/or oxygen-induced breakdown of composition components, and the like. When a preservative is included, it may range in concentration from about 0.01% w/w to about 1.5% w/w of the composition.

Additional components that can also be included in a transdermal delivery formulation are fatty acids, terpenes, lipids, and cationic, and anionic detergents. In some embodiments, a transdermal delivery formulation further comprises tranexamic acid in an amount less than 2% w/w, 5% w/w, or 10% w/w of the formulation. In some embodiments, a transdermal delivery formulation further comprises a polar solvent in an amount less than 2% w/w, 5% w/w, 10% w/w, or 20% w/w of the transdermal delivery formulation. In some embodiments, a transdermal delivery formulation further comprises a humectant, an emulsifier, an emollient, or a combination thereof. In some cases the humectant is propylene glycol. In some cases, the emulsifier is polyglyceryl-4-laurate, cetyl alcohol or Durosoft PK-SG. In some cases, the emollient is derived from almond oil. In some embodiments, a transdermal delivery formulation further comprises almond oil in an amount less than about 5% w/w. In some embodiments, a formulation further comprises a mixture of thermoplastic polyurethane and polycarbonate in an amount less than about 5% w/w. In some embodiments, a transdermal delivery formulation further comprises phosphatidylethanolamine in an amount less than about 5% w/w. In some embodiments, a transdermal delivery formulation further comprises an inositol phosphatide in an amount less than about 5% w/w.

Other solvents and related compounds that can be used in some embodiments include acetamide and derivatives, acetone, n-alkanes (chain length from 7 to 16), alkanols, diols, short chain fatty acids, cyclohexyl-1,1-dimethylethanol, dimethyl acetamide, dimethyl formamide, ethanol, ethanol/d-limonene combination, 2-ethyl-1,3-hexanediol, ethoxydiglycol (Transcutol® by Gattefosse, Lyon, France), glycerol, glycols, lauryl chloride, limonene N-methylformamide, 2-phenylethanol, 3-phenyl-1-propanol, 3-phenyl-2-propen-1-ol, polyethylene glycol, polyoxyethylene sorbitan monoesters, polypropylene glycol 425, primary alcohols (tridecanol), 1,2-propane diol, butanediol, C₃-C₆ triols or their mixtures and a polar lipid compound selected from C₁₆ or C₁₈ monounsaturated alcohol, C₁₆ or C₁₈ branched saturated alcohol and their mixtures, propylene glycol, sorbitan monolaurate sold as Span® 20 by Sigma-Aldrich, squalene, triacetin, trichloroethanol, trifluoroethanol, trimethylene glycol and xylene.

Fatty alcohols, fatty acids, fatty esters, are bilayer fluidizers that can be used in some embodiments. Examples of suitable fatty alcohols include aliphatic alcohols, decanol, lauryl alcohol (dodecanol), unolenyl alcohol, nerolidol, 1-nonanol, n-octanol, and oleyl alcohol. Examples of suitable fatty acid esters include butyl acetate, cetyl lactate, decyl N,N-dimethylamino acetate, decyl N,N-dimethylamino isopropionate, diethyleneglycol oleate, diethyl sebacate, diethyl succinate, diisopropyl sebacate, dodecyl N,N-dimethyamino acetate, dodecyl (N,N-dimethylamino)-butyrate, dodecyl N,N-dimethylamino isopropionate, dodecyl 2-(dimethyamino) propionate, E0-5-oleyl ether, ethyl acetate, ethylaceto acetate, ethyl propionate, glycerol monoethers, glycerol monolaurate, glycerol monooleate, glycerol monolinoleate, isopropyl isostearate, isopropyl linoleate, isopropyl myristate, isopropyl myristate/fatty acid monoglyceride combination, isopropyl palmitate, methyl acetate, methyl caprate, methyl laurate, methyl propionate, methyl valerate, 1-monocaproyl glycerol, monoglycerides (medium chain length), nicotinic esters (benzyl), octyl acetate, octyl N,N-dimethylamino acetate, oleyl oleate, n-pentyl N-acetylprolinate, propylene glycol monolaurate, sorbitan dilaurate, sorbitan dioleate, sorbitan monolaurate, sorbitan monooleate, sorbitan trilaurate, sorbitan trioleate, sucrose coconut fatty ester mixtures, sucrose monolaurate, sucrose monooleate, tetradecyl N.N-dimethylamino acetate. Examples of suitable fatty acid include alkanoic acids, caprid acid, diacid, ethyloctadecanoic acid, hexanoic acid, lactic acid, lauric acid, linoelaidic acid, linoleic acid, linolenic acid, neodecanoic acid, oleic acid, palmitic acid, pelargonic acid, propionic acid, and vaccenic acid. Examples of suitable fatty alcohol ethers include a-monoglyceryl ether, E0-2-oleyl ether, E0-5-oleyl ether, E0-10-oleyl ether, ether derivatives of polyglycerols and alcohols, and (1-O-dodecyl-3-O-methyl-2-O-(2′,3′-dihydroxypropyl glycerol).

Examples of completing agents that can be used in some embodiments include β- and γ-cyclodextrin complexes, hydroxypropyl methylcellulose (e.g., Carbopol® 934), liposomes, naphthalene diamide diimide, and naphthalene diester diimide.

One or more anti-oxidants can be included, such as vitamin C, vitamin E, proanthocyanidin and a-lipoic acid typically in concentrations of 0.1%-2.5% w/w.

In some applications, it is desirable to adjust the pH of a transdermal delivery formulation to assist in permeation or to adjust the nature of the target compounds in the subject. In some instances, the pH is adjusted to a level of pH 9-11 or 10-11 which can be done by providing appropriate buffers or simply adjusting the pH with base.

A transdermal delivery formulation can include other components that act as excipients or serve purposes other than for muscle performance and recovery. For example, preservatives like antioxidants e.g., ascorbic acid or a-lipoic acid and antibacterial agents may be included. Other components apart from therapeutically active ingredients and components that are the primary effectors of dermal penetration may include those provided for aesthetic purposes such as menthol or other aromatics, and components that affect the physical state of the composition such as emulsifiers, for example, Durosoft®. Typically, these ingredients are present in very small percentages of the compositions. It is understood that these latter ancillary agents are neither therapeutically ingredients nor are they components that are primarily responsible for penetration of the skin. The components that primarily effect skin penetration have been detailed as described above. However, some of these substances have some capability for effecting skin penetration. See, for example, Kunta, J. R. et al, J. Pharm. Sci. (1997) 86:1369-1373, describing penetration properties of menthol.

The application method is determined by the nature of the treatment but may be less critical than the nature of the formulation itself. If the application is to a skin area, it may be helpful in some instances to prepare the skin by cleansing or exfoliation. In some instances, it is helpful to adjust the pH of the skin area prior to application of a transdermal delivery formulation itself. The application of a transdermal delivery formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

In addition to the compositions and formulations of the invention per se, the methods can employ a subsequent treatment with linoleic acid. As transdermal treatments generally open up the skin barrier, which is, indeed, their purpose, it is useful to seal the area of application after the treatment is finished. Thus, treatment with a transdermal delivery formulation may be followed by treating the skin area with a composition comprising linoleic acid to seal off the area of application. The application of linoleic acid is applicable to any transdermal procedure that results in impairing the ability of the skin to act as a protective layer. Indeed, most transdermal treatments have this effect as their function is to allow the active component to pass through the epidermis to the dermis at least, and, if systemic administration is achieved, through the dermis itself.

Additional therapeutic agents can be included in the compositions. For example, hydrocortisone or hydrocortisone acetate may be included in an amount ranging from 0.25% w/w to about 0.5% w/w. Menthol, phenol, and terpenoids, e.g., camphor, can be incorporated for cooling pain relief. For example, menthol can be included in an amount ranging from about 0.1% w/w to about 1.0% w/w.

The compositions containing anesthetics are useful for temporary relief of pain and itching associated with minor burns, cuts, scrapes, skin irritations, inflammation and rashes due to soaps, detergents or cosmetics, or, as noted above, pain associated with removal of fat deposits.

Certain embodiments of a transdermal delivery formulation provided herein may be supplemented with formulation components described in greater detail in the inventor's related applications, including U.S. application Ser. No. 16/132,358 filed Sep. 14, 2018, entitled ‘Methods and Formulations For Transdermal Administration Of Buffering Agents’, International Patent Application No. PCT/US18/51250 filed Sep. 14, 2018, entitled ‘Methods of Administration and Treatment’, and International Patent Application PCT/US18/28017 by Bruce Sand filed Apr. 17, 2018, entitled ‘Parental non-systemic administration of buffering agents for inhibiting metastasis of solid tumors, hyperpigmentation and gout’, all incorporated by reference in their entirety herein.

In some particular embodiments it is desirable to adjust the pH of a transdermal delivery formulation and the pH is adjusted to a level of pH 9-11 or 10-11, which can be done by providing appropriate buffers or simply adjusting the pH with base. In other embodiments, it is desirable to adjust the pH of a transdermal delivery formulation to a level of pH 4-6, which can be done by providing appropriate buffers or simply adjusting the pH with an acid.

In some applications a formulation for transdermal delivery may, for example, comprise: Aveeno®, for example in an amount from about 10 to about 95% w/w; from about 20 to about 85% w/w, from about 20 to about −75% w/w, from about 20 to about 50% w/w.

In another aspect, certain embodiments are directed to a sustained release drug delivery platform releases a therapeutic compound or compounds disclosed and made as a formulation described herein over a period of, without limitation, about 3 days after administration, about 7 days after administration, about 10 days after administration, about 15 days after administration, about 20 days after administration, about 25 days after administration, about 30 days after administration, about 45 days after administration, about 60 days after administration, about 75 days after administration, or about 90 days after administration. In other aspects of this embodiment, a sustained release drug delivery platform releases a therapeutic compound or compounds disclosed herein with substantially first order release kinetics over a period of, without limitation, at least 3 days after administration, at least 7 days after administration, at least 10 days after administration, at least 15 days after administration, at least 20 days after administration, at least 25 days after administration, at least 30 days after administration, at least 45 days after administration, at least 60 days after administration, at least 75 days after administration, or at least 90 days after administration.

The formulation described in this specification may also comprise more than one therapeutic compound as desired for the particular indication being treated, preferably those with complementary activities that do not adversely affect the other proteins. A transdermal delivery formulation to be used for in vivo administration can be sterile. This can be accomplished, for instance, without limitation, by filtration through sterile filtration membranes, prior to, or following, preparation of a transdermal delivery formulation or other methods known in the art, including without limitation, pasteurization.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

In certain embodiments, kits can comprise, without limitation, one or more cream or lotion comprising one or more formulations described herein. In various embodiments, the kit can comprise formulation components for transdermal, topical, or subcutaneous administration, formulated to be administered as an emulsion coated patch. In all of these embodiments and others, the kits can contain one or more lotion, cream, patch, or the like in accordance with any of the foregoing, wherein each patch contains a single unit dose for administration to a subject.

Imaging components can optionally be included, and the packaging also can include written or web-accessible instructions for using a transdermal delivery formulation. A container can include, for example, a vial, bottle, patch, syringe, pre-filled syringe, tube or any of a variety of formats well known in the art for multi-dispenser packaging.

In an aspect, the concentration of poloxamer 407 in a transdermal delivery formulation is at least 10%, at least 15%, at least 20%, at least 25%, at least 28.75%, at least 30%, at least 35%, at least 40% or more. In an aspect, the concentration of poloxamer 407 in a transdermal delivery formulation is not more than 10%, not more than 15%, not more than 20%, not more than 25%, not more than 28.75%, not more than 30%, not more than 35%, not more than 40% or more. In an aspect, the concentration of poloxamer 407 in a transdermal delivery formulation is about 10%, about 15%, about 20%, about 25%, at least 28.75%, about 30%, about 35%, about 40% or more. In an aspect, the concentration of poloxamer 407 in a transdermal delivery formulation is from 10% to 40%, is from 15% to 35%, is from 20% to 30%, is from 25% to 30%, is from 28% to 29%.

A transdermal delivery formulation can include mixtures wherein the components interact synergistically and induce skin permeation enhancements better than that induced by the individual components. Synergies between chemicals can be exploited to design potent permeation enhancers that overcome the efficacy limitations of single enhancers. Several embodiments disclosed herein utilize three to five distinct permeation enhancers.

### Administration and Dosing

Methods for treating, preventing or ameliorating a disease, disorder, a condition, or a symptom thereof or a condition related thereto are provided herein using a transdermal delivery formulation for transdermal delivery described herein. The methods provided herein may comprise or consist of topically administering one or more of a transdermal delivery formulation described herein to skin of a subject in need thereof. Preferred, but non-limiting embodiments are directed to methods for treating, preventing, inhibiting or ameliorating a disease, disorder, a condition, or a symptom described herein.

A formulation for transdermal delivery may comprise one or more medicaments (e.g., of Table 1) and a penetrant portion.

A transdermal delivery formulation provided herein can be topically administered in any form. For administration for the treatment of skin conditions a sufficient amount of the topical composition can be applied onto a desired area and surrounding skin, for example, in an amount sufficient to cover a desired skin surface. A transdermal delivery formulation can be applied to any skin surface, including for example, facial skin, and the skin of the hands, neck, chest and/or scalp.

In applying a transdermal delivery formulation of the invention, a transdermal delivery formulation itself is simply placed on the skin and spread across the surface and/or massaged to aid in penetration. The amount of transdermal delivery formulation used is typically sufficient to cover a desired surface area. In some embodiments, a protective cover is placed over the formulation once it is applied and left in place for a suitable amount of time, i.e., 5 minutes, 10 minutes, 20 minutes or more; in some embodiments an hour or two. The protective cover can simply be a bandage including a bandage supplied with a cover that is impermeable to moisture. This essentially locks in the contact of a transdermal delivery formulation to the skin and prevents distortion of a transdermal delivery formulation by evaporation in some cases. The composition may be applied to the skin using standard procedures for application such as a brush, a syringe, a gauze pad, a dropper, or any convenient applicator. More complex application methods, including the use of delivery devices, may also be used, but are not required. In an alternative to administering topically to intact skin, the surface of the skin may also be disrupted mechanically by the use of spring systems, laser powered systems, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed formulations that penetrate intact skin may be applied using occlusive patches, such as those in the form micro-patches. External reservoirs of the formulations for extended administration may also be employed.

In an alternative to administering topically to intact skin, the surface of the skin may also be disrupted mechanically by the use of spring systems, laser powered systems, use of iontophoresis, systems propelled by Lorentz force or by gas or shock waves including ultrasound and may employ microdermabrasion such as by the use of sandpaper or its equivalent or using microneedles or electroporation devices. Simple solutions of the agent(s) as well as the above-listed transdermal delivery formulations that penetrate intact skin may be applied using occlusive patches, such as those in the form of micro-patches. External reservoirs of the formulations for extended administration may also be employed.

Accordingly, in certain embodiments alternative methods of administering one or more therapeutic compounds or agents (e.g. medicaments) through intact skin are provided. As nonlimiting examples, these alternative methods might be selected from the following lists: on basis of working mechanism, spring systems, laser powered, energy-propelled, Lorentz force, gas/air propelled, shock wave (including ultrasound), on basis of type of load, liquid, powder, projectile, on basis of drug delivery mechanism, nano-patches, sandpaper (microdermabrasion), iontophoresis enabled, microneedles, on basis of site of delivery, intradermal, intramuscular, and subcutaneous injection. Other suitable delivery mechanisms include, without limitation, microneedle drug delivery, such as 3M Systems, Glide SDI (pushes drug as opposed to “firing” drug), MIT low pressure injectors, micropatches (single use particle insertion device), microelectro mechanical systems (MEMS), dermoelectroporation devices (DEP), transderm ionto system (DEP), TTS transdermal therapeutic systems, membrane-moderated systems (drug reservoir totally encapsulated in a shallow compartment), adhesive diffusion-controlled system (drug reservoir in a compartment fabricated from drug-impermeable metallic plastic backing), matrix dispersion type system (drug reservoir formed by homogeneously dispersing drug solids in a hydrophilic or lipophilic polymer matrix molder into medicated disc), and microreservoir system (combination of reservoir and matrix dispersion-type drug delivery system).

The application method is determined by the nature of the treatment but may be less critical than the nature of a transdermal delivery formulation itself. If the application is to a skin area, it may be helpful in some instances to prepare the skin by cleansing or exfoliation. In some instances, it is helpful to adjust the pH of the skin area prior to application of the formulation itself. The application of a transdermal delivery formulation may be by simple massaging onto the skin or by use of devices such as syringes or pumps. Patches could also be used. In some cases, it is helpful to cover the area of application to prevent evaporation or loss of a transdermal delivery formulation.

Where the application area is essentially skin, it is helpful to seal-off the area of application subsequent to supplying a transdermal delivery formulation and allowing the penetration to occur so as to restore the skin barrier. A convenient way to do this is to apply a composition comprising linoleic acid which effectively closes the entrance pathways that were provided by the penetrants of the invention. This application, too, is done by straightforward smearing onto the skin area or can be applied more precisely in measured amounts.

A transdermal delivery formulation can be applied in a single, one-time application, once a week, once a bi-week, once a month, or from one to twelve times daily, for a period of time sufficient to alleviate a condition, disease, disorder, symptoms, for example, for a period of time of one week, from 1 to 12 weeks or more, from 1 to 6 weeks, from 2 to 12 weeks, from 2 to 8 weeks, from 2 to 6 weeks, from 2 to 4 weeks, from 4 to 12 weeks, from 4 to 8 weeks, or from 4 to 6 weeks. The present compositions can be administered, for example, at a frequency of once per day to hourly if needed. The presently described formulations can be topically administered once or more per day for a period of time from 1 week to 4 weeks, of from 1 week to 2 weeks, for 1 week, for 2 weeks, for 3 weeks, or for 4 weeks or more. In some instances, it may also be desirable to continue treatment indefinitely, for example, to inhibit recurring inflammation. A suitable administration for a transdermal delivery formulation comprising a skin cream, lotion or ointment, for example is once, twice, three, four times daily, or hourly if needed.

As described above, if desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the transdermal delivery formulation may be prepared. Single dose, unit dose, and once-daily disposable containers of the transdermal delivery formulation are also provided.

The present transdermal delivery formulation remains stable in storage for periods including up to about 5 years, from about 3 months to about 5 years, from about 3 months to about 4 years, from about 3 months to about 3 years, and alternately any time period from about 6 months to about 3 years.

A transdermal delivery formulation described herein remains stable for up to at least 3 years at a temperature of less than or equal to 40° C. In an embodiment, the presently described transdermal delivery formulation remains stable for at least 2 years at a temperature of less than or equal to 40° C. In an embodiment, the presently described transdermal delivery formulation remains stable for at least 3 years at a temperature of less than or equal to 40° C. and at a humidity of up to 75% RH, for at least 2 years at a temperature of less than or equal to 40° C. and at a humidity of up to 75% RH, or for at least 3 years at a temperature of less than or equal to 30° C. and at a humidity of up to 75% RH. In a further embodiment, the presently described transdermal delivery formulation in accordance with the subject matter described herein remains stable for an extended period of time when packaged in a multi-use container such as a bottle dispenser or the like, and exhibits equal to or even greater stability when packaged in a single-use package.

In another aspect, the transdermal delivery formulation of certain embodiments comprises a daily dose of particular buffering compound (e.g. sodium bicarbonate, sodium carbonate, magnesium carbonate, potassium carbonate, potassium bicarbonate, TRIS, Lysine, IEPA, etc.). A daily dose for topical or transdermal administration of a transdermal delivery formulation depends on the compound and animal and may be easily determined by the skilled artisan, a suitable amount is about 1 mg/kg to about 5 g/kg, and more typically the daily dose is about 10 mg/kg to about 5 g/kg, about 25 mg/kg to about 2000 mg/kg, about 50 mg/kg to about 2000 mg/kg, about 25 mg/kg to about 1000 mg/kg, about 50 mg/kg to about 1000 mg/kg, about 100 mg/kg to about 700 mg/kg, about 100 mg/kg to about 500 mg/kg, about 150 mg/kg to about 500 mg/kg, about 150 mg/kg to about 400 mg/kg, about 200 mg/kg to about 500 mg/kg, about 200 mg/kg to about 450 mg/kg, about 200 mg/kg to about 400 mg/kg, about 250 mg/kg to about 450 mg/kg, about 250 mg/kg to about 400 mg/kg, about 250 mg/kg to about 350 mg/kg, and about 275 mg/kg to about 325 mg/kg.

If desired, other therapeutic agents can be employed in conjunction with those provided in the above-described compositions. The amount of active ingredients that may be combined with the carrier materials to produce a single dosage form will vary depending upon the host treated, the nature of the disease, disorder, or condition, and the nature of the active ingredients.

It is understood that a specific dose level for any particular patient will vary depending upon a variety of factors, including the activity of the specific active agent; the age, body weight, general health, sex and diet of the patient; the time of administration; the rate of excretion; possible drug combinations; the severity of the particular condition being treated; the area to be treated and the form of administration. One of ordinary skill in the art would appreciate the variability of such factors and would be able to establish specific dose levels using no more than routine experimentation.

Pharmacokinetic parameters such as bioavailability, absorption rate constant, apparent volume of distribution, unbound fraction, total clearance, fraction excreted unchanged, first-pass metabolism, elimination rate constant, half-life, and mean residence time can be determined by methods well known in the art.

A transdermal delivery formulation in accordance with the subject matter described herein may be a topical dosage form packaged in, for example, a multi-use or single-use package, including for example, a tube, a bottle, a pump, a container or bottle, a vial, a jar, a packet, or a blister package.

Single dosage kits and packages containing a once per day amount of the transdermal delivery formulation may be prepared. Single dose, unit dose, and once-daily disposable containers of the transdermal delivery formulation are also provided.

Alternatively, a suitable dose for topical or transdermal administration of each of one or more particular buffering compound (e.g. sodium bicarbonate, sodium carbonate, magnesium carbonate, potassium carbonate, potassium bicarbonate, TRIS, Lysine, IEPA, etc.) for subject is at least about 100 mg, at least about 500 mg, at least about 1 g, at least about 5 g, at least about 10 g, at least about 15 g, at least about 16 g, at least about 17 g, at least about 18 g, at least about 19 g, at least about 20 g, at least about 21 g, at least about 22 g, at least about 23 g, at least about 24 g, at least about 25 g, at least about 26 g, at least about 27 g, at least about 28 g, at least about 29 g, at least about 30 g, at least about 35 g, at least about 40 g, at least about 45 g, at least about 50 g, at least about 60 g, at least about 75 g, at least about 100 g, at least about 200 g, at least about 500 g, or at least about 1.0 kg. This dose may be administered daily, twice a day, three times a day, four times a day, five times a day, or more than five times a day.

Aspects of the present specification disclose that the symptoms associated with a disease or disorder described herein are reduced following application of a transdermal delivery formulation by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced following application of a transdermal delivery formulation by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

Aspects of the present specification disclose that the symptoms associated with a disease or disorder described herein are reduced following administration of a transdermal delivery formulation of the present invention by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% and the severity associated with a disease or disorder described herein is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. Aspects of the present specification disclose the symptoms associated with disease or disorder are reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

A transdermal delivery formulation as described herein can be used in the manufacture of medicaments and for the treatment of humans and other animals by administration in accordance with conventional procedures.

Dosing can be single dosage or cumulative (serial dosing), and can be readily determined by one skilled in the art. A transdermal delivery formulation of the present invention may be administered once, twice, three, four, five, six, seven, eight, nine, ten, eleven, twelve, thirteen, fourteen, fifteen, sixteen, seventeen, eighteen, nineteen, twenty or more times to a subject. For instance, treatment of a disease may comprise a one-time administration of an effective dose of a transdermal delivery formulation as disclosed herein. Alternatively, treatment of a disease may comprise multiple administrations of an effective dose of a transdermal delivery formulation as carried out over a range of time periods, such as, e.g., once daily, twice daily, thrice daily, once every few days, or once weekly. The timing of administration can vary from individual to individual, depending upon such factors as the severity of an individual's symptoms. For example, an effective dose of a transdermal delivery formulation as disclosed herein can be administered to an individual once daily for an indefinite period of time, or until the individual no longer requires therapy. A person of ordinary skill in the art will recognize that the condition of the individual can be monitored throughout the course of treatment and that the effective amount of a transdermal delivery formulation disclosed herein that is administered can be adjusted accordingly. In one embodiment, a transdermal delivery formulation as disclosed herein is capable of decreasing the time to resolve the symptoms of a disease, including in an individual suffering from a disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In a further embodiment, a transdermal delivery formulation and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a transdermal delivery formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a transdermal delivery formulation disclosed herein reduces or alleviates symptoms (e.g. muscle pain or cramping) of an ailment such as muscle cramping or soreness in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a transdermal delivery formulation disclosed herein reduces or alleviates symptoms of an ailment in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a transdermal delivery formulation disclosed herein reduces or alleviates symptoms of an ailment in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A transdermal delivery formulation disclosed herein may comprise a transdermal delivery formulation in a therapeutically effective amount. As used herein, the term “effective amount” is synonymous with “therapeutically effective amount”, “effective dose”, or “therapeutically effective dose” and when used in reference to reducing or alleviating symptoms of an ailment in an individual refers to the minimum dose of a therapeutic disclosed herein necessary to achieve the desired therapeutic effect and includes a dose sufficient to reduce or alleviates symptoms of an ailment in an individual. The effectiveness of a transdermal delivery formulation disclosed herein capable of reducing or alleviating symptoms of an ailment in an individual can be determined by observing an improvement in an individual based upon one or more clinical symptoms, and/or physiological indicators associated with improvements in muscle performance, reduced soreness and/or overall health. Maintenance or a reduction of symptoms of an ailment can also be subjective to a patient. The effectiveness of a transdermal delivery formulation disclosed herein in an individual can be determined by observing an improvement in an individual based upon one or more clinical symptoms, and/or physiological indicators associated with signs/symptoms, muscle performance and general health. The effectiveness of a transdermal delivery formulation disclosed herein is also capable of prolonging the life of an individual as compared to the same individual if the transdermal delivery formulation is not administered. The effectiveness of the transdermal delivery formulation disclosed herein is also capable of enhancing the quality of life of an individual as compared to the same individual if the transdermal delivery formulation is not administered.

The appropriate effective amount of a transdermal delivery formulation disclosed herein to be administered to an individual can be determined by a person of ordinary skill in the art by taking into account factors, including, without limitation, an improvement in the individual based upon one or more clinical symptoms, and/or physiological indicators associated with improvements in muscle performance, reduced soreness and/or overall health, the particular characteristics, history and risk factors of the patient, such as, e.g., age, weight, general health and the like, or any combination thereof. Additionally, where repeated administration of a transdermal delivery formulation is used, an effective amount of a transdermal delivery formulation will further depend upon factors, including, without limitation, the frequency of administration, the half-life of the transdermal delivery formulation, or any combination thereof. It is known by a person of ordinary skill in the art that an effective amount of a transdermal delivery formulation disclosed herein can be extrapolated from in vitro assays and in vivo administration studies using animal models prior to administration to humans or animals.

Wide variations in the necessary effective amount are to be expected in view of the differing efficiencies of the various routes of administration. For instance, oral administration of a transdermal delivery formulation disclosed herein generally would be expected to require higher dosage levels than administration by inhalation. Similarly, systemic administration of a transdermal delivery formulation disclosed herein would be expected to require higher dosage levels than a local administration. Variations in these dosage levels can be adjusted using standard empirical routines of optimization, which are well-known to a person of ordinary skill in the art. The precise therapeutically effective dosage levels and patterns are preferably determined by the attending physician in consideration of the above-identified factors. One skilled in the art will recognize that the condition of the individual can be monitored throughout the course of therapy and that the effective amount of a therapeutic disclosed herein that is administered can be adjusted accordingly.

Aspects of the present specification disclose, in part, a reduction or alleviation of symptoms of an ailment such as muscle cramping or soreness in an individual. As used herein, the term “treating,” refers to reduction or alleviation of muscle soreness or cramping as well as improved muscle performance and recovery. For example, the term “treating” can mean reduction or alleviation of symptoms in an individual by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with an ailment such as muscle cramping and soreness are well known and can be determined by a person of ordinary skill in the art by using commonly known testing means. Those of skill in the art will know the appropriate symptoms or indicators associated with health and muscle performance and will know how to determine if an individual is a candidate for treatment as disclosed herein.

In an embodiment, a first transdermal delivery formulation is administered to an individual and at a later date, a second transdermal delivery formulation is administered to the same individual. In an embodiment, a first transdermal delivery formulation is administered to an individual at the same time as a second transdermal delivery formulation is administered to the individual.

A transdermal delivery formulation as disclosed herein is administered to an individual. An individual is typically a human being, but can be an animal, including, but not limited to, dogs, cats, birds, cattle, horses, sheep, goats, reptiles and other animals, whether domesticated or not.

In one aspect, disclosed herein is a formulation for transdermal delivery of an active agent through the skin, nail or hair follicle of a subject, wherein the formulation comprises a) a transdermal delivery formulation in an amount less than about 60% w/w, comprising i. one or more phosphatides, ii. glucose, and iii. one or more fatty acids; and b) water in an amount less than about 50% w/w.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises benzyl alcohol in an amount from about 0.5 to about 5% w/w.

In some embodiments, the transdermal delivery formulation comprises benzyl alcohol in an amount less than 5% w/w of the formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises Isopropyl Palmitate in an amount from about 5 to about −20% w/w.

In some embodiments, the water is deionized water and/or purified water.

In some embodiments, the water is in an amount from about 15 to about 40% w/w of the formulation.

In some embodiments, the one or more phosphatides in an amount from about 0.5 to about 55% w/w of the transdermal delivery formulation.

In some embodiments, the transdermal delivery formulation comprises phosphatidylcholine, hydrogenated phosphatidylcholine, phosphatidylserine, phosphatidylethanolamine, phosphatidylinositol, or a combination thereof in amount less than 30% w/w of the formulation.

In some embodiments, the one or more phosphatides comprises phosphatidylcholine of the transdermal delivery formulation.

In some embodiments, the one or more fatty acids in an amount from about 1 to about 35% w/w of the transdermal delivery formulation.

In some embodiments, the one or more fatty acids in an amount from about 5 to about 35% w/w of the transdermal delivery formulation.

In some embodiments, the one or more fatty acids comprises Linoleic Acid, Oleic Acid, Stearic Acid, safflower oil, or a combination thereof.

In some embodiments, the one or more fatty acids comprises Linoleic Acid.

In some embodiments, the one or more fatty acids comprises Oleic Acid.

In some embodiments, the one or more fatty acids comprises Stearic Acid.

In some embodiments, the one or more phosphatides are derived from a seed oil in an amount from about 0.5 to about 55% w/w of the transdermal delivery formulation.

In some embodiments, the one or more phosphatides are derived from a seed oil in an amount from about 5 to about 35% w/w of the transdermal delivery formulation.

In some embodiments, the one or more phosphatides are derived from a safflower oil in an amount from about 0.5 to about 55% w/w of the transdermal delivery formulation.

In some embodiments, the one or more phosphatides are derived from a safflower oil in an amount from about 5 to about 35% w/w of the transdermal delivery formulation.

In some embodiments, the one or more phosphatides are derived from an almond oil in an amount from about 0.5 to about 55% w/w of the transdermal delivery formulation.

In some embodiments, the one or more phosphatides are derived from an almond oil in an amount from about 5 to about 35% w/w of the transdermal delivery formulation.

In some embodiments, the one or more phosphatides comprises one or more fatty acids derived from soy lecithin.

In some embodiments, the glucose in an amount from about 0.05 to about 10% w/w of the transdermal delivery formulation.

In some embodiments, the glucose is anhydrous dextrose in an amount from about 0.05 to about 10% w/w of the transdermal delivery formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a nonionic surfactant in an amount from about 2 to about 25% w/w of the transdermal delivery formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a polar solvent at least in an amount in molar excess of the nonionic surfactant.

In some embodiments, the nonionic surfactant is a poloxamer and the polar solvent is water.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a polar solvent in an amount less than 5% w/w of the formulation.

In some embodiments, the transdermal delivery formulation further comprises a detergent portion in an amount from about 1 to about 30% w/w of the transdermal delivery formulation.

In some embodiments, the detergent portion comprises a nonionic surfactant in an amount from about 2 to about 25% w/w of the transdermal delivery formulation; and a polar solvent in an amount less than 5% w/w of the transdermal delivery formulation.

In some embodiments, the transdermal delivery formulation is in an amount from about 10 to about 60% w/w of the transdermal delivery formulation.

In some embodiments, the transdermal delivery formulation comprises an alcohol in an amount less than 10% w/w of the transdermal delivery formulation.

In some embodiments, the transdermal delivery formulation further comprises an alcohol, a surfactant, and a polar solvent.

In some embodiments, the transdermal delivery formulation comprises cetyl alcohol in amount less than 5% w/w of the formulation.

In some embodiments, the transdermal delivery formulation comprises ethanol in an amount less than 5% w/w of the formulation.

In some embodiments, the transdermal delivery formulation comprises glycerine in an amount less than 5% w/w of the formulation.

In some embodiments, the transdermal delivery formulation comprises propylene glycol in an amount less than 8% w/w of the formulation.

In some embodiments, the formulation comprises a gelling agent in an amount less than 20% w/w of the formulation.

In some embodiments, the formulation comprises menthol in an amount from about 0.05 to about 5% w/w of the formulation.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises a humectant, an emulsifier, an emollient, or a combination thereof.

In some embodiments, the formulation has a pH of 9-11.

In some embodiments, the formulation has a pH of 7-10.5.

In some embodiments, the formulation comprises a) a transdermal delivery formulation in an amount less than about 60% w/w, comprising i. one or more phosphatides, ii. glucose, and iii. one or more fatty acids; and b) water in an amount less than about 50% w/w, further comprises an active agent.

In some embodiments, the formulation comprises a) a transdermal delivery formulation in an amount less than about 60% w/w, comprising i. one or more phosphatides, ii. glucose, iii. one or more fatty acids; b) water in an amount less than about 50% w/w, and an active agent component in an amount less than about 60% w/w.

In some embodiments, the formulation comprises a) a transdermal delivery formulation in an amount less than about 60% w/w, comprising i. one or more phosphatides, ii. glucose, and iii. one or more fatty acids; b) water in an amount less than about 50% w/w, and an active agent component in an amount less than about 60% w/w, wherein the active agent is a medicament.

In another aspect disclosed herein is a method to effect transdermal delivery of an active ingredient comprising applying to the skin, nails or hair follicles of a subject an effective amount of the formulation comprising a) a transdermal delivery formulation in an amount less than about 60% w/w, comprising i. one or more phosphatides, ii. glucose, and iii. one or more fatty acids; and b) water in an amount less than about 50% w/w, further comprises an active agent.

### Combination Therapies

In some aspects, the present disclosure provides a method for treating a disease or disorder or reducing a symptom thereof, in which the method comprises steps of administering to a subject in need thereof a herein disclosed transdermal formulation and administering to the subject in need thereof a composition comprising an one or more medicaments selected from Table 1.

In embodiments, the transdermal formulation is administered before, contemporary with, or after the composition is administered.

In some embodiments, the amount of the one or more medicaments is the effective dose of the medicament as described in Table 1.

In various embodiments, the composition is administered by the standard route for the one or more medicament, e.g., the standard route is oral, topical, enteral, parenteral, by intravenous injection or infusion, by intraperitoneal injection, by intramuscular injection, or by subcutaneous injection.

In embodiments, the composition is a liquid, a suspension, a gel, a geltab, a semisolid, a tablet, a sachet, a lozenge, a pill, or a capsule.

### Formulations Comprising Buffering Agents

An approach to make electrolyte balancing formulations is to avoid electrolyte imbalances by incorporating different buffers in different amount or ratios. Non-limiting examples of buffering agents that can be used together in different amounts or ratios include potassium bicarbonate, sodium bicarbonate, calcium carbonate, magnesium carbonate, and potassium carbonate. Mixtures of particular buffering agents including 2, 3, 4, 5, or more buffering agents are used depending on the formulation. Further, the relative amounts or ratio of each buffering agent may vary, for example, where the relative amounts are from 1:1.10 w/w; 1:1.15 w/w; 1:1.20 w/w; 1:1.25 w/w; 1:1.30 w/w; 1:1.35 w/w; 1:1.40 w/w; 1:1.45 w/w; 1:1.50 w/w; 1:1.55 w/w; 1:1.60 w/w; 1:1.65 w/w; 1:1.70 w/w; 1:1.75 w/w; 1:1.80 w/w; 1:1.85 w/w; 1:1.90 w/w; 1:1.95 w/w; 1:2 w/w; 1:2.5 w/w; 1:3 w/w; 1:3.5 w/w; 1:4 w/w, 1:4.5 w/w; 1:5 w/w, 1:5.5 w/w; 1:6 w/w; 1:6.5 w/w; 1:7 w/w; 1:8 w/w; 1:9 w/w; or 1:10 w/w. These ratios of buffering agents are applicable when two buffering agents are present, or more than two and the ratios are applicable between any two buffering agents.

For example, a suitable formulation may comprise about 10-56% w/w buffering agent and a penetrant. In one aspect, disclosed herein is a transdermal delivery formulation for transdermal delivery of one or more buffering agent through the skin of a subject, comprising: a buffering agent comprising a carbonate salt in an amount from about 10 to about 56% w/w; a transdermal delivery formulation in an amount from about 5 to about 55% w/w; a detergent portion in an amount of at least 1% w/w; and wherein the formulation comprises water in an amount from none up to about 77% w/w.

In an embodiment, a carbonate, including sodium bicarbonate in a transdermal delivery formulation is in an amount of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more w/w.

In another aspect, disclosed herein is a method for transdermal delivery of a carbonate salt of a %, at least comprising: a buffering agent comprising a carbonate salt in an amount from about 10 to about 45% w/w; a transdermal delivery formulation in an amount from about 5 to about 55% w/w; a detergent portion in an amount from about 1 to about 15% w/w; and wherein the formulation comprises water in an amount from about 15 to about 65% w/w, through the skin of a subject, wherein the carbonate salt of the formulation is in an amount from about 15 to about 32% w/w of the formulation.

In another embodiment, a buffering agent comprising a carbonate salt, including sodium bicarbonate in a transdermal delivery formulation is in an amount of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more w/w.

In another embodiment, a buffering agent comprising a carbonate salt, including sodium bicarbonate in a transdermal delivery formulation is in an amount of at least 1%, at least 2%, at least 3%, at least 4%, at least 5%, at least 6%, at least 7%, at least 8%, at least 9%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or more w/w.

In one aspect, disclosed herein is a formulation for transdermal delivery of one or more buffering agent through the skin of a subject, comprising: a buffering agent comprising a carbonate salt in an amount between about 10-45% w/w; a transdermal delivery formulation in an amount between about 5 to 55% w/w; a detergent portion in an amount between about 1 to 15% w/w; and wherein the formulation comprises water in an amount between about 15 to 65% w/w, and wherein the formulation comprises less than about 12% w/w of the transdermal delivery formulation.

In yet another aspect, disclosed herein is a formulation for transdermal delivery of a therapeutic agent through the skin of a subject, wherein the formulation comprises at least one active agent in an amount effective for treatment of a condition in the subject and the formulation comprising: a buffering agent comprising a carbonate salt in an amount from about 10 to about 45% w/w; a transdermal delivery formulation in an amount from about 5 to about 55% w/w; a detergent portion in an amount from about 1 to about 15% w/w; wherein the formulation comprises water in an amount from about 15 to about 65% w/w, through the skin of a subject, wherein the carbonate salt of the formulation is in an amount from about 15 to about 32% w/w of the formulation, therapeutic, and wherein the alkalinity of the formulation enhances penetration of the therapeutic agent.

In one aspect, disclosed herein is a formulation for transdermal delivery of one or more buffering agent through the skin of a subject, comprising: a buffering agent comprising a carbonate salt in an amount from about 10-45% w/w; a transdermal delivery formulation in an amount from about 5 to about 55% w/w; a detergent portion in an amount from about 1 to about 15% w/w; and wherein the formulation comprises water in an amount from about 15 to about 65% w/w, and wherein the formulation comprises less than about 12% w/w of the transdermal delivery formulation.

In another aspect, disclosed herein is a method for transdermal delivery of a carbonate salt of the formulation comprising: a buffering agent comprising a carbonate salt in an amount from about 10 to about 45% w/w; a transdermal delivery formulation in an amount from about 5 to about 55% w/w; a detergent portion in an amount from about 1 to about 15% w/w; and wherein the formulation comprises water in an amount from about 15 to about 65% w/w, and wherein the formulation comprises less than about 12% w/w of the transdermal delivery formulation, through the skin of a subject, wherein the carbonate salt of the formulation is in an amount from about 15 to about 32% w/w of the formulation, wherein the formulation comprises less than about 12% w/w of the transdermal delivery formulation, and wherein the alkalinity of the formulation enhances penetration of the therapeutic agent.

In yet another aspect, disclosed herein is a formulation for transdermal delivery of a therapeutic agent through the skin of a subject, wherein the formulation comprises at least one active agent in an amount effective for treatment of a condition in the subject and the formulation comprising: a buffering agent comprising a carbonate salt in an amount from about 10 to about 45% w/w; a transdermal delivery formulation in an amount from about 5 to about 55% w/w; a detergent portion in an amount from about 1 to about 15% w/w; wherein the formulation comprises water in an amount from about 15 to about 65% w/w, through the skin of a subject, wherein the carbonate salt of the formulation is in an amount from about 15 to about 32% w/w of the formulation, and wherein the formulation comprises less than about 12% w/w of the transdermal delivery formulation.

In some embodiments, a suitable transdermal delivery formulation comprises: Siligel™ in an amount less than about 5% w/w; water in an amount from about 10 to about 65% w/w; isopropyl palmitate in an amount from about 0.5 to about 10% w/w; stearic acid in an amount from about 0.25 to about 10% w/w; cetyl alcohol in an amount from about 0.25 to about 10% w/w; glycerin in an amount from about 0.25 to about 5% w/w; a transdermal delivery formulation in an amount from about 0.25 to about 10% w/w; ethanol in an amount less than about 5% w/w; benzyl alcohol in an amount less than about 5% w/w; sodium hydroxide 50% w/v in an amount from about 0.1 to about 5% w/w; and sodium bicarbonate in an amount from about 1 to about 32% w/w.

In some embodiments, a suitable transdermal delivery formulation comprises Aveeno® in an amount from about 20 to about 85% w/w; and sodium bicarbonate (3DF) in an amount from about 15 to about 45% w/w.

In some embodiments, a transdermal delivery formulation comprises Aveeno® in an amount from about 20 to about 85% w/w; and sodium bicarbonate in an amount from about 15 to about 45% w/w.

In some embodiments, a suitable transdermal delivery formulation comprises: Siligel™ in an amount less than about 5% w/w; water in an amount from about 10 to about 55% w/w; isopropyl palmitate in an amount from about 0.5 to about 10% w/w; stearic Acid in an amount from about 0.25 to about 5% w/w; Cetyl alcohol in an amount from about 0.25 to about 10% w/w; almond oil in an amount from about 0.5-10% w/w; propylene glycol in an amount from about 0.25 to about 10% w/w; ethanol in an amount less than about 5% w/w; benzyl alcohol in an amount less than about 5% w/w; sodium hydroxide 50% w/v in an amount from about 0.1 to about 5% w/w; and sodium bicarbonate in an amount from about 1 to about 32% w/w.

The surprising effects achieved by the formulations and methods of the present invention are in part attributable to an improved transdermal delivery formulation that enhances delivery of a carbonate salt through the skin. The present transdermal delivery formulations may include a nonionic surfactant. Applicant has found that by employing carbonate salts with particle sizes as disclosed herein, delivered with the penetrants as disclosed herein, and in some embodiments providing a combination of a nonionic surfactant and a polar gelling agent, the penetration capabilities of the carbonate salts of the resulting formulation and the effective level of delivery of the carbonate salts has been enhanced.

In a transdermal delivery formulation, penetrants are based on combinations of an alcohol, such as benzyl alcohol to provide a concentration of 0.5-20% w/w of the final formulation with a transdermal delivery formulation present to provide 25-70% w/w of the formulation. These penetrants are also useful when the agent is a buffer, such as sodium bicarbonate, but less of a transdermal delivery formulation may be required—e.g. less than 12% w/w when the sodium bicarbonate is present at high concentration as disclosed herein.

In some embodiments, the buffering component is any mildly basic compound or combination that will result in a pH of 7-8. In some embodiments, the formulation has a pH of 7-10. Such buffers, in addition to carbonate and/or bicarbonate salts, include lysine buffers, chloroacetate buffers, tris buffers (i.e., buffers employing tris (hydroxymethyl) aminoethane), phosphate buffers and buffers employing non-natural amino acids with similar pKa values to lysine. In some embodiments, the carbonate and/or bicarbonate salt is in an amount from about 7 to about 32% w/w of the formulation. For example, the enantiomers of native forms of such amino acids or analogs of lysine with longer or shorter carbon chains or branched forms thereof. Histidine buffers may also be used. Typically, the concentration of buffer in the compositions is in the range of 10-50% w/w. More typical ranges for sodium bicarbonate or sodium carbonate or both are 10-35% w/w. In some embodiments, the carbonate salt is in an amount from about 15 to about 32% w/w of the formulation.

Alternatively, the penetrant component comprises a completion component as well as one or more electrolytes sufficient to impart viscosity and viscoelasticity, one or more surfactants and an alcohol. The completion component can be a polar liquid, a non-polar liquid or an amphiphilic substance.

The percentage of carbonate salt in a transdermal delivery formulation will depend upon the amount required to be delivered in order to have a useful effect on treating the disorder. In general, the carbonate salt may be present in the formulation in an amount as low as 1% w/w up to about 50% w/w. Typical concentrations may include 15-32% w/w. Since the required percentage of carbonate salt depends on the frequency of administration, as well as the time allotted for administration for each application, the level of carbonate salt may be varied over a wide range. In some embodiments, the carbonate salt is sodium carbonate and/or sodium bicarbonate milled to a particle size less than 200 μm. In some embodiments, the carbonate salt is sodium carbonate and/or sodium bicarbonate milled to a particle size less than 70 μm. In some embodiments, the carbonate salt is sodium carbonate and/or sodium bicarbonate milled to a particle size less than 70 μm, wherein the sodium bicarbonate is solubilized in the formulation in an amount less than 20% w/w of a transdermal delivery formulation. In some embodiments, the carbonate salt is sodium carbonate and/or sodium bicarbonate milled to a particle size less than 70 μm, wherein particle sizes less than about 10 μm have an enhanced penetration thru the skin of a subject. In some embodiments, the sodium carbonate and/or sodium bicarbonate are jet milled to a particle size less than about 70 μm. In some embodiments, the sodium bicarbonate is Sodium Bicarbonate USP Grade 3DF that has a particle size distribution less than 70 μm.

A transdermal delivery formulation of the disclosure may be prepared in a number of ways. Typically, the components of a transdermal delivery formulation are simply mixed together in the required amounts. However, it is also desirable in some instances to, for example, carry out dissolution of a carbonate salt and then add a separate preparation containing the components aiding the delivery of the carbonate salts in the form of a carrier. The concentrations of these components in the carrier, then, will be somewhat higher than the concentrations required in a final transdermal delivery formulation. Thus, sodium bicarbonate may first be dissolved in water and then added to a carrier comprising an alcohol, a transdermal delivery formulation and optionally a combination of a nonionic surfactant and polar gelling agent, or of ionic detergent. Alternatively, some subset of these components can first be mixed and then “topped off” with the remaining components either simultaneously or sequentially. The precise manner of preparing a transdermal delivery formulation will depend on the choice of carbonates and the percentages of the remaining components that are desirable with respect to that carbonate salt. In some embodiments, the water is in an amount from about 10 to about 85% w/w, from about 15 to about 50% w/w, or from about 15 to about 45% w/w of the formulation.

The transdermal delivery formulation is a multi-component mixture, whereby the particular concentrations of the penetration enhancers are informed in part by the molecular mass of the sodium bicarbonate, or sodium bicarbonate and the therapeutic agent to be transported. A transdermal delivery formulation enables the sodium bicarbonate and/or therapeutic agent to become bio-available to the target site within minutes of topical administration. A transdermal delivery formulation permit the use of minimal concentrations of therapeutic agents, as little as 1/1000th of concentrations required of alternative processes, while enabling bioactivity and positive clinical outcomes simultaneously. In some embodiments, the transdermal delivery formulation comprises an alcohol in an amount less than 5% w/w of the formulation.

### ADDITIONAL EMBODIMENTS

Embodiment 1. A formulation for transdermal delivery of a medicament through the skin of a subject, comprising a therapeutically effective amount of a medicament and a penetrant portion, wherein the penetrant portion comprises: a) phosphatidylcholine (3-15% w/w); b) isopropyl palmitate (5-20% w/w); c) stearic acid (0.1-5% w/w); d) benzyl alcohol (0.5-5% w/w); e) polyglyceryl-4 laurate (0.5-10% w/w); and f) poloxamer 407 (5-20% w/w).

Embodiment 2. The formulation of Embodiment 1, wherein the medicament is at least one of (3S,4S)-8-(6-amino-5-((2-amino-3-chloropyridin-4-yl)thio)pyrazin-2-yl)-3-methyl-2-oxa-8-azaspiro[4.5]decan-4-amine; (S)-3-amino-6-methoxy-N-(3,3,3-trifluoro-2-hydroxy-2-methylpropyl)-5-(trifluoromethyl)picolinamide; 2-fluoro-N-methyl-4-[7-quinolin-6-yl-methyl)-imidazo[1,2-B][1,2,4]triazin-2yl]benzamide; 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide; 4-(7-Hydroxy-2-isopropyl-4-oxo-4H-quinazolin-3-yl)-benzonitrile; Abacavir; Abiraterone Acetate; abiratone acetate; abobotulinum toxin A; Acalabrutinib; Acarbose; Acetaminophen; Acetazolamide; Acetylsalicylic Acid; acitretin; acyclovir; adalimumab; adapalene; adapalene; Afoxolaner; agalsidase beta; Albendazole; Alectinib; Alectinib; alendronate sodium; alglucosidase alfa; alitretinoin; Alogliptin; alpelisib; Aluminum Hydroxide; Ambrisentan; Amiodarone; Amitriptyline; Amlodipine; Amlodipine; Amlodipine besylate; amlodopine besylate; amorolfine hydrochloride; Amoxicillin; Amphotericin B; Amphotericin B liposome; ampicillin; Amprenavir; antihemophilic factor, Fc fusion protein; apalutamide; Apatinib; apixaban; apremilast; Aprepitant; Aripiprazole; Artemether; aspirin; Atazanavir; atenolol; Atomoxetine; Atorvastatin; Atorvastatin; Avapritinib; Axitinib (Inlyta); Azacitidine; Azathioprine; azelaic acid; Azilsartan medoxomil; azithromycin; baclofen; baricitinib; basiliximab; Batimastat (BB-94); becaplermin; Belumosudil; Bendamustine; benzoyl peroxide; benzoyl peroxide; betamethasone dipropionate; betamethasone valerate; bexarotene; Bictegravir; Bimatoprost; binimetinib (Mektovi); Bortezomib; Bortezomib (Velcade); Bosentan; Bosutinib (Bosulif); botulinum toxin A; branched chain amino acids; Brexpiprazole; Brimonidine; brimonidine tartrate; brodalumab; buproprion; Cabazitaxel; Cabozantinib; Cabozantinib (Cometriz); Calcifediol; calcipotriene; Calcipotriene; Calcipotriol; calcitonin; calcitriol; calcium carbonate; Canagliflozin; canakinumab; Capecitabine; Capmatinib; capsaicin; Carbamazepine; Carbidopa; Carfilzomib; Carfilzomib (Kyprolis); Cariprazine; carvedilol; CD-12681; CEE-321; cefazolin; cefepime; Cefoperazone; cefotaxime sodium; celecoxib; celecoxib; cemiplimab; Ceritinib; Chlorothiazide; Chlorpromazine; Chlorthalidone; cinacalcet; ciprofloxacin; Cisapride; citalopram; citric acid; clarithromycin; Clavulanate; Clindamycin phosphate; Clobazam; clobetasol propionate; Clofazimine; clomiphene; clonidine; Clopidogrel; clopidogrel bisulfate; cobicistat; Cobicistat; Colchocine; Colistin; Corticotropin; crisaborole; crizanlizumab; Crizotinib (Xalkori); cyclobenzaprine; cyclosporine; Dabigatran Etexilate; Dabrafenib; Dabrafenib; dabrafenib; dabrafenib; dacomitinib (Vizimpro); dalbavancin; dalfopristin; Danazol; Dapagliflozin; Dapsone; daptomycin; Darunavir; Dasatinib; decitabine; defatted Peanut (Arachis hypogaea) Flour; deferasirox; delgocitinib; deoxycholic acid; Desipramine; desonide; Dexlansoprazole; Dexmethylphenidate; dextroamphetamine/amphetamine salts; Diclofenac; diclofenac sodium; diflucortolone; Diflucortolone; Difunisal; Digoxin; Diloxanide; Dimethyl Fumarate; diphenhydramine hydrochloride; diphtheria vaccine; docetaxel; Dolutegravir; Dolutegravir; Donepezil; Doxercalciferol; doxycycline; doxycycline ER; Doxycycline Hyclate (B); Doxycycline Monohydrate (A); Dronabinol; Dronedarone; Drospirenone; Dukoral/ShanChol cholera vaccine; Duloxetine; dupilumab; durvalumab; Dutasteride; Duvelisib; ecallantide; Edoxaban; Efavirenz; Efavirenz; Elafibranor; elagolix sodium; Elbasvir; Eletriptan; Eltrombopag; Elvitegravir; Empagliflozin; Emtricitabine; emtricitabine; Emtricitabine; Emtricitabine; Emtricitabine; Emtricitabine; Emtricitabine; Emtricitabine; encorafenib (Braftovi); enfuviritide; enoxaparin sodium; Entecavir; entrectinib; Enzalutamide; Epinephrine; erdafitinib; erenumab; Eribulin; Erlotinib; Erlotinib HCl (Tarceva); Ertapenem; Erythromycin; escitalopram; esketamine (ketamine); Esomeprazole; esopremazole; estradiol; Estrogens—conjugated; etanercept; Ethinyl Estradiol; Ethinyl Estradiol; Ethinyl Estradiol; Etonogestrel; Etonogestrel; Etravirine; etretinate; Everolimus; Everolimus (Afinitor); Everolimus (Votubia, Zortress/Certican); Ezetimibe; Ezetimibe; Ezetimibe; Famotidine; fasinumab; Febuxostat; Fedratinib; Fenofibrate; Ferric Carboxymaltose; fexofenadine hydrochloride; filgotinib; filgrastim; fingolimod; fluocinolone acetonide; fluocinolone acetonide; fluoxetine; Flurbiprofen; Folic acid; Forskolin; Fostamatinib; Fulvestrant; furosemide; fusidic acid; fusidic acid; gabapentin; Ganetespib; Gefitinib (Iressa); Gilteritinib; glasdegib (Daurismo) or glasdegib maleate; Glecaprevir; Glibenclamide; Glimepiride; glipizide; glucagon; glucosamine; glutaric anhydride; glycopyrrolate; Goserelin; goserelin LA; granisetron; Grazoprevir; Griseofulvin; halobetasol propionate; Haloperidol; heparin sodium; heplisav-B vaccine; hyaluronic acid; Hydrochlorothiazide; Hydrochlorothiazide; Hydrochlorothiazide; Hydrochlorothiazide; hydrocortisone; hydrocortisone acetate; hydroquinone; hydroxychloroquine; Hydroxyprogesterone; Ibrutinib; Ibrutinib (Imbruvica); ibuprofen; icatibant acetate; Idelalisib; Imatinib; Imatinib (Gleevec); imatinib mesylate; imiglucerase; inclisiran; Indinavir; Indomethacin; infliximab; ingenol mebutate; inotersen; insulin A; insuline glargine; interferon beta-lb; Iopanoic acid; Irbesartan; isoconazole; isoconazole; Isotretinoin; Itraconazole; Ivacaftor; Ivacaftor; ivermectin; Ixazomib; ketoconazole; ketones; ketoprofen; Keyruvia; Lacosamide; lamictal; Lamivudine; Lansoprazole; Lapatinib; Lapatinib (Tykerb); larotrectinib; Ledipasvir; leflunomide; Lenalidomide; Lenvatinib; LEO-138559; LEO-152020; Leuprolide; Levetiracetam; levodopa; Levodopa/Benserazide; Levonorgestrel; Levothyroxine; Linagliptin; linezolid; lisinopril; L-lysine free base; lofexidine; Lopinavir; Loratadine; lorlatinib (Lorviqua); Losartan; Lovastatin; Lubiprostone; Lumacaftor/Ivacaftor; Lumefantrine; Lurasidone; Luspatercept; lymecycline; Macitentan; magnesium; magnesium lactate; Marimastat (BB-2516); Marizomib (NPI-0052); Mebendazole; Mefloquine; melatonin; meloxicam; memantine hydrochloride; meningococcal[serotype b] vaccine; Mesalazine; Metformin; Metformin; metformin; Metformin; methotrexate; methyl aminolevulinate hydrochloride; Methylphenidate; methylprednisolone; Metoprolol succinate; metronidazole; Midostaurin; minoxidil; Mirabegron; mizolastine MR; Montelukast; Mycophenolate Mofetil; N-(3-(2-(2-hydroxyethoxy)-6-morpholinopyridin-4-yl)-4-methylphenyl)-2-(trifluoromethyl)-isonicotinamide; N-(3-(6-Amino-5-(2-(N-methylacrylamido)ethoxy)pyrimidin-4-yl)-5-fluoro-2-methylphenyl)-4-cyclopropyl-2-fluorobenzamide; N-[4-(Chlorodifluoromethoxy)phenyl]-6-[(3R)-3-hydroxpyrrolidin-1-yl]-5-(1H-pyrazol-5-yl)-pyridine-3-carboxamide hydrochloride; Nalidixic acid (Quinolone antibiotic); naloxone; Naproxen; Navitoclax (ABT-263); Nebivolol; Nelfinavir; nemolizumab; neomycin; Neovastat (AE-941); Neratinib—Free Base; Neratinib (Nerlynx); Neratinib Maleate; Nevirapine; Niclosamide; Nifedipine; nilotinib; Nilotinib; Nilotinib (Tasigna); Nintedanib; niratimib; Nitric Oxide; Nitrofurantoin; Norethindrone Acetate; nusinersen; NVP-AUY922; Nystatin; Obatoclax (GX15-070); octreotide acetate; ofatumumab; Ofloxacin; Olanzapine; olaparib; Olmesartan Medoxomil; olumacostat; omalizumab; Oseltamivir; Osimertinib; osimertinib mesylate; Oxaprozin; Ozanimod; Paclitaxel; Paclitaxel Protein Bound; Palbociclib (Ibrance); Paliperidone Palmitate; Pantoprazole; parathyroid hormone; Paricalcitol; patisiran; pazopanib; Pazopanib (Votrient); pazopanib hydrochloride; Peficitinib; pegfilgrastim; Pembrolizumab (Keytruda); Pemetrexed; Pemigatinib; Perifosine; Pexidartinib; Phenazopyridine; Phenytoin; Pibrentasvir; Piperacillin; Pirfenidone; Piroxicam; Pomalidomide; Ponatinib; Ponatinib (lclusig); Posaconazole; Pralsetinib; pravastatin; prednicarbate; prednisone; pregabalin; pregabalin; Prinomastat (AG-3340); Progesterone; propanolol; protamine sulfate; psilocybin; Pyrantel; Pyrimethamine; Quetiapine; Quinupristin; Raloxifene; Raltegravir; ranibizumab; ranitidine; Ranolazine; Rebimastat (BMS-275291); Regorafenib; Regorafenib (Stivarga); relugolix (Orgovyx); remibrutinib; resiquimod; retinal; Retinol; ribociclib; ribociclib succinate; Rifampin; Rilpivirine; Rilpivirine; Rilpivirine; Rimegepant; Riociguat; Risdiplam; Risperidone; Ritonavir; Rivaroxaban; Rosuvastatin; rosuvastatin calcium; Rotigotine; roxadustat; rucaparib camsylate (Rubraca); ruxolitinib; Ruxolitinib (Jafaki); Sacubitril, valsartan; salicylic acid; Sapropterin; Saquinavir; sarilumab; Saxagliptin, Metformin; secukinumab; Selexipag; selinexor; Selpercatinib; selpercatinib (LOXO-292); Selumetinib; semaglutide; sertraline; Sevoflurane; sildenafil; Sildenafil Citrate; Simvastatin; siponimod; Siremadlin; Sirolimus; Sitagliptin; Sitagliptin phosphate monohydrate; Sodium bicarbonate or sodium carbonate; sodium deoxycholate; sodium nitrate; Sofosbuvir; Sofosbuvir; Sofosbuvir; Solifenacin; somatropin; sonidegib phosphate; Sorafenib; Sorafenib (Nexavar); spartalizumab; Spironolactone; sufentanil citrate; Sugammadex; sulbactam; Sulbactam; Sulfadiazine; Sulfamethoxazole; sulfasalazine; sumatriptan; Sunitinib; Sunitinib; Sunitinib (sutent); sunitinib malate (Sutent); Tacrolimus; TAF; TAF; TAF; TAF; talazoparib—Talzenna); Talinolol; Tamoxifen; Tamsulosin; tazarotene; tazobactam; TDF; TDF; Temsirolimus (CCl-779, Torisel); Teneligliptin; tenofovir alafenamide; Tenofovir Disoproxil Fumarate; Tepotinib; terbinafine; Terfenadine; Teriflunomide; Testosterone; Tezacaftor; THC; ticagrelor; tigecycline; Timolol; timolol maleate; Tipranavir; tisagenlecleucel; Tisagenlecleucel (Kymriah); Tivozanib; tofacitinib citrate; Tolvaptan; tralokinumab; tramadol; Trametinib; Trametinib; trametinib; Trametinib; Trametinib (Mekinist); tranexamic acid; Travoprost; trazodone; tretinoin; tretinoin; triancinolone; Triclabendazole; trifarotene; Trimethoprim; Triptorelin; tris; Tucatinib; ubrogepant; Umbralisib; upadacitinib; valproic acid; Valsartan; valsartan; Valsartan; valsartan; vancomycin; Vancomycin Hydrochloride (A); Vancomycin Hydrochloride (B); Vandetanib; Vandetenib (Caprelsa); Varenicline; Vasopressin; Velpatasvir; Velpatasvir; Vemurafenib; Venetoclax; Verapamil hydrochloride; Vilazodone; Vildagliptin; vismodegib; vitamin B6; vitamin D; Vonoprazan; Voriconazole; Vortioxetine; Voxilaprevir; Warfarin Sodium; Zanubrutinib; Zinc; zoledronic acid; and Zolpidem.

Embodiment 3. The formulation of Embodiment 1 or Embodiment 2, wherein the penetrant portion further comprises water in an amount of about 50% or 60% w/w.

Embodiment 4. The formulation of any one of Embodiments 1 to 3, wherein the penetrant portion is in an amount from about 70 to about 98% w/w of the formulation.

Embodiment 5. The formulation of any one of Embodiments 1 to 4, wherein the penetrant portion further comprises a mixture of xanthan gum, lecithin, sclerotium gum, pullulan, or a combination thereof in an amount less than 5% w/w of the formulation.

Embodiment 6. The formulation of any one of Embodiments 1 to 5, wherein the penetrant portion further comprises a mixture of caprylic triglycerides and capric triglycerides in amount less than 8% w/w of the formulation.

Embodiment 7. The formulation of any one of Embodiments 1 to 6, wherein the penetrant portion further comprises hydrogenated phosphatidylcholine, phosphatidylserine, phosphatidylethanolamine, phosphatidylinositol, one or more phosphatides, one or more Inositol phosphatides, or combinations thereof, in amount less than 12% w/w of the formulation.

Embodiment 8. The formulation of any one of Embodiments 1 to 7, wherein the penetrant portion further comprises cetyl alcohol in amount less than 5% w/w of the formulation.

Embodiment 9. The formulation of any one of Embodiments 1 to 8, wherein the formulation further comprises a gelling agent in an amount less than 10% w/w of the formulation.

Embodiment 10. The formulation of any one of Embodiments 1 to 9, further comprising a detergent portion comprised of a nonionic surfactant in an amount from about 2 to about 25% w/w of the penetrant portion; and a polar solvent in an amount less than 5% w/w of the penetrant portion.

Embodiment 11. The formulation of any one of Embodiments 1 to 10, further comprising a carbonate salt comprised of sodium bicarbonate milled to a particle size less than 70 μm, wherein the sodium bicarbonate is solubilized in the formulation in an amount less than 10% w/w of the formulation.

Embodiment 12. The formulation of any one of Embodiments 1 to 11, further comprising a polar solvent in an amount less than 5% w/w of the formulation.

Embodiment 13. The formulation of any one of Embodiments 1 to 12, further comprising a humectant, an emulsifier, an emollient, or a combination thereof.

Embodiment 14. The formulation of any one of Embodiments 1 to 13, wherein the formulation has a pH of 7-10.5.

Embodiment 15. A method for transdermal delivery of the formulation of any one of Embodiments 1 to 14, through the skin of a subject.

Embodiment 16. A transdermal delivery formulation for transdermal delivery of a medicament through the skin, nail or hair follicle of a subject, wherein the transdermal delivery formulation comprises: a) a therapeutically effective amount of a medicament; b) a transdermal delivery formulation in an amount less than about 60% w/w, comprising: i. one or more phosphatides, ii. one or more alcohols, iii. one or more fatty acids, iv. a surfactant and c) water in an amount less than about 65% w/w.

Embodiment 17. The transdermal delivery formulation of Embodiment 16, wherein the one or more phosphatides are selected from phosphatidylcholine, hydrogenated phosphatidylcholine, phosphatidylserine, phosphatidylethanolamine, phosphatidylinositol, one or more phosphatides, one or more Inositol phosphatides, or combinations thereof, in amount less than 12% w/w of the formulation.

Embodiment 18. The transdermal delivery formulation of Embodiment 16 or Embodiment 17, wherein the one or more alcohols are selected from benzyl alcohol, cetyl alcohol and fatty alcohols.

Embodiment 19. The transdermal delivery formulation of any one of Embodiments 16 to 18, wherein the one or more fatty acids are selected from linoleic acid, oleic acid, stearic acid and safflower oil.

Embodiment 20. The transdermal delivery formulation of any one of Embodiments 16 to 19, wherein the surfactant is one or more of Poloxamer 407 and Polyglyceryl-4 Laurate.

Embodiment 21. The transdermal delivery formulation of any one of Embodiments 16 to 20, wherein the medicament is at least one of (3S,4S)-8-(6-amino-5-((2-amino-3-chloropyridin-4-yl)thio)pyrazin-2-yl)-3-methyl-2-oxa-8-azaspiro[4.5]decan-4-amine; (S)-3-amino-6-methoxy-N-(3,3,3-trifluoro-2-hydroxy-2-methylpropyl)-5-(trifluoromethyl)picolinamide; 2-fluoro-N-methyl-4-[7-quinolin-6-yl-methyl)-imidazo[1,2-B][1,2,4]triazin-2yl]benzamide; 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide; 4-(7-Hydroxy-2-isopropyl-4-oxo-4H-quinazolin-3-yl)-benzonitrile; Abacavir; Abiraterone Acetate; abiratone acetate; abobotulinum toxin A; Acalabrutinib; Acarbose; Acetaminophen; Acetazolamide; Acetylsalicylic Acid; acitretin; acyclovir; adalimumab; adapalene; adapalene; Afoxolaner; agalsidase beta; Albendazole; Alectinib; Alectinib; alendronate sodium; alglucosidase alfa; alitretinoin; Alogliptin; alpelisib; Aluminum Hydroxide; Ambrisentan; Amiodarone; Amitriptyline; Amlodipine; Amlodipine; Amlodipine besylate; amlodopine besylate; amorolfine hydrochloride; Amoxicillin; Amphotericin B; Amphotericin B liposome; ampicillin; Amprenavir; antihemophilic factor, Fc fusion protein; apalutamide; Apatinib; apixaban; apremilast; Aprepitant; Aripiprazole; Artemether; aspirin; Atazanavir; atenolol; Atomoxetine; Atorvastatin; Atorvastatin; Avapritinib; Axitinib (Inlyta); Azacitidine; Azathioprine; azelaic acid; Azilsartan medoxomil; azithromycin; baclofen; baricitinib; basiliximab; Batimastat (BB-94); becaplermin; Belumosudil; Bendamustine; benzoyl peroxide; benzoyl peroxide; betamethasone dipropionate; betamethasone valerate; bexarotene; Bictegravir; Bimatoprost; binimetinib (Mektovi); Bortezomib; Bortezomib (Velcade); Bosentan; Bosutinib (Bosulif); botulinum toxin A; branched chain amino acids; Brexpiprazole; Brimonidine; brimonidine tartrate; brodalumab; buproprion; Cabazitaxel; Cabozantinib; Cabozantinib (Cometriz); Calcifediol; calcipotriene; Calcipotriene; Calcipotriol; calcitonin; calcitriol; calcium carbonate; Canagliflozin; canakinumab; Capecitabine; Capmatinib; capsaicin; Carbamazepine; Carbidopa; Carfilzomib; Carfilzomib (Kyprolis); Cariprazine; carvedilol; CD-12681; CEE-321; cefazolin; cefepime; Cefoperazone; cefotaxime sodium; celecoxib; celecoxib; cemiplimab; Ceritinib; Chlorothiazide; Chlorpromazine; Chlorthalidone; cinacalcet; ciprofloxacin; Cisapride; citalopram; citric acid; clarithromycin; Clavulanate; Clindamycin phosphate; Clobazam; clobetasol propionate; Clofazimine; clomiphene; clonidine; Clopidogrel; clopidogrel bisulfate; cobicistat; Cobicistat; Colchocine; Colistin; Corticotropin; crisaborole; crizanlizumab; Crizotinib (Xalkori); cyclobenzaprine; cyclosporine; Dabigatran Etexilate; Dabrafenib; Dabrafenib; dabrafenib; dabrafenib; dacomitinib (Vizimpro); dalbavancin; dalfopristin; Danazol; Dapagliflozin; Dapsone; daptomycin; Darunavir; Dasatinib; decitabine; defatted Peanut (Arachis hypogaea) Flour; deferasirox; delgocitinib; deoxycholic acid; Desipramine; desonide; Dexlansoprazole; Dexmethylphenidate; dextroamphetamine/amphetamine salts; Diclofenac; diclofenac sodium; diflucortolone; Diflucortolone; Difunisal; Digoxin; Diloxanide; Dimethyl Fumarate; diphenhydramine hydrochloride; diphtheria vaccine; docetaxel; Dolutegravir; Dolutegravir; Donepezil; Doxercalciferol; doxycycline; doxycycline ER; Doxycycline Hyclate (B); Doxycycline Monohydrate (A); Dronabinol; Dronedarone; Drospirenone; Dukoral/ShanChol cholera vaccine; Duloxetine; dupilumab; durvalumab; Dutasteride; Duvelisib; ecallantide; Edoxaban; Efavirenz; Efavirenz; Elafibranor; elagolix sodium; Elbasvir; Eletriptan; Eltrombopag; Elvitegravir; Empagliflozin; Emtricitabine; emtricitabine; Emtricitabine; Emtricitabine; Emtricitabine; Emtricitabine; Emtricitabine; Emtricitabine; encorafenib (Braftovi); enfuviritide; enoxaparin sodium; Entecavir; entrectinib; Enzalutamide; Epinephrine; erdafitinib; erenumab; Eribulin; Erlotinib; Erlotinib HCl (Tarceva); Ertapenem; Erythromycin; escitalopram; esketamine (ketamine); Esomeprazole; esopremazole; estradiol; Estrogens—conjugated; etanercept; Ethinyl Estradiol; Ethinyl Estradiol; Ethinyl Estradiol; Etonogestrel; Etonogestrel; Etravirine; etretinate; Everolimus; Everolimus (Afinitor); Everolimus (Votubia, Zortress/Certican); Ezetimibe; Ezetimibe; Ezetimibe; Famotidine; fasinumab; Febuxostat; Fedratinib; Fenofibrate; Ferric Carboxymaltose; fexofenadine hydrochloride; filgotinib; filgrastim; fingolimod; fluocinolone acetonide; fluocinolone acetonide; fluoxetine; Flurbiprofen; Folic acid; Forskolin; Fostamatinib; Fulvestrant; furosemide; fusidic acid; fusidic acid; gabapentin; Ganetespib; Gefitinib (Iressa); Gilteritinib; glasdegib (Daurismo) or glasdegib maleate; Glecaprevir; Glibenclamide; Glimepiride; glipizide; glucagon; glucosamine; glutaric anhydride; glycopyrrolate; Goserelin; goserelin LA; granisetron; Grazoprevir; Griseofulvin; halobetasol propionate; Haloperidol; heparin sodium; heplisav-B vaccine; hyaluronic acid; Hydrochlorothiazide; Hydrochlorothiazide; Hydrochlorothiazide; Hydrochlorothiazide; hydrocortisone; hydrocortisone acetate; hydroquinone; hydroxychloroquine; Hydroxyprogesterone; Ibrutinib; Ibrutinib (Imbruvica); ibuprofen; icatibant acetate; Idelalisib; Imatinib; Imatinib (Gleevec); imatinib mesylate; imiglucerase; inclisiran; Indinavir; Indomethacin; infliximab; ingenol mebutate; inotersen; insulin A; insuline glargine; interferon beta-Ib; Iopanoic acid; Irbesartan; isoconazole; isoconazole; Isotretinoin; Itraconazole; Ivacaftor; Ivacaftor; ivermectin; Ixazomib; ketoconazole; ketones; ketoprofen; Keyruvia; Lacosamide; lamictal; Lamivudine; Lansoprazole; Lapatinib; Lapatinib (Tykerb); larotrectinib; Ledipasvir; leflunomide; Lenalidomide; Lenvatinib; LEO-138559; LEO-152020; Leuprolide; Levetiracetam; levodopa; Levodopa/Benserazide; Levonorgestrel; Levothyroxine; Linagliptin; linezolid; lisinopril; L-lysine free base; lofexidine; Lopinavir; Loratadine; lorlatinib (Lorviqua); Losartan; Lovastatin; Lubiprostone; Lumacaftor/Ivacaftor; Lumefantrine; Lurasidone; Luspatercept; lymecycline; Macitentan; magnesium; magnesium lactate; Marimastat (BB-2516); Marizomib (NPI-0052); Mebendazole; Mefloquine; melatonin; meloxicam; memantine hydrochloride; meningococcal[serotype b] vaccine; Mesalazine; Metformin; Metformin; metformin; Metformin; methotrexate; methyl aminolevulinate hydrochloride; Methylphenidate; methylprednisolone; Metoprolol succinate; metronidazole; Midostaurin; minoxidil; Mirabegron; mizolastine MR; Montelukast; Mycophenolate Mofetil; N-(3-(2-(2-hydroxyethoxy)-6-morpholinopyridin-4-yl)-4-methylphenyl)-2-(trifluoromethyl)-isonicotinamide; N-(3-(6-Amino-5-(2-(N-methylacrylamido)ethoxy)pyrimidin-4-yl)-5-fluoro-2-methylphenyl)-4-cyclopropyl-2-fluorobenzamide; N-[4-(Chlorodifluoromethoxy)phenyl]-6-[(3R)-3-hydroxpyrrolidin-1-yl]-5-(1H-pyrazol-5-yl)-pyridine-3-carboxamide hydrochloride; Nalidixic acid (Quinolone antibiotic); naloxone; Naproxen; Navitoclax (ABT-263); Nebivolol; Nelfinavir; nemolizumab; neomycin; Neovastat (AE-941); Neratinib—Free Base; Neratinib (Nerlynx); Neratinib Maleate; Nevirapine; Niclosamide; Nifedipine; nilotinib; Nilotinib; Nilotinib (Tasigna); Nintedanib; niratimib; Nitric Oxide; Nitrofurantoin; Norethindrone Acetate; nusinersen; NVP-AUY922; Nystatin; Obatoclax (GX15-070); octreotide acetate; ofatumumab; Ofloxacin; Olanzapine; olaparib; Olmesartan Medoxomil; olumacostat; omalizumab; Oseltamivir; Osimertinib; osimertinib mesylate; Oxaprozin; Ozanimod; Paclitaxel; Paclitaxel Protein Bound; Palbociclib (Ibrance); Paliperidone Palmitate; Pantoprazole; parathyroid hormone; Paricalcitol; patisiran; pazopanib; Pazopanib (Votrient); pazopanib hydrochloride; Peficitinib; pegfilgrastim; Pembrolizumab (Keytruda); Pemetrexed; Pemigatinib; Perifosine; Pexidartinib; Phenazopyridine; Phenytoin; Pibrentasvir; Piperacillin; Pirfenidone; Piroxicam; Pomalidomide; Ponatinib; Ponatinib (lclusig); Posaconazole; Pralsetinib; pravastatin; prednicarbate; prednisone; pregabalin; pregabalin; Prinomastat (AG-3340); Progesterone; propanolol; protamine sulfate; psilocybin; Pyrantel; Pyrimethamine; Quetiapine; Quinupristin; Raloxifene; Raltegravir; ranibizumab; ranitidine; Ranolazine; Rebimastat (BMS-275291); Regorafenib; Regorafenib (Stivarga); relugolix (Orgovyx); remibrutinib; resiquimod; retinal; Retinol; ribociclib; ribociclib succinate; Rifampin; Rilpivirine; Rilpivirine; Rilpivirine; Rimegepant; Riociguat; Risdiplam; Risperidone; Ritonavir; Rivaroxaban; Rosuvastatin; rosuvastatin calcium; Rotigotine; roxadustat; rucaparib camsylate (Rubraca); ruxolitinib; Ruxolitinib (Jafaki); Sacubitril, valsartan; salicylic acid; Sapropterin; Saquinavir; sarilumab; Saxagliptin, Metformin; secukinumab; Selexipag; selinexor; Selpercatinib; selpercatinib (LOXO-292); Selumetinib; semaglutide; sertraline; Sevoflurane; sildenafil; Sildenafil Citrate; Simvastatin; siponimod; Siremadlin; Sirolimus; Sitagliptin; Sitagliptin phosphate monohydrate; Sodium bicarbonate or sodium carbonate; sodium deoxycholate; sodium nitrate; Sofosbuvir; Sofosbuvir; Sofosbuvir; Solifenacin; somatropin; sonidegib phosphate; Sorafenib; Sorafenib (Nexavar); spartalizumab; Spironolactone; sufentanil citrate; Sugammadex; sulbactam; Sulbactam; Sulfadiazine; Sulfamethoxazole; sulfasalazine; sumatriptan; Sunitinib; Sunitinib; Sunitinib (sutent); sunitinib malate (Sutent); Tacrolimus; TAF; TAF; TAF; TAF; talazoparib—Talzenna); Talinolol; Tamoxifen; Tamsulosin; tazarotene; tazobactam; TDF; TDF; Temsirolimus (CCl-779, Torisel); Teneligliptin; tenofovir alafenamide; Tenofovir Disoproxil Fumarate; Tepotinib; terbinafine; Terfenadine; Teriflunomide; Testosterone; Tezacaftor; THC; ticagrelor; tigecycline; Timolol; timolol maleate; Tipranavir; tisagenlecleucel; Tisagenlecleucel (Kymriah); Tivozanib; tofacitinib citrate; Tolvaptan; tralokinumab; tramadol; Trametinib; Trametinib; trametinib; Trametinib; Trametinib (Mekinist); tranexamic acid; Travoprost; trazodone; tretinoin; tretinoin; triancinolone; Triclabendazole; trifarotene; Trimethoprim; Triptorelin; tris; Tucatinib; ubrogepant; Umbralisib; upadacitinib; valproic acid; Valsartan; valsartan; Valsartan; valsartan; vancomycin; Vancomycin Hydrochloride (A); Vancomycin Hydrochloride (B); Vandetanib; Vandetenib (Caprelsa); Varenicline; Vasopressin; Velpatasvir; Velpatasvir; Vemurafenib; Venetoclax; Verapamil hydrochloride; Vilazodone; Vildagliptin; vismodegib; vitamin B6; vitamin D; Vonoprazan; Voriconazole; Vortioxetine; Voxilaprevir; Warfarin Sodium; Zanubrutinib; Zinc; zoledronic acid; and Zolpidem.

Embodiment 22. The transdermal delivery formulation of any one of Embodiments 16 to 21, wherein the one or more alcohols is benzyl alcohol in an amount from about 0.5 to about 5% w/w.

Embodiment 23. The transdermal delivery formulation of any one of Embodiments 16 to 22, further comprising isopropyl palmitate in an amount from about 1 to about 15% w/w.

Embodiment 24. The transdermal delivery formulation of any one of Embodiments 16 to 23, wherein the water is deionized water and/or purified water.

Embodiment 25. The transdermal delivery formulation of any one of Embodiments 16 to 24, wherein the water is in an amount from about 50 to about 70% w/w of the formulation.

Embodiment 26. The transdermal delivery formulation of any one of Embodiments 16 to 25, wherein the one or more phosphatides is from about 0.5 to about 55% w/w of the transdermal delivery formulation.

Embodiment 27. The transdermal delivery formulation of any one of Embodiments 16 to 26, wherein the one or more phosphatides comprises phosphatidylcholine, hydrogenated phosphatidylcholine, phosphatidylserine, phosphatidylethanolamine, phosphatidylinositol, or a combination thereof in amount less than 30% w/w of the formulation.

Embodiment 28. The transdermal delivery formulation of any one of Embodiments 16 to 27, wherein the one or more phosphatides comprises phosphatidylcholine.

Embodiment 29. The transdermal delivery formulation of any one of Embodiments 16 to 28, wherein the one or more fatty acids is from about 0.5 to about 2% w/w of the transdermal delivery formulation.

Embodiment 30. The transdermal delivery formulation of any one of Embodiments 16 to 29, wherein the one or more fatty acids is from about 1 to about 5% w/w of the transdermal delivery formulation.

Embodiment 31. The transdermal delivery formulation of any one of Embodiments 16 to 30, wherein the one or more phosphatides are derived from a seed oil in an amount from about 0.5 to about 15% w/w of the transdermal delivery formulation.

Embodiment 32. The transdermal delivery formulation of any one of Embodiments 16 to 31, wherein the one or more phosphatides are derived from a seed oil, and the one or more phosphatides are from about 5 to about 15% w/w of the transdermal delivery formulation.

Embodiment 33. The transdermal delivery formulation of any one of Embodiments 16 to 32, wherein the one or more phosphatides are derived from a safflower oil, and the one or more phosphatides are from about 0.5 to about 15% w/w of the transdermal delivery formulation.

Embodiment 34. The transdermal delivery formulation of any one of Embodiments 16 to 33, wherein the one or more phosphatides are derived from a safflower oil, and the one or more phosphatides are from about 5 to about 15% w/w of the transdermal delivery formulation.

Embodiment 35. The transdermal delivery formulation of any one of Embodiments 16 to 34, wherein the one or more phosphatides are derived from an almond oil, and the one or more phosphatides are from about 0.5 to about 51% w/w of the transdermal delivery formulation.

Embodiment 36. The transdermal delivery formulation of any one of Embodiments 16 to 35, wherein the one or more phosphatides are derived from an almond oil, and the one or more phosphatides are from about 5 to about 15% w/w of the transdermal delivery formulation.

Embodiment 37. The transdermal delivery formulation of any one of Embodiments 16 to 36, wherein the one or more phosphatides comprises one or more fatty acids derived from soy lecithin.

Embodiment 38. The transdermal delivery formulation of any one of Embodiments 16 to 37, wherein the surfactant is a nonionic surfactant in an amount from about 2 to about 25% w/w of the transdermal delivery formulation.

Embodiment 39. The transdermal delivery formulation of Embodiment 38, further comprising a polar solvent at least in an amount in molar excess of the nonionic surfactant.

Embodiment 40. The transdermal delivery formulation of Embodiment 38 or 39, wherein the nonionic surfactant is a poloxamer and the polar solvent is water.

Embodiment 41. The transdermal delivery formulation of any one of Embodiments 16 to 40, further comprising a polar solvent in an amount less than 5% w/w of the formulation.

Embodiment 42. The transdermal delivery formulation of any one of Embodiments 16 to 41, wherein the transdermal delivery formulation further comprises a detergent portion in an amount from about 1 to about 20% w/w of the transdermal delivery formulation.

Embodiment 43. The transdermal delivery formulation of Embodiment 42, wherein the detergent portion comprises a nonionic surfactant in an amount from about 2 to about 25% w/w of the transdermal delivery formulation; and a polar solvent in an amount less than 5% w/w of the transdermal delivery formulation.

Embodiment 44. The transdermal delivery formulation of any one of Embodiments 16 to 43, wherein the alcohol is less than 10% w/w of the transdermal delivery formulation.

Embodiment 45. The transdermal delivery formulation of any one of Embodiments 16 to 44, wherein the transdermal delivery formulation further comprises glycerine in an amount less than 5% w/w of the formulation.

Embodiment 46. The transdermal delivery formulation of any one of Embodiments 16 to 45, wherein the transdermal delivery formulation further comprises propylene glycol in an amount less than 8% w/w of the formulation.

Embodiment 47. The transdermal delivery formulation of any one of Embodiments 16 to 46, wherein the formulation comprises a gelling agent in an amount less than 20% w/w of the formulation.

Embodiment 48. The transdermal delivery formulation of any one of Embodiments 16 to 47, wherein the formulation comprises menthol in an amount from about 0.05 to about 5% w/w of the formulation.

Embodiment 49. The transdermal delivery formulation of any one of Embodiments 16 to 48, further comprising a humectant, an emulsifier, an emollient, or a combination thereof.

Embodiment 50. The transdermal delivery formulation of any one of Embodiments 16 to 49, wherein the formulation has a pH of 9-11.

Embodiment 51. The transdermal delivery formulation of any one of Embodiments 16 to 50, wherein the formulation has a pH of 7-10.5.

Embodiment 52. The transdermal delivery formulation of any one of Embodiments 16 to 51, further comprising an at least second medicament.

Embodiment 53. The transdermal delivery formulation of Embodiment 52, wherein the first and the at least second medicament are selected from the group consisting of Abacavir and Dolutegravir and Lamivudine; adapalene and benzoyl peroxide; Amlodipine and Valsartan; amlodopine besylate and valsartan; Amoxicillin and Clavulanate; ampicillin and sulbactam; Artemether and Lumefantrine; betamethasone valerate and fusidic acid; Bictegravir and Emtricitabine and TAF; Bimatoprost and timolol maleate; Brimonidine and Timolol; Calcipotriene and betamethasone dipropionate; Canagliflozin and Metformin; Carbidopa and levodopa; Cefoperazone and Sulbactam; Dabrafenib and Trametinib; Darunavir and cobicistat and emtricitabine and tenofovir alafenamide; Diflucortolone and isoconazole; Drospirenone and Ethinyl Estradiol; Efavirenz and Emtricitabine and TDF; Elbasvir and Grazoprevir; Elvitegravir and Cobicistat and Emtricitabine and TAF; Emtricitabine and Rilpivirine and TAF; Emtricitabine and TAF; Emtricitabine and Tenofovir Disoproxil Fumarate; Ethinyl Estradiol and Etonogestrel; Ezetimibe and Atorvastatin; Ezetimibe and Simvastatin; fluocinolone acetonide and hydroquinone and tretinoin; fusidic acid and hydrocortisone acetate; Glecaprevir and Pibrentasvir; Hydrochlorothiazide and Valsartan; Irbesartan and Hydrochlorothiazide; Ledipasvir and Sofosbuvir; Linagliptin and Metformin; Losartan and Hydrochlorothiazide; Metformin and Sitagliptin; Norethindrone Acetate and Ethinyl Estradiol; Piperacillin and tazobactam; pregabalin and celecoxib; Quinupristin and dalfopristin; Rilpivirine and Emtricitabine and TDF; Sofosbuvir and Velpatasvir; Sofosbuvir and Velpatasvir and Voxilaprevir; Tezacaftor and Ivacaftor; trametinib and dabrafenib; Trametinib and dabrafenib; and Zinc and magnesium and vitamin B6.

Embodiment 54. A method of transdermal delivery an active ingredient comprising a step of applying to the skin of a subject an effective amount of the formulation of any one of Embodiments 16 to 52.

Embodiment 55. A method of treating an ailment, the method comprising a step of administering a medicament to an area of skin of a subject, wherein the medicament is administered with a transdermal formulation, wherein the transdermal formulation comprises 3-15% w/w phosphatidylcholine, 5-20% w/w isopropyl palmitate, about 0.1-5% w/w stearic acid, about 0.5-5% w/w benzyl alcohol, 0.5-10% w/w polyglyceryl-4 laurate and 5-20% w/w poloxamer 407.

Embodiment 56. The method of Embodiment 55, wherein the medicament is at least one of (3S,4S)-8-(6-amino-5-((2-amino-3-chloropyridin-4-yl)thio)pyrazin-2-yl)-3-methyl-2-oxa-8-azaspiro[4.5]decan-4-amine; (S)-3-amino-6-methoxy-N-(3,3,3-trifluoro-2-hydroxy-2-methylpropyl)-5-(trifluoromethyl)picolinamide; 2-fluoro-N-methyl-4-[7-quinolin-6-yl-methyl)-imidazo[1,2-B][1,2,4]triazin-2yl]benzamide; 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide; 4-(7-Hydroxy-2-isopropyl-4-oxo-4H-quinazolin-3-yl)-benzonitrile; Abacavir; Abiraterone Acetate; abiratone acetate; abobotulinum toxin A; Acalabrutinib; Acarbose; Acetaminophen; Acetazolamide; Acetylsalicylic Acid; acitretin; acyclovir; adalimumab; adapalene; adapalene; Afoxolaner; agalsidase beta; Albendazole; Alectinib; Alectinib; alendronate sodium; alglucosidase alfa; alitretinoin; Alogliptin; alpelisib; Aluminum Hydroxide; Ambrisentan; Amiodarone; Amitriptyline; Amlodipine; Amlodipine; Amlodipine besylate; amlodopine besylate; amorolfine hydrochloride; Amoxicillin; Amphotericin B; Amphotericin B liposome; ampicillin; Amprenavir; antihemophilic factor, Fc fusion protein; apalutamide; Apatinib; apixaban; apremilast; Aprepitant; Aripiprazole; Artemether; aspirin; Atazanavir; atenolol; Atomoxetine; Atorvastatin; Atorvastatin; Avapritinib; Axitinib (Inlyta); Azacitidine; Azathioprine; azelaic acid; Azilsartan medoxomil; azithromycin; baclofen; baricitinib; basiliximab; Batimastat (BB-94); becaplermin; Belumosudil; Bendamustine; benzoyl peroxide; benzoyl peroxide; betamethasone dipropionate; betamethasone valerate; bexarotene; Bictegravir; Bimatoprost; binimetinib (Mektovi); Bortezomib; Bortezomib (Velcade); Bosentan; Bosutinib (Bosulif); botulinum toxin A; branched chain amino acids; Brexpiprazole; Brimonidine; brimonidine tartrate; brodalumab; buproprion; Cabazitaxel; Cabozantinib; Cabozantinib (Cometriz); Calcifediol; calcipotriene; Calcipotriene; Calcipotriol; calcitonin; calcitriol; calcium carbonate; Canagliflozin; canakinumab; Capecitabine; Capmatinib; capsaicin; Carbamazepine; Carbidopa; Carfilzomib; Carfilzomib (Kyprolis); Cariprazine; carvedilol; CD-12681; CEE-321; cefazolin; cefepime; Cefoperazone; cefotaxime sodium; celecoxib; celecoxib; cemiplimab; Ceritinib; Chlorothiazide; Chlorpromazine; Chlorthalidone; cinacalcet; ciprofloxacin; Cisapride; citalopram; citric acid; clarithromycin; Clavulanate; Clindamycin phosphate; Clobazam; clobetasol propionate; Clofazimine; clomiphene; clonidine; Clopidogrel; clopidogrel bisulfate; cobicistat; Cobicistat; Colchocine; Colistin; Corticotropin; crisaborole; crizanlizumab; Crizotinib (Xalkori); cyclobenzaprine; cyclosporine; Dabigatran Etexilate; Dabrafenib; Dabrafenib; dabrafenib; dabrafenib; dacomitinib (Vizimpro); dalbavancin; dalfopristin; Danazol; Dapagliflozin; Dapsone; daptomycin; Darunavir; Dasatinib; decitabine; defatted Peanut (Arachis hypogaea) Flour; deferasirox; delgocitinib; deoxycholic acid; Desipramine; desonide; Dexlansoprazole; Dexmethylphenidate; dextroamphetamine/amphetamine salts; Diclofenac; diclofenac sodium; diflucortolone; Diflucortolone; Difunisal; Digoxin; Diloxanide; Dimethyl Fumarate; diphenhydramine hydrochloride; diphtheria vaccine; docetaxel; Dolutegravir; Dolutegravir; Donepezil; Doxercalciferol; doxycycline; doxycycline ER; Doxycycline Hyclate (B); Doxycycline Monohydrate (A); Dronabinol; Dronedarone; Drospirenone; Dukoral/ShanChol cholera vaccine; Duloxetine; dupilumab; durvalumab; Dutasteride; Duvelisib; ecallantide; Edoxaban; Efavirenz; Efavirenz; Elafibranor; elagolix sodium; Elbasvir; Eletriptan; Eltrombopag; Elvitegravir; Empagliflozin; Emtricitabine; emtricitabine; Emtricitabine; Emtricitabine; Emtricitabine; Emtricitabine; Emtricitabine; Emtricitabine; encorafenib (Braftovi); enfuviritide; enoxaparin sodium; Entecavir; entrectinib; Enzalutamide; Epinephrine; erdafitinib; erenumab; Eribulin; Erlotinib; Erlotinib HCl (Tarceva); Ertapenem; Erythromycin; escitalopram; esketamine (ketamine); Esomeprazole; esopremazole; estradiol; Estrogens—conjugated; etanercept; Ethinyl Estradiol; Ethinyl Estradiol; Ethinyl Estradiol; Etonogestrel; Etonogestrel; Etravirine; etretinate; Everolimus; Everolimus (Afinitor); Everolimus (Votubia, Zortress/Certican); Ezetimibe; Ezetimibe; Ezetimibe; Famotidine; fasinumab; Febuxostat; Fedratinib; Fenofibrate; Ferric Carboxymaltose; fexofenadine hydrochloride; filgotinib; filgrastim; fingolimod; fluocinolone acetonide; fluocinolone acetonide; fluoxetine; Flurbiprofen; Folic acid; Forskolin; Fostamatinib; Fulvestrant; furosemide; fusidic acid; fusidic acid; gabapentin; Ganetespib; Gefitinib (Iressa); Gilteritinib; glasdegib (Daurismo) or glasdegib maleate; Glecaprevir; Glibenclamide; Glimepiride; glipizide; glucagon; glucosamine; glutaric anhydride; glycopyrrolate; Goserelin; goserelin LA; granisetron; Grazoprevir; Griseofulvin; halobetasol propionate; Haloperidol; heparin sodium; heplisav-B vaccine; hyaluronic acid; Hydrochlorothiazide; Hydrochlorothiazide; Hydrochlorothiazide; Hydrochlorothiazide; hydrocortisone; hydrocortisone acetate; hydroquinone; hydroxychloroquine; Hydroxyprogesterone; Ibrutinib; Ibrutinib (Imbruvica); ibuprofen; icatibant acetate; Idelalisib; Imatinib; Imatinib (Gleevec); imatinib mesylate; imiglucerase; inclisiran; Indinavir; Indomethacin; infliximab; ingenol mebutate; inotersen; insulin A; insuline glargine; interferon beta-lb; Iopanoic acid; Irbesartan; isoconazole; isoconazole; Isotretinoin; Itraconazole; Ivacaftor; Ivacaftor; ivermectin; Ixazomib; ketoconazole; ketones; ketoprofen; Keyruvia; Lacosamide; lamictal; Lamivudine; Lansoprazole; Lapatinib; Lapatinib (Tykerb); larotrectinib; Ledipasvir; leflunomide; Lenalidomide; Lenvatinib; LEO-138559; LEO-152020; Leuprolide; Levetiracetam; levodopa; Levodopa/Benserazide; Levonorgestrel; Levothyroxine; Linagliptin; linezolid; lisinopril; L-lysine free base; lofexidine; Lopinavir; Loratadine; lorlatinib (Lorviqua); Losartan; Lovastatin; Lubiprostone; Lumacaftor/Ivacaftor; Lumefantrine; Lurasidone; Luspatercept; lymecycline; Macitentan; magnesium; magnesium lactate; Marimastat (BB-2516); Marizomib (NPI-0052); Mebendazole; Mefloquine; melatonin; meloxicam; memantine hydrochloride; meningococcal[serotype b] vaccine; Mesalazine; Metformin; Metformin; metformin; Metformin; methotrexate; methyl aminolevulinate hydrochloride; Methylphenidate; methylprednisolone; Metoprolol succinate; metronidazole; Midostaurin; minoxidil; Mirabegron; mizolastine MR; Montelukast; Mycophenolate Mofetil; N-(3-(2-(2-hydroxyethoxy)-6-morpholinopyridin-4-yl)-4-methylphenyl)-2-(trifluoromethyl)-isonicotinamide; N-(3-(6-Amino-5-(2-(N-methylacrylamido)ethoxy)pyrimidin-4-yl)-5-fluoro-2-methylphenyl)-4-cyclopropyl-2-fluorobenzamide; N-[4-(Chlorodifluoromethoxy)phenyl]-6-[(3R)-3-hydroxpyrrolidin-1-yl]-5-(1H-pyrazol-5-yl)-pyridine-3-carboxamide hydrochloride; Nalidixic acid (Quinolone antibiotic); naloxone; Naproxen; Navitoclax (ABT-263); Nebivolol; Nelfinavir; nemolizumab; neomycin; Neovastat (AE-941); Neratinib—Free Base; Neratinib (Nerlynx); Neratinib Maleate; Nevirapine; Niclosamide; Nifedipine; nilotinib; Nilotinib; Nilotinib (Tasigna); Nintedanib; niratimib; Nitric Oxide; Nitrofurantoin; Norethindrone Acetate; nusinersen; NVP-AUY922; Nystatin; Obatoclax (GX15-070); octreotide acetate; ofatumumab; Ofloxacin; Olanzapine; olaparib; Olmesartan Medoxomil; olumacostat; omalizumab; Oseltamivir; Osimertinib; osimertinib mesylate; Oxaprozin; Ozanimod; Paclitaxel; Paclitaxel Protein Bound; Palbociclib (Ibrance); Paliperidone Palmitate; Pantoprazole; parathyroid hormone; Paricalcitol; patisiran; pazopanib; Pazopanib (Votrient); pazopanib hydrochloride; Peficitinib; pegfilgrastim; Pembrolizumab (Keytruda); Pemetrexed; Pemigatinib; Perifosine; Pexidartinib; Phenazopyridine; Phenytoin; Pibrentasvir; Piperacillin; Pirfenidone; Piroxicam; Pomalidomide; Ponatinib; Ponatinib (lclusig); Posaconazole; Pralsetinib; pravastatin; prednicarbate; prednisone; pregabalin; pregabalin; Prinomastat (AG-3340); Progesterone; propanolol; protamine sulfate; psilocybin; Pyrantel; Pyrimethamine; Quetiapine; Quinupristin; Raloxifene; Raltegravir; ranibizumab; ranitidine; Ranolazine; Rebimastat (BMS-275291); Regorafenib; Regorafenib (Stivarga); relugolix (Orgovyx); remibrutinib; resiquimod; retinal; Retinol; ribociclib; ribociclib succinate; Rifampin; Rilpivirine; Rilpivirine; Rilpivirine; Rimegepant; Riociguat; Risdiplam; Risperidone; Ritonavir; Rivaroxaban; Rosuvastatin; rosuvastatin calcium; Rotigotine; roxadustat; rucaparib camsylate (Rubraca); ruxolitinib; Ruxolitinib (Jafaki); Sacubitril, valsartan; salicylic acid; Sapropterin; Saquinavir; sarilumab; Saxagliptin, Metformin; secukinumab; Selexipag; selinexor; Selpercatinib; selpercatinib (LOXO-292); Selumetinib; semaglutide; sertraline; Sevoflurane; sildenafil; Sildenafil Citrate; Simvastatin; siponimod; Siremadlin; Sirolimus; Sitagliptin; Sitagliptin phosphate monohydrate; Sodium bicarbonate or sodium carbonate; sodium deoxycholate; sodium nitrate; Sofosbuvir; Sofosbuvir; Sofosbuvir; Solifenacin; somatropin; sonidegib phosphate; Sorafenib; Sorafenib (Nexavar); spartalizumab; Spironolactone; sufentanil citrate; Sugammadex; sulbactam; Sulbactam; Sulfadiazine; Sulfamethoxazole; sulfasalazine; sumatriptan; Sunitinib; Sunitinib; Sunitinib (sutent); sunitinib malate (Sutent); Tacrolimus; TAF; TAF; TAF; TAF; talazoparib—Talzenna); Talinolol; Tamoxifen; Tamsulosin; tazarotene; tazobactam; TDF; TDF; Temsirolimus (CCl-779, Torisel); Teneligliptin; tenofovir alafenamide; Tenofovir Disoproxil Fumarate; Tepotinib; terbinafine; Terfenadine; Teriflunomide; Testosterone; Tezacaftor; THC; ticagrelor; tigecycline; Timolol; timolol maleate; Tipranavir; tisagenlecleucel; Tisagenlecleucel (Kymriah); Tivozanib; tofacitinib citrate; Tolvaptan; tralokinumab; tramadol; Trametinib; Trametinib; trametinib; Trametinib; Trametinib (Mekinist); tranexamic acid; Travoprost; trazodone; tretinoin; tretinoin; triancinolone; Triclabendazole; trifarotene; Trimethoprim; Triptorelin; tris; Tucatinib; ubrogepant; Umbralisib; upadacitinib; valproic acid; Valsartan; valsartan; Valsartan; valsartan; vancomycin; Vancomycin Hydrochloride (A); Vancomycin Hydrochloride (B); Vandetanib; Vandetenib (Caprelsa); Varenicline; Vasopressin; Velpatasvir; Velpatasvir; Vemurafenib; Venetoclax; Verapamil hydrochloride; Vilazodone; Vildagliptin; vismodegib; vitamin B6; vitamin D; Vonoprazan; Voriconazole; Vortioxetine; Voxilaprevir; Warfarin Sodium; Zanubrutinib; Zinc; zoledronic acid; and Zolpidem.

Embodiment 57. The method of Embodiment 55 or 56, wherein the transdermal formulation further comprises an at least second medicament.

Embodiment 58. The method of Embodiment 57, wherein the first and the at least second medicament are selected from the group consisting of Abacavir and Dolutegravir and Lamivudine; adapalene and benzoyl peroxide; Amlodipine and Valsartan; amlodopine besylate and valsartan; Amoxicillin and Clavulanate; ampicillin and sulbactam; Artemether and Lumefantrine; betamethasone valerate and fusidic acid; Bictegravir and Emtricitabine and TAF; Bimatoprost and timolol maleate; Brimonidine and Timolol; Calcipotriene and betamethasone dipropionate; Canagliflozin and Metformin; Carbidopa and levodopa; Cefoperazone and Sulbactam; Dabrafenib and Trametinib; Darunavir and cobicistat and emtricitabine and tenofovir alafenamide; Diflucortolone and isoconazole; Drospirenone and Ethinyl Estradiol; Efavirenz and Emtricitabine and TDF; Elbasvir and Grazoprevir; Elvitegravir and Cobicistat and Emtricitabine and TAF; Emtricitabine and Rilpivirine and TAF; Emtricitabine and TAF; Emtricitabine and Tenofovir Disoproxil Fumarate; Ethinyl Estradiol and Etonogestrel; Ezetimibe and Atorvastatin; Ezetimibe and Simvastatin; fluocinolone acetonide and hydroquinone and tretinoin; fusidic acid and hydrocortisone acetate; Glecaprevir and Pibrentasvir; Hydrochlorothiazide and Valsartan; Irbesartan and Hydrochlorothiazide; Ledipasvir and Sofosbuvir; Linagliptin and Metformin; Losartan and Hydrochlorothiazide; Metformin and Sitagliptin; Norethindrone Acetate and Ethinyl Estradiol; Piperacillin and tazobactam; pregabalin and celecoxib; Quinupristin and dalfopristin; Rilpivirine and Emtricitabine and TDF; Sofosbuvir and Velpatasvir; Sofosbuvir and Velpatasvir and Voxilaprevir; Tezacaftor and Ivacaftor; trametinib and dabrafenib; Trametinib and dabrafenib; and Zinc and magnesium and vitamin B6.

Embodiment 59. The formulation of any one of Embodiments 1 to 53, wherein the penetrant portion further comprises safflower seed oil in an amount of from about 1% to about 5% w/w.

Embodiment 60. The formulation of any one of Embodiments 1 to 53, wherein the penetrant portion further comprises oleic acid in an amount of from about 0.1% to about 2% w/w.

Embodiment 61. The formulation of any one of Embodiments 1 to 53, wherein the penetrant portion further comprises hydrochloric acid in an amount of from about 0.01% to about 1% w/w.

Embodiment 62. The formulation of any one of Embodiments 1 to 62, wherein transdermal delivery provides systemic administration of the medicament.

Embodiment 63. A method for treating a disease or disorder or reducing a symptom thereof, the method comprising: administering to a subject in need thereof a transdermal formulation of any one of Embodiments 1 to 62 and administering to the subject in need thereof a composition comprising an one or more medicaments selected from Table 1.

Embodiment 64. The method of Embodiment 63, wherein the transdermal formulation is administered before, contemporary with, or after the composition is administered.

Embodiment 65. The method of Embodiment 63 or Embodiment 64, wherein the amount of the one or more medicament is the effective dose of the medicament as described in Table 1.

Embodiment 66. The method of any one of Embodiments 63 to 65, wherein the composition is administered by the standard route for the medicament.

Embodiment 67. The method of Embodiment 66, wherein the standard route is oral, topical, enteral, parenteral, by intravenous injection or infusion, by intraperitoneal injection, by intramuscular injection, or by subcutaneous injection.

Embodiment 68. The method of Embodiment 66 or Embodiment 67, wherein the composition is a liquid, a suspension, a gel, a geltab, a semisolid, a tablet, a sachet, a lozenge, a pill, or a capsule.

Any aspect or embodiment described herein can be combined with any other aspect or embodiment as disclosed herein.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can in certain instances be used interchangeably.

As used in the specification and claims, the singular forms “a”, “an” and “the” include plural references unless the context clearly dictates otherwise.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” mean A alone, B alone, C alone, A and B together, A and C together, B and C together, or A, B and C together.

As used herein, “or” may refer to “and”, “or,” or “and/or” and may be used both exclusively and inclusively. For example, the term “A or B” may refer to “A or B”, “A but not B”, “B but not A”, and “A and B”. In some cases, context may dictate a particular meaning.

As used herein, the term “about” a number refers to that number plus or minus 10% of that number and/or within one standard deviation (plus or minus) from that number. The term “about” a range refers to that range minus 10% of its lowest value and plus 10% of its greatest value and that range minus one standard deviation its lowest value and plus one standard deviation of its greatest value.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The terms “increased”, “increasing”, or “increase” are used herein to generally mean an increase by a statically significant amount relative to a reference level. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease in a value relative to a reference level. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “medicament,” “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

In an embodiment, a “pharmaceutical composition” is intended to include the combination of an active agent with a carrier, inert or active, in a sterile composition suitable for diagnostic or therapeutic use in vitro, in vivo or ex vivo. In one aspect, the pharmaceutical composition is substantially free of endotoxins or is non-toxic to recipients at the dosage or concentration employed.

In an embodiment, “an effective amount” refers to the amount of the defined component sufficient to achieve the desired chemical composition or the desired biological and/or therapeutic result. In an embodiment, that result can be the desired pH or chemical or biological characteristic, e.g., stability of the formulation. In other embodiments, the desired result is the alleviation or amelioration of the signs, symptoms, or causes of a disease, or any other desired alteration of a biological system. When the desired result is a therapeutic response, the effective amount will vary depending upon the specific disease or symptom to be treated or alleviated, the age, gender and weight of the subject to be treated, the dosing regimen of the formulation, the severity of the disease condition, the manner of administration and the like, all of which can be determined readily by one of skill in the art. A desired effect may, without necessarily being therapeutic, also be a cosmetic effect, in particular for treatment for disorders of the skin or muscles.

In an embodiment, as used herein, the terms “treating,” “treatment” and the like are used herein to mean obtaining a desired pharmacologic and/or physiologic effect. The effect may be prophylactic in terms of completely or partially preventing a disorder or sign or symptom thereof, and/or may be therapeutic in terms of amelioration of the symptoms of the disease or infection, or a partial or complete cure for a disorder and/or adverse effect attributable to the disorder.

The term “bioavailability” refers to the fraction of an administered dose of unchanged drug that reaches the systemic circulation. For example, when a medication is administered intravenously, its bioavailability is 100%. However, when a medication is administered via other routes (such as orally), its bioavailability generally decreases due to incomplete absorption and first-pass metabolism. Bioavailability is one of the essential tools in pharmacokinetics, as bioavailability must be considered when calculating dosages for non-intravenous routes of administration.

The term “lecithin organogel” or “LO” refers to a micellar system for the delivery of bioactive agents through the skin. LOs can be composed of hydrated phospholipids and appropriate organic liquid. Several therapeutic agents have been formulated as LOs for their facilitated transport through topical route (for dermal or transdermal effect). The improved topical drug delivery has mainly been attributed to the biphasic drug solubility, the desired drug partitioning, and the modification of skin barrier function by the organogel components.

For purposes herein, the terms lecithin and lecithin organogel are used interchangeably and both refer to, include and cover a lecithin organogel that comprises any group of yellow-brownish fatty substances occurring in animal and plant tissues which are amphiphilic and include a mixture of one or more of glycerophospholipids including phosphatidylcholine, phosphatidylethanolamine, phosphatidylinositol, phosphatidylserine, and phosphatidic acid.

Additionally, the use of particular formulations can disrupt the balance of electrolytes and cations, including those such as the Na/K ratio. For example, the administration of formulations containing calcium carbonate can reduce the amount of sodium or other ions which can decrease the potential for reaching a hyponatremic state. Also, the use of calcium carbonate can also increase the serum levels of calcium which can reduce the amount of calcium leeched from the body by high sodium concentrations.

The formulations and methods of use provided herein take these complexities of electrolyte balance into account. One approach utilized herein in making formulations that avoid electrolyte imbalance and cation overload is to use non-metal buffers or buffers without counterions. Suitable buffering agents for these embodiments include Lysine (free base), TRIS, and IEPA.

For transdermal topical administration in particular for agents other than buffer, a suitable formulation typically involves a penetrant that enhances penetration of the skin and is, in some embodiments, composed of chemical permeation enhancers (CPEs). In some cases, it can also include peptides designed to penetrate cells i.e. cell penetrating peptides (CPPs) also known as skin penetrating peptides (SPPs). The formulation can be applied for example in the form of topical lotions, creams, and the like, as described herein. Many known and useful compounds and the like can be found in Remington's Pharmaceutical Sciences (13^{th} Ed), Mack Publishing Company, Easton, PA—a standard reference for various types of administration. As used herein, the term “formulation(s)” means a combination of at least one active ingredient with one or more other ingredient, also commonly referred to as excipients, which may be independently active or inactive. The term “formulation” may or may not refer to a pharmaceutically acceptable composition for administration to humans or animals and may include compositions that are useful intermediates for storage or research purposes.

For purposes herein, a formulation, a formulation for transdermal delivery and a transdermal delivery formulation are each a formulation for transdermal delivery, including, the transdermal delivery of an active ingredient for the treatment of a syndrome and or a disease in an individual.

The section headings used herein are for organizational purposes only and are not to be construed as limiting the subject matter described.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

In one embodiment, a transdermal delivery formulation disclosed herein is capable of reducing the signs/symptoms associated with an ailment in an individual suffering from the ailment by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an anti-cancer transdermal delivery formulation is capable of reducing the number of cancer cells or tumor size in an individual suffering from a cancer by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In a further embodiment, a transdermal delivery formulation and its derivatives have half-lives of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In an embodiment, the period of administration of a transdermal delivery formulation is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In aspects of this embodiment, a therapeutically effective amount of a transdermal delivery formulation disclosed herein reduces or maintains signs/symptoms associated with an ailment in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a transdermal delivery formulation disclosed herein reduces or maintains signs/symptoms associated with an ailment by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a transdermal delivery formulation disclosed herein reduces or maintains signs/symptoms associated with an ailment by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

### Example 1: Transdermal Administration of Prednisone

Prednisone is used for many different autoimmune diseases and inflammatory conditions, including asthma and gout. In this example, a patient seeks treatment for gout after negligible improvements from other treatments. A health care professional suggests that the patient try transdermal prednisone. Conventionally, it is consumed at a single dose of 10 to 60 mg/day (orally).

Prednisone can be provided in a transdermal lotion or cream. The formulations described herein allow for effective transdermal administration of the compound. In this example, prednisone is included in the transdermal formulation detailed herein.

Prednisone Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius (safflower) oil | 2.93% |
| Oleic Acid | 0.97% |
| Polyglyceryl-4 Laurate | 1.06% |
| Deionized Water | 57.84% |
| Prednisone | 5.00% |
| Poloxamer 407 | 9.25% |
| Total | 100.00% |

Transdermal administration allows direct absorption into a specific area. For example, a lotion can be applied to an area close to where pain/inflammation is present. In this example, the patient applies the lotion to the feet.

The lotion can include a transdermal delivery formulation and the active agent (collectively referred to as the formulation). In this example, the dose of the active agent (i.e., prednisone) is 5% w/w of the solution. A transdermal medicament presents several benefits. The lack of interference with food and alcohol is one advantage. Topical delivery avoids the GI tract and can increase bioavailability. Increased bioavailability permits lower doses which reduce the risk of side effects. Prednisone topical cream obviates the need for the invasive nature and side effects of the injection of drugs. Topical administration also allows for the patient to increase the volume and incidence of application based on need/symptoms.

### Example 2: Transdermal Administration of Olmesartan Medoxomil

In this example, a patient seeks treatment for high blood pressure. The patient has already been treated unsuccessfully with other therapies including calcium channel blockers. A healthcare provider prescribes Olmesartan Medoxomil. Conventionally, it is administered orally as a single-dose of 20 mg.

Olmesartan Medoxomil can be provided in a transdermal lotion or cream. The formulations described herein allow for effective transdermal administration of the compound. In this example, Olmesartan Medoxomil is included in the transdermal formulation detailed herein.

Olmesartan Medoxomil Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius (safflower) oil | 2.93% |
| Oleic Acid | 0.97% |
| Polyglyceryl-4 Laurate | 1.06% |
| Deionized Water | 59.84% |
| Olmesartan Medoxomil | 3.00% |
| Poloxamer 407 | 9.25% |
| Total | 100.00% |

The lotion or cream can include a transdermal delivery formulation and the active agent, Olmesartan Medoxomil. In this example, the active agent is 3% w/w of the solution. There are several benefits to transdermal administration. A topical cream obviates the need for the invasive nature and side effects of drugs taken orally. Moreover, the patient can apply the drug at increasing frequencies as tolerated and as directed by a physician.

### Example 3: Transdermal Administration of Acyclovir

Acyclovir can be used to treat herpes virus infections, including shingles and genital herpes. In this example, a patient is ill from chicken pox. A health can provider prescribes acyclovir. Conventionally, ticagrelor is administered at a dose of 200 mg taken five timer per day for ten days.

In this example, acyclovir is provided in a transdermal lotion or cream. The formulations described herein allow for effective transdermal administration of the compound. In this example, acyclovir is included in the transdermal formulation detailed herein. Transdermal administration allows direct absorption into a specific area. For example, a lotion can be applied to a near the areas where lesions or inflammation is present.

Acyclovir Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius (safflower) oil | 2.69% |
| Oleic Acid | 0.89% |
| Polyglyceryl-4 Laurate | 1.00% |
| Deionized Water | 55.85% |
| Acyclovir | 10.00% |
| Poloxamer 407 | 8.49% |
| Total | 100.00% |

The lotion or cream can include a transdermal delivery formulation and the active agent, acyclovir. In this example, acyclovir is 10% w/w of the solution. The lotion/cream can be applied daily and/or as recommended by a physician. It can be reapplied as needed.

### Example 4: Transdermal Administration of Azithromycin

In this example, a patient is diagnosed with hepatocellular carcinoma (HCC). The patient is treated with sorafenib but there is no significant improvement. A healthcare provider recommends azithromycin as an alternative treatment. Conventionally, azithromycin is administered at 1200 mg orally once per week.

Azithromycin can be provided in a transdermal lotion or cream. The formulations described herein allow for effective transdermal administration of the compound. In this example, azithromycin is included in the transdermal formulation detailed herein.

Azithromycin Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius (safflower) oil | 2.69% |
| Oleic Acid | 0.89% |
| Polyglyceryl-4 Laurate | 1.00% |
| Deionized Water | 50.85% |
| Azithromycin | 15.00% |
| Poloxamer 407 | 8.49% |
| Total | 100.00% |

Transdermal administration allows for the patient to apply the drug daily and continue administration at the discretion of a physician. It also obviates the need for the invasive nature and side effects of the injection of drugs. The lotion can include a transdermal delivery formulation and the active agent (collectively referred to as the formulation). It can be applied at or near the location of a tumor. In this example, the active agent (i.e., azithromycin) is 15% of the lotion.

### Example 5: Transdermal Administration of Selpercatinib

In this example, a patient is diagnosed with thyroid cancer. A healthcare provider recommends selpercatinib as an alternative treatment. Conventionally, it is administered at around 80 mg BID (orally).

Selpercatinib is provided in a transdermal lotion or cream. The formulations described herein allow for effective transdermal administration of the compound. In this example, selpercatinib is included in the transdermal formulation detailed herein.

Selpercatinib Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius (safflower) oil | 2.69% |
| Oleic Acid | 0.89% |
| Polyglyceryl-4 Laurate | 1.00% |
| Deionized Water | 50.85% |
| Selpercatinib | 15.00% |
| Poloxamer 407 | 8.49% |
| Total | 100.00% |

Transdermal administration can be more effective with the formulations described herein. For example, when applied with the transdermal delivery formulation, the active agent is absorbed without being subjected to the gastrointestinal tract. Imatinib topical cream can be applied in small amounts, thus reducing the side effects. The formulation can be applied to the skin around the patient's neck, close to the tumor. Further, when more of the active agent is absorbed, it can be more effective at reducing tumor size/growth.

### Example 6: Transdermal Administration of Levetiracetam

In this example, a patient is diagnosed with epilepsy after experiencing a seizure. The patient suffers from side-effects when administered traditional anti-seizure medications. A health care professional suggests that the patient try levetiracetam. FC is conventionally administered as orally (500 mg) every twelve hours.

Levetiracetam can be provided in a transdermal lotion or cream. The formulations described herein allow for effective transdermal administration of the compound. In this example, levetiracetam is included in the transdermal formulation detailed herein.

Levetiracetam Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius | 2.69% |
| (safflower) oil | |
| Oleic Acid | 0.89% |
| Polyglyceryl-4 | 1.00% |
| Laurate | |
| Deionized Water | 35.85% |
| Levetiracetam | 30.00% |
| Poloxamer 407 | 8.49% |
| Total | 100.00% |

Transdermal administration allows direct absorption into the blood stream. Transdermal administration also allows for the patient to apply the drug daily and continue regular administration at the discretion of a physician. The patient experiences minimal side-effects compared to oral use of the agent. In this example, the active agent (i.e., levetiracetam) is 30% of the transdermal formulation.

### Example 7: Transdermal Administration of Neratinib

In this example, a patient is diagnosed with metastatic HER-2 positive breast cancer. The patient has been receiving trastuzumab based therapy to treat the patient's breast cancer. A health care professional suggests that the patient start taking neratinib to complement the trastuzumab. Neratinib is administered each day orally (40 mg-360 mg) every day.

Neratinib can be provided in a transdermal lotion or cream. The formulations described herein allow for effective transdermal administration of the compound. In this example, neratinib is included in the transdermal formulation detailed herein.

Neratinib Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius | 2.69% |
| (safflower) oil | |
| Oleic Acid | 0.89% |
| Polyglyceryl-4 | 1.00% |
| Laurate | |
| Deionized Water | 35.85% |
| Neratinib | 30.00% |
| Poloxamer 407 | 8.49% |
| Total | 100.00% |

Transdermal administration allows direct absorption into the blood stream. Transdermal administration also allows for the patient to apply the drug daily and continue regular administration at the discretion of a physician. The patient experiences minimal side-effects compared to oral use of the agent. In this example, the active agent (i.e., neratinib) is provided at a dose of 240 mg per dose of the transdermal formulation.

In addition, the following formulations of neratinib were developed for transdermal administration, as detailed herein.

Neratinib Formulation

| Ingredient | Weight (%) |
|---|---|
| Phosphatidylcholine | 6.63% |
| Isopropyl Palmitate | 11.54% |
| Stearic Acid | 0.54% |
| Benzyl Alcohol | 1.21% |
| Carthamus Tinctorius | 2.54% |
| (safflower) oil | |
| Oleic Acid | 0.84% |
| Polyglyceryl-4 | 0.92% |
| Laurate | |
| Deionized Water | 52.77% |
| Neratinib | 14.99% |
| Poloxamer 407 | 8.02% |
| Total | 100.00% |

Neratinib Formulation

| Ingredient | Weight (%) |
|---|---|
| Phosphatidylcholine | 7.48% |
| Isopropyl Palmitate | 13.02% |
| Stearic Acid | 0.60% |
| Benzyl Alcohol | 1.36% |
| Carthamus Tinctorius | 2.87% |
| (safflower) oil | |
| Oleic Acid | 0.95% |
| Polyglyceryl-4 | 1.04% |
| Laurate | |
| Deionized Water | 59.30% |
| Neratinib | 4.23% |
| Poloxamer 407 | 9.05% |
| hydrochloric acid | 0.10% |
| Total | 100.00% |

Individual ingredients of the formulations listed above may be substituted with other reagents. For example, isopropyl palmitate may be substituted with isopropyl myristate. Benzyl alcohol may be substituted with ethanol. Poloxamer 407 may be substituted with poloxamer 188 or poloxamer 124. Stearic acid may be substituted with palmitic acid. Safflower seed oil may be substituted with almond oil, linoleic acid, macadamia oil, or an oil with high linoleic composition. Polyglyceryl-4-laurate may be substituted with another oil/water emulsifier.

The term “neratinib” also known as “neratinib maleate” and Nerlynx refers to a medication that acts as an inhibitor of HER-2 receptor tyrosine kinase, epidermal growth factor (“EGFR”) and the proliferation of EGFR-dependent cells for the treatment of neoplastic activity, including HER-2 positive breast cancer. Neratinimb is used in the extended adjuvant therapy of early-stage breast cancer. Neratinib is associated with a low rate of transient elevations in serum aminotransferase levels during therapy. Neratinib is a quinoline compound having a cyano group at the 3-position, a 3-chloro-4-(2-pyridylmethoxy)anilino group at the 4-position, a 4-dimethylamino-trans-but-2-enoylamido group at the 6-position, and an ethoxy group at the 7-position. Its IUPAC name is (E)-N-[4-[3-chloro-4-(pyridin-2-ylmethoxy)anilino]-3-cyano-7-ethoxyquinolin-6-yl]-4-(dimethylamino)but-2-enamide. Neratinib can be administered along with trastuzumab based therapy and/or capecitabine.

Neratinib dosing is from about 40 mg to about 360 mg. In some embodiments, the dose of neratinib is about 40 mg, about 60 mg, about 80 mg, about 100 mg, about 120 mg, about 140 mg, about 160 mg, about 180 mg, about 200 mg, about 220 mg, about 240 mg, about 260 mg, about 280 mg, about 300 mg, about 320 mg, about 340 mg, or about 360 mg, and any dose therebetween.

In various embodiments, the dose of neratinib is about 40 mg to about 100 mg, about 40 mg to about 60 mg, about 40 mg to about 80 mg, about 60 mg to about 100 mg, about 60 mg to about 100 mg, about 60 mg to about 120 mg, about 60 mg to about 80 mg, about 80 mg to about 100 mg, about 80 mg to about 120 mg, about 80 mg to about 140 mg, about 100 mg to about 120 mg, about 100 mg to about 140 mg, about 100 mg to about 160 mg, about 120 mg to about 140 mg, about 120 mg to about 160 mg, about 120 mg to about 180 mg, about 140 mg to about 160 mg, about 140 mg to about 180 mg, about 140 mg to about 200 mg, about 160 mg to about 180 mg, about 160 mg to about 200 mg, about 160 mg to about 220 mg, about 180 mg to about 200 mg, about 180 mg to about 220 mg, about 180 mg to about 240 mg, about 200 mg to about 220 mg, about 200 mg to about 220 mg, about 200 mg to about 240 mg, about 200 mg to about 260 mg, about 220 mg to about 240 mg, about 220 mg to about 260 mg, about 220 mg to about 260 mg, about 220 mg to about 280 mg, about 240 mg to about 260 mg, about 240 mg to about 280 mg, about 240 mg to about 300 mg, about 260 mg to about 280 mg, about 260 mg to about 300 mg, about 260 mg to about 320 mg, about 280 mg to about 300 mg, about 280 mg to about 320 mg, about 280 mg to about 340 mg, about 300 mg to about 320 mg, about 300 mg to about 340 mg, about 300 mg to about 360 mg, about 320 mg to about 340 mg, or about 340 mg to about 360 mg.

Neratinib dosing is about 40 mg per day, about 60 mg per day, about 80 mg per day, about 100 mg per day, about 120 mg per day, about 140 mg per day, about 160 mg per day, about 180 mg per day, about 200 mg per day, about 220 mg per day, about 240 mg per day, about 260 mg per day, about 280 mg per day, about 300 mg per day, about 320 mg per day, about 340 mg per day, or about 360 mg per day.

In some cases, the daily dose of neratinib is 240 mg.

In various embodiments, a formulation comprising neratinib is about 2% (w/w) neratinib to about 30% (w/w) neratinib. In some embodiments, a formulation comprising neratinib is about 2% (w/w) neratinib, about 3% (w/w) neratinib, about 4% (w/w) neratinib, about 5% (w/w) neratinib, about 6% (w/w) neratinib, about 7% (w/w) neratinib, about 8% (w/w) neratinib, about 9% (w/w) neratinib, about 10% (w/w) neratinib, about 11% (w/w) neratinib, about 12% (w/w) neratinib, about 13% (w/w) neratinib, about 14% (w/w) neratinib, about 15% (w/w) neratinib, about 16% (w/w) neratinib, about 17% (w/w) neratinib, about 18% (w/w) neratinib, about 19% (w/w) neratinib, about 20% (w/w) neratinib, about 21% (w/w) neratinib, about 22% (w/w) neratinib, about 23% (w/w) neratinib, about 24% (w/w) neratinib, about 25% (w/w) neratinib, about 26% (w/w) neratinib, about 27% (w/w) neratinib, about 28% (w/w) neratinib, about 29% (w/w) neratinib, or about 30% (w/w) neratinib, and any percentage of neratinib therebetween.

In embodiments, a formulation comprising neratinib is about 15% (w/w) neratinib.

In some embodiments, a formulation comprising neratinib is about 2% to about 3%, about 2% to about 4%, about 2% to about 5%, about 3% to about 4%, about 3% to about 5%, about 3% to about 6%, about 4% to about 5%, about 4% to about 6%, about 4% to about 7%, about 5% to about 6%, about 5% to about 7%, about 5% to about 8%, about 6% to about 7%, about 6% to about 8%, about 6% to about 9%, about 7% to about 8%, about 7% to about 9%, about 7% to about 10%, about 8% to about 9%, about 8% to about 10%, about 8% to about 11%, about 9% to about 10%, about 9% to about 11%, about 9% to about 12%, about 10% to about 11%, about 10% to about 12%, about 10% to about 13%, about 11% to about 12%, about 11% to about 13%, about 11% to about 14%, about 12% to about 13%, about 12% to about 14%, about 12% to about 15%, about 13% to about 14%, about 13% to about 15%, about 13% to about 16%, about 14% to about 15%, about 14% to about 16%, about 14% to about 17%, about 15% to about 16%, about 15% to about 17%, about 15% to about 18%, about 16% to about 17%, about 16% to about 18%, about 16% to about 19%, about 17% to about 18%, about 17% to about 19%, about 17% to about 20%, about 18% to about 19%, about 18% to about 20%, or about 19% to about 20%, about 20% to about 21%, about 20% to about 22%, about 20% to about 23%, about 20% to about 24%, about 21% to about 22%, about 21% to about 23%, about 21% to about 24%, about 21% to about 25%, about 22% to about 23%, about 22% to about 24%, about 22% to about 25%, about 22% to about 26%, about 23% to about 24%, about 23% to about 25%, about 23% to about 26%, about 23% to about 27%, about 24% to about 25%, about 24% to about 26%, about 24% to about 27%, about 24% to about 28%, about 25% to about 26%, about 25% to about 27%, about 25% to about 28%, about 25% to about 29%, about 26% to about 27%, about 26% to about 28%, about 26% to about 29%, about 26% to about 30%, about 27% to about 28%, about 27% to about 29%, about 27% to about 30%, about 28% to about 29%, about 28% to about 30%, or about 29% to about 30% (w/w) neratinib.

The Neratinib formulations disclosed herein were tested in rodents to evaluate their effects of transdermal delivery of active reagents. Pharmacokinetics (PK) data from animal models demonstrated the transdermal delivery of active reagents at clinically relevant blood levels in light of the effects of oral delivery based on a list of parameters, including Cmax (peak concentration), AUC (area under the curve), Tmax (the time taken to reach Cmax), T1/2 (elimination half time), AUCinf (area under plasma concentration-time curve from time 0 to infinity), AUClast (Area under the plasma concentration-time curve from time zero to time of last measurable concentration), and surrogate delivery markers, as shown below.

| | | Applied | Cmax | | | AUCinf | AUClast |
|---|---|---|---|---|---|---|---|
| Formulation | | Active | (ng/ | Tmax | T 1/2 | (h*ng/ | (h*ng/ |
| Coding | Description | Dose (ug) | mL) | (h) | (h) | mL) | mL) |
| Neratinib | 15% neratinib free | 15000 | 2290 | 4 | nd | nd | 13737 |
| (Free Base) A | base in W/O | | | | | | |
| Neratinib | Breadth base, pH | 3500 | 1382 | 4 | 2.61 | 8640 | 7493 |
| (Free Base) B | adjusted, 3.5% | | | | | | |
| | neratinib free base at | | | | | | |
| | end | | | | | | |
| Neratinib | 3.5% neratinib free | 3500 | 2498 | 2 | 1.96 | 12993 | 12045 |
| (Free Base) C | base in W/O | | | | | | |
| Neratinib | 4.23% neratinib | 4230 | 1742 | 1 | nd | nd | 3401 |
| (Maleate) D | maleate in W/O | | | | | | |
| Neratinib | Breadth base, pH | 4230 | 1175 | 4 | 2.52 | 7964 | 7076 |
| (Maleate) E | adjusted, 4.23% | | | | | | |
| | neratinib maleate at | | | | | | |
| | end | | | | | | |
| Neratinib | Breadth base, pH | 4230 | 2636 | 0.5 | nd | nd | 11615 |
| (Maleate) F | adjusted, 4.23% | | | | | | |
| | neratinib maleate mix | | | | | | |
| | on site | | | | | | |

### Example 8: Pharmacokinetics Study of Cream Formulations for Total Vancomycin Concentration in Adult Mice

In this example, five formulations of a topical vancomycin cream was applied to adult mice and plasma pharmacokinetics (PK) values for vancomycin were measured.

Each of the five formulations included the following components: Phosphatidylcholine, Isopropyl Palmitate, Stearic Acid, Benzyl Alcohol, Carthamus Tinctorius (safflower) oil, Oleic Acid, Polyglyceryl-4 Laurate, Deionized Water, Vancomycin, and Poloxamer 407. The five formulations differed, at least, in the amount of vancomycin present with Formulation C, Formulation 2A, and Formulation 2B comprising 8% vancomycin; Formulation 2C comprising 15% vancomycin; and Formulation 2D comprising 20% vancomycin, such that Formulations C, 2A, and 2B provided a dose of 533.3 mg vancomycin/kg animal; Formulation 2C provided a dose of 1000 mg vancomycin/kg animal; and Formulation 2D provided a dose of 1333.3 mg vancomycin/kg animal, each when administered 100 μL. Formulations C, 2A, and 2B, each comprising 8% vancomycin, differed slightly in the carrier in an effort to determine if the changes improved delivery. One was simply a standard formulation, another was the standard formulation with a basic excipient added to increase the pH, and yet another was the standard formulation with an additional excipient that was added to potentially enhance penetration.

The formulations used in this example were similar (e.g., in the relative amounts of each component) to those described in the previous examples, yet including vancomycin rather than the other listed compound. Generally, a vancomycin formulation comprised the following ingredients and at the listed weight percentages:

General Vancomycin Formulations

| Ingredient | Weight (%) |
|---|---|
| Phosphatidylcholine | 5-15% |
| Isopropyl Palmitate | 10-20% |
| Stearic Acid | 0.5-2% |
| Benzyl Alcohol | 0.5-2% |
| Carthamus Tinctorius | 1-5% |
| (safflower) oil | |
| Oleic Acid | 0.5-2% |
| Polyglyceryl-4 Laurate | 0.5-2% |
| Deionized Water | 20-80% |
| Poloxamer 407 | 5-15% |
| Vancomycin | 8-20% |

In this example, six week or older, male mice were used in the study. A region ˜2×3 cm on the backs of each mouse was shaved 24 hours before experiment. Animals were food deprived for one hour before topical administration and for four hours during the study, and with no limit of access to water. Each mouse was topically applied one formulation at a vancomycin dose of 100 μL/animal. The tested topical formulations were rubbed in at the selected dose. After rubbing, the animals were monitored for 15-20 min to avoid licking of the formulations. If licking is was observed, an Elizabethan collar was put on the mouse to prevent licking off the formulation. Three animals per formulation and per time point were used. Blood samples were collected after drug administration in the following time points by cardiopuncture:

| # of animal | Experimental | |
|---|---|---|
| 1, 2, 3 | Pre-application | |
| 4, 5, 6 | 30 | min |
| 7, 8, 9 | 1 | hour |
| 10, 11, 12 | 2 | hours |
| 13, 14, 15 | 4 | hours |
| 16, 17, 18 | 8 | hours |

Blood samples, approximately 0.5 mL each, were collected from each animal into tubes with coagulation activators. The samples were incubated at RT for ˜40 min and centrifuged (for ˜15 minutes at 3000 xg). The serum was harvested into a single tube for each animal at each time point. The serum samples were frozen (˜−80° C.) after centrifugation and transferred to Bioanalytical Laboratory.

Analyses were performed using an assay kit and multifunctional plate reader Infinite® M1000 PRO (Tecan) according to the manufacturer's protocol. Total vancomycin concentrations were calculated using a standard curve prepared according to the manufacturer's protocol from the kit.

Using the calibration curve, concentrations of vancomycin in the serum samples were calculated. Pharmacokinetic analysis was performed using WinNonlin Professional 6.3 software package (Pharsight Corporation, USA). The following parameters were estimated as a result of the studies: Cₘₐₓ (ng/ml for serum or ng/g for tissues)—the maximum concentration; Tₘₐₓ (h)—the time of maximum concentration; AUC_{0→∞} (ng*min/ml)—AUCinf—AUC from time zero extrapolated to infinity; AUC_{0→t} (ng*min/ml)—AUClast—AUC from time zero to the time of the last non-zero Y value (24 h); Kₑₗ (l/h)—first order rate constant associated with the terminal (log-linear) elimination phase; T_{1/2} (h)—HL_Lambda_z=0.693/Lambda Z; and MRTlast (h)—mean residence time from the time of dosing to the time of the last measurable concentration. Statistical analysis of the concentrations was performed by descriptive statistics. The following values were calculated: Arithmetic mean (Mean); Standard deviation (SD); Standard error of the mean (SEM); Coefficient of variation (CV). For the concentration outliers detecting and excluding Grubbs statistical test was used. (See, the World Wide Web (at) graphpad.com/support/faqid/1598/).

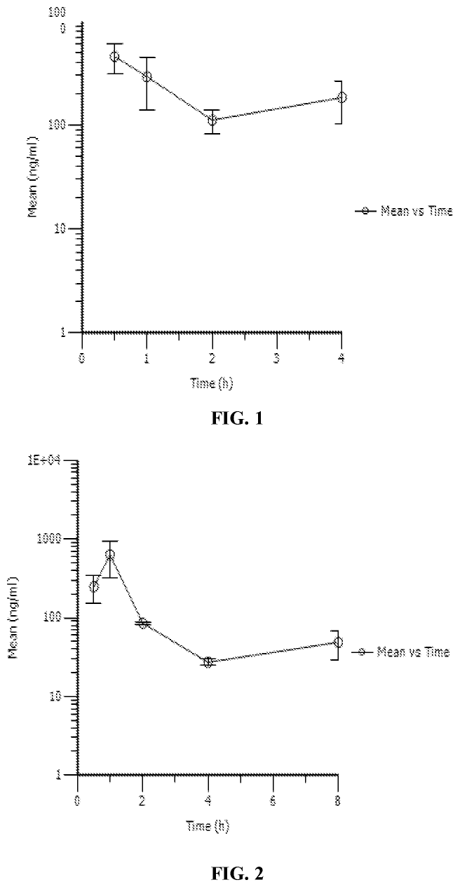

Data from the Formulation C mice is shown below in the following tables and the PK data is summarized in FIG. 1.

### Vancomycin, PK in Mouse Plasma, Dermal, 100 ul/Mouse, Vancomycin Creme Formulation C

| Time (h) | N | Mean (ng/ml) | SD (ng/ml) | SE (ng/ml) | CV % |
|---|---|---|---|---|---|
| 0 | 2 | 0.00 | 0.00 | 0.00 | |
| 0.5 | 3 | 458 | 250 | 144 | 54.5 |
| 1 | 3 | 292 | 263 | 152 | 90.0 |
| 2 | 3 | 112 | 49.6 | 28.6 | 44.3 |
| 4 | 3 | 184 | 139.1 | 80.3 | 75.6 |

| Parameter | Units | Estimate |
|---|---|---|
| kel | 1/h | nd |
| t½ | h | nd |
| Tmax | h | 0.5 |
| Cmax | ng/ml | 458 |
| AUClast | h*ng/ml | 800 |
| AUCINF | h*ng/ml | nd |
| MRTlast | h | 1.76 |

Data from the Formulation 2A mice is shown below in the following tables and the PK data is summarized in FIG. 2.

### Vancomycin, PK in Mouse Plasma, Dermal, 100 ul/Mouse, Vancomycin Cream Formulation 2 (A)

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 249 | 165 | 95.3 | 66.3 |
| 1 | 3 | 633 | 537 | 310 | 84.8 |
| 2 | 3 | 85.3 | 5.34 | 3.08 | 6.26 |
| 4 | 3 | 27.3 | 4.60 | 2.65 | 16.9 |
| 8 | 3 | 48.9 | 34.9 | 20.2 | 71.5 |

| Parameter | Units | Estimate |
|---|---|---|
| kel | 1/h | nd |
| t½ | h | nd |
| Tmax | h | 1 |
| Cmax | ng/ml | 633 |
| AUClast | h*ng/ml | 907 |
| AUCINF | h*ng/ml | nd |
| MRTlast | h | 2.10 |

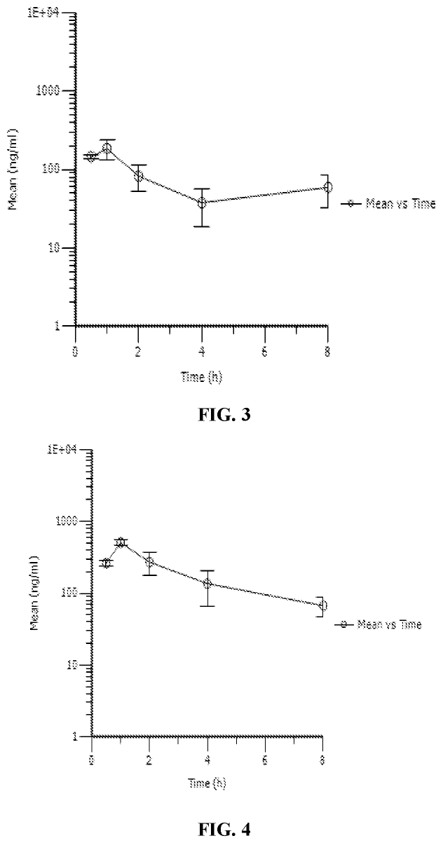

Data from the Formulation 2B mice is shown below in the following tables and the PK data is summarized in FIG. 3.

### Vancomycin, PK in Mouse Plasma, Dermal, 100 ul/Mouse, Vancomycin Cream Formulation 2B)

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 146 | 13.1 | 7.55 | 8.96 |
| 1 | 2 | 185 | 77.8 | 55.0 | 42.0 |
| 2 | 3 | 82.4 | 53.0 | 30.6 | 64.3 |
| 4 | 3 | 37.8 | 33.1 | 19.1 | 87.5 |
| 8 | 3 | 59.2 | 45.6 | 26.3 | 77.1 |

| Parameter | Units | Estimate |
|---|---|---|
| kel | 1/h | nd |
| t½ | h | nd |
| Tmax | h | 1 |
| Cmax | ng/ml | 185 |
| AUClast | h*ng/ml | 567 |
| AUCINF | h*ng/ml | nd |
| MRTlast | h | 3.21 |

Data from the Formulation 2C mice is shown below in the following tables and the PK data is summarized in FIG. 4.

### Vancomycin, PK in Mouse Plasma, Dermal, 100 ul/Mouse, Vancomycin Cream Formulation 2 (C)

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 263 | 43.6 | 25.1 | 16.6 |
| 1 | 3 | 509 | 79.3 | 45.8 | 15.6 |
| 2 | 3 | 271 | 165 | 95 | 60.8 |
| 4 | 3 | 136 | 123 | 70.9 | 90.0 |
| 8 | 3 | 67.3 | 35.5 | 20.5 | 52.8 |

| Parameter | Units | Estimate |
|---|---|---|
| kel | 1/h | 0.22 |
| t½ | h | 3.09 |
| Tmax | h | 1 |
| Cmax | ng/ml | 509 |
| AUClast | h*ng/ml | 1464 |
| AUCINF | h*ng/ml | 1764 |
| MRTlast | h | 2.71 |

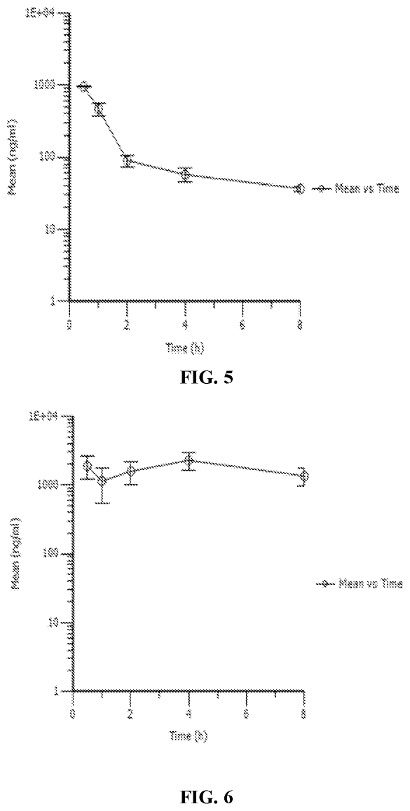

Data from the Formulation 2D mice is shown below in the following tables and the PK data is summarized in FIG. 5.

### Vancomycin, PK in Mouse Plasma, Dermal, 100 ul/Mouse, Vancomycin Cream Formulation 2 (D)

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 955 | 51.8 | 29.9 | 5.42 |
| 1 | 3 | 466 | 157 | 90.5 | 33.7 |
| 2 | 3 | 90.1 | 28.6 | 16.5 | 31.7 |
| 4 | 3 | 57.4 | 20.9 | 12.1 | 36.4 |
| 8 | 3 | 36.7 | 4.86 | 2.80 | 13.2 |

| Parameter | Units | Estimate |
|---|---|---|
| kel | 1/h | 0.14 |
| t½ | h | 4.80 |
| Tmax | h | 0.5 |
| Cmax | ng/ml | 955 |
| AUClast | h*ng/ml | 1208 |
| AUCINF | h*ng/ml | 1462 |
| MRTlast | h | 1.77 |

### Example 9: Pharmacokinetics Study of Cream Formulations for Total Neratinib Concentration in Adult Mice

In this example, six formulations of a topical neratinib cream was applied to adult mice and plasma pharmacokinetics (PK) values for neratinib were measured.

Each of the six formulations generally included the following components: Phosphatidylcholine, Isopropyl Palmitate, Stearic Acid, Benzyl Alcohol, Carthamus Tinctorius (safflower) oil, Oleic Acid, Polyglyceryl-4 Laurate, Deionized Water, neratinib, and Poloxamer 407. The six formulations differed, at least, in the amount of neratinib according to the following: Formulation A: 15% free base neratinib in general formulation, varied emulsion type (˜15 mg neratinib applied when administered 100 μL); Formulation B: 3.5% free base neratinib in general formulation (˜3.5 mg neratinib applied when administered 100 μL); Formulation C: 3.5% free base neratinib in general formulation, varied emulsion type (˜3.5 mg neratinib applied when administered 100 μL); Formulation D: 4.23% neratinib maleate (equivalent to 3.5% free base neratinib when administered 100 μL) in general formulation, varied emulsion type (˜4.23 mg neratinib maleate applied when administered 100 μL); Formulation E: 4.23% neratinib maleate in general formulation (˜4.23 mg neratinib maleate applied when administered 100 μL); Formulation F: DMAX base with 4.23% neratinib maleate added at point of use to prevent degradation (˜4.23 mg neratinib maleate applied when administered 100 μL). In these experiments, two different forms of the active neratinib were tested but blood levels of pure neratinib was assayed. In particular, the 3.5% free base and 4.23% neratinib maleate formulations are understood to have the same amount of pure neratinib in them.

The formulations used in this example were similar (e.g., in the relative amounts of each component) to those described in the previous examples, yet including neratinib rather than the other listed compound. Generally, a neratinib formulation comprised the following ingredients and at the listed weight percentages:

General Neratinib Formulations

| Ingredient | Weight (%) |
|---|---|
| Phosphatidylcholine | 5-15% |
| Isopropyl Palmitate | 10-20% |
| Stearic Acid | 0.5-2% |
| Benzyl Alcohol | 0.5-2% |
| Carthamus Tinctorius | 1-5% |
| (safflower) oil | |
| Oleic Acid | 0.5-2% |
| Polyglyceryl-4 Laurate | 0.5-2% |
| Deionized Water | 20-80% |
| Poloxamer 407 | 5-15% |
| Neratinib | 3-15% |

Additionally, a formulation for oral gavage was provided; this formulation comprised ˜110 mg/kg neratinib maleate in suspension in 0.5% methylcellulose and span 80 (˜1.65 mg neratinib maleate delivered per os).

In this example, six week or older, male mice were used in the study. A region ˜2×3 cm on the backs of each mouse was shaved 24 hours before experiment. Animals were food deprived for one hour before topical administration and for four hours during the study, and with no limit of access to water. Each mouse was topically applied one formulation at a neratinib dose of 100 μL/animal. The tested topical formulations were rubbed in at the selected dose. After rubbing, the animals were monitored for 15-20 min to avoid licking of the formulations. If licking is was observed, an Elizabethan collar was put on the mouse to prevent licking off the formulation. Three animals per formulation and per time point were used. Blood samples were collected after drug administration in the following time points by cardiopuncture:

| # of animal | Experimental | |
|---|---|---|
| 1, 2, 3 | Pre-application | |
| 4, 5, 6 | 30 | min |
| 7, 8, 9 | 1 | hour |
| 10, 11, 12 | 2 | hours |
| 13, 14, 15 | 4 | hours |
| 16, 17, 18 | 8 | hours |

Blood samples, approximately 0.5 mL each, were collected from each animal into tubes with coagulation activators. The samples were incubated at RT for ˜40 min and centrifuged (for ˜15 minutes at 3000 xg). The serum was harvested into a single tube for each animal at each time point. The serum samples were frozen (˜−80° C.) after centrifugation and transferred to Bioanalytical Laboratory.

Analyses were performed using an assay kit and multifunctional plate reader Infinite® M1000 PRO (Tecan) according to the manufacturer's protocol. Total neratinib concentrations were calculated using a standard curve prepared according to the manufacturer's protocol from the kit.

Using the calibration curve, concentrations of neratinib in the serum samples were calculated. Pharmacokinetic analysis was performed using WinNonlin Professional 6.3 software package (Pharsight Corporation, USA). The following parameters were estimated as a result of the studies: Cₘₐₓ (ng/ml for serum or ng/g for tissues)—the maximum concentration; Tₘₐₓ (h)—the time of maximum concentration; AUC_{0→∞} (ng*min/ml)—AUCinf—AUC from time zero extrapolated to infinity; AUC_{0→t} (ng*min/ml)—AUClast—AUC from time zero to the time of the last non-zero Y value (24 h); Kₑₗ (l/h) first order rate constant associated with the terminal (log-linear) elimination phase; T_{1/2} (h)—HL_Lambda_z=0.693/Lambda Z; and MRTlast (h)—mean residence time from the time of dosing to the time of the last measurable concentration. Statistical analysis of the concentrations was performed by descriptive statistics. The following values were calculated: Arithmetic mean (Mean); Standard deviation (SD); Standard error of the mean (SEM); Coefficient of variation (CV). For the concentration outliers detecting and excluding Grubbs statistical test was used. (See, the World Wide Web (at) graphpad.com/support/faqid/1598/).

Data from the Formulation A mice is shown below in the following tables and the PK data is summarized in FIG. 6.

### Neratinib, in Mouse Plasma, Dermal, 100 ul/Mouse, Creme Formulation A

| Time (h) | N | Mean (ng/ml) | SD (ng/ml) | SE (ng/ml) | CV % |
|---|---|---|---|---|---|
| 0 | 3 | 0 | 0 | 0 | |
| 0.5 | 3 | 1905 | 1233 | 712 | 64.7 |
| 1 | 3 | 1139 | 1048 | 605 | 92.0 |
| 2 | 3 | 1580 | 1006 | 581 | 63.7 |
| 4 | 3 | 2290 | 1158 | 668 | 50.5 |
| 8 | 3 | 1345 | 672 | 388 | 50.0 |

| Parameter | Units | Estimate |
|---|---|---|
| Tmax | h | 4 |
| Cmax | ng/ml | 2290 |
| AUClast | h*ng/ml | 13737 |
| MRTlast | h | 4.01 |

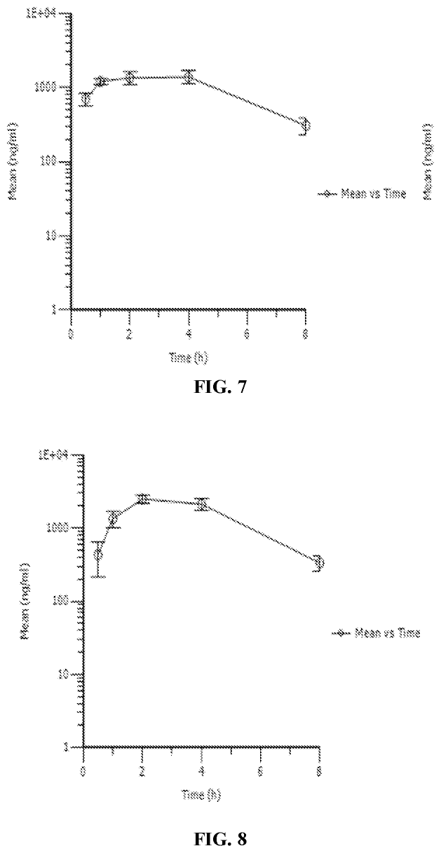

Data from the Formulation B mice is shown below in the following tables and the PK data is summarized in FIG. 7.

### Neratinib, in Mouse Plasma, Dermal, 100 ul/Mouse Creme Formulation B

| Time (h) | N | Mean (ng/ml) | SD (ng/ml) | SE (ng/ml) | CV % |
|---|---|---|---|---|---|
| 0 | 3 | 0 | 0 | 0 | |
| 0.5 | 3 | 695 | 222 | 128 | 32.0 |
| 1 | 3 | 1203 | 191 | 110 | 15.9 |
| 2 | 3 | 1341 | 463 | 267 | 34.5 |
| 4 | 3 | 1382 | 477 | 275 | 34.5 |
| 8 | 3 | 305 | 128 | 73.7 | 41.9 |

| Parameter | Units | Estimate |
|---|---|---|
| kel | 1/h | 0.27 |
| t½ | h | 2.61 |
| Tmax | h | 4 |
| Cmax | ng/ml | 1382 |
| AUClast | h*ng/ml | 7493 |
| AUCINF_obs | h*ng/ml | 8640 |
| MRTlast | h | 3.52 |

Data from the Formulation C mice is shown below in the following tables and the PK data is summarized in FIG. 8.

### Neratinib, in Mouse Plasma, Dermal, 100 ul/Mouse, Creme Formulation C

| Time (h) | N | Mean (ng/ml) | SD (ng/ml) | SE (ng/ml) | CV % |
|---|---|---|---|---|---|
| 0 | 3 | 0 | 0 | 0 | |
| 0.5 | 3 | 433 | 382 | 220.6 | 88.2 |
| 1 | 3 | 1349 | 565 | 326 | 41.9 |
| 2 | 2 | 2498 | 407 | 288 | 82.7 |
| 4 | 3 | 2133 | 678 | 391 | 31.8 |
| 8 | 3 | 335 | 139 | 80.0 | 41.4 |

| Parameter | Units | Estimate |
|---|---|---|
| kel | 1/h | 0.35310608 |
| t½ | h | 1.96 |
| Tmax | h | 2 |
| Cmax | ng/ml | 2498 |
| AUClast | h*ng/ml | 12045 |
| AUCINF_obs | h*ng/ml | 12993 |
| MRTlast | h | 3.29 |

Data from the Formulation D mice is shown below in the following tables and the PK data is summarized in FIG. 9.

### Neratinib in Mouse Plasma, Dermal, 100 ul/Mouse Creme Formulation D

| Time (h) | N | Mean (ng/ml) | SD (ng/ml) | SE (ng/ml) | CV % |
|---|---|---|---|---|---|
| 0 | 3 | 43.2 | 8.23 | 4.75 | 19.0 |
| 0.5 | 3 | 1157 | 253 | 146 | 21.9 |
| 1 | 3 | 1742 | 2094 | 1209 | 120 |
| 2 | 3 | 119 | 116 | 66.9 | 97.5 |
| 4 | 3 | 234 | 173 | 100 | 74.2 |
| 8 | 3 | 313 | 259 | 149 | 82.7 |

| Parameter | Units | Estimate |
|---|---|---|
| Tmax (≈) | h | 1 |
| Cmax | ng/ml | 1742 |
| AUClast | h*ng/ml | 3401 |
| MRTlast | h | 2.87 |

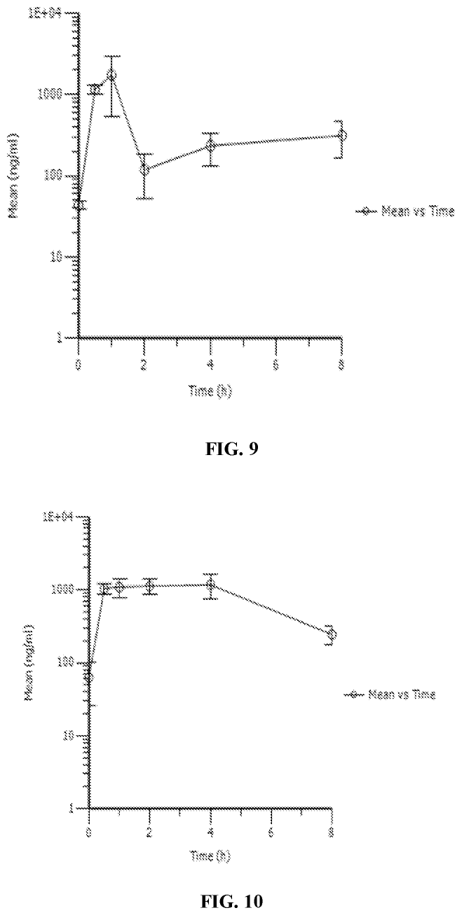

Data from the Formulation E mice is shown below in the following tables and the PK data is summarized in FIG. 10.

### Neratinib, in Mouse Plasma, Dermal, 100 ul/Mouse, Creme Formulation E

| Time (h) | N | Mean (ng/ml) | SD (ng/ml) | SE (ng/ml) | CV % |
|---|---|---|---|---|---|
| 0 | 3 | 63.3 | 64.1 | 37.0 | 101 |
| 0.5 | 3 | 1050 | 311 | 179 | 29.6 |
| 1 | 3 | 1096 | 554 | 320 | 50.6 |
| 2 | 3 | 1132 | 473 | 273 | 41.8 |
| 4 | 3 | 1175 | 740 | 427 | 62.9 |
| 8 | 3 | 244 | 121 | 70.1 | 49.7 |

| Parameter | Units | Estimate |
|---|---|---|
| kel | 1/h | 0.275 |
| t½ | h | 2.52 |
| Tmax | h | 4 |
| Cmax | ng/ml | 1175 |
| AUClast | h*ng/ml | 7076 |
| AUCINF_obs | h*ng/ml | 7964 |
| MRTlast | h | 3.18 |

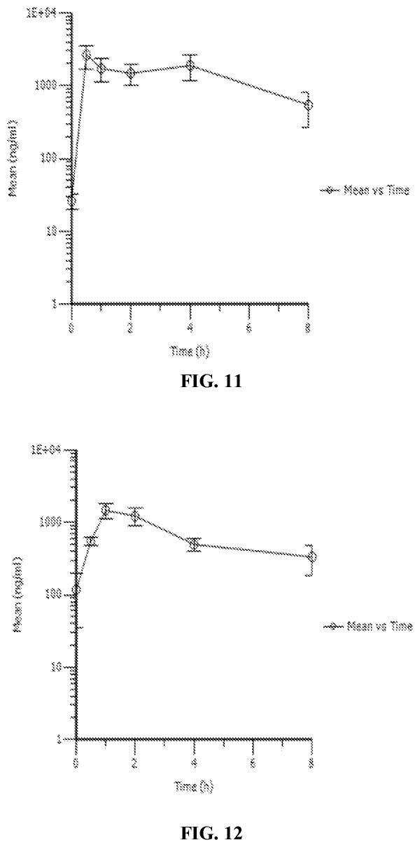

Data from the Formulation F mice is shown below in the following tables and the PK data is summarized in FIG. 11.

### Neratinib in Mouse Plasma, Dermal, 100 ul/Mouse Creme Formulation F

| Time (h) | N | Mean (ng/ml) | SD (ng/ml) | SE (ng/ml) | CV % |
|---|---|---|---|---|---|
| 0 | 3 | 26.6 | 10.8 | 6.22 | 40.5 |
| 0.5 | 3 | 2636 | 1591 | 918 | 60.3 |
| 1 | 3 | 1726 | 1037 | 598 | 60.1 |
| 2 | 3 | 1483 | 794 | 459 | 53.6 |
| 4 | 3 | 1894 | 1268 | 732 | 67.0 |
| 8 | 3 | 544 | 476 | 275 | 87.5 |

| Parameter | Units | Estimate |
|---|---|---|
| Tmax | h | 0.5 |
| Cmax | ng/ml | 2636 |
| AUClast | h*ng/ml | 11615 |
| MRTlast | h | 3.26 |

Data from the mice gavaged with a neratinib solution mice is shown below in the following tables and the PK data is summarized in FIG. 12.

### Neratinib, in Mouse Plasma, PO, 110 mg/kg

| Time (h) | N | Mean (ng/ml) | SD (ng/ml) | SE (ng/ml) | CV % |
|---|---|---|---|---|---|
| 0 | 3 | 118 | 144 | 82.9 | 121 |
| 0.5 | 3 | 553 | 120 | 69.1 | 21.6 |
| 1 | 3 | 1460 | 580 | 335 | 39.8 |
| 2 | 3 | 1228 | 545 | 315 | 44.4 |
| 4 | 3 | 497 | 171 | 98.5 | 34.4 |
| 8 | 3 | 334 | 261 | 151 | 78.1 |

| Parameter | Units | Estimate |
|---|---|---|
| kel | 1/h | 0.200 |
| t½ | h | 3 |
| Tmax | h | 1 |
| Cmax | ng/ml | 1460 |
| AUClast | h*ng/ml | 5401 |
| AUCINF_obs | h*ng/ml | 7069 |
| MRTlast | h | 3.003 |

### Example 10: Pharmacokinetics Study of Cream Formulations for Total Doxycycline Concentration in Adult Mice

In this example, six formulations of a topical doxycycline cream was applied to adult mice and plasma pharmacokinetics (PK) values for doxycycline were measured.

Each of the five formulations generally included the following components: Phosphatidylcholine, Isopropyl Palmitate, Stearic Acid, Benzyl Alcohol, Carthamus Tinctorius (safflower) oil, Oleic Acid, Polyglyceryl-4 Laurate, Deionized Water, doxycycline, and Poloxamer 407. The five formulations differed, at least, in the amount of doxycycline according to the following: Formulation A1: General formulation with 15.61% doxycycline monohydrate added at point of use to prevent degradation, varied emulsion type (˜7.5 mg pure doxycycline applied when administered 50 μL); Formulation A2: General formulation with 15.61% doxycycline monohydrate added at point of use to prevent degradation (˜7.5 mg pure doxycycline applied when administered 50 μL); Formulation A3: General formulation with 31.22% doxycycline monohydrate added at point of use (˜15 mg pure doxycycline applied when administered 50 μL); Formulation B: General formulation with 17.31% doxycycline hyclate added at point of use (˜7.5 mg pure doxycycline applied when administered 50 μL); and Formulation C: 15.61% doxycycline monohydrate in General formulation, varied emulsion type (˜7.5 mg pure doxycycline applied when administered 50 μL). In these experiments, two different forms of the active doxycycline were tested but blood levels of pure doxycycline was assayed. In particular, the 15.6% doxycycline monohydrate and 17.31% doxycycline hyclate formulations are understood to have the same amount of pure doxycycline in them.

The formulations used in this example were similar (e.g., in the relative amounts of each component) to those described in the previous examples, yet including doxycycline rather than the other listed compound. Generally, a doxycycline formulation comprised the following ingredients and at the listed weight percentages:

General Doxycycline Formulations

| Ingredient | Weight (%) |
|---|---|
| Phosphatidylcholine | 5-15% |
| Isopropyl Palmitate | 10-20% |
| Stearic Acid | 0.5-2% |
| Benzyl Alcohol | 0.5-2% |
| Carthamus Tinctorius | 1-5% |
| (safflower) oil | |
| Oleic Acid | 0.5-2% |
| Polyglyceryl-4 Laurate | 0.5-2% |
| Deionized Water | 20-80% |
| Poloxamer 407 | 5-15% |
| Doxycycline | 15-35% |

Additionally, a formulation for oral gavage was provided; this formulation comprised 205 mg/kg doxycycline monohydrate delivered by oral suspension in 0.5% methylcellulose and span 80 (˜3.075 mg pure doxycycline delivered per os).

In this example, six week or older, male mice were used in the study. A region ˜2×3 cm on the backs of each mouse was shaved 24 hours before experiment. Animals were food deprived for one hour before topical administration and for four hours during the study, and with no limit of access to water. Each mouse was topically applied one formulation at a doxycycline dose of 50 μL/animal. The tested topical formulations were rubbed in at the selected dose. After rubbing, the animals were monitored for 15-20 min to avoid licking of the formulations. If licking is was observed, an Elizabethan collar was put on the mouse to prevent licking off the formulation. Three animals per formulation and per time point were used. Blood samples were collected after drug administration in the following time points by cardiopuncture:

| # of animal | Experimental | |
|---|---|---|
| 1, 2, 3 | Pre-application | |
| 4, 5, 6 | 30 | min |
| 7, 8, 9 | 1 | hour |
| 10, 11, 12 | 2 | hours |
| 13, 14, 15 | 4 | hours |
| 16, 17, 18 | 8 | hours |

Blood samples, approximately 0.5 mL each, were collected from each animal into tubes with coagulation activators. The samples were incubated at RT for ˜40 min and centrifuged (for ˜15 minutes at 3000 xg). The serum was harvested into a single tube for each animal at each time point. The serum samples were frozen (˜−80° C.) after centrifugation and transferred to Bioanalytical Laboratory.

Analyses were performed using an assay kit and multifunctional plate reader Infinite® M1000 PRO (Tecan) according to the manufacturer's protocol. Total doxycycline concentrations were calculated using a standard curve prepared according to the manufacturer's protocol from the kit.

Using the calibration curve, concentrations of doxycycline in the serum samples were calculated. Pharmacokinetic analysis was performed using WinNonlin Professional 6.3 software package (Pharsight Corporation, USA). The following parameters were estimated as a result of the studies: Cₘₐₓ (ng/ml for serum or ng/g for tissues)—the maximum concentration; Tₘₐₓ (h)—the time of maximum concentration; AUC_{0→∞} (ng*min/ml)—AUCinf—AUC from time zero extrapolated to infinity; AUC_{0→t} (ng*min/ml)—AUClast—AUC from time zero to the time of the last non-zero Y value (24 h); Kₑₗ (l/h)—first order rate constant associated with the terminal (log-linear) elimination phase; T_{1/2} (h)—HL_Lambda_z=0.693/Lambda Z; and MRTlast (h)—mean residence time from the time of dosing to the time of the last measurable concentration. Statistical analysis of the concentrations was performed by descriptive statistics. The following values were calculated: Arithmetic mean (Mean); Standard deviation (SD); Standard error of the mean (SEM); Coefficient of variation (CV). For the concentration outliers detecting and excluding Grubbs statistical test was used. (See, the World Wide Web (at) graphpad.com/support/faqid/1598/).

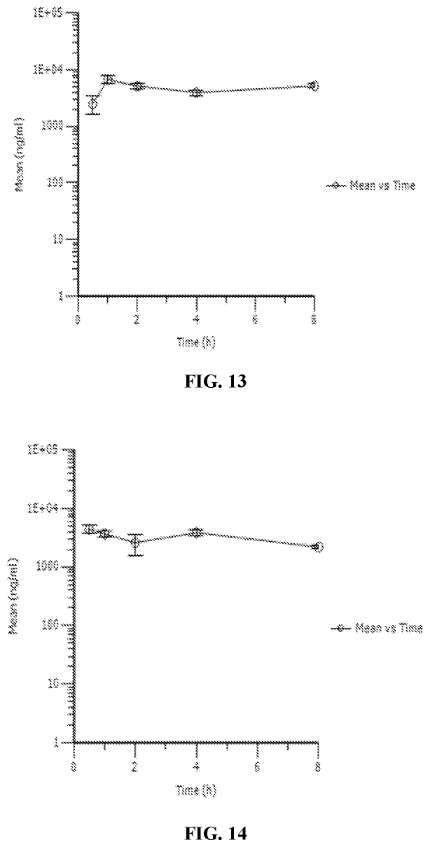

Data from the Formulation A1 mice is shown below in the following tables and the PK data is summarized in FIG. 13.

### Doxycycline, in Mouse Plasma, Dermal, 50 ul/Mouse, Creme Formulation A1 (Monohydrate)

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 2493 | 1494 | 862 | 59.9 |
| 1 | 2 | 6640 | 1464 | 1035 | 22.0 |
| 2 | 3 | 5103 | 961 | 555 | 18.8 |
| 4 | 3 | 3932 | 794 | 458 | 20.2 |
| 8 | 2 | 5143 | 675 | 478 | 13.1 |

| Parameter | Units | Estimate |
|---|---|---|
| kel | 1/h | nd |
| t½ | h | nd |
| Tmax | h | 1.00 |
| Cmax | ng/ml | 6640 |
| AUClast | h*ng/ml | 35769 |
| AUCINF | h*ng/ml | nd |
| MRTlast | h | 4.12 |

Data from the Formulation A2 mice is shown below in the following tables and the PK data is summarized in FIG. 14.

### Doxycycline, in Mouse Plasma, Dermal, 50 ul/Mouse, Creme Formulation A2 (Monohydrate)

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 2 | 4410 | 1032 | 730 | 23.4 |
| 1 | 2 | 3663 | 520 | 368 | 14.2 |
| 2 | 3 | 2595 | 1767 | 1020 | 68.1 |
| 4 | 3 | 3855 | 652 | 376 | 16.9 |
| 8 | 3 | 2182 | 228 | 132 | 10.5 |

| Parameter | Units | Estimate |
|---|---|---|
| kel | 1/h | nd |
| t½ | h | nd |
| Tmax | h | 0.50 |
| Cmax | ng/ml | 4410 |
| AUClast | h*ng/ml | 24773 |
| AUCINF | h*ng/ml | nd |
| MRTlast | h | 3.75 |

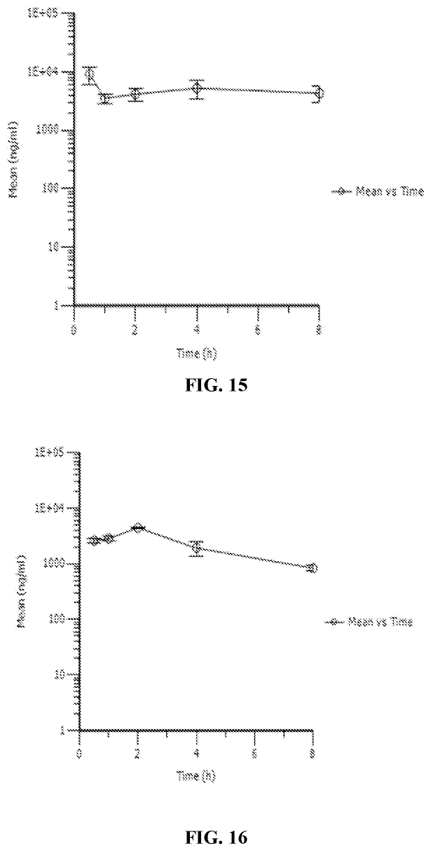

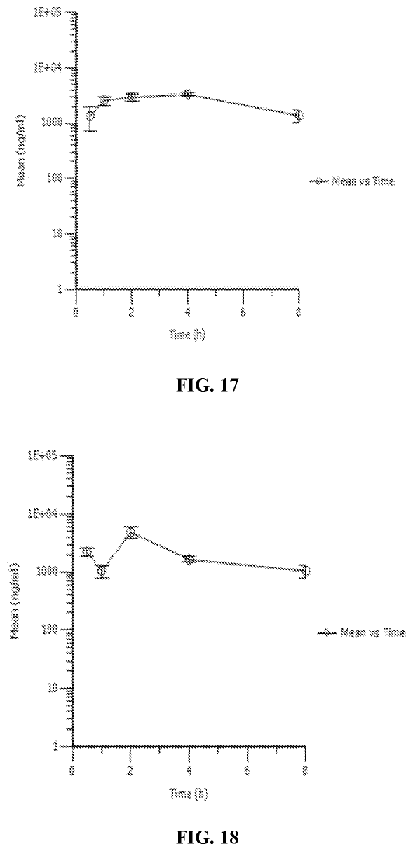

Data from the Formulation A3 mice is shown below in the following tables and the PK data is summarized in FIG. 15.

### Doxycycline, in Mouse Plasma, Dermal, 50 ul/Mouse, Creme Formulation A3 (Monohydrate)

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 9028 | 5282 | 3049 | 58.5 |
| 1 | 3 | 3520 | 1089 | 629 | 30.9 |
| 2 | 3 | 4147 | 1695 | 979 | 40.9 |
| 4 | 3 | 5252 | 3255 | 1879 | 62.0 |
| 8 | 3 | 4300 | 2424 | 1399 | 56.4 |

| Parameter | Units | Estimate |
|---|---|---|
| kel | 1/h | nd |
| t½ | h | nd |
| Tmax | h | 0.50 |
| Cmax | ng/ml | 9028 |
| AUClast | h*ng/ml | 37729 |
| AUCINF | h*ng/ml | nd |
| MRTlast | h | 3.95 |

Data from the mice gavaged with a doxycycline solution is shown below in the following tables and the PK data is summarized in FIG. 16.

### Doxycycline Monohydrate, in Mouse Plasma, PA 205 Mg/Kg

| Time (h) | N | Mean (ng/ml) | SD (ng/ml) | SE (ng/ml) | CV % |
|---|---|---|---|---|---|
| 0 | 3 | 0 | 0 | 0 | |
| 0.5 | 3 | 2603 | 344 | 199 | 13.2 |
| 1 | 3 | 2823 | 451 | 260 | 16.0 |
| 2 | 3 | 4413 | 151 | 87 | 3.42 |
| 4 | 3 | 1917 | 989 | 571 | 51.6 |
| 8 | 3 | 836 | 161 | 93 | 19.2 |

| Parameter | Units | Estimate |
|---|---|---|
| kel | 1/h | 0.27 |
| t½ | h | 2.59 |
| Tmax | h | 2.00 |
| Cmax | ng/ml | 4413 |
| AUClast | h*ng/ml | 17461 |
| AUCINF | h*ng/ml | 20589 |
| MRTlast | h | 3.00 |

Data from the Formulation B mice is shown below in the following tables and the PK data is summarized in FIG. 17.

### Doxycycline, in Mouse Plasma, Dermal, 50 ul/Mouse, Crème B+Doxycycline

| Time (h) | N | Mean (ng/ml) | SD (ng/ml) | SE (ng/ml) | CV % |
|---|---|---|---|---|---|
| 0 | 3 | 0 | 0 | 0 | |
| 0.5 | 3 | 1351 | 1128 | 651 | 83.5 |
| 1 | 3 | 2558 | 822 | 474 | 32.1 |
| 2 | 2 | 2918 | 682 | 483 | 23.4 |
| 4 | 3 | 3310 | 331 | 191 | 10.0 |
| 8 | 3 | 1364 | 607 | 350 | 44.5 |

| Parameter | Units | Estimate |
|---|---|---|
| kel | 1/h | 0.14 |
| t½ | h | 4.94 |
| Tmax | h | 4.00 |
| Cmax | ng/ml | 3310 |
| AUClast | h*ng/ml | 19629 |
| AUCINF | h*ng/ml | 29358 |
| MRTlast | h | 3.70 |

Data from the Formulation C mice is shown below in the following tables and the PK data is summarized in FIG. 18.

### Doxycycline, in Mouse Plasma, Dermal, 50 ul/Mouse, Creme Formulation C (Monohydrate)

| Time (h) | N | Mean (ng/ml) | SD (ng/ml) | SE (ng/ml) | CV % |
|---|---|---|---|---|---|
| 0 | 3 | 0 | 0 | 0 | |
| 0.5 | 3 | 2215 | 582 | 336 | 26.3 |
| 1 | 3 | 1045 | 479 | 277 | 45.9 |
| 2 | 3 | 4810 | 1721 | 993 | 35.77 |
| 4 | 3 | 1645 | 319 | 184 | 19.4 |
| 8 | 3 | 1042 | 439 | 254 | 42.1 |

| Parameter | Units | Estimate |
|---|---|---|
| kel | 1/h | 0.23 |
| t½ | h | 2.95 |
| Tmax | h | 2.00 |
| Cmax | ng/ml | 4810 |
| AUClast | h*ng/ml | 16126 |
| AUCINF | h*ng/ml | 20566 |
| MRTlast | h | 3.24 |

### Example 11: Pharmacokinetics Study of Cream Formulations for Total Cefepime Concentration in Adult Mice

In this example, three formulations of a topical cefepime cream was applied to adult mice and plasma pharmacokinetics (PK) values for cefepime were measured.

Each of the three formulations included the following components: Phosphatidylcholine, Isopropyl Palmitate, Stearic Acid, Benzyl Alcohol, Carthamus Tinctorius (safflower) oil, Oleic Acid, Polyglyceryl-4 Laurate, Deionized Water, Cefepime, and Poloxamer 407. Formulations A, B, and C, each comprising 3% cefepime (approximately 3 mg cefepime applied when administered 100 μL), differed slightly in the carrier in an effort to determine if the changes improved delivery. Formula A was simply a standard formulation, Formula B was the standard formulation with cefepime added at point of use to prevent degradation, and Formula C was the standard formulation with an additional excipient that was added to potentially enhance penetration.

The formulations used in this example were similar (e.g., in the relative amounts of each component) to those described in the previous examples, yet including cefepime rather than the other listed compound. Generally, a cefepime formulation comprised the following ingredients and at the listed weight percentages:

General Cefepime Formulations

| Ingredient | Weight (%) |
|---|---|
| Phosphatidylcholine | 5-15% |
| Isopropyl Palmitate | 10-20% |
| Stearic Acid | 0.5-2% |
| Benzyl Alcohol | 0.5-2% |
| Carthamus Tinctorius | 1-5% |
| (safflower) oil | |
| Oleic Acid | 0.5-2% |
| Polyglyceryl-4 Laurate | 0.5-2% |
| Deionized Water | 20-80% |
| Poloxamer 407 | 5-15% |
| Cefepime | 2-7% |

Additionally, a formulation for intravenous administration was provided; this formulation comprised an IV dose of 3 mg cefepime in 60 μL saline)

In this example, six week or older, male mice were used in the study. A region ˜2×3 cm on the backs of each mouse was shaved 24 hours before experiment. Animals were food deprived for one hour before topical administration and for four hours during the study, and with no limit of access to water. Each mouse was topically applied one formulation at a cefepime dose of 100 μL/animal. The tested topical formulations were rubbed in at the selected dose. After rubbing, the animals were monitored for 15-20 min to avoid licking of the formulations. If licking is was observed, an Elizabethan collar was put on the mouse to prevent licking off the formulation. Three animals per formulation and per time point were used. Blood samples were collected after drug administration in the following time points by cardiopuncture:

| # of animal | Experimental | |
|---|---|---|
| 1, 2, 3 | Pre-application | |
| 4, 5, 6 | 30 | min |
| 7, 8, 9 | 1 | hour |
| 10, 11, 12 | 2 | hours |
| 13, 14, 15 | 4 | hours |

Blood samples, approximately 0.5 mL each, were collected from each animal into tubes with coagulation activators. The samples were incubated at RT for ˜40 min and centrifuged (for ˜15 minutes at 3000 xg). The serum was harvested into a single tube for each animal at each time point. The serum samples were frozen (˜−80° C.) after centrifugation and transferred to Bioanalytical Laboratory.

Analyses were performed using an assay kit and multifunctional plate reader Infinite® M1000 PRO (Tecan) according to the manufacturer's protocol. Total cefepime concentrations were calculated using a standard curve prepared according to the manufacturer's protocol from the kit.

Using the calibration curve, concentrations of cefepime in the serum samples were calculated. Pharmacokinetic analysis was performed using WinNonlin Professional 6.3 software package (Pharsight Corporation, USA). The following parameters were estimated as a result of the studies: Cₘₐₓ (ng/ml for serum or ng/g for tissues)—the maximum concentration; Tₘₐₓ (h)—the time of maximum concentration; AUC_{0→∞} (ng*min/ml)—AUCinf—AUC from time zero extrapolated to infinity; AUC_{0→t} (ng*min/ml)—AUClast—AUC from time zero to the time of the last non-zero Y value (24 h); Kₑₗ (l/h) first order rate constant associated with the terminal (log-linear) elimination phase; T_{1/2} (h)—HL_Lambda_z=0.693/Lambda Z; and MRTlast (h)—mean residence time from the time of dosing to the time of the last measurable concentration. Statistical analysis of the concentrations was performed by descriptive statistics. The following values were calculated: Arithmetic mean (Mean); Standard deviation (SD); Standard error of the mean (SEM); Coefficient of variation (CV). For the concentration outliers detecting and excluding Grubbs statistical test was used. (See, the World Wide Web (at) graphpad.com/support/faqid/1598/).

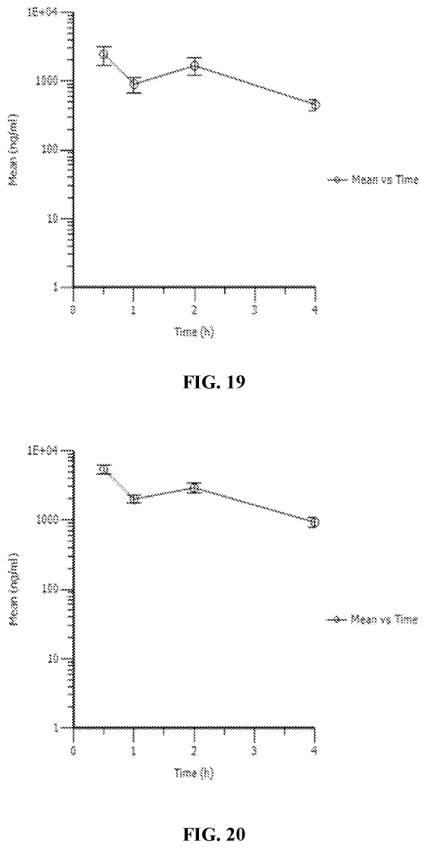

Data from the Formulation A mice is shown below in the following tables and the PK data is summarized in FIG. 19.

### Cefepime, PK in Mouse Plasma, Dermal, 100 ul/Mouse, Creme Formulation A

| Time (h) | N | Mean (ng/ml) | SD (ng/ml) | SE (ng/ml) | CV % |
|---|---|---|---|---|---|
| 0 | 3 | BQL | 0.85 | 0.49 | 4.80 |
| 0.5 | 3 | 2453 | 1310 | 756 | 53.4 |
| 1 | 3 | 899 | 409 | 236 | 45.5 |
| 2 | 3 | 1680 | 817 | 472 | 48.6 |
| 4 | 3 | 454 | 145 | 83.8 | 32.0 |

| Parameter | Units | Estimate |
|---|---|---|
| kel | 1/h | nd |
| t½ | h | nd |
| Tmax | h | 0.5 |
| Cmax | ng/ml | 2453 |
| AUClast | h*ng/ml | 4879 |
| AUCINF | h*ng/ml | nd |
| MRTlast | h | 1.67 |

Data from the Formulation B mice is shown below in the following tables and the PK data is summarized in FIG. 20.

### Cefepime, PK in Mouse Plasma, Dermal, 100 ul/Mouse, Creme Formulation B

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 5370 | 1403 | 810 | 26.1 |
| 1 | 3 | 1998 | 416 | 240 | 20.8 |
| 2 | 3 | 2913 | 806 | 465 | 27.7 |
| 4 | 3 | 926 | 268 | 155 | 28.9 |

| Parameter | Units | Estimate |
|---|---|---|
| Tmax | h | 0.5 |
| Cmax | ng/ml | 5370 |
| AUClast | h*ng/ml | 9480 |
| MRTlast | h | 1.61 |

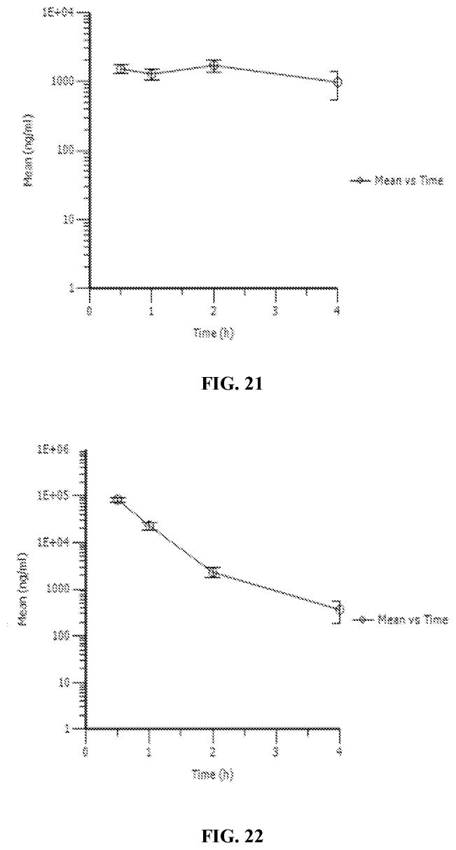

Data from the Formulation C mice is shown below in the following tables and the PK data is summarized in FIG. 21.

### Cefepime, PK in Mouse Plasma, Dermal, 100 Ul/Mouse, Creme Formulation C

| Time (h) | N | Mean (ng/ml) | SD (ng/ml) | SE (ng/ml) | CV % |
|---|---|---|---|---|---|
| 0 | 2 | 0.00 | 0.00 | 0.00 | |
| 0.5 | 3 | 1523 | 396 | 229 | 26.0 |
| 1 | 3 | 1286 | 397 | 229 | 30.9 |
| 2 | 3 | 1711 | 609 | 352 | 35.6 |
| 4 | 3 | 972 | 739 | 427 | 76.0 |

| Parameter | Units | Estimate |
|---|---|---|
| Tmax | h | 2 |
| Cmax | ng/ml | 1711 |
| AUClast | h*ng/ml | 5265 |
| MRTlast | h | 1.97 |

Data from the mice IV injected a cefepime solution is shown below in the following tables and the PK data is summarized in FIG. 22.

### Cefepime, PK in Mouse Plasma, IV, 250 mg/kg

| Time (h) | N | Mean (ng/ml) | SD (ng/ml) | SE (ng/ml) | CV % |
|---|---|---|---|---|---|
| 0 | 3 | BQL | | | |
| 0.5 | 3 | 83733 | 17322 | 10001 | 20.7 |
| 1 | 3 | 23133 | 7646 | 4414 | 33.1 |
| 2 | 3 | 2318 | 971 | 561 | 41.9 |
| 4 | 3 | 365 | 310 | 179 | 85.1 |

| Parameter | Units | Estimate |
|---|---|---|
| kel | 1/h | 1.32 |
| t½ | h | 0.53 |
| Tmax | h | 0.50 |
| Cmax | ng/ml | 83733 |
| C0 | ng/ml | 303081 |
| AUClast | h*ng/ml | 138829 |
| AUCINF | h*ng/ml | 139106 |
| Vz | ml/kg | 1364 |
| Cl | ml/h/kg | 1797 |
| MRTlast | h | 0.34 |
| Vss | ml/kg | 620 |

### Example 12: Pharmacokinetics Study of Cream Formulations for Total Eletriptan Concentration in Adult Mice

In this example, three formulations of a topical eletriptan cream was applied to adult mice and plasma pharmacokinetics (PK) values for eletriptan were measured.

Each of the three formulations generally included the following components: Phosphatidylcholine, Isopropyl Palmitate, Stearic Acid, Benzyl Alcohol, Carthamus Tinctorius (safflower) oil, Oleic Acid, Polyglyceryl-4 Laurate, Deionized Water, Eletriptan, and Poloxamer 407. The three formulations differed, at least, in the amount of eletriptan according to the following: Formulation A: 4% eletriptan hydrobromide in a general formulation (˜2 mg eletriptan applied when administered 50 μL); Formulation B: 8% eletriptan hydrobromide in a general formulation (˜4 mg eletriptan applied when administered 50 μL); and Formulation C: 3.85% eletriptan hydrobromide in a general formulation, varied emulsion type (˜1.92 mg applied when administered 50 μL).

The formulations used in this example were similar (e.g., in the relative amounts of each component) to those described in the previous examples, yet including eletriptan rather than the other listed compound. Generally, an eletriptan formulation comprised the following ingredients and at the listed weight percentages:

General Eletriptan Formulations

| Ingredient | Weight (%) |
|---|---|
| Phosphatidylcholine | 5-15% |
| Isopropyl Palmitate | 10-20% |
| Stearic Acid | 0.5-2% |
| Benzyl Alcohol | 0.5-2% |
| Carthamus Tinctorius | 1-5% |
| (safflower) oil | |
| Oleic Acid | 0.5-2% |
| Polyglyceryl-4 Laurate | 0.5-2% |
| Deionized Water | 20-80% |
| Poloxamer 407 | 5-15% |
| Eletriptan | 3-10% |

Additionally, a formulation for oral gavage was provided; this formulation comprised 52 mg/kg eletriptan hydrobromide delivered by oral suspension in 0.5% methylcellulose and span 80 (0.78 mg eletriptan hydrobromide delivered per os).

In this example, six week or older, male mice were used in the study. A region ˜2×3 cm on the backs of each mouse was shaved 24 hours before experiment. Animals were food deprived for one hour before topical administration and for four hours during the study, and with no limit of access to water. Each mouse was topically applied one formulation at an eletriptan dose of 50 μL/animal. The tested topical formulations were rubbed in at the selected dose. After rubbing, the animals were monitored for 15-20 min to avoid licking of the formulations. If licking is was observed, an Elizabethan collar was put on the mouse to prevent licking off the formulation. Three animals per formulation and per time point were used. Blood samples were collected after drug administration in the following time points by cardiopuncture:

| # of animal | Experimental | |
|---|---|---|
| 1, 2, 3 | Pre-application | |
| 4, 5, 6 | 30 | min |
| 7, 8, 9 | 1 | hour |
| 10, 11, 12 | 2 | hours |
| 13, 14, 15 | 4 | hours |
| 16, 17, 18 | 8 | hours |

Blood samples, approximately 0.5 mL each, were collected from each animal into tubes with coagulation activators. The samples were incubated at RT for ˜40 min and centrifuged (for ˜15 minutes at 3000 xg). The serum was harvested into a single tube for each animal at each time point. The serum samples were frozen (˜−80° C.) after centrifugation and transferred to Bioanalytical Laboratory.

Analyses were performed using an assay kit and multifunctional plate reader Infinite® M1000 PRO (Tecan) according to the manufacturer's protocol. Total eletriptan concentrations were calculated using a standard curve prepared according to the manufacturer's protocol from the kit.

Using the calibration curve, concentrations of eletriptan in the serum samples were calculated. Pharmacokinetic analysis was performed using WinNonlin Professional 6.3 software package (Pharsight Corporation, USA). The following parameters were estimated as a result of the studies: Cₘₐₓ (ng/ml for serum or ng/g for tissues)—the maximum concentration; Tₘₐₓ (h)—the time of maximum concentration; AUC_{0→∞} (ng*min/ml)—AUCinf—AUC from time zero extrapolated to infinity; AUC_{0→∞} (ng*min/ml)—AUClast—AUC from time zero to the time of the last non-zero Y value (24 h); Kₑₗ (l/h)—first order rate constant associated with the terminal (log-linear) elimination phase; T_{1/2} (h)—HL_Lambda_z=0.693/Lambda Z; and MRTlast (h)—mean residence time from the time of dosing to the time of the last measurable concentration. Statistical analysis of the concentrations was performed by descriptive statistics. The following values were calculated: Arithmetic mean (Mean); Standard deviation (SD); Standard error of the mean (SEM); Coefficient of variation (CV). For the concentration outliers detecting and excluding Grubbs statistical test was used. (See, the World Wide Web (at) graphpad.com/support/faqid/1598/).

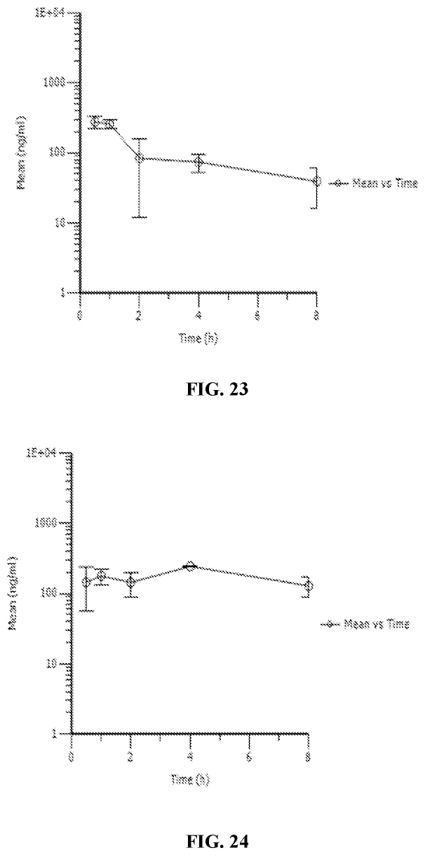

Data from the Formulation A mice is shown below in the following tables and the PK data is summarized in FIG. 23.

### Eletriptan, in Mouse Plasma, Dermal, 50 ul/Mouse, Creme Formulation A

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 276 | 99.4 | 57.4 | 36.1 |
| 1 | 3 | 258 | 60.7 | 35.1 | 23.5 |
| 2 | 3 | 84.0 | 125 | 72.2 | 149 |
| 4 | 3 | 74.0 | 37.7 | 21.8 | 51.0 |
| 8 | 3 | 39.0 | 39.3 | 22.7 | 101 |

| Parameter | Units | Estimate |
|---|---|---|
| Tmax | h | 0.5 |
| Cmax | ng/ml | 276 |
| AUClast | h*ng/ml | 758 |
| MRTlast | h | 2.68 |

Data from the Formulation B mice is shown below in the following tables and the PK data is summarized in FIG. 24.

### Eletriptan, in Mouse Plasma, Dermal, 50 ul/Mouse, Creme Formulation B

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 146 | 156 | 89.9 | 107 |
| 1 | 3 | 177 | 79.6 | 45.9 | 45.0 |
| 2 | 3 | 144 | 95.1 | 54.9 | 66.0 |
| 4 | 3 | 243 | 11.2 | 6.49 | 4.6 |
| 8 | 3 | 129 | 73.0 | 42.2 | 56.6 |

| Parameter | Units | Estimate |
|---|---|---|
| Tmax | h | 4.00 |
| Cmax | ng/ml | 243 |
| AUClast | h*ng/ml | 1410 |
| MRTlast | h | 3.96 |

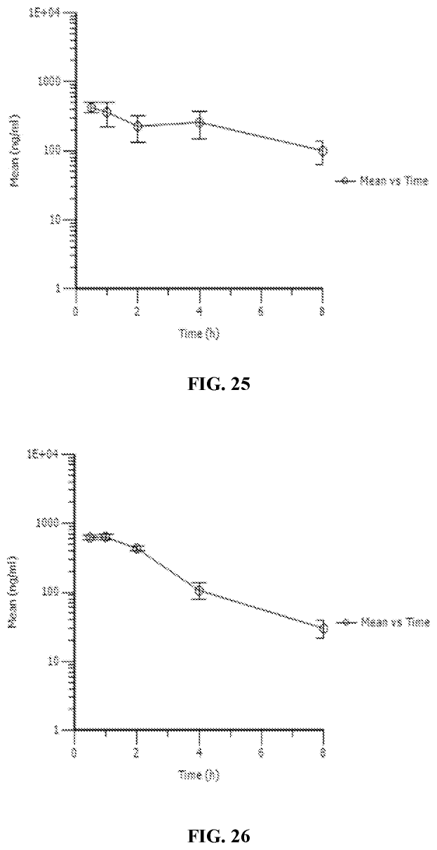

Data from the Formulation C mice is shown below in the following tables and the PK data is summarized in FIG. 25.

### Eletriptan, in Mouse Plasma, Dermal, 50 ul/Mouse, Creme Formulation C

| Time (h) | N | Mean (ng/ml) | SD (ng/ml) | SE (ng/ml) | CV % |
|---|---|---|---|---|---|
| 0.5 | 3 | 424 | 122 | 70.3 | 28.7 |
| 1 | 3 | 363 | 241 | 139 | 66.4 |
| 2 | 3 | 226 | 161 | 92.7 | 71.2 |
| 4 | 3 | 258 | 193 | 111 | 74.8 |
| 8 | 3 | 100 | 65.8 | 38.0 | 65.8 |

| Parameter | Units | Estimate |
|---|---|---|
| Tmax | h | 0.5 |
| Cmax | ng/ml | 424 |
| AUClast | h*ng/ml | 1798 |
| MRTlast | h | 3.20 |

Data from the mice gavaged with an eletriptan solution is shown below in the following tables and the PK data is summarized in FIG. 26.

### Eletriptan Hydrobromide, in Mouse Plasma, PO, 52 mg/kg

| Time (h) | N | Mean (ng/ml) | SD (ng/ml) | SE (ng/ml) | CV % |
|---|---|---|---|---|---|
| 0 | 3 | 0.00 | 0.00 | 0.00 | |
| 0.5 | 3 | 623 | 85.3 | 49.3 | 13.7 |
| 1 | 3 | 634 | 105 | 60.6 | 16.5 |
| 2 | 3 | 431 | 49.9 | 28.8 | 11.6 |
| 4 | 3 | 107 | 49.9 | 28.8 | 46.7 |
| 8 | 3 | 30.0 | 14.4 | 8.33 | 48.1 |

| Parameter | Units | Estimate |
|---|---|---|
| kel | 1/h | 0.43 |
| t½ | h | 1.63 |
| Tmax | h | 1.00 |
| Cmax | ng/ml | 634 |
| AUClast | h*ng/ml | 1814 |
| AUCINF | h*ng/ml | 1885 |

### Example 13: Transdermal Administration of Apremilast

In this example, a patient seeks treatment for moderate to severe rheumatoid arthritis (RA) that manifests as joint pain and swelling. A health care professional suggests that the patient try tofacitinib, a Janus kinase (JAK) inhibitor that helps disrupt JAK pathways from inside the cells, which are believed to play a role in inflammation. Conventionally, tofacitinib is prescribed at doses of 11 mg for oral administration.

In this example, tofacitinib is provided in a transdermal lotion or cream. The formulations described herein allow for effective transdermal administration of the compound. In this example, tofacitinib is included in the transdermal formulation detailed in Table 3A. Tofacitinib is substituted for ketoconazole.

Tofacitinib Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 61.84% |
| Tofacitinib | 1.00% |
| Poloxamer 407 | 9.25% |
| Total | 100.00% |

Transdermal administration allows direct absorption into a specific area. For example, a lotion can be applied to a specific joint where the patient has inflammation or pain.

The lotion or cream can include a transdermal delivery formulation and the active agent, tofacitinib. In this example, the dose of the active agent is 500 mg so that it is 15-25% of the solution. The transdermal delivery formulation can include less than about 60% w/w of one or more phosphatides, glucose, one or more fatty acids and water. The lotion/cream can be used to treat pain/inflammation from RA and used daily. It can be reapplied as needed.

### Example 14: Transdermal Administration of Celecoxib

Celecoxib is a non-steroidal anti-inflammatory drug (NAID), specifically a COX-2 inhibitor, which relieves pain and swelling (i.e. inflammation). It can be used to treat arthritis, acute pain, and menstrual pain and discomfort. It is conventionally administered orally. A transdermal formulation offers a more practical option.

In this example, a patient seeks treatment for moderate to severe rheumatoid arthritis (RA) that manifests as joint pain and swelling. A health care professional suggests that the patient try celecoxib. Conventionally, tofacitinib is prescribed at doses of 200 mg per day administered as a single dose or as 100 mg twice daily for oral administration.

In this example, celecoxib is provided in a transdermal lotion or cream. The formulations described herein allow for effective transdermal administration of the compound. In this example, celecoxib is included in the transdermal formulation detailed in Table 3A. Transdermal administration allows direct absorption into a specific area. For example, a lotion can be applied to a specific joint where the patient has inflammation or pain.

Celecoxib Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 42.84% |
| Celecoxib | 20.00% |
| Poloxamer 407 | 9.25% |
| Total | 100.00% |

The lotion or cream can include a transdermal delivery formulation and the active agent, celecoxib. In this example, the dose of the active agent is 500 mg so that it is 15-25% of the solution. The transdermal delivery formulation can include less than about 60% w/w of one or more phosphatides, glucose, one or more fatty acids and water. The lotion/cream can be used to treat pain/inflammation from RA and used daily. It can be reapplied as needed.

### Example 15: Transdermal Administration of Deoxycholic Acid

In this example, a patient seeks cosmetic treatment for a double chin (i.e. convexity or fullness associated with submental fat). A health care professional suggests that the patient try deoxycholic acid. Conventionally, deoxycholic acid is prescribed at doses 10 mg/mL (supplied in 2 mL vials) for localized injection. A single treatment consists of up to a maximum of 50 injections, 0.2 mL each (up to a total of 10 mL), spaced 1 cm apart. Up to 6 single treatments may be administered at intervals no less than 1 month apart

In this example, deoxycholic acid is provided in a transdermal lotion or cream. The formulations described herein allow for effective transdermal administration of the compound. In this example, deoxycholic acid is included in the transdermal formulation detailed in Table 3A.

Deoxycholic acid Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 52.84% |
| Deoxycholic acid | 10.00% |
| Poloxamer 407 | 9.25% |
| Total | 100.00% |

Transdermal administration allows direct absorption into a specific area. For example, a lotion can be applied to the chin at or near the submental fat. The lotion can include a transdermal delivery formulation and the active agent (collectively referred to as the formulation). In this example, the dose of the active agent (i.e. deoxycholic acid) is 10 mg/mL. The transdermal delivery formulation can include less than about 60% w/w of one or more phosphatides, glucose, one or more fatty acids and water.

A transdermal medicament is the ideal for several reasons. Deoxycholic acid topical cream obviates the need for the invasive nature and side effects of the injection of drugs. Topical administration also allows for the patient to apply the drug daily and continue administration until the submental fat adequately dissipates.

### Example 16: Transdermal Administration of Minoxidil

In this example, a patient seeks treatment for a pattern baldness (i.e. hair loss). A health care professional suggests that the patient try minoxidil, which is marketed under the tradename Rogaine. Conventionally, minoxidil is prescribed at doses 5% w/w for topical administration. It is recommended to be applied daily to the top of the scalp.

In this example, minoxidil is provided in a transdermal lotion or cream. The formulations described herein allow for effective transdermal administration of the compound. In this example, minoxidil is included in the transdermal formulation detailed in Table 3A.

Minoxidil Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 56.84% |
| Minoxidil | 5.00% |
| Poloxamer 407 | 9.25% |
| Total | 100.00% |

Transdermal administration can be more effective with the formulations described herein. For example, when applied with the transdermal delivery formulation of Table 3A, more of the active agent is absorbed. Minoxidil topical cream can be applied in smaller amounts, thus reducing the side effects. Further, when more of the active agent is absorbed, it can be more effective at promoting hair growth and reducing hair loss.

### Example 17: Transdermal Administration of Botulinum Toxin A

In this example, a patient seeks cosmetic treatment for facial wrinkles (e.g. crows feet). A health care professional suggests that the patient try botulinum toxin (BT). BT is conventionally supplied as a powder to be reconstituted at the time of treatment into a solution of sterile normal saline (i.e. 0.9% sodium chloride). Dilution volumes range from 1 to 4 ml per 100-unit vial. Based on the product insert, it is recommended that 100-unit vial be diluted in 2.50 ml. The resulting dose is 4.0 units per 0.1 ml. The dose is generally determined according to recommendations provided by the manufacture or distributor.

For Botox®, the approved dose for treatment of forehead lines (20 Units) in conjunction with glabellar lines (20 Units) is 40 Units. For simultaneous treatment of all three areas of the face, the dose is 20+24+20 Units (20 Units for forehead lines, 24 Units for lateral canthal lines, and 20 Units for glabellar lines) for a total dose of 64 Units.

In this example, BT is provided in a transdermal lotion or cream. The formulations described herein allow for effective transdermal administration of the compound. In this example, BT is included in the transdermal formulation detailed herein.

Botulinum Toxin Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 57.84% |
| Botulinum Toxin | 5.00% |
| Poloxamer 407 | 9.25% |
| Total | 100.00% |

Transdermal administration allows direct absorption into a specific area. For example, a lotion can be applied to the chin at or near the submental fat. The lotion can include a transdermal delivery formulation and the active agent (collectively referred to as the formulation). In this example, the dose of the active agent (i.e. BT) is 10 mg/mL. The transdermal delivery formulation can include less than about 60% w/w of one or more phosphatides, glucose, one or more fatty acids and water.

A transdermal medicament is the ideal for several reasons. BT topical cream obviates the need for the invasive nature and side effects of the injection of drugs. Rather than return to a healthcare provider's office for injections, the patient can apply the BT to the face periodically, as needed. Topical administration also allows for the patient to apply the drug daily and continue administration until wrinkles dissipate.

### Example 18: Transdermal Administration of Diclofenac Sodium

In this example, a patient seeks treatment for Gout. Gout can often be effectively treated and managed. Prompt diagnosis and initiation of treatment can alleviate acute gout symptoms (i.e. pain, swelling and inflammation in the affected joint). A health care professional suggests that the patient try diclofenac.

Diclofenac is a non-steroidal anti-inflammatory drug (NAID) which relieves pain and swelling (i.e. inflammation). It can be used to treat arthritis, acute pain, and menstrual pain and discomfort. It is conventionally administered orally. For acute pain in adults, it is generally prescribed at 18 or 35 milligrams (mg) three times per day. A transdermal formulation offers a more practical option.

In this example, diclofenac is provided in a transdermal lotion or cream. The formulations described herein allow for effective transdermal administration of the compound. It is included in the transdermal formulation detailed in Table 3A. Transdermal administration allows direct absorption into a specific area. For example, a lotion can be applied to a specific joint where the patient has inflammation or pain.

Diclofenac Sodium Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 61.84% |
| Diclofenac Sodium | 1.00% |
| Poloxamer 407 | 9.25% |
| Total | 100.00% |

The lotion or cream can include a transdermal delivery formulation and the active agent, diclofenac. In this example, the dose of the active agent is 500 mg so that it is 15-25% of the solution. The transdermal delivery formulation can include less than about 60% w/w of one or more phosphatides, glucose, one or more fatty acids and water. The lotion/cream can be used to treat pain/inflammation from RA and used daily. It can be reapplied as needed.

A transdermal medicament is the ideal for several reasons. The lack of interference with food and alcohol is one advantage. Topical delivery avoids the GI tract. Increased bioavailability permits lower doses which reduce the risk of side effects. Diclofenac topical cream obviates the need for the invasive nature and side effects of the injection of drugs. Topical administration also allows for the patient to increase the volume and incidence of application based on need/symptoms.

### Example 19: Transdermal Administration of Rimegepant

In this example, a patient seeks treatment for acute migraine with aura. A health care professional suggests that the patient try rimegepant as an alternative to an opiod drug. Rimegepant is non-opioid and non-addictive. Conventionally, it is consumed at a single dose of 75 mg (orally). It is taken once per day non-prophylactically.

Rimegepant can be provided in a transdermal lotion or cream. The formulations described herein allow for effective transdermal administration of the compound. In this example, rimegepant is included in the transdermal formulation detailed in herein.

Rimegepant Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 57.84% |
| Rimegepant | 5.00% |
| Poloxamer 407 | 9.25% |
| Total | 100.00% |

Transdermal administration allows direct absorption into a specific area. For example, a lotion can be applied to an area close to where pain is present. In this example, the patient applies the lotion to the top front and sides of the head.

The lotion can include a transdermal delivery formulation and the active agent (collectively referred to as the formulation). In this example, the dose of the active agent (i.e. rimegepant) is 5% w/w of the solution. A transdermal medicament presents several benefits. The lack of interference with food and alcohol is one advantage. Topical delivery avoids the GI tract and can increase bioavailability. Increased bioavailability permits lower doses which reduce the risk of side effects. Rimegepant topical cream obviates the need for the invasive nature and side effects of the injection of drugs. Topical administration also allows for the patient to increase the volume and incidence of application based on need/symptoms.

### Example 20: Transdermal Administration of Carfilzomib

In this example, a patient seeks treatment for multiple myeloma. The patient has already been treated unsuccessfully with other therapies including bortezomib and lenalidomide. A healthcare provider prescribes carfilzomib. Conventionally, it is administered prescribed at a single dose of 10 mg (intravenously). Sold commercially as Kyprolis, it is a sterile, white to off-white lyophilized powder and is available as a single-dose 10 mg, 30 mg or 60 mg vial.

Carfilzomib can be provided in a transdermal lotion or cream. The formulations described herein allow for effective transdermal administration of the compound. In this example, tofacitinib is included in the transdermal formulation detailed herein.

Carfilzomib Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 59.84% |
| Carfilzomib | 3.00% |
| Poloxamer 407 | 9.25% |
| Total | 100.00% |

The lotion or cream can include a transdermal delivery formulation and the active agent, carfilzomib. In this example, the active agent is 3% w/w of the solution. There are several benefits to transdermal administration. A topical cream obviates the need for the invasive nature and side effects of the injection of drugs. Rather than return to a healthcare provider's office for injections, the patient can apply the drug at increasing frequencies as tolerated and as directed by a physician.

### Example 21: Transdermal Administration of Ticagrelor

Ticagrelor is a blood thinner that can help reduce the risk of stroke, heart attack and other heart problems. In this example, a patient has suffered a heart attack. After recovery, a health can provider prescribes ticagrelor to help reduce the risk of a future heart attack or similar episode. Conventionally, ticagrelor is administered at a dose of 90 mg taken twice per day.

In this example, ticagrelor is provided in a transdermal lotion or cream. The formulations described herein allow for effective transdermal administration of the compound. In this example, ticagrelor is included in the transdermal formulation detailed herein. Transdermal administration allows direct absorption into a specific area. For example, a lotion can be applied to a near the arteries around the chest/heart region for increased bioavailability

Ticagrelor Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius | 2.69% |
| (safflower) oil | |
| Oleic Acid | 0.89% |
| Polyglyceryl-4 | 1.00% |
| Laurate | |
| Deionized Water | 55.85% |
| Ticagrelor | 10.00% |
| Poloxamer 407 | 8.49% |
| Total | 100.00% |

The lotion or cream can include a transdermal delivery formulation and the active agent, ticagrelor. In this example, ticagrelor is 10% w/w of the solution. The lotion/cream can be applied daily and/or as recommended by a physician. It can be reapplied as needed.

### Example 22: Transdermal Administration of Regorafenib

In this example, a patient is diagnosed with hepatocellular carcinoma (HCC). The patient is treated with sorafenib but there is no significant improvement. A healthcare provider recommends regorafenib as an alternative treatment. The patient is administered 160 mg to be taken orally once daily.

Regorafenib can be provided in a transdermal lotion or cream. The formulations described herein allow for effective transdermal administration of the compound. In this example, regorafenib is included in the transdermal formulation detailed herein.

Regorafenib Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius | 2.69% |
| (safflower) oil | |
| Oleic Acid | 0.89% |
| Polyglyceryl-4 | 1.00% |
| Laurate | |
| Deionized Water | 50.85% |
| Regorafenib | 15.00% |
| Poloxamer 407 | 8.49% |
| Total | 100.00% |

Transdermal administration allows for the patient to apply the drug daily and continue administration at the discretion of a physician. It also obviates the need for the invasive nature and side effects of the injection of drugs. The lotion can include a transdermal delivery formulation and the active agent (collectively referred to as the formulation). In this example, the active agent (i.e., regorafenib) is 15% of the lotion.

### Example 23: Transdermal Administration of Imatinib

In this example, a patient is diagnosed with gastrointestinal stromal tumors. A healthcare provider recommends imatinib as an alternative treatment. The patient is administered 400 mg to be taken once daily. Conventionally, it is taken orally.

Imatinib is provided in a transdermal lotion or cream. The formulations described herein allow for effective transdermal administration of the compound. In this example, imatinib is included in the transdermal formulation detailed in herein.

Imatinib Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius | 2.69% |
| (safflower) oil | |
| Oleic Acid | 0.89% |
| Polyglyceryl-4 | 1.00% |
| Laurate | |
| Deionized Water | 50.85% |
| imatinib | 15.00% |
| Poloxamer 407 | 8.49% |
| Total | 100.00% |

Transdermal administration can be more effective with the formulations described herein. For example, when applied with the transdermal delivery formulation, the active agent is absorbed without being subjected to the gastrointestinal tract. Imatinib topical cream can be applied in small amounts, thus reducing the side effects. Further, when more of the active agent is absorbed, it can be more effective at reducing tumor size/growth.

### Example 24: Transdermal Administration of Ferric Carboxymaltose

In this example, a patient is diagnosed with iron-deficiency anemia. The patient suffers from side-effects when administered oral iron supplements. A health care professional suggests that the patient try ferric carboxymaltose (FC). FC is conventionally administered as an injection (750 mg).

FC can be provided in a transdermal lotion or cream. The formulations described herein allow for effective transdermal administration of the compound. In this example, FC is included in the transdermal formulation detailed in herein.

Ferric Carboxymaltose Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius | 2.69% |
| (safflower) oil | |
| Oleic Acid | 0.89% |
| Polyglyceryl-4 | 1.00% |
| Laurate | |
| Deionized Water | 35.85% |
| Ferric carboxymaltose | 30.00% |
| Poloxamer 407 | 8.49% |
| Total | 100.00% |

Transdermal administration allows direct absorption into the blood stream. Transdermal administration also allows for the patient to apply the drug daily and continue regular administration at the discretion of a physician. It also obviates the need for the invasive nature and side effects of the injection of drugs. In this example, the active agent (i.e., FC) is 30% of the formulation.

### Example 25: Transdermal Administration of Methotrexate

In this example, a patient is diagnosed with rheumatoid arthritis (RA). The patient has tried self-care (e.g., heating pads) NSAID's and various therapies with little or no effect. A health care professional suggests that the patient try methotrexate. It is conventionally administered at 7.5 mg once per week (orally).

Methotrexate can be provided in a transdermal lotion or cream. The formulations described herein allow for effective transdermal administration of the compound. In this example, methotrexate is included in the transdermal formulation detailed below.

Methotrexate Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 | 1.06% |
| Laurate | |
| Deionized Water | 62.59% |
| Methotrexate | 0.25% |
| Poloxamer 407 | 9.25% |
| Total | 100.00% |

### Example 26: Transdermal Administration of Erlotinib

Erlotinib is an inhibitor of epidermal growth factor receptor (EGFR) tyrosine kinase that selectively and reversibly inhibits EGFR-associated intracellular autophosphorylation of tyrosine kinase. Erlotinib inhibits purified EGFR tyrosine kinase and EGFR autophosphorylation intact cells with 50% inhibition concentration IC50 values of 2 nmol/L and 20 nmol/L respectively. Erlotinib competes for the ATP-binding sits on the intracellular domain of EGFR resulting in the inhibition of downstream signaling pathway involved in angiogenesis, cell propagation and cell survival. Erlotinib concentration-dependently inhibits EGFR-mediated propagation signals transduction, displays prominent anti-tumor activity against neoplasms harboring EGFR expression and exhibits a tolerable toxicologic profile.

In this example, a patient seeks treatment for pancreatic cancer. A health care professional prescribes erlotinib. Conventionally, it is administered orally at 100 mg/day with gemcitabine (a chemotherapy medication).

In this example, erlotinib is provided in a transdermal lotion or cream. The formulations described herein allow for effective transdermal administration of the compound. In this example, erlotinib is included in the transdermal formulation as described in Table 3A above.

Erlotinib Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius | 2.69% |
| (safflower) oil | |
| Oleic Acid | 0.89% |
| Polyglyceryl-4 Laurate | 1.00% |
| Deionized Water | 55.85% |
| Erlotinib | 10.00% |
| Poloxamer 407 | 8.49% |
| Total | 100.00% |

Transdermal administration allows direct absorption into a specific area. For example, a lotion can be applied to the abdominal area near the pancreas.

The lotion can include a transdermal delivery formulation and the active agent (collectively referred to as the formulation). In this example, the active agent (i.e., erlotinib) is 10% w/w of the solution. The transdermal delivery formulation can include phosphatidylcholine, isopropyl palmitate, stearic acid, benzyl alcohol, polyglyceryl-4 laurate and poloxamer 407.

A transdermal medicament is the ideal for several reasons. The lack of interference with food and alcohol is one advantage. Topical delivery avoids the GI tract. Increased bioavailability permits lower doses which reduce the risk of side effects. Erlotinib topical cream obviates the need for the invasive nature and side effects of the injection of drugs. Topical administration also allows for the patient to increase the volume and incidence of application based on need/symptoms.

### Example 27: Transdermal Administration of Everolimus

Everolimus is a medication used as an immunosuppressant to prevent rejection of organ transplants and in the treatment of renal cell cancer and other tumors. Much research has also been conducted on everolimus and other mTOR inhibitors as targeted therapy for use in a number of cancers.

In this example, a patient seeks treatment for renal cell cancer. A health care professional prescribes everolimus. Everolimus works by interfering with the growth of cancer cells, which are eventually destroyed by the body. Since the growth of normal body cells may also be affected, other unwanted effects will also occur. Conventionally, tofacitinib is administered orally in solution (i.e., water) at a dose of 10 mg per day.

In this example, everolimus is provided in a transdermal lotion or cream. The formulations described herein allow for effective transdermal administration of the compound. In this example, everolimus is included in the transdermal formulation detailed in Table 3A.

Everolimus Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 Laurate | 1.06% |
| Deionized Water | 61.84% |
| Everolimus | 1.00% |
| Poloxamer 407 | 9.25% |
| Total | 100.00% |

Transdermal administration allows direct absorption into a specific area. For example, a lotion can be applied to the abdominal region of the patient, near the kidneys.

The lotion or cream can include a transdermal delivery formulation and the active agent, everolimus. In this example, the active agent is 1% of the solution. The transdermal delivery formulation can include phosphatidylcholine, isopropyl palmitate, stearic acid, benzyl alcohol, polyglyceryl-4 laurate and poloxamer 407.

### Example 28: Transdermal Administration of Ruxolitinib

Primary Myelofibrosis is a condition characterized by the buildup of scar tissue (fibrosis) in the bone marrow, the tissue that produces blood cells. Because of the fibrosis, the bone marrow is unable to make enough normal blood cells. Ruxolitinib targets and binds to the tyrosine kinase receptors and inhibits Janus Associated Kinases (JAK1 and JAK2) which mediate the signaling of a number of cytokines and growth factors that are important for hematopoiesis and immune function. By binding to these receptors, ruxolitinib blocks important pathways that promote cell division. Myelofibrosis (MF) is a myeloproliferative neoplasm (MPN) known to be associated with dysregulated JAK1 and JAK2 signaling. Patients with MF who either carry the JAK2 V617 mutation or does not have the JAK2 V617F mutation may both respond to ruxolitinib. It is conventionally administered orally. A transdermal formulation offers a more practical option.

In this example, a health care professional prescribes ruxolitinib to a patient suffering from primary myelofibrosis. Conventionally, ruxolitinib is prescribed orally at doses ranging from 5 mg to 20 mg PO BID (e.g. 20 mg PO BID for a patient with a platelet count >200×10⁹/L).

In this example, ruxolitinib is provided in a transdermal lotion or cream. The formulations described herein allow for effective transdermal administration of the compound. Ruxolitinib is included in the transdermal formulation detailed in Table 3A. Transdermal administration allows direct absorption into a specific area. For example, a lotion can be applied to specific areas (e.g. near bones) where the fibrosis is most apparent.

Ruxolitinib Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 Laurate | 1.06% |
| Deionized Water | 60.84% |
| Ruxolitinib | 2.00% |
| Poloxamer 407 | 9.25% |
| Total | 100.00% |

The lotion or cream can include a transdermal delivery formulation and the active agent, ruxolitinib. In this example, the agent is 2% of the solution. The transdermal delivery formulation can include phosphatidylcholine, isopropyl palmitate, stearic acid, benzyl alcohol, polyglyceryl-4 laurate and poloxamer 407.

### Example 29: Transdermal Administration of Carfilzomib

Carfilzomib, sold under the brand name Kyprolis, is an anti-cancer medication acting as a selective proteasome inhibitor. Carfilzomib covalently irreversibly binds to and inhibits the chymotrypsin-like activity of the 20S proteasome, an enzyme that degrades unwanted cellular proteins. Carfilzomib displays minimal interactions with non-proteasomal targets, thereby improving safety profiles over bortezomib. Inhibition of proteasome-mediated proteolysis results in a build-up of polyubiquitinated proteins, which may cause cell cycle arrest, apoptosis, and inhibition of tumor growth.

In this example, a health care professional prescribes carfilzomib to a patient suffering from multiple myeloma. Conventionally, it is administered intravenously (20 mg/m2) over 2 to 10 minutes, on two consecutive days, each week for three weeks (Days 1, 2, 8, 9, 15, and 16), followed by a 12-day rest period (Days 17 to 28).

In this example, carfilzomib is provided in a transdermal lotion or cream. The formulations described herein allow for effective transdermal administration of the compound. Carfilzomib is included in the transdermal formulation detailed in Table 3A. Transdermal administration allows direct absorption without the need to visit a clinic for intravenous administration. A transdermal lotion can be applied twice daily to ensure a more consistent levels of the agent circulate through the bloodstream.

Carfilzomib Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 Laurate | 1.06% |
| Deionized Water | 57.84% |
| Carfilzomib | 5.00% |
| Poloxamer 407 | 9.25% |
| Total | 100.00% |

### Example 30: Transdermal Administration of Ganetespib

Ganetespib is a HSP90 inhibitor. The inhibition of heat shock protein 90, which leads to the simultaneous degradation of multiple proteins involved in oncogenic signaling pathways, is a strategy to treat a variety of cancer types.

In this example, a health care professional prescribes carfilzomib to a patient suffering from multiple myeloma. Conventionally, it is administered intravenously (20 mg/m2) over 2 to 10 minutes, on two consecutive days, each week for three weeks (Days 1, 2, 8, 9, 15, and 16), followed by a 12-day rest period (Days 17 to 28).

In this example, carfilzomib is provided in a transdermal lotion or cream. The formulations described herein allow for effective transdermal administration of the compound. Carfilzomib is included in the transdermal formulation detailed in Table 3A. Transdermal administration allows direct absorption without the need to visit a clinic for intravenous administration. A transdermal lotion can be applied twice daily to ensure a more consistent levels of the agent circulate through the bloodstream.

Thyroid cancer is the most common endocrine malignancy and its incidence has increased in recent decades. In this example, a patient is treated with ganetespib to treat thyroid cancer. The formulations described herein allow for effective transdermal administration of the compound. In this example, ganetespib is included in the transdermal formulation detailed in Table 3A.

Ganetespib Formulation

| Ingredient | Weight (%) |
|---|---|
| Phosphatidylcholine | 7.02% |
| Isopropyl Palmitate | 12.21% |
| Stearic Acid | 0.57% |
| Benzyl Alcohol | 1.28% |
| Carthamus Tinctorius | 2.69% |
| (safflower) oil | |
| Oleic Acid | 0.89% |
| Polyglyceryl-4 Laurate | 1.00% |
| Deionized Water | 50.85% |
| Ganetespib | 15.00% |
| Poloxamer 407 | 8.49% |
| Total | 100.00% |

Transdermal administration can be more effective with the formulations described herein. For example, when applied with the transdermal delivery formulation of Table 3A, more of the active agent is absorbed. Ganetespib topical cream can be applied to the neck, near the patient's thyroid, thus reducing the side effects

Transdermal administration can be more effective with the formulations described herein. For example, when applied with the transdermal delivery formulation of Table 3A, more of the active agent is absorbed. Ganetespib topical cream can be applied to the neck, near the patient's thyroid, thus reducing the side effects.

### Example 31: Transdermal Administration of Obatoclax

Obatoclax Mesylate (GX15-070) is an antagonist of Bel-2. Currently, obatoclax is a clinical stage drug candidate that has been proposed to target and inhibit prosurvival members of the Bel-2 family, and thereby contribute to cancer cell lethality. It has been proposed to treat various cancers including leukemia, lymphoma, myelofibrosis.

In this example, a health care professional prescribes obatoclax to a patient suffering from leukemia. Obatoclax is provided in a transdermal lotion or cream. The formulations described herein allow for effective transdermal administration of the compound. Obatoclax is included in the transdermal formulation detailed in Table 3A. A transdermal lotion can be applied twice daily to ensure a more consistent levels of the agent circulate through the bloodstream.

Obatoclax is provided in a transdermal lotion or cream. The formulations described herein allow for effective transdermal administration of the compound. In this example, obatoclax is included in the transdermal formulation detailed in Table 3A.

Obatoclax Formulation

| Ingredient | Weight (%) |
|---|---|
| Phosphatidylcholine | 7.64% |
| Isopropyl Palmitate | 13.30% |
| Stearic Acid | 0.62% |
| Benzyl Alcohol | 1.39% |
| Carthamus Tinctorius | 2.93% |
| (safflower) oil | |
| Oleic Acid | 0.97% |
| Polyglyceryl-4 Laurate | 1.06% |
| Deionized Water | 61.84% |
| Obatoclax | 1.00% |
| Poloxamer 407 | 9.25% |
| Total | 100.00% |

A transdermal medicament is the ideal for several reasons. Obatoclax topical cream obviates the need for the invasive nature and side effects of the injection of drugs. Rather than return to a healthcare provider's office for injections, the patient can apply the agent directly through the skin. Topical administration also allows for the patient to apply the drug daily (or as prescribed) without the side-effects or oral consumption.

### Example 32: Transdermal Delivery of Aqueous Insoluble Molecules

Various transdermal formulations of the present disclosure are able to successfully deliver in vivo (at relatively high bioavailability) a range of actives that are either entirely insoluble, at least partially, or majority insoluble. This ability to deliver insoluble actives is contrary to the commonly-held belief that actives generally need to be solubilized for transdermal drug delivery.

There are a number of pharmacologically active medicaments which are poorly soluble in in aqueous media and/or acidic solutions which have proved challenging to deliver to a subject either transdermally or orally, due to the inability of the drug to be absorbed by the small intestine, or inability of the medicament to be solubilized in a transdermal formulation and penetrate the skin. In general, it has previously been thought that pharmacologically active medicaments must be wholly or partially dissolved in an aqueous solution in order to be absorbed by a subject and achieve a reasonably high bioavailability in a subject. In the case of orally administered medicaments, it is thought a drug must be wholly or partially soluble in an aqueous medium in order dissolved in the stomach and absorbed by the small intestine. In the case of transdermally administered drugs, it is thought that a medicament must be wholly or mostly soluble in the formulation in order to penetrate the skin and achieve a high or relatively high bioavailability in a subject.

Examples such medicaments which are poorly soluble in aqueous medium are drugs which fall into Class 2 and Class 4 of the Biopharmaceutical Classification System (BCS). The BCS is a scientific framework which predicts in vivo drug performance via in vitro measurements of solubility and permeation. Solubility is the extent a drug can dissolve in gastrointestinal (GI) fluid and permeation is the extent solubilized drug can pass across membranes of cells lining the GI tract. Medicaments have a high solubility according to BCS if less than 250 mL of an aqueous media (pH between 1-7.5) dissolves the highest API prescription dose. Class 2 drugs and Class 4 drugs have low solubilities as they do not meet this solubility criteria. Class 2 drugs have high permeability whereas class 4 drugs have low permeability.

Various transdermal formulations of the present disclosure are effective in delivering class 2 and class 4 insoluble molecules at a high or relatively high bioavailability in a subject. In this example, mice were transdernally treated with formulations of the present disclosure comprising essentially insoluble class 2 or class 4 molecules. Surprisingly, the essentially insoluble molecules were delivered at a high or relatively high bioavailability in the mice. The following molecules were included in a transdermal formulation comprising one of Hydrocortisone, Sildenafil Citrate, Cyclosporine, Eletriptan, Neratinib Maleate, or Free Base Neratinib. Notably, the following molecules normally do not solubilize in aqueous medium at >2% (hydrocortisone), >1% (sildenafil citrate, cyclosporine, or apixaban), >0.1% (neratinib maleate or neratinib free base), or >0.002% (Eetriptan). Also, in this example, other molecules were delivered at an improved bioavailability in mice. These molecules comprising sodium bicarbonate (which can achieve an 9% in solution); apixaban (which is soluble at <1% in aqueous medium); vancomycin (Formulation A which was can form a 71% solution, Formulation B which can form a 35% solution); or doxycycline (Formulation A which was can form a <1% solution, Formulation B which can form a 15% solution). These results are summarized in the below Table.

| | Active | | Applied | Estimated | | | | | |
|---|---|---|---|---|---|---|---|---|---|
| | Conc. | Application | Active | Active | Cmax | | | AUCinf | AUClast |
| | (w/w | Volume | Dose | % in | (ng/ | Tmax | T1/2 | (h*ng/ | (h*ng/ |
| Active | %) | (uL) | (mg) | Solution | mL) | (h) | (h) | mL) | mL) |
| Hydrocortisone | 1.00% | 100 | 1.00 | 2% | 2132 | 1.0 | 3.0 | 4859 | — |
| Sildenafil | 3.60% | 100 | 3.60 | <1% | 4388 | 0.5 | 0.94 | 9664 | — |
| Citrate | | | | | | | | | |
| Cyclosporine | 2.00% | 100 | 2.00 | <1% | 736 | 0.5 | 6.21 | 10079 | — |
| Eletriptan | 3.84% | 50 | 1.92 | <0.1% | 424 | 0.5 | nd | nd | 1798 |
| Neratinib | 4.23% | 100 | 4.23 | <1% | 2636 | 0.5 | nd | nd | 11615 |
| Maleate | | | | | | | | | |
| Free Base | 3.50% | 100 | 3.50 | <1% | 2498 | 2 | 1.96 | 12993 | 12045 |
| Neratinib | | | | | | | | | |
| Vancomycin | 8.00% | 100 | 8.00 | 71% | 458 | 0.5 | nd | nd | 800 |
| Hydrochloride | | | | | | | | | |
| (A) | | | | | | | | | |
| Vancomycin | 15.00% | 100 | 15.00 | 35% | 955 | 0.5 | 4.8 | 1462 | 1208 |
| Hydrochloride | | | | | | | | | |
| (B) | | | | | | | | | |
| Apixaban | 3.00% | 50 | 1.50 | <1% | 1878 | 0.5 | nd | nd | 3933 |
| Doxycycline | 31.22% | 50 | 15.61 | <1% | 9028 | 0.5 | nd | nd | 37729 |
| Monohydrate | | | | | | | | | |
| (A) | | | | | | | | | |
| Doxycycline | 17.31% | 50 | 8.66 | 15% | 3310 | 4 | 4.94 | 29358 | 19629 |
| Hyclate (B) | | | | | | | | | |

These data demonstrate that transdermal formulations of the present disclosure can result in systemic administration of an active agent and especially a relative insoluble active agent. These data are relevant to delivery of the insoluble molecules described in the above table described elsewhere (with respect to the Class 2 molecules and Class 4 molecules). In particular, the transdermal formulations of the present disclosure can provide a higher concentration of a molecule (especially one that is insoluble) and can provide systemic administration for a molecule that would be poorly absorbed by the gut epithelium. Together, molecules that are not suitable for enteral delivery or are suitable but at a low dosage can be systemically administered via transdermal formulations of the present disclosure.

Accordingly, any herein-disclosed transdermal formulation can provide systemic administration of a medicament via transdermal delivery of the medicament.

### Example 33 Transdermal Formulations of the Present Disclosure Provide Systemic Administration of Active Agents

Additionally, the transdermal formulations of the present disclosure are able to deliver an active agent into an animal's bloodstream and thereby provide systemic administration of the agent. Here, sodium bicarbonate was used as an illustrative active agent. Transdermal formulations comprising sodium bicarbonate allowed penetration of the active agent thru the skin and into the blood stream. The circulating sodium bicarbonate was removed from blood by the kidneys and affected the pH of the resulting urine. In in vivo experiments, mice treated transdermally with formulations comprising sodium bicarbonate had urine with a higher pH than control mice. More specifically, untreated mice had a urine pH of 5.88 and mice provided an oral 9% sodium bicarbonate solution had a urine pH of 6.05. On the other hand, mice transdermally administered one of four formulations (numbered Candidate 1, Candidate 2, Candidate 3, and Candidate 4) according to the present disclosure had a urine pH of 7.05, 7.43, 8.0, and 8.68 respectively.

The formulation of Candidate 1 comprised the following:

| Ingredient | % by Mass (of Total) |
|---|---|
| Menthol | 0.50% |
| Benzyl Alcohol | 1.00% |
| Ethanol | 1.50% |
| LIPMAX | 30.00% |
| Durosoft PK | 1.00% |
| 30% Pluronic Gel | 30.5555% |
| Deionized Water | 0.9445% |
| Sodium Bicarbonate | 33.20% |
| Lime Basil Fragrance | 0.30% |
| NaOH 50% Solution | 1.00% |

The mice were dosed 1110 μl (3×2×185 μl)/day with the formulation of Candidate 1

The formulation of Candidate 2 comprised the following:

| Ingredient | % by Mass |
|---|---|
| LIP | 25.00% |
| Benzyl Alcohol | 1.00% |
| Menthol | 0.90% |
| Durosoft | 2.00% |
| Pluronic Granules | 10.00% |
| Water | 45.26% |
| Sodium Carbonate | 15.84% |

The mice were dosed 150 μl (3×50 μl)/day with the formulation of Candidate 2.

The formulation of Candidate 3 comprised the following:

| Ingredient | % by Mass |
|---|---|
| Phospholipon 90G | 4.03% |
| Isopropyl Palmitate | 7.00% |
| Benzyl Alcohol | 1.68% |
| Stearic Acid | 0.32% |
| Cetyl Alcohol | 2.00% |
| Ethanol | 1.50% |
| Safflower Oil (USP) | 1.55% |
| Oleic Acid (USP) | 0.50% |
| Almond Oil | 3.00% |
| Propylene Glycol | 5.00% |
| Deionized Water | 21.06% |
| Dextrose Anhydrous | 0.35% |
| 30% Pluronic Gel | 18.00% |
| Sodium Bicarbonate | 33.00% |
| (3DF) | |
| Durosoft PK-SG | 1.00% |

The mice were dosed 150 μl (3×50 μl)/day with the formulation of Candidate 3.

The formulation of Candidate 4 comprised the following:

| Ingredient | % by Mass |
|---|---|
| Phospholipon 90G | 4.03% |
| Isopropyl Palmitate | 7.00% |
| Benzyl Alcohol | 1.68% |
| Stearic Acid | 0.32% |
| Cetyl Alcohol | 2.00% |
| Menthol | 0.50% |
| Ethanol | 1.50% |
| Safflower Oil (USP) | 1.55% |
| Oleic Acid (USP) | 0.50% |
| Almond Oil | 3.00% |
| Propylene Glycol | 5.00% |
| Deionized Water | 20.56% |
| Dextrose Anhydrous | 0.35% |
| 30% Pluronic Gel | 18.00% |
| Sodium Bicarbonate (3DF) | 33.00% |
| Durosoft PK-SG | 1.00% |

The mice were dosed 150 μl (3×50 μl)/day with the formulation of Candidate 4.

These data demonstrate that transdermal formulations of the present disclosure can result in systemic administration of an active agent, e.g., any medicament disclosed in Table 1. These data are relevant to delivery of the insoluble molecules described in Example 32 and described elsewhere (with respect to the Class 2 molecules and Class 4 molecules). In particular, the transdermal formulations of the present disclosure can provide a higher concentration of a molecule (especially one that is insoluble) and can provide systemic administration for a molecule that would be poorly absorbed by the gut epithelium. Together, molecules that are not suitable for enteral delivery or are suitable but at a low dosage can be systemically administered via transdermal formulations of the present disclosure.

Accordingly, any herein-disclosed transdermal formulation can provide systemic administration of a medicament via transdermal delivery of the medicament.

### Example 34 Transdermal Formulations of the Present Disclosure Provide Systemic Administration of Active Agents

In various embodiments, a transdermal delivery formulation comprises the components of the below table:

General Formulation

| Ingredient | Weight (%) |
|---|---|
| Phospholipid | 3-15% |
| Fatty acid ester | 5-20% |
| Long-chain fatty acids | 0.1-10% |
| Water | 30-90% |
| Medicament | 0.001%-30%. |
| Optionally: | |
| viscosity-improving agent | 5-20% |
| penetration enhancer | 0.5-5% |
| emulsifier | 0.5-10% |
| Total | 100.00% |

In the above table, where an ingredient has weight percent that ranges from 5 to 20% (e.g., for the Fatty acid ester and viscosity-improving agent) as an example, that ingredient may be present in the formulation at any percentage (w/w or w/v) from about 5% to about 20%. The weight percentage may be about 5% to about 20%. The weight percentage may be about 5% to about 6%, about 5% to about 7%, about 5% to about 8%, about 5% to about 9%, about 5% to about 10%, about 5% to about 11%, about 5% to about 12%, about 5% to about 13%, about 5% to about 14%, about 5% to about 15%, about 6% to about 7%, about 6% to about 8%, about 6% to about 9%, about 6% to about 10%, about 6% to about 11%, about 6% to about 12%, about 6% to about 13%, about 6% to about 14%, about 6% to about 15%, about 7% to about 8%, about 7% to about 9%, about 7% to about 10%, about 7% to about 11%, about 7% to about 12%, about 7% to about 13%, about 7% to about 14%, about 7% to about 15%, about 8% to about 9%, about 8% to about 10%, about 8% to about 11%, about 8% to about 12%, about 8% to about 13%, about 8% to about 14%, about 8% to about 15%, about 9% to about 10%, about 9% to about 11%, about 9% to about 12%, about 9% to about 13%, about 9% to about 14%, about 9% to about 15%, about 10% to about 11%, about 10% to about 12%, about 10% to about 13%, about 10% to about 14%, about 10% to about 15%, about 10% to about 16%, about 10% to about 17%, about 10% to about 18%, about 10% to about 19%, about 10% to about 20%, about 11% to about 12%, about 11% to about 13%, about 11% to about 14%, about 11% to about 15%, about 11% to about 16%, about 11% to about 17%, about 11% to about 18%, about 11% to about 19%, about 11% to about 20%, about 12% to about 13%, about 12% to about 14%, about 12% to about 15%, about 12% to about 16%, about 12% to about 17%, about 12% to about 18%, about 12% to about 19%, about 12% to about 20%, about 13% to about 14%, about 13% to about 15%, about 13% to about 16%, about 13% to about 17%, about 13% to about 18%, about 13% to about 19%, about 13% to about 20%, about 14% to about 15%, about 14% to about 16%, about 14% to about 17%, about 14% to about 18%, about 14% to about 19%, about 14% to about 20%, about 15% to about 16%, about 15% to about 17%, about 15% to about 18%, about 15% to about 19%, about 15% to about 20%, about 16% to about 17%, about 16% to about 18%, about 16% to about 19%, about 16% to about 20%, about 17% to about 18%, about 17% to about 19%, about 17% to about 20%, about 18% to about 19%, about 18% to about 20%, or about 19% to about 20%, and any range therebetween. The weight percentage may be about 5%, about 6%, about 7%, about 8%, about 9%, about 10%, about 11%, about 12%, about 13%, about 14%, about 15%, about 16%, about 17%, about 18%, about 19%, or about 20%. The weight percentage may be at least about 5%, about 6%, about 7%, about 8%, about 9%, about 10%, about 11%, about 12%, about 13%, or about 14%. The weight percentage may be at most about 6%, about 7%, about 8%, about 9%, about 10%, about 11%, about 12%, about 13%, about 14%, about 15%, about 16%, about 17%, about 18%, about 19%, or about 20%. Moreover, the weight percentage may be about 5% to about 6%. The weight percentage may be about 5% to about 5.1%, about 5% to about 5.2%, about 5% to about 5.3%, about 5% to about 5.4%, about 5% to about 5.5%, about 5% to about 5.6%, about 5% to about 5.7%, about 5% to about 5.8%, about 5% to about 5.9%, about 5% to about 6%, about 5.1% to about 5.2%, about 5.1% to about 5.3%, about 5.1% to about 5.4%, about 5.1% to about 5.5%, about 5.1% to about 5.6%, about 5.1% to about 5.7%, about 5.1% to about 5.8%, about 5.1% to about 5.9%, about 5.1% to about 6%, about 5.2% to about 5.3%, about 5.2% to about 5.4%, about 5.2% to about 5.5%, about 5.2% to about 5.6%, about 5.2% to about 5.7%, about 5.2% to about 5.8%, about 5.2% to about 5.9%, about 5.2% to about 6%, about 5.3% to about 5.4%, about 5.3% to about 5.5%, about 5.3% to about 5.6%, about 5.3% to about 5.7%, about 5.3% to about 5.8%, about 5.3% to about 5.9%, about 5.3% to about 6%, about 5.4% to about 5.5%, about 5.4% to about 5.6%, about 5.4% to about 5.7%, about 5.4% to about 5.8%, about 5.4% to about 5.9%, about 5.4% to about 6%, about 5.5% to about 5.6%, about 5.5% to about 5.7%, about 5.5% to about 5.8%, about 5.5% to about 5.9%, about 5.5% to about 6%, about 5.6% to about 5.7%, about 5.6% to about 5.8%, about 5.6% to about 5.9%, about 5.6% to about 6%, about 5.7% to about 5.8%, about 5.7% to about 5.9%, about 5.7% to about 6%, about 5.8% to about 5.9%, about 5.8% to about 6%, or about 5.9% to about 6%. The weight percentage may be about 5%, about 5.1%, about 5.2%, about 5.3%, about 5.4%, about 5.5%, about 5.6%, about 5.7%, about 5.8%, about 5.9%, or about 6%. The weight percentage may be at least about 5%, about 5.1%, about 5.2%, about 5.3%, about 5.4%, about 5.5%, about 5.6%, about 5.7%, about 5.8%, or about 5.9%. The weight percentage may be at most about 5.1%, about 5.2%, about 5.3%, about 5.4%, about 5.5%, about 5.6%, about 5.7%, about 5.8%, about 5.9%, or about 6%. Further, the weight percentage may be about 5% to about 5.1%. The weight percentage may be about 5% to about 5.01%, about 5% to about 5.02%, about 5% to about 5.03%, about 5% to about 5.04%, about 5% to about 5.05%, about 5% to about 5.06%, about 5% to about 5.07%, about 5% to about 5.08%, about 5% to about 5.09%, about 5% to about 5.1%, about 5.01% to about 5.02%, about 5.01% to about 5.03%, about 5.01% to about 5.04%, about 5.01% to about 5.05%, about 5.01% to about 5.06%, about 5.01% to about 5.07%, about 5.01% to about 5.08%, about 5.01% to about 5.09%, about 5.01% to about 5.1%, about 5.02% to about 5.03%, about 5.02% to about 5.04%, about 5.02% to about 5.05%, about 5.02% to about 5.06%, about 5.02% to about 5.07%, about 5.02% to about 5.08%, about 5.02% to about 5.09%, about 5.02% to about 5.1%, about 5.03% to about 5.04%, about 5.03% to about 5.05%, about 5.03% to about 5.06%, about 5.03% to about 5.07%, about 5.03% to about 5.08%, about 5.03% to about 5.09%, about 5.03% to about 5.1%, about 5.04% to about 5.05%, about 5.04% to about 5.06%, about 5.04% to about 5.07%, about 5.04% to about 5.08%, about 5.04% to about 5.09%, about 5.04% to about 5.1%, about 5.05% to about 5.06%, about 5.05% to about 5.07%, about 5.05% to about 5.08%, about 5.05% to about 5.09%, about 5.05% to about 5.1%, about 5.06% to about 5.07%, about 5.06% to about 5.08%, about 5.06% to about 5.09%, about 5.06% to about 5.1%, about 5.07% to about 5.08%, about 5.07% to about 5.09%, about 5.07% to about 5.1%, about 5.08% to about 5.09%, about 5.08% to about 5.1%, or about 5.09% to about 5.1%. The weight percentage may be about 5%, about 5.01%, about 5.02%, about 5.03%, about 5.04%, about 5.05%, about 5.06%, about 5.07%, about 5.08%, about 5.09%, or about 5.1%. The weight percentage may be at least about 5%, about 5.01%, about 5.02%, about 5.03%, about 5.04%, about 5.05%, about 5.06%, about 5.07%, about 5.08%, or about 5.09%. The weight percentage may be at most about 5.01%, about 5.02%, about 5.03%, about 5.04%, about 5.05%, about 5.06%, about 5.07%, about 5.08%, about 5.09%, or about 5.1%.

The other ranges (e.g., 3-15% for the phospholipids; 0.1-10% for the Long-chain fatty acids; 30-90% for the water; 0.05%-5% for the PDE5 inhibitor; 0.5-5% for the penetration enhancer; and 0.5-10% for the emulsifier) recited in the above table include similar ranges and subranges and values within ranges. The present disclosure contemplates all similar ranges and subranges and values within ranges for each ingredient included in a formulation.

In some cases, the medicament is in an amount from about 0.001% to about 0.01% w/w of the formulation, in an amount from about 0.011% to about 0.1% w/w of the formulation, in an amount from about 0.11% to about 1.0% w/w of the formulation, in an amount from about 1% to about 10% w/w of the formulation, in an amount from about 11% to about 20% w/w of the formulation, or in an amount from about 21% to about 30% w/w of the formulation. The present disclosure contemplates all similar ranges and subranges and values within ranges for the medicament or medicaments included in a formulation

The medicament is selected from Table 1. The amount of the medicament in is as recited in Table 1.

In some cases, more than one medicament is included in a transdermal formulation. In these cases, the first and the second medicament are as disclosed in Table 1 and the amounts of the first and the second medicament is as recited in Table 1.

### Example 35 Combination Therapies

Provided herein is a method for treating a disease or disorder or reducing a symptom thereof, in which the method comprises steps of administering to a subject in need thereof a herein disclosed transdermal formulation (e.g., as disclosed in Example 34 and elsewhere herein) and administering to the subject in need thereof a composition comprising an one or more medicaments selected from Table 1.

The transdermal formulation may be administered before, contemporary with, or after the composition is administered.

The amount of the one or more medicaments is the effective dose of the medicament as described in Table 1.

The composition is administered by the standard route for the one or more medicament, e.g., the standard route is oral, topical, enteral, parenteral, by intravenous injection or infusion, by intraperitoneal injection, by intramuscular injection, or by subcutaneous injection.

The composition may be a liquid, a suspension, a gel, a geltab, a semisolid, a tablet, a sachet, a lozenge, a pill, or a capsule.

1. A formulation for transdermal delivery of a medicament through the skin of a subject, the formulation comprising a therapeutically effective amount of a medicament and a penetrant portion,
wherein the penetrant portion comprises: a phospholipid, a fatty acid ester formed from a low molecular weight alcohol, and a long-chain fatty acids, and, optionally, one or more of a viscosity-improving agent, a penetration enhancer, and an emulsifier;
wherein the formulation lacks a natural lecithin.

2. The formulation of claim 1, wherein the phospholipid is selected from phosphatidylcholine, hydrogenated phosphatidylcholine, phosphatidylserine, phosphatidylethanolamine, phosphatidylinositol, inositol phosphatide, and sphingomyelin.

3-4. (canceled)

5. The formulation of claim 1, wherein the phospholipid is in an amount from about 3% to about 15% w/w of the formulation.

6-7. (canceled)

8. The formulation of claim 1, wherein the fatty acid ester formed from a low molecular weight alcohol is selected from isopropyl palmitate, isopropyl myristate, isopropyl linoleate, isopropyl oleate, ethyl laurate, and ethyl myristate.

9-10. (canceled)

11. The formulation of claim 1, wherein the fatty acid ester formed from a low molecular weight alcohol is in an amount from about 5% to about 20% w/w of the formulation.

12. The formulation of claim 1, wherein the long-chain fatty acid is selected from a linoleic, oleic, stearic acid, linolenic, palmitic, arachidonic, palmitoleic, myristic, eicosenoic, benehic, euricic, and lignoceric acid or is obtained from safflower oil or almond oil.

13-16. (canceled)

17. The formulation of claim 1, wherein the long-chain fatty acid is in an amount from about 0.10% to about 10% w/w of the formulation.

18. The formulation of claim 1, wherein the penetrant portion comprises two or more long-chain fatty acids.

19. The formulation of claim 1, wherein the penetrant portion comprises a viscosity-improving agent which is a poloxamer selected from poloxamer 407, poloxamer 188, poloxamer 184, and poloxamer 124.

20-25. (canceled)

26. The formulation of claim 1, wherein the viscosity-improving agent is in an amount from about 5% to about 20% w/w of the formulation.

27. The formulation of claim 1, wherein the penetrant portion comprises a penetration enhancer selected from benzyl alcohol, ethanol, propylene glycol, polyethylene glycol, limonene, menthol, borneol, and camphor.

28-33. (canceled)

34. The formulation of a claim 1, wherein the penetration enhancer is in an amount from about 0.5% to about 5% w/w of the formulation.

35. (canceled)

36. The formulation of claim 1, wherein the penetrant portion comprises an emulsifier selected from polyglyceryl-4-laurate, polyglyceryl-4-oleate, span 60, cetyl alcohol, and polyglyceryl-3-oleate.

37-38. (canceled)

39. The formulation of claim 1, wherein the emulsifier is in an amount from about 0.5 to about 10% w/w of the formulation.

40-43. (canceled)

44. The formulation of claim 1,
wherein the penetrant portion comprises: a phospholipid, a fatty acid ester formed from a low molecular weight alcohol, and a long-chain fatty acids, and one or more of a viscosity-improving agent, a penetration enhancer, and an emulsifier,
wherein phosphatidylcholine, hydrogenated phosphatidylcholine, phosphatidylserine, phosphatidylethanolamine, phosphatidylinositol, inositol phosphatide, or sphingomyelin is the phospholipid;
wherein isopropyl palmitate, isopropyl myristate, isopropyl linoleate, isopropyl oleate, ethyl laurate, or ethyl myristate is the fatty acid ester formed from a low molecular weight alcohol; and
wherein a linoleic, oleic, stearic acid, linolenic, palmitic, arachidonic, palmitoleic, myristic, eicosenoic, benehic, euricic, or lignoceric acid is the long-chain fatty acid or the long-chain fatty acid is obtained from safflower oil or almond oil;
wherein polyglyceryl-4-laurate, polyglyceryl-4-oleate, span 60, cetyl alcohol, or polyglyceryl-3-oleate is the emulsifier;
wherein a poloxamer (e.g., poloxamer 407, poloxamer 188, poloxamer 184, and poloxamer 124) or sodium lauryl sulfate is the viscosity-improving agent; and
wherein benzyl alcohol, ethanol, propylene glycol, polyethylene glycol, limonene, menthol, borneol, or camphor is the penetration enhancer.

45-46. (canceled)

47. The formulation of claim 1, wherein the penetrant portion comprises water in an amount from about 50% to about 80% w/w of the formulation.

48. (canceled)

49. The formulation of claim 1, wherein the medicament is in an amount from about 0.0010% to about 30% w/w of the formulation.

50-64. (canceled)

65. The formulation of claim 1, wherein the medicament is at least one of (3S,4S)-8-(6-amino-5-((2-amino-3-chloropyridin-4-yl)thio)pyrazin-2-yl)-3-methyl-2-oxa-8-azaspiro[4.5]decan-4-amine; (S)-3-amino-6-methoxy-N-(3,3,3-trifluoro-2-hydroxy-2-methylpropyl)-5-(trifluoromethyl)picolinamide; 2-fluoro-N-methyl-4-[7-quinolin-6-yl-methyl)-imidazo[1,2-B][1,2,4]triazin-2yl]benzamide; 4-(3-amino-6-((1S,3S,4S)-3-fluoro-4-hydroxycyclohexyl)pyrazin-2-yl)-N—((S)-1-(3-bromo-5-fluorophenyl)-2-(methylamino)ethyl)-2-fluorobenzamide; 4-(7-Hydroxy-2-isopropyl-4-oxo-4H-quinazolin-3-yl)-benzonitrile; Abacavir; Abiraterone Acetate; abiratone acetate; abobotulinum toxin A; Acalabrutinib; Acarbose; Acetaminophen; Acetazolamide; Acetylsalicylic Acid; acitretin; acyclovir; adalimumab; adapalene; adapalene; Afoxolaner; agalsidase beta; Albendazole; Alectinib; Alectinib; alendronate sodium; alglucosidase alfa; alitretinoin; Alogliptin; alpelisib; Aluminum Hydroxide; Ambrisentan; Amiodarone; Amitriptyline; Amlodipine; Amlodipine; Amlodipine besylate; amlodopine besylate; amorolfine hydrochloride; Amoxicillin; Amphotericin B; Amphotericin B liposome; ampicillin; Amprenavir; antihemophilic factor, Fc fusion protein; apalutamide; Apatinib; apixaban; apremilast; Aprepitant; Aripiprazole; Artemether; aspirin; Atazanavir; atenolol; Atomoxetine; Atorvastatin; Atorvastatin; Avapritinib; Axitinib (Inlyta); Azacitidine; Azathioprine; azelaic acid; Azilsartan medoxomil; azithromycin; baclofen; baricitinib; basiliximab; Batimastat (BB-94); becaplermin; Belumosudil; Bendamustine; benzoyl peroxide; benzoyl peroxide; betamethasone dipropionate; betamethasone valerate; bexarotene; Bictegravir; Bimatoprost; binimetinib (Mektovi); Bortezomib; Bortezomib (Velcade); Bosentan; Bosutinib (Bosulif); botulinum toxin A; branched chain amino acids; Brexpiprazole; Brimonidine; brimonidine tartrate; brodalumab; buproprion; Cabazitaxel; Cabozantinib; Cabozantinib (Cometriz); Calcifediol; calcipotriene; Calcipotriene; Calcipotriol; calcitonin; calcitriol; calcium carbonate; Canagliflozin; canakinumab; Capecitabine; Capmatinib; capsaicin; Carbamazepine; Carbidopa; Carfilzomib; Carfilzomib (Kyprolis); Cariprazine; carvedilol; CD-12681; CEE-321; cefazolin; cefepime; Cefoperazone; cefotaxime sodium; celecoxib; celecoxib; cemiplimab; Ceritinib; Chlorothiazide; Chlorpromazine; Chlorthalidone; cinacalcet; ciprofloxacin; Cisapride; citalopram; citric acid; clarithromycin; Clavulanate; Clindamycin phosphate; Clobazam; clobetasol propionate; Clofazimine; clomiphene; clonidine; Clopidogrel; clopidogrel bisulfate; cobicistat; Cobicistat; Colchocine; Colistin; Corticotropin; crisaborole; crizanlizumab; Crizotinib (Xalkori); cyclobenzaprine; cyclosporine; Dabigatran Etexilate; Dabrafenib; Dabrafenib; dabrafenib; dabrafenib; dacomitinib (Vizimpro); dalbavancin; dalfopristin; Danazol; Dapagliflozin; Dapsone; daptomycin; Darunavir; Dasatinib; decitabine; defatted Peanut (Arachis hypogaea) Flour; deferasirox; delgocitinib; deoxycholic acid; Desipramine; desonide; Dexlansoprazole; Dexmethylphenidate; dextroamphetamine/amphetamine salts; Diclofenac; diclofenac sodium; diflucortolone; Diflucortolone; Difunisal; Digoxin; Diloxanide; Dimethyl Fumarate; diphenhydramine hydrochloride; diphtheria vaccine; docetaxel; Dolutegravir; Dolutegravir; Donepezil; Doxercalciferol; doxycycline; doxycycline ER; Doxycycline Hyclate (B); Doxycycline Monohydrate (A); Dronabinol; Dronedarone; Drospirenone; Dukoral/ShanChol cholera vaccine; Duloxetine; dupilumab; durvalumab; Dutasteride; Duvelisib; ecallantide; Edoxaban; Efavirenz; Efavirenz; Elafibranor; elagolix sodium; Elbasvir; Eletriptan; Eltrombopag; Elvitegravir; Empagliflozin; Emtricitabine; emtricitabine; Emtricitabine; Emtricitabine; Emtricitabine; Emtricitabine; Emtricitabine; Emtricitabine; encorafenib (Braftovi); enfuviritide; enoxaparin sodium; Entecavir; entrectinib; Enzalutamide; Epinephrine; erdafitinib; erenumab; Eribulin; Erlotinib; Erlotinib HCl (Tarceva); Ertapenem; Erythromycin; escitalopram; esketamine (ketamine); Esomeprazole; esopremazole; estradiol; Estrogens—conjugated; etanercept; Ethinyl Estradiol; Ethinyl Estradiol; Ethinyl Estradiol; Etonogestrel; Etonogestrel; Etravirine; etretinate; Everolimus; Everolimus (Afinitor); Everolimus (Votubia, Zortress/Certican); Ezetimibe; Ezetimibe; Ezetimibe; Famotidine; fasinumab; Febuxostat; Fedratinib; Fenofibrate; Ferric Carboxymaltose; fexofenadine hydrochloride; filgotinib; filgrastim; fingolimod; fluocinolone acetonide; fluocinolone acetonide; fluoxetine; Flurbiprofen; Folic acid; Forskolin; Fostamatinib; Fulvestrant; furosemide; fusidic acid; fusidic acid; gabapentin; Ganetespib; Gefitinib (Iressa); Gilteritinib; glasdegib (Daurismo) or glasdegib maleate; Glecaprevir; Glibenclamide; Glimepiride; glipizide; glucagon; glucosamine; glutaric anhydride; glycopyrrolate; Goserelin; goserelin LA; granisetron; Grazoprevir; Griseofulvin; halobetasol propionate; Haloperidol; heparin sodium; heplisav-B vaccine; hyaluronic acid; Hydrochlorothiazide; Hydrochlorothiazide; Hydrochlorothiazide; Hydrochlorothiazide; hydrocortisone; hydrocortisone acetate; hydroquinone; hydroxychloroquine; Hydroxyprogesterone; Ibrutinib; Ibrutinib (Imbruvica); ibuprofen; icatibant acetate; Idelalisib; Imatinib; Imatinib (Gleevec); imatinib mesylate; imiglucerase; inclisiran; Indinavir; Indomethacin; infliximab; ingenol mebutate; inotersen; insulin A; insuline glargine; interferon beta-lb; Iopanoic acid; Irbesartan; isoconazole; isoconazole; Isotretinoin; Itraconazole; Ivacaftor; Ivacaftor; ivermectin; Ixazomib; ketoconazole; ketones; ketoprofen; Keyruvia; Lacosamide; lamictal; Lamivudine; Lansoprazole; Lapatinib; Lapatinib (Tykerb); larotrectinib; Ledipasvir; leflunomide; Lenalidomide; Lenvatinib; LEO-138559; LEO-152020; Leuprolide; Levetiracetam; levodopa; Levodopa/Benserazide; Levonorgestrel; Levothyroxine; Linagliptin; linezolid; lisinopril; L-lysine free base; lofexidine; Lopinavir; Loratadine; lorlatinib (Lorviqua); Losartan; Lovastatin; Lubiprostone; Lumacaftor/Ivacaftor; Lumefantrine; Lurasidone; Luspatercept; lymecycline; Macitentan; magnesium; magnesium lactate; Marimastat (BB-2516); Marizomib (NPI-0052); Mebendazole; Mefloquine; melatonin; meloxicam; memantine hydrochloride; meningococcal[serotype b] vaccine; Mesalazine; Metformin; Metformin; metformin; Metformin; methotrexate; methyl aminolevulinate hydrochloride; Methylphenidate; methylprednisolone; Metoprolol succinate; metronidazole; Midostaurin; minoxidil; Mirabegron; mizolastine MR; Montelukast; Mycophenolate Mofetil; N-(3-(2-(2-hydroxyethoxy)-6-morpholinopyridin-4-yl)-4-methylphenyl)-2-(trifluoromethyl)-isonicotinamide; N-(3-(6-Amino-5-(2-(N-methylacrylamido)ethoxy)pyrimidin-4-yl)-5-fluoro-2-methylphenyl)-4-cyclopropyl-2-fluorobenzamide; N-[4-(Chlorodifluoromethoxy)phenyl]-6-[(3R)-3-hydroxpyrrolidin-1-yl]-5-(1H-pyrazol-5-yl)-pyridine-3-carboxamide hydrochloride; Nalidixic acid (Quinolone antibiotic); naloxone; Naproxen; Navitoclax (ABT-263); Nebivolol; Nelfinavir; nemolizumab; neomycin; Neovastat (AE-941); Neratinib—Free Base; Neratinib (Nerlynx); Neratinib Maleate; Nevirapine; Niclosamide; Nifedipine; nilotinib; Nilotinib; Nilotinib (Tasigna); Nintedanib; niratimib; Nitric Oxide; Nitrofurantoin; Norethindrone Acetate; nusinersen; NVP-AUY922; Nystatin; Obatoclax (GX15-070); octreotide acetate; ofatumumab; Ofloxacin; Olanzapine; olaparib; Olmesartan Medoxomil; olumacostat; omalizumab; Oseltamivir; Osimertinib; osimertinib mesylate; Oxaprozin; Ozanimod; Paclitaxel; Paclitaxel Protein Bound; Palbociclib (Ibrance); Paliperidone Palmitate; Pantoprazole; parathyroid hormone; Paricalcitol; patisiran; pazopanib; Pazopanib (Votrient); pazopanib hydrochloride; Peficitinib; pegfilgrastim; Pembrolizumab (Keytruda); Pemetrexed; Pemigatinib; Perifosine; Pexidartinib; Phenazopyridine; Phenytoin; Pibrentasvir; Piperacillin; Pirfenidone; Piroxicam; Pomalidomide; Ponatinib; Ponatinib (lclusig); Posaconazole; Pralsetinib; pravastatin; prednicarbate; prednisone; pregabalin; pregabalin; Prinomastat (AG-3340); Progesterone; propanolol; protamine sulfate; psilocybin; Pyrantel; Pyrimethamine; Quetiapine; Quinupristin; Raloxifene; Raltegravir; ranibizumab; ranitidine; Ranolazine; Rebimastat (BMS-275291); Regorafenib; Regorafenib (Stivarga); relugolix (Orgovyx); remibrutinib; resiquimod; retinal; Retinol; ribociclib; ribociclib succinate; Rifampin; Rilpivirine; Rilpivirine; Rilpivirine; Rimegepant; Riociguat; Risdiplam; Risperidone; Ritonavir; Rivaroxaban; Rosuvastatin; rosuvastatin calcium; Rotigotine; roxadustat; rucaparib camsylate (Rubraca); ruxolitinib; Ruxolitinib (Jafaki); Sacubitril, valsartan; salicylic acid; Sapropterin; Saquinavir; sarilumab; Saxagliptin, Metformin; secukinumab; Selexipag; selinexor; Selpercatinib; selpercatinib (LOXO-292); Selumetinib; semaglutide; sertraline; Sevoflurane; sildenafil; Sildenafil Citrate; Simvastatin; siponimod; Siremadlin; Sirolimus; Sitagliptin; Sitagliptin phosphate monohydrate; Sodium bicarbonate or sodium carbonate; sodium deoxycholate; sodium nitrate; Sofosbuvir; Sofosbuvir; Sofosbuvir; Solifenacin; somatropin; sonidegib phosphate; Sorafenib; Sorafenib (Nexavar); spartalizumab; Spironolactone; sufentanil citrate; Sugammadex; sulbactam; Sulbactam; Sulfadiazine; Sulfamethoxazole; sulfasalazine; sumatriptan; Sunitinib; Sunitinib; Sunitinib (sutent); sunitinib malate (Sutent); Tacrolimus; TAF; TAF; TAF; TAF; talazoparib—Talzenna); Talinolol; Tamoxifen; Tamsulosin; tazarotene; tazobactam; TDF; TDF; Temsirolimus (CCl-779, Torisel); Teneligliptin; tenofovir alafenamide; Tenofovir Disoproxil Fumarate; Tepotinib; terbinafine; Terfenadine; Teriflunomide; Testosterone; Tezacaftor; THC; ticagrelor; tigecycline; Timolol; timolol maleate; Tipranavir; tisagenlecleucel; Tisagenlecleucel (Kymriah); Tivozanib; tofacitinib citrate; Tolvaptan; tralokinumab; tramadol; Trametinib; Trametinib; trametinib; Trametinib; Trametinib (Mekinist); tranexamic acid; Travoprost; trazodone; tretinoin; tretinoin; triancinolone; Triclabendazole; trifarotene; Trimethoprim; Triptorelin; tris; Tucatinib; ubrogepant; Umbralisib; upadacitinib; valproic acid; Valsartan; valsartan; Valsartan; valsartan; vancomycin; Vancomycin Hydrochloride (A); Vancomycin Hydrochloride (B); Vandetanib; Vandetenib (Caprelsa); Varenicline; Vasopressin; Velpatasvir; Velpatasvir; Vemurafenib; Venetoclax; Verapamil hydrochloride; Vilazodone; Vildagliptin; vismodegib; vitamin B6; vitamin D; Vonoprazan; Voriconazole; Vortioxetine; Voxilaprevir; Warfarin Sodium; Zanubrutinib; Zinc; zoledronic acid; and Zolpidem.

66. (canceled)

67. The transdermal delivery formulation of claim 65, further comprising an at least second medicament, wherein the first and the at least second medicament are selected from the group consisting of Abacavir and Dolutegravir and Lamivudine; adapalene and benzoyl peroxide; Amlodipine and Valsartan; amlodopine besylate and valsartan; Amoxicillin and Clavulanate; ampicillin and sulbactam; Artemether and Lumefantrine; betamethasone valerate and fusidic acid; Bictegravir and Emtricitabine and TAF; Bimatoprost and timolol maleate; Brimonidine and Timolol; Calcipotriene and betamethasone dipropionate; Canagliflozin and Metformin; Carbidopa and levodopa; Cefoperazone and Sulbactam; Dabrafenib and Trametinib; Darunavir and cobicistat and emtricitabine and tenofovir alafenamide; Diflucortolone and isoconazole; Drospirenone and Ethinyl Estradiol; Efavirenz and Emtricitabine and TDF; Elbasvir and Grazoprevir; Elvitegravir and Cobicistat and Emtricitabine and TAF; Emtricitabine and Rilpivirine and TAF; Emtricitabine and TAF; Emtricitabine and Tenofovir Disoproxil Fumarate; Ethinyl Estradiol and Etonogestrel; Ezetimibe and Atorvastatin; Ezetimibe and Simvastatin; fluocinolone acetonide and hydroquinone and tretinoin; fusidic acid and hydrocortisone acetate; Glecaprevir and Pibrentasvir; Hydrochlorothiazide and Valsartan; Irbesartan and Hydrochlorothiazide; Ledipasvir and Sofosbuvir; Linagliptin and Metformin; Losartan and Hydrochlorothiazide; Metformin and Sitagliptin; Norethindrone Acetate and Ethinyl Estradiol; Piperacillin and tazobactam; pregabalin and celecoxib; Quinupristin and dalfopristin; Rilpivirine and Emtricitabine and TDF; Sofosbuvir and Velpatasvir; Sofosbuvir and Velpatasvir and Voxilaprevir; Tezacaftor and Ivacaftor; trametinib and dabrafenib; Trametinib and dabrafenib; and Zinc and magnesium and vitamin B6.

67. (canceled)

68. The formulation of claim 1, wherein transdermal delivery provides systemic administration of the medicament.

69. A method for transdermally delivering at least one medicament, the method comprising a step of applying to the skin of a subject an effective amount of the formulation of claim 1.

70-77. (canceled)

78. The formulation of claim 1, wherein the formulation lacks a natural plant derived lecithin or lacks a natural animal derived lecithin.
